(19) United States
(12) Patent Application Publication
(10) Pub. No.: US 2003/0003601 A1
(43) Pub. Date: Jan. 2, 2003

(54) Biochemical analysis system and biochemical analysis unit

(75) Inventors: Masato Some, Kaisei-machi (JP); Katsuaki Muraishi, Kaisei-machi (JP); Hitoshi Shimizu, Kaisei-machi (JP); Nobuhiko Ogura, Kaisei-machi (JP); Tohru Tsuchiya, Kaisei-machi (JP); Hirohiko Tsuzuki, Kaisei-machi (JP)

Correspondence Address: SUGHRUE MION, PLLC, 2100 Pennsylvania Avenue, NW, Washington, DC, 20037-3213, US

(73) Assignee: FUJI PHOTO FILM CO., LTD.

(21) Appl. No.: 10/184,982

(22) Filed: Jul. 1, 2002

(30) Foreign Application Priority Data
Jul. 2, 2001 (JP) 201103/2001
Jul. 31, 2001 (JP) 231701/2001
Sep. 28, 2001 (JP) 303175/2001
Sep. 28, 2001 (JP) 303325/2001

(51) Int. Cl. G01N 33/543; C12M 1/34

(52) U.S. Cl. 436/518; 435/287.2

(57) ABSTRACT

A biochemical analysis unit is obtained, which comprises a base plate having spot-shaped regions and has identification information, specific binding substances having been fixed respectively to the spot-shaped regions. A labeled organism-originating substance having identification information is subjected to specific binding to the specific binding substances. A label signal corresponding to a position of the spot-shaped region having thus been labeled selectively is read out to form data for a biochemical analysis. The identification information of the biochemical analysis unit and the identification information of the organism-originating substance are acquired and fed into an analysis apparatus, such that the correspondence relationship between the two pieces of the identification information is clear.

### BACKGROUND OF THE INVENTION

1. Field of the Invention

This invention relates to a biochemical analysis system. This invention particularly relates to a biochemical analysis system, wherein a structure or characteristics of an organism-originating substance are analyzed by use of a biochemical analysis unit, which comprises a base plate and a plurality of spot-shaped regions located at different positions on or in the base plate, each of multiple kinds of specific binding substances, which are capable of specifically binding to organism-originating substances and whose structures or characteristics are known, having been fixed to one of the spot-shaped regions. This invention also relates to a biochemical analysis unit, a method and a system for managing a biochemical analysis unit, and a method and a system for managing a biochemical analysis medium.

2. Description of the Related Art

Recently, as systems for analyzing structures or characteristics of organism-originating substances, micro array analysis systems utilizing micro arrays have been proposed. With the micro array analysis systems, a micro array is prepared by, for example, spotting multiple kinds of specific binding substances, which are capable of specifically binding to organism-originating substances and whose base sequences, base lengths, compositions, and the like, are known, onto different positions on a surface of a carrier, such as a slide glass plate or a membrane filter, by use of a spotting apparatus and thereby forming a plurality of independent spots on the surface of the carrier. Examples of the specific binding substances include hormones, tumor markers, enzymes, antibodies, antigens, abzymes, other proteins, nucleic acids, cDNA's, DNA's, and RNA's. Thereafter, an organism-originating substance, which has been sampled from an organism through extraction, isolation, or the like, or has been subjected to chemical treatment, chemical modification treatment, or the like, and which has been labeled with a fluorescent labeling substance, such as a fluorescent substance or a fluoro chrome, is subjected to hybridization with the specific binding substances, which have been fixed to the spots on the micro array. The organism-originating substance is thus specifically bound to one of the specific binding substances on the micro array. Examples of the organism-originating substances include hormones, tumor markers, enzymes, antibodies, antigens, abzymes, other proteins, nucleic acids, cDNA's, DNA's, and mRNA's. Excitation light is then irradiated to the micro array, and the fluorescence produced by the labeling substance, such as the fluorescent substance or the fluoro chrome, with which the organism-originating substance has been labeled, is photoelectrically detected. In accordance with the results of the photoelectric detection, the organism-originating substance is analyzed. The micro array analysis systems, wherein the spots of the multiple kinds of the specific binding substances are formed at a high density at different positions on the surface of the carrier, such as the slide glass plate or the membrane filter, and wherein the organism-originating substance, which has been labeled with the labeling substance, is subjected to the hybridization with the specific binding substances, has the advantages in that the organism-originating substance is capable of being analyzed quickly.

Besides the micro array analysis systems utilizing the fluorescent substance as the labeling substance, micro array analysis systems utilizing a radioactive labeling substance have also been proposed. With the micro array analysis systems utilizing the radioactive labeling substance, a micro array is prepared by, for example, spotting multiple kinds of specific binding substances, which are capable of specifically binding to organism-originating substances and whose base sequences, base lengths, compositions, and the like, are known, onto different positions on a surface of a carrier, such as a membrane filter, by use of a spotting apparatus and thereby forming a plurality of independent spots on the surface of the carrier. Examples of the specific binding substances include hormones, tumor markers, enzymes, antibodies, antigens, abzymes, other proteins, nucleic acids, cDNA's, DNA's, and RNA's. Thereafter, an organism-originating substance, which has been sampled from an organism through extraction, isolation, or the like, or has been subjected to chemical treatment, chemical modification treatment, or the like, and which has been labeled with a radioactive labeling substance, is subjected to hybridization with the specific binding substances, which have been fixed to the spots on the micro array. The organism-originating substance is thus specifically bound to one of the specific binding substances on the micro array. Examples of the organism-originating substances include hormones, tumor markers, enzymes, antibodies, antigens, abzymes, other proteins, nucleic acids, cDNA's, DNA's, and mRNA's. The micro array is then brought into close contact with a stimulable phosphor sheet comprising a stimulable phosphor layer containing a stimulable phosphor, and the stimulable phosphor layer is thereby exposed to radiation radiated out from the radioactive labeling substance, with which the organism-originating substance has been labeled. Thereafter, the stimulable phosphor layer is exposed to stimulating rays, which cause the stimulable phosphor layer to emit light in proportion to the amount of energy stored on the stimulable phosphor layer during its exposure to the radiation. The light emitted by the stimulable phosphor layer is photoelectrically detected, and an image signal for a biochemical analysis is thereby obtained. In accordance with the thus obtained image signal for a biochemical analysis, the organism-originating substance is analyzed.

Further, micro array analysis systems utilizing a labeling substance, which is capable of producing chemiluminescence when being brought into contact with a chemiluminescence substrate, have been proposed. With the proposed micro array analysis systems, a micro array is prepared by forming spot-shaped regions of multiple kinds of specific binding substances at a high density at different positions on a surface of a carrier. Thereafter, an organism-originating substance, which has been labeled with the labeling substance, which is capable of producing the chemiluminescence when being brought into contact with a chemiluminescence substrate, is subjected to specific binding with the specific binding substances at the spot-shaped regions on the micro array. The organism-originating substance is thus specifically bound to one of the specific binding substances on the micro array. The micro array is then brought into contact with the chemiluminescence substrate, and the chemiluminescence having wavelengths falling within the visible light wavelength range is thereby emitted by the labeling substance, with which the organism-originating substance has been labeled. The chemiluminescence is photoelectrically detected, and a digital image signal is thereby obtained. The thus obtained digital image signal is then subjected to image processing, and the image signal having been obtained from the image processing is utilized for reproducing a chemiluminescence image on displaying means, such as a cathode ray tube (CRT) display device, or a recording material, such as photographic film. In accordance with the reproduced chemiluminescence image, information concerning the organism-originating substance, such as genetic information, is obtained.

Besides the micro array analysis systems utilizing the radioactive labeling substance, the fluorescent labeling substance, or the labeling substance, which is capable of producing the chemiluminescence when being brought into contact with a chemiluminescence substrate, there have also been proposed micro array analysis systems utilizing all of the radioactive labeling substance, the fluorescent labeling substance, and the labeling substance, which is capable of producing the chemiluminescence when being brought into contact with a chemiluminescence substrate. There have further been proposed micro array analysis systems utilizing a combination of two kinds of the labeling substances among the radioactive labeling substance, the fluorescent labeling substance, and the labeling substance, which is capable of producing the chemiluminescence when being brought into contact with a chemiluminescence substrate.

However, the micro arrays before being subjected to the binding to organism-originating substances vary in kind of each of specific binding substances which form the spots on the micro arrays, spot location pattern, and the like. (The micro array comprising the carrier, such as the support, and a plurality of spots, which are located at different positions on the carrier and to which multiple kinds of specific binding substances have respectively been fixed, will hereinbelow be referred to as the biochemical analysis unit.) Therefore, in order for the structure and the characteristics of the organism-originating substance acting as the object of the analysis to be analyzed, it is necessary to find which biochemical analysis unit was utilized with respect to the organism-originating substance. Accordingly, in the micro array analysis systems described above, the correspondence relationship between the organism-originating substance acting as the object of the analysis and the biochemical analysis unit utilized with respect to the organism-originating substance must be managed. For such purposes, the operator of the analysis has had to memorize which organism-originating substance was taken as the object of the analysis and which biochemical analysis unit was utilized with respect to the organism-originating substance. Also, when an ultimate analysis is to be performed, the operator of the analysis has had to perform processing for specifying the correspondence relationship between the organism-originating substance and the biochemical analysis unit in accordance with the memory of the operator of the analysis, a memorandum made by the operator of the analysis, or the like. Therefore, there is the risk that an error will occur due to a mistake made in memorization or inputting, and correct results of analysis cannot be obtained. In the medical field for making tumor examinations, and the like, incorrect results of analysis lead to fatal results. Therefore, it is important that artificial mistakes be avoided, and the correspondence relationship between the organism-originating substance acting as the object of the examination and the biochemical analysis unit utilized with respect to the organism-originating substance be specified accurately.

Also, for the same reasons, in the analysis systems utilizing the stimulable phosphor sheets, it is necessary that, besides the correspondence relationship between the organism-originating substance and the biochemical analysis unit utilized with respect to the organism-originating substance, the correspondence relationship between the biochemical analysis unit and the stimulable phosphor sheet utilized with respect to the biochemical analysis unit be specified accurately.

Further, in some cases, when a biochemical analysis is to be made, a biochemical analysis unit, such as a commercially available biochemical analysis unit, wherein the spots have already been formed with the specific binding substances on the base plate, may be utilized. In other cases, only the base plate, such as the membrane, is purchased, the specific binding substances, which are selected in accordance with the kind of the organism-originating substance, the purpose of the analysis, or the like, are spotted onto the base plate by use of a spotter apparatus, or the like, and the biochemical analysis unit, on which the spots have thus been formed with the specific binding substances, is utilized. In cases where the biochemical analysis unit, on which the spots have been formed, is utilized, it often occurs that the biochemical analysis unit having been utilized one time for the analysis is washed, and only the base plate is reused. In such cases, in order for correct results of analysis to be obtained, it is necessary that the correspondence relationship between the prepared biochemical analysis unit and spot information (i.e., the information representing the kinds of the specific binding substances, the positions, to which the specific binding substances have respectively been spotted, and the like) with respect to the prepared biochemical analysis unit be specified accurately.

### SUMMARY OF THE INVENTION

The primary object of the present invention is to provide a biochemical analysis system, wherein artificial mistakes are avoided, and correct results of analysis are capable of being obtained.

Another object of the present invention is to provide a biochemical analysis unit, which is constituted such that data inherent to a biochemical analysis unit, such as a micro array or a macro array, is capable of being managed reliably, and reliability of a biochemical analysis is capable of being enhanced markedly.

A further object of the present invention is to provide a method of managing a biochemical analysis unit, wherein a biochemical analysis unit having been used for a biochemical analysis, in which an organism-originating substance having been labeled with a radioactive labeling substance is subjected to hybridization with specific binding substances contained in the biochemical analysis unit, is capable of being managed reliably until a level of radioactivity of the radioactive labeling substance attenuates to a level equal to at most a predetermined level.

A still further object of the present invention is to provide a system for carrying out the method of managing a biochemical analysis unit.

Another object of the present invention is to provide a method of managing a biochemical analysis medium, wherein a biochemical analysis unit having been used for a biochemical analysis, in which an organism-originating substance having been labeled with a radioactive labeling substance is subjected to hybridization with specific binding substances contained in the biochemical analysis unit, or a stimulable phosphor sheet having been used for a biochemical analysis, in which a stimulable phosphor layer of the stimulable phosphor sheet is exposed to radiation radiated out from the radioactive labeling substance, is capable of being managed reliably until a level of radioactivity of the radioactive labeling substance attenuates to a level equal to at most a predetermined level.

A further object of the present invention is to provide a system for carrying out the method of managing a biochemical analysis medium.

The present invention provides a biochemical analysis system, comprising:

i) specific binding means for:

obtaining a biochemical analysis unit, which comprises a base plate and a plurality of spot-shaped regions located at different positions on or in the base plate, and which is provided with inherent identification information, each of multiple kinds of specific binding substances, which are capable of specifically binding to organism-originating substances and whose structures or characteristics are known, having been fixed to one of the spot-shaped regions, and

subjecting an organism-originating substance, which has been labeled with at least one labeling substance selected from the group consisting of a radioactive labeling substance, a fluorescent labeling substance, and a labeling substance capable of producing chemiluminescence when being brought into contact with a chemiluminescence substrate, and which has inherent identification information, to selective, specific binding to the multiple kinds of the specific binding substances, each of which has been fixed to one of the spot-shaped regions of the biochemical analysis unit, at least one spot-shaped region among the plurality of the spot-shaped regions of the biochemical analysis unit being thereby selectively labeled with the labeling substance,

ii) label signal forming means for receiving the biochemical analysis unit, which has been processed by the specific binding means, and forming a label signal, which corresponds to a position of the spot-shaped region having been selectively labeled with the labeling substance, by use of the biochemical analysis unit,

iii) read-out means for reading out the label signal, which has been formed by the label signal forming means, in order to form data for a biochemical analysis, and

iv) analysis means for executing a biochemical analysis in accordance with the data for a biochemical analysis,

wherein the biochemical analysis system further comprises first correspondence relationship specifying means for acquiring the identification information of the biochemical analysis unit and the identification information of the organism-originating substance, and feeding the two pieces of the identification information into the analysis means such that the correspondence relationship between the two pieces of the identification information is clear.

The term “spot-shaped region” as used herein means the specific binding substance, which has been spotted onto the base plate, and the site of the specific binding substance. In this specification, it should be noted that the term “spot-shaped region” has the same meaning as the term “spot.”

Also, the term “identification information of a biochemical analysis unit” as used herein means the information directly or indirectly representing the information concerning the biochemical analysis unit, which information is necessary when the structure, the characteristics, and the like, of the organism-originating substance are to be analyzed with the analysis means. Examples of the identification information of the biochemical analysis unit include the information representing the positions of the spot-shaped regions on the biochemical analysis unit, and the information representing the kinds of the specific binding substances which form the spot-shaped regions. (The information representing the positions of the spot-shaped regions on the biochemical analysis unit, the information representing the kinds of the specific binding substances which form the spot-shaped regions, and the like, will hereinbelow be referred to as the constitution information of the biochemical analysis unit.) For example, in a biochemical analysis system, wherein an identification (ID) number of the biochemical analysis unit and the constitution information of the biochemical analysis unit are managed such that the correspondence relationship between the ID number and the constitution information of the biochemical analysis unit is clear, the identification information of the biochemical analysis unit may be the ID number, which indirectly represents the constitution information of the biochemical analysis unit. In a biochemical analysis system, wherein the correspondence relationship between the ID number of the biochemical analysis unit, or the like, and the constitution information of the biochemical analysis unit is not managed, the constitution information itself of the biochemical analysis unit may be employed as the identification information of the biochemical analysis unit.

The term “identification information of an organism-originating substance” as used herein means the information with which the organism-originating substance (hereinbelow referred to also as the sample) is capable of being identified. Examples of the identification information of the organism-originating substance include the information, with which the date of sampling of the sample, the name of the sampling object (e.g., the patient, from which the cells of a tumor part was taken as the sample), the site of the sampling object, and the like, are capable of being specified. (The information, with which the date of sampling of the sample, the name of the sampling object, the site of the sampling object, and the like, are capable of being specified, will hereinbelow be referred to as the sample specifying information.) For example, in a biochemical analysis system, wherein an ID number of the sample, or the like, and the sample specifying information are managed such that the correspondence relationship between the ID number of the sample, or the like, and the sample specifying information is clear, the identification information of the organism-originating substance may be the ID number of the sample, or the like, which indirectly represents the sample specifying information of the organism-originating substance. In a biochemical analysis system, wherein the correspondence relationship between the ID number of the sample, or the like, and the sample specifying information of the organism-originating substance is not managed, the sample specifying information itself of the organism-originating substance may be employed as the identification information of the organism-originating substance.

The term “specific binding of an organism-originating substance to a specific binding substance” as used herein means, for example, the binding (i.e., hybridization), wherein a stable duplex strand is formed between complementary nucleotide sequences as in the cases of a DNA and an RNA, and the markedly specific binding, wherein selective reaction with a specific substance alone occurs, as in the cases of the binding between an antigen and an antibody, the binding between biotin and avidin, or the like.

The biochemical analysis system in accordance with the present invention should preferably be modified such that the base plate of the biochemical analysis unit is made from a material having radiation attenuating properties and/or light attenuating properties and has a plurality of holes,

an adsorptive region is formed in each of the plurality of the holes of the base plate, and

each of the multiple kinds of the specific binding substances is fixed to one of the adsorptive regions in order to form one of the spot-shaped regions.

Also, the biochemical analysis system in accordance with the present invention should preferably be modified such that the organism-originating substance is labeled with the radioactive labeling substance,

the label signal forming means comprises:

a) exposure means for superposing a stimulable phosphor sheet, which has inherent identification information, and the biochemical analysis unit, which has been processed by the specific binding means, upon each other in order to expose the stimulable phosphor sheet to radiation radiated out from the radioactive labeling substance, and

b) stimulable phosphor sheet stimulating means for irradiating stimulating rays to the stimulable phosphor sheet, which has been exposed to the radiation radiated out from the radioactive labeling substance, the stimulating rays causing the stimulable phosphor sheet to emit light in proportion to the amount of energy stored on the stimulable phosphor sheet during the exposure of the stimulable phosphor sheet to the radiation, the light emitted by the stimulable phosphor sheet acting as the label signal, and

the read-out means photoelectrically detects the light, which is emitted by the stimulable phosphor sheet, in order to form the data for a biochemical analysis.

The stimulable phosphor sheet comprises a support and a stimulable phosphor layer formed on or in the support. When the stimulable phosphor sheet and the biochemical analysis unit are superposed upon each other, the stimulable phosphor sheet and the biochemical analysis unit are brought into close contact with each other such that the stimulable phosphor layer of the stimulable phosphor sheet stands facing the spot-shaped region side of the biochemical analysis unit.

In such cases, the biochemical analysis system in accordance with the present invention should more preferably be modified such that the stimulable phosphor sheet comprises a support and a plurality of dot-shaped stimulable phosphor layer regions, which are located at a spacing from one another on the support, and

positions and sizes of the plurality of the dot-shaped stimulable phosphor layer regions correspond respectively to the positions and the sizes of the spot-shaped regions of the biochemical analysis unit.

Also, in such cases, the biochemical analysis system in accordance with the present invention should more preferably be modified such that the biochemical analysis system further comprises second correspondence relationship specifying means for acquiring the identification information of the biochemical analysis unit and the identification information of the stimulable phosphor sheet, and feeding the two pieces of the identification information into the analysis means such that the correspondence relationship between the two pieces of the identification information is clear.

The term “identification information of a stimulable phosphor sheet” as used herein means the information, with which the stimulable phosphor sheet is capable of being specified. By way of example, a sheet number may be employed as the identification information of the stimulable phosphor sheet.

Further, the biochemical analysis system in accordance with the present invention may be modified such that the organism-originating substance is labeled with the fluorescent labeling substance,

the label signal forming means is fluorescent substance exciting means for irradiating excitation light to the biochemical analysis unit, which has been processed by the specific binding means, the excitation light causing the fluorescent labeling substance to produce fluorescence, the fluorescence produced by the fluorescent labeling substance acting as the label signal, and

the read-out means photoelectrically detects the fluorescence, which is produced by the fluorescent labeling substance, in order to form the data for a biochemical analysis.

Furthermore, the biochemical analysis system in accordance with the present invention may be modified such that the organism-originating substance is labeled with the labeling substance capable of producing the chemiluminescence when being brought into contact with a chemiluminescence substrate,

the label signal forming means is chemiluminescence producing means for bringing the chemiluminescence substrate into contact with the biochemical analysis unit, which has been processed by the specific binding means, the chemiluminescence substrate causing the labeling substance to produce chemiluminescence, the chemiluminescence produced by the labeling substance acting as the label signal, and

the read-out means photoelectrically detects the chemiluminescence, which is produced by the labeling substance, in order to form the data for a biochemical analysis.

Also, the biochemical analysis system in accordance with the present invention should preferably be modified such that the biochemical analysis system further comprises spotting means for spotting each of the multiple kinds of the specific binding substances, which are capable of specifically binding to organism-originating substances and whose structures or characteristics are known, to one of different positions on or in the base plate in order to form the plurality of independent spot-shaped regions, and thereby preparing the biochemical analysis unit, and

third correspondence relationship specifying means for feeding spot information, which contains information representing the position of each of the spot-shaped regions, information representing the kind of the specific binding substance which forms each of the spot-shaped regions, and the like, and the identification information of the biochemical analysis unit into the analysis means such that the correspondence relationship between the spot information and the identification information of the biochemical analysis unit is clear.

The spot information may be of the same type as the constitution information of the biochemical analysis unit.

Further, the biochemical analysis system in accordance with the present invention should preferably be modified such that the identification information of the biochemical analysis unit contains identification information of the base plate.

In the biochemical analysis system in accordance with the present invention (and the biochemical analysis unit in accordance with the present invention, the method and system for managing a biochemical analysis unit in accordance with the present invention, and the method and system for managing a biochemical analysis medium in accordance with the present invention, which will be described later), the biochemical analysis unit should preferably be modified in various ways as described below.

Specifically, the biochemical analysis unit should preferably be modified such that the base plate is provided with a plurality of adsorptive regions, which are located at a spacing from one another and in two-dimensional directions.

With the biochemical analysis unit, wherein the base plate is provided with the plurality of the adsorptive regions, which are located at a spacing from one another and in two-dimensional directions, the problems are capable of being efficiently prevented from occurring in that the organism-originating substance, which is to be subjected to the specific binding to the specific binding substance contained in one of the adsorptive regions, is subjected to the specific binding to a specific binding substance contained in an adjacent adsorptive region. Therefore, the accuracy, with which the biochemical analysis is made, is capable of being enhanced.

Also, the biochemical analysis unit should preferably be modified such that the base plate is provided with a plurality of holes, which are located at a spacing from one another and in two-dimensional directions, and

each of the plurality of the adsorptive regions is formed by filling an adsorptive material in one of the holes.

Further, the biochemical analysis unit should preferably be modified such that the base plate is provided with a plurality of through-holes, which are located at a spacing from one another and in two-dimensional directions, and

each of the plurality of the adsorptive regions is formed by press-fitting an adsorptive film, which contains an adsorptive material, into one of the plurality of the through-holes of the base plate.

In such cases, the biochemical analysis unit is capable of being provided with the plurality of the adsorptive regions merely by press-fitting the adsorptive film, which contains the adsorptive material, into one of the plurality of the through-holes of the base plate. Therefore, the biochemical analysis unit is capable of being produced easily.

Also, the biochemical analysis unit should preferably be modified such that the base plate is provided with a plurality of recesses, which are located at a spacing from one another and in two-dimensional directions, and

each of the plurality of the adsorptive regions is constituted of an adsorptive material layer, which is formed on an inner wall surface of one of the plurality of the recesses of the base plate.

Further, the biochemical analysis unit should preferably be modified such that the plurality of the adsorptive regions is formed on a surface of the base plate.

Furthermore, the biochemical analysis unit should preferably be modified such that the base plate is provided with a plurality of protrusions, which are located at a spacing from one another and in two-dimensional directions, and

each of the plurality of the adsorptive regions is formed in the vicinity of a top end of one of the plurality of the protrusions of the base plate.

Also, the biochemical analysis unit should preferably be modified such that the base plate is made from an adsorptive material,

a perforated plate having a plurality of through-holes is located on at least either one of two surfaces of the base plate such that the perforated plate is in close contact with the one surface of the base plate, and

each of the plurality of the adsorptive regions is constituted of the adsorptive material, which is exposed within one of the plurality of the through-holes of the perforated plate.

Further, the biochemical analysis unit should preferably be modified such that a perforated plate having a plurality of through-holes is located on each of two surfaces of the base plate, which is made from an adsorptive material, such that the perforated plate is in close contact with the surface of the base plate.

With the biochemical analysis unit, wherein the perforated plate having the plurality of the through-holes is located on each of the two surfaces of the base plate, which is made from the adsorptive material, such that the perforated plate is in close contact with the surface of the base plate, at the time of the spotting of the specific binding substances, the hybridization, and the operation for exposing the stimulable phosphor sheet to radiation, the biochemical analysis unit is capable of being processed very easily.

Furthermore, the biochemical analysis unit should preferably be modified such that the base plate is provided with at least 10 adsorptive regions.

The biochemical analysis unit should more preferably be modified such that the base plate is provided with at least 50 adsorptive regions.

The biochemical analysis unit should more preferably be modified such that the base plate is provided with at least 100 adsorptive regions.

The biochemical analysis unit should more preferably be modified such that the base plate is provided with at least 500 adsorptive regions.

The biochemical analysis unit should more preferably be modified such that the base plate is provided with at least 1,000 adsorptive regions.

The biochemical analysis unit should more preferably be modified such that the base plate is provided with at least 5,000 adsorptive regions.

The biochemical analysis unit should more preferably be modified such that the base plate is provided with at least 10,000 adsorptive regions.

The biochemical analysis unit should more preferably be modified such that the base plate is provided with at least 50,000 adsorptive regions.

The biochemical analysis unit should most preferably be modified such that the base plate is provided with at least 100,000 adsorptive regions.

Also, the biochemical analysis unit should preferably be modified such that each of the plurality of the adsorptive regions has a size smaller than 5 mm².

The biochemical analysis unit should more preferably be modified such that each of the plurality of the adsorptive regions has a size smaller than 1 mm².

The biochemical analysis unit should more preferably be modified such that each of the plurality of the adsorptive regions has a size smaller than 0.5 mm².

The biochemical analysis unit should more preferably be modified such that each of the plurality of the adsorptive regions has a size smaller than 0.1 mm².

The biochemical analysis unit should more preferably be modified such that each of the plurality of the adsorptive regions has a size smaller than 0.05 mm².

The biochemical analysis unit should most preferably be modified such that each of the plurality of the adsorptive regions has a size smaller than 0.01 mm².

Further, the biochemical analysis unit should preferably be modified such that the base plate is provided with the plurality of the adsorptive regions at a density of at least 10 regions/cm².

The biochemical analysis unit should more preferably be modified such that the base plate is provided with the plurality of the adsorptive regions at a density of at least 50 regions/cm².

The biochemical analysis unit should more preferably be modified such that the base plate is provided with the plurality of the adsorptive regions at a density of at least 100 regions/cm².

The biochemical analysis unit should more preferably be modified such that the base plate is provided with the plurality of the adsorptive regions at a density of at least 500 regions/cm².

The biochemical analysis unit should more preferably be modified such that the base plate is provided with the plurality of the adsorptive regions at a density of at least 1,000 regions/cm².

The biochemical analysis unit should more preferably be modified such that the base plate is provided with the plurality of the adsorptive regions at a density of at least 5,000 regions/cm².

The biochemical analysis unit most preferably be modified such that the base plate is provided with the plurality of the adsorptive regions at a density of at least 10,000 regions/cm².

Furthermore, the biochemical analysis unit should preferably be modified such that the adsorptive regions are located in a regular pattern.

Also, the biochemical analysis unit should preferably be modified such that the base plate has radiation attenuating properties.

With the biochemical analysis unit, wherein the base plate of the biochemical analysis unit has the radiation attenuating properties, the advantages described below are capable of being obtained. Specifically, in cases where each of the specific binding substances is spotted onto one of the adsorptive regions of the biochemical analysis unit, the organism-originating substance having been labeled with the radioactive labeling substance is then subjected to the selective, specific binding to the specific binding substances contained in the plurality of the adsorptive regions, and thereafter the stimulable phosphor layer of the stimulable phosphor sheet is exposed to the radiation radiated out from the radioactive labeling substance, which has thus been contained selectively in at least one adsorptive region among the plurality of the adsorptive regions, the problems are capable of being efficiently prevented from occurring in that electron rays (beta rays), which have been radiated out from the radioactive labeling substance contained in one of the adsorptive regions, are scattered within the base plate of the biochemical analysis unit and impinge upon a stimulable phosphor layer region, which is to be exposed to the electron rays (the beta rays) radiated out from an adjacent adsorptive region. Therefore, the problems are capable of being efficiently prevented from occurring in that noise due to the scattering of the electron rays (the beta rays) is formed in the data for a biochemical analysis. Accordingly, the data for a biochemical analysis having good quantitative characteristics is capable of being obtained.

Further, the biochemical analysis unit should preferably be modified such that the base plate has properties such that, when radiation has passed through the base plate by a distance equal to the distance between two adjacent adsorptive regions, the base plate attenuates energy of the radiation to a level at most ⅕ times as high as the level of energy of the radiation before passing through the base plate by the distance.

The biochemical analysis unit should more preferably be modified such that the base plate has the properties such that, when the radiation has passed through the base plate by the distance equal to the distance between two adjacent adsorptive regions, the base plate attenuates energy of the radiation to a level at most 1/10 times as high as the level of energy of the radiation before passing through the base plate by the distance.

The biochemical analysis unit should more preferably be modified such that the base plate has the properties such that, when the radiation has passed through the base plate by the distance equal to the distance between two adjacent adsorptive regions, the base plate attenuates energy of the radiation to a level at most 1/50 times as high as the level of energy of the radiation before passing through the base plate by the distance.

The biochemical analysis unit should more preferably be modified such that the base plate has the properties such that, when the radiation has passed through the base plate by the distance equal to the distance between two adjacent adsorptive regions, the base plate attenuates energy of the radiation to a level at most 1/100 times as high as the level of energy of the radiation before passing through the base plate by the distance.

The biochemical analysis unit should more preferably be modified such that the base plate has the properties such that, when the radiation has passed through the base plate by the distance equal to the distance between two adjacent adsorptive regions, the base plate attenuates energy of the radiation to a level at most 1/500 times as high as the level of energy of the radiation before passing through the base plate by the distance.

The biochemical analysis unit should most preferably be modified such that the base plate has the properties such that, when the radiation has passed through the base plate by the distance equal to the distance between two adjacent adsorptive regions, the base plate attenuates energy of the radiation to a level at most 1/1,000 times as high as the level of energy of the radiation before passing through the base plate by the distance.

Furthermore, the biochemical analysis unit should preferably be modified such that the base plate has light attenuating properties.

With the biochemical analysis unit, wherein the base plate of the biochemical analysis unit has the light attenuating properties, the advantages described below are capable of being obtained. Specifically, in cases where each of the specific binding substances is spotted onto one of the adsorptive regions of the biochemical analysis unit, the organism-originating substance, which has been labeled with the fluorescent labeling substance or the labeling substance capable of producing the chemiluminescence when being brought into contact with a chemiluminescence substrate, is then subjected to the selective, specific binding to the specific binding substances contained in the plurality of the adsorptive regions, and thereafter the data for a biochemical analysis is formed by irradiating the excitation light to the plurality of the adsorptive regions and photoelectrically detecting the fluorescence produced by the fluorescent labeling substance, or by photoelectrically detecting the chemiluminescence produced by the labeling substance capable of producing the chemiluminescence when being brought into contact with a chemiluminescence substrate, the problems are capable of being efficiently prevented from occurring in that the fluorescence or the chemiluminescence produced from one of the adsorptive regions is scattered within the base plate of the biochemical analysis unit and mixes with the fluorescence or the chemiluminescence produced from an adjacent adsorptive region. Therefore, the problems are capable of being efficiently prevented from occurring in that noise due to the scattering of the fluorescence or the chemiluminescence is formed in the data for a biochemical analysis. Accordingly, the data for a biochemical analysis having good quantitative characteristics is capable of being obtained.

Also, the biochemical analysis unit should preferably be modified such that the base plate has properties such that, when light has passed through the base plate by a distance equal to the distance between two adjacent adsorptive regions, the base plate attenuates energy of the light to a level at most ⅕ times as high as the level of energy of the light before passing through the base plate by the distance.

The biochemical analysis unit should more preferably be modified such that the base plate has properties such that, when light has passed through the base plate by a distance equal to the distance between two adjacent adsorptive regions, the base plate attenuates energy of the light to a level at most 1/10 times as high as the level of energy of the light before passing through the base plate by the distance.

The biochemical analysis unit should more preferably be modified such that the base plate has properties such that, when light has passed through the base plate by a distance equal to the distance between two adjacent adsorptive regions, the base plate attenuates energy of the light to a level at most 1/50 times as high as the level of energy of the light before passing through the base plate by the distance.

The biochemical analysis unit should more preferably be modified such that the base plate has properties such that, when light has passed through the base plate by a distance equal to the distance between two adjacent adsorptive regions, the base plate attenuates energy of the light to a level at most 1/100 times as high as the level of energy of the light before passing through the base plate by the distance.

The biochemical analysis unit should more preferably be modified such that the base plate has properties such that, when light has passed through the base plate by a distance equal to the distance between two adjacent adsorptive regions, the base plate attenuates energy of the light to a level at most 1/500 times as high as the level of energy of the light before passing through the base plate by the distance.

The biochemical analysis unit should more preferably be modified such that the base plate has properties such that, when light has passed through the base plate by a distance equal to the distance between two adjacent adsorptive regions, the base plate attenuates energy of the light to a level at most 1/1,000 times as high as the level of energy of the light before passing through the base plate by the distance.

Further, the biochemical analysis unit should preferably be modified such that the perforated plate described above has radiation attenuating properties.

With the biochemical analysis unit, wherein the perforated plate of the biochemical analysis unit has the radiation attenuating properties, the advantages described below are capable of being obtained. Specifically, in cases where each of the specific binding substances is spotted onto one of the adsorptive regions of the biochemical analysis unit, the organism-originating substance having been labeled with the radioactive labeling substance is then subjected to the selective, specific binding to the specific binding substances contained in the plurality of the adsorptive regions, and thereafter the stimulable phosphor layer of the stimulable phosphor sheet is exposed to the radiation radiated out from the radioactive labeling substance, which has thus been contained selectively in at least one adsorptive region among the plurality of the adsorptive regions, the problems are capable of being efficiently prevented from occurring in that the electron rays (the beta rays), which have been radiated out from the radioactive labeling substance contained in one of the adsorptive regions, are scattered within the perforated plate of the biochemical analysis unit and impinge upon a stimulable phosphor layer region, which is to be exposed to the electron rays (the beta rays) radiated out from an adjacent adsorptive region. Therefore, the problems are capable of being efficiently prevented from occurring in that noise due to the scattering of the electron rays (the beta rays) is formed in the data for a biochemical analysis. Accordingly, the data for a biochemical analysis having good quantitative characteristics is capable of being obtained.

Furthermore, the biochemical analysis unit should preferably be modified such that the perforated plate has properties such that, when radiation has passed through the perforated plate by a distance equal to the distance between two adjacent adsorptive regions, the perforated plate attenuates energy of the radiation to a level at most ⅕ times as high as the level of energy of the radiation before passing through the perforated plate by the distance described above.

The biochemical analysis unit should more preferably be modified such that the perforated plate has properties such that, when radiation has passed through the perforated plate by a distance equal to the distance between two adjacent adsorptive regions, the perforated plate attenuates energy of the radiation to a level at most 1/10 times as high as the level of energy of the radiation before passing through the perforated plate by the distance described above.

The biochemical analysis unit should more preferably be modified such that the perforated plate has properties such that, when radiation has passed through the perforated plate by a distance equal to the distance between two adjacent adsorptive regions, the perforated plate attenuates energy of the radiation to a level at most 1/50 times as high as the level of energy of the radiation before passing through the perforated plate by the distance described above.

The biochemical analysis unit should more preferably be modified such that the perforated plate has properties such that, when radiation has passed through the perforated plate by a distance equal to the distance between two adjacent adsorptive regions, the perforated plate attenuates energy of the radiation to a level at most 1/100 times as high as the level of energy of the radiation before passing through the perforated plate by the distance described above.

The biochemical analysis unit should more preferably be modified such that the perforated plate has properties such that, when radiation has passed through the perforated plate by a distance equal to the distance between two adjacent adsorptive regions, the perforated plate attenuates energy of the radiation to a level at most 1/500 times as high as the level of energy of the radiation before passing through the perforated plate by the distance described above.

The biochemical analysis unit should most preferably be modified such that the perforated plate has properties such that, when radiation has passed through the perforated plate by a distance equal to the distance between two adjacent adsorptive regions, the perforated plate attenuates energy of the radiation to a level at most 1/1,000 times as high as the level of energy of the radiation before passing through the perforated plate by the distance described above.

Also, the biochemical analysis unit should preferably be modified such that the perforated plate described above has light attenuating properties.

With the biochemical analysis unit, wherein the perforated plate of the biochemical analysis unit has the light attenuating properties, the advantages described below are capable of being obtained. Specifically, in cases where each of the specific binding substances is spotted onto one of the adsorptive regions of the biochemical analysis unit, the organism-originating substance, which has been labeled with the fluorescent labeling substance or the labeling substance capable of producing the chemiluminescence when being brought into contact with a chemiluminescence substrate, is then subjected to the selective, specific binding to the specific binding substances contained in the plurality of the adsorptive regions, and thereafter the data for a biochemical analysis is formed by irradiating the excitation light to the plurality of the adsorptive regions and photoelectrically detecting the fluorescence produced by the fluorescent labeling substance, or by photoelectrically detecting the chemiluminescence produced by the labeling substance capable of producing the chemiluminescence when being brought into contact with a chemiluminescence substrate, the problems are capable of being efficiently prevented from occurring in that the fluorescence or the chemiluminescence produced from one of the adsorptive regions is scattered within the perforated plate of the biochemical analysis unit and mixes with the fluorescence or the chemiluminescence produced from an adjacent adsorptive region. Therefore, the problems are capable of being efficiently prevented from occurring in that noise due to the scattering of the fluorescence or the chemiluminescence is formed in the data for a biochemical analysis. Accordingly, the data for a biochemical analysis having good quantitative characteristics is capable of being obtained.

Further, the biochemical analysis unit should preferably be modified such that the perforated plate has properties such that, when light has passed through the perforated plate by a distance equal to the distance between two adjacent adsorptive regions, the perforated plate attenuates energy of the light to a level at most ⅕ times as high as the level of energy of the light before passing through the perforated plate by the distance described above.

The biochemical analysis unit should more preferably be modified such that the perforated plate has properties such that, when light has passed through the perforated plate by a distance equal to the distance between two adjacent adsorptive regions, the perforated plate attenuates energy of the light to a level at most 1/10 times as high as the level of energy of the light before passing through the perforated plate by the distance described above.

The biochemical analysis unit should more preferably be modified such that the perforated plate has properties such that, when light has passed through the perforated plate by a distance equal to the distance between two adjacent adsorptive regions, the perforated plate attenuates energy of the light to a level at most 1/50 times as high as the level of energy of the light before passing through the perforated plate by the distance described above.

The biochemical analysis unit should more preferably be modified such that the perforated plate has properties such that, when light has passed through the perforated plate by a distance equal to the distance between two adjacent adsorptive regions, the perforated plate attenuates energy of the light to a level at most 1/100 times as high as the level of energy of the light before passing through the perforated plate by the distance described above.

The biochemical analysis unit should more preferably be modified such that the perforated plate has properties such that, when light has passed through the perforated plate by a distance equal to the distance between two adjacent adsorptive regions, the perforated plate attenuates energy of the light to a level at most 1/500 times as high as the level of energy of the light before passing through the perforated plate by the distance described above.

The biochemical analysis unit should most preferably be modified such that the perforated plate has properties such that, when light has passed through the perforated plate by a distance equal to the distance between two adjacent adsorptive regions, the perforated plate attenuates energy of the light to a level at most 1/1,000 times as high as the level of energy of the light before passing through the perforated plate by the distance described above.

The material for the formation of the base plate or the perforated plate of the biochemical analysis unit should preferably have the radiation attenuating properties and/or the light attenuating properties. The material for the formation of the base plate or the perforated plate of the biochemical analysis unit may be selected from a wide variety of inorganic compound materials and organic compound materials, and should preferably be selected from metal materials, ceramic materials, and plastic materials.

Examples of the inorganic compound materials, which may be utilized preferably for the formation of the base plate or the perforated plate of the biochemical analysis unit, include metals, such as gold, silver, copper, zinc, aluminum, titanium, tantalum, chromium, iron, nickel, cobalt, lead, tin, and selenium; alloys, such as brass, stainless steel, and bronze; silicon materials, such as silicon, amorphous silicon, glass, quartz, silicon carbide, and silicon nitride; metal oxides, such as aluminum oxide, magnesium oxide, and zirconium oxide; and inorganic salts, such as tungsten carbide, calcium carbonate, calcium sulfate, hydroxyapatite, and gallium arsenide. The above-enumerated materials may have a single crystalline structure, an amorphous structure, or a structure of a polycrystalline sintered material, such as a ceramic material.

As the organic compound materials, which may be utilized for the formation of the base plate or the perforated plate of the biochemical analysis unit, high-molecular weight compounds are preferable. Examples of the high-molecular weight compounds, which may be utilized preferably for the formation of the base plate or the perforated plate of the biochemical analysis unit, include polyolefins, such as a polyethylene and a polypropylene; acrylic resins, such as a polymethyl methacrylate and a butyl acrylate-methyl methacrylate copolymer; polyacrylonitriles; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; nylons, such as a 6-nylon, a 6,6-nylon, and a 4,10-nylon; polyimides; polysulfones; polyphenylene sulfides; silicon resins, such as a polydiphenyl siloxane; phenolic resins, such as novolak; epoxy resins; polyurethanes; polystyrenes; butadiene-styrene copolymers; polysaccharides, such as cellulose, cellulose acetate, nitrocellulose, starch, calcium alginate, and hydroxypropylmethylcellulose; chitin; chitosan; Japanese lacquer; polyamides, such as gelatin, collagen, and keratin; and copolymers of the above-enumerated high-molecular weight compounds. The above-enumerated organic compound materials may be composite materials. When necessary, the above-enumerated organic compound materials may be loaded with metal oxide particles, glass fibers, and the like. Also, the above-enumerated organic compound materials may be blended with organic compound materials.

Ordinarily, as the specific gravity of the material becomes large, the radiation attenuating capability of the material becomes high. Therefore, the base plate or the perforated plate of the biochemical analysis unit should preferably be made from a compound material or a composite material, which has a specific gravity of at least 1.0 g/cm³. The base plate or the perforated plate should more preferably be made from a compound material or a composite material, which has a specific gravity falling within the range of 1.5 g/cm³ to 23 g/cm³.

Also, ordinarily, the material scatters and/or absorbs more of light, the light attenuating capability of the material becomes high. Therefore, the base plate or the perforated plate of the biochemical analysis unit should preferably has a light absorbance of at least 0.3 per 1 cm thickness, and should more preferably has a light absorbance of at least 1 per 1 cm thickness. The light absorbance may be obtained by locating an integrating sphere just behind a plate-shaped material having a thickness of Tcm, measuring a transmission light intensity A with respect to the wavelength of probe light or emission light, which is utilized for measurement, by use of a spectro-photometer, and calculate the value of A/T. In order for the light attenuating capability to be enhanced, a light scattering material and/or a light absorbing material may be contained in the base plate or the perforated plate of the biochemical analysis unit. As the light scattering material, fine particles of a material, which is different from the material constituting the base plate or the perforated plate of the biochemical analysis unit, may be employed. As the light absorbing material, a pigment or a dye may be employed.

Also, the biochemical analysis unit should preferably be modified such that the base plate is constituted of an adsorptive plate, which is made from an adsorptive material.

As the adsorptive material for the formation of the adsorptive regions or the adsorptive plate of the biochemical analysis unit, a porous material or a fiber material may be utilized preferably. The porous material and the fiber material may be utilized in combination in order to form the adsorptive regions or the adsorptive plate of the biochemical analysis unit.

The porous material, which may be utilized for the formation of the adsorptive regions or the adsorptive plate of the biochemical analysis unit, may be an organic material, an inorganic material, or an organic-inorganic composite material.

The organic porous material, which may be utilized for the formation of the adsorptive regions or the adsorptive plate of the biochemical analysis unit, may be selected from a wide variety of materials. However, the organic porous material should preferably be a carbon material, such as active carbon, or a material capable of forming a membrane filter. Examples of the organic porous materials include nylons, such as a 6-nylon, a 6,6-nylon, and a 4,10-nylon; cellulose derivatives, such as nitrocellulose, cellulose acetate, and cellulose acetate butyrate; collagen; alginic acids, such as alginic acid, calcium alginate, and an alginic acid-polylysine polyion complex; polyolefins, such as a polyethylene and a polypropylene; polyvinyl chlorides; polyvinylidene chlorides; polyfluorides, such as a polyvinylidene fluoride and a polytetrafluoride; and copolymers or composite materials of the above-enumerated materials.

The inorganic porous material, which may be utilized for the formation of the adsorptive regions or the adsorptive plate of the biochemical analysis unit, may be selected from a wide variety of materials. Examples of the inorganic porous materials, which may be utilized preferably, include metals, such as platinum, gold, iron, silver, nickel, and aluminum; metal oxides, such as alumina, silica, titania, and zeolite; metal salts, such as hydroxyapatite and calcium sulfate; and composite materials of the above-enumerated materials.

The fiber material, which may be utilized for the formation of the adsorptive regions or the adsorptive plate of the biochemical analysis unit, may be selected from a wide variety of materials. Examples of the fiber materials, which may be utilized preferably, include nylons, such as a 6-nylon, a 6,6-nylon, and a 4,10-nylon; and cellulose derivatives, such as nitrocellulose, cellulose acetate, and cellulose acetate butyrate.

Further, the biochemical analysis unit should preferably be modified such that the base plate is made from a slide glass plate.

The adsorptive regions of the biochemical analysis unit may be formed with surface treatment, e.g., oxidation treatment, such as electrolytic treatment, plasma treatment, or arc discharging; primer treatment using a silane coupling agent, a titanium coupling agent, or the like; or surface-active agent treatment.

The present invention also provides a biochemical analysis unit, comprising a base plate and a data recording layer containing a data-rewritable recording medium, which data recording layer is formed on or in the base plate.

With the biochemical analysis unit in accordance with the present invention, wherein the data recording layer containing the data-rewritable recording medium is formed on or in the base plate of the biochemical analysis unit, data which is required to be managed such that the correspondence relationship to the biochemical analysis unit is clear, such as data concerning the kinds of the specific binding substances spotted onto the biochemical analysis unit and the positions, to which the specific binding substances were spotted, or data concerning the number of times of use of the biochemical analysis unit, is capable of being recorded on the data recording layer. In this manner, the data is capable of being managed reliably such that the correspondence relationship between the data and the biochemical analysis unit is clear.

The biochemical analysis unit in accordance with the present invention should preferably be modified such that the base plate is provided with a plurality of spot-shaped regions, the spot-shaped regions having been formed by spotting specific binding substances, which are capable of specifically binding to organism-originating substances and whose base sequences, base lengths, compositions, and the like, are known, onto the base plate.

Also, in the biochemical analysis unit in accordance with the present invention, the data recording layer should preferably be constituted of a magnetic recording medium.

With the biochemical analysis unit in accordance with the present invention, wherein the data recording layer is constituted of the magnetic recording medium, in cases where the biochemical analysis unit is subjected to treatment with a liquid, such as the hybridization, the data having been recorded on the data recording layer is capable of being retained.

Alternatively, in the biochemical analysis unit in accordance with the present invention, the data recording layer may be constituted of an optical recording medium.

With the biochemical analysis unit in accordance with the present invention, wherein the data recording layer is constituted of the optical recording medium, in cases where the biochemical analysis unit is subjected to treatment with a liquid, such as the hybridization, the data having been recorded on the data recording layer is capable of being retained.

Further, the biochemical analysis unit in accordance with the present invention should preferably be modified such that the data recording layer comprises a first data recording region, which is protected from data rewriting conducted by a user, and a second data recording region, on which the data is capable of being rewritten by the user.

With the biochemical analysis unit in accordance with the present invention, wherein the data recording layer comprises the first data recording region, which is protected from data rewriting conducted by the user, and the second data recording region, on which the data is capable of being rewritten by the user, the data, which is essential to the management of the biochemical analysis unit and is not to be rewritten arbitrarily by the user, such as the data concerning the kinds of the specific binding substances spotted onto the biochemical analysis unit and the positions, to which the specific binding substances were spotted, or the data concerning the number of times of use of the biochemical analysis unit, may be recorded on the first data recording region of the data recording layer. In such cases, the use of the biochemical analysis unit in a desired manner is capable of being guaranteed, and the reliability of the biochemical analysis is capable of being enhanced. Also, the user is capable of writing personally necessary data on the second data recording region of the data recording layer. Therefore, the efficiency, with which the biochemical analysis is made, is capable of being enhanced markedly.

Furthermore, the biochemical analysis unit in accordance with the present invention should preferably be modified such that data concerning kinds of the specific binding substances and positions of the spot-shaped regions, each of which contains one of the specific binding substances, is recorded on the first data recording region of the data recording layer.

Also, the biochemical analysis unit in accordance with the present invention should preferably be modified such that data concerning a number of times of use of the biochemical analysis unit is capable of being recorded on the first data recording region of the data recording layer.

Ordinarily, if the biochemical analysis unit is used a predetermined number of times or more, the problems will occur in that part of the specific binding substance spotted onto the biochemical analysis unit separates from the biochemical analysis unit, the accuracy with which the biochemical analysis is made, becomes markedly low, and reliable analysis results cannot be obtained. However, with the biochemical analysis unit in accordance with the present invention, wherein the data concerning the number of times of use of the biochemical analysis unit is capable of being recorded on the first data recording region of the data recording layer, which region is protected from data rewriting conducted by the user, the problems are capable of being efficiently prevented from occurring in that the user uses the biochemical analysis unit a predetermined number of times or more by mistake.

Further, the biochemical analysis unit in accordance with the present invention should preferably be modified such that data concerning a day of use of the biochemical analysis unit is capable of being recorded on the first data recording region and/or the second data recording region of the data recording layer.

With the biochemical analysis unit in accordance with the present invention, wherein the data concerning the day of use of the biochemical analysis unit is capable of being recorded on the first data recording region and/or the second data recording region of the data recording layer, in cases where the organism-originating substance having been labeled with the radioactive labeling substance has been subjected to the hybridization with the specific binding substances having been spotted onto the biochemical analysis unit, the date and hour, at which the biochemical analysis unit is capable of being scrapped, is capable of being managed reliably.

Furthermore, the biochemical analysis unit in accordance with the present invention should preferably be modified such that data concerning a number of times of recycling of the biochemical analysis unit is capable of being recorded on the first data recording region of the data recording layer.

With the biochemical analysis unit in accordance with the present invention, wherein the data concerning the number of times of recycling of the biochemical analysis unit is capable of being recorded on the first data recording region of the data recording layer, a biochemical analysis unit maker is capable of utilizing the base plate to the utmost limit in accordance with the durability of the base plate and thereby achieving saving of resources. Also, the reliability of the biochemical analysis unit is capable of being kept.

Furthermore, the biochemical analysis unit in accordance with the present invention should preferably be modified such that data concerning the plurality of the adsorptive regions is capable of being recorded on the first data recording region of the data recording layer.

In cases where the base plate is provided with the plurality of the adsorptive regions, which are located at a spacing from one another, it is not easy to form all of the adsorptive regions with the same size. In cases where the sizes of the adsorptive regions vary, noise due to variation in size among the adsorptive regions will occur in the data for a biochemical analysis. However, with the biochemical analysis unit in accordance with the present invention, wherein the data concerning the plurality of the adsorptive regions is capable of being recorded on the first data recording region of the data recording layer, the data for a biochemical analysis is capable of being corrected in accordance with the data concerning the plurality of the adsorptive regions, which data has been recorded on the first data recording region of the data recording layer, and noise is thus capable of being removed from the data for a biochemical analysis. Therefore, the data for a biochemical analysis having good quantitative characteristics is capable of being formed.

The present invention further provides a method of managing a biochemical analysis unit comprising a plurality of adsorptive regions located at a spacing from one another, each of the plurality of the adsorptive regions containing one of specific binding substances, whose structures or characteristics are known, the method comprising the step of:

recording management data that contains data concerning time acting as reference time for a calculation of an attenuation period, at which radiation from a radioactive labeling substance attenuates, on the biochemical analysis unit when a biochemical analysis is executed by use of the biochemical analysis unit.

With the method of managing a biochemical analysis unit in accordance with the present invention, when the biochemical analysis is executed by use of the biochemical analysis unit, the management data that contains the data concerning the time acting as the reference time for the calculation of the attenuation period, at which the radiation from the radioactive labeling substance attenuates, is recorded on the biochemical analysis unit. Therefore, in accordance with the data concerning the time acting as the reference time for the calculation of the attenuation period, at which the radiation from the radioactive labeling substance attenuates, the data having been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

The method of managing a biochemical analysis unit in accordance with the present invention should preferably be modified such that, when an organism-originating substance, which has been labeled with a radioactive labeling substance, is subjected by a hybridization apparatus to selective hybridization with the specific binding substances, each of which is contained in one of the plurality of the adsorptive regions of the biochemical analysis unit,

the management data that contains the data concerning the time acting as the reference time for the calculation of the attenuation period, at which the radiation from the radioactive labeling substance attenuates, is recorded on the biochemical analysis unit by the hybridization apparatus.

With the aforesaid modification of the method of managing a biochemical analysis unit in accordance with the present invention, when the organism-originating substance, which has been labeled with the radioactive labeling substance, is subjected by the hybridization apparatus to the selective hybridization with the specific binding substances, each of which is contained in one of the plurality of the adsorptive regions of the biochemical analysis unit, the management data that contains the data concerning the time acting as the reference time for the calculation of the attenuation period, at which the radiation from the radioactive labeling substance attenuates, is recorded on the biochemical analysis unit by the hybridization apparatus. Therefore, in accordance with the data concerning the time acting as the reference time for the calculation of the attenuation period, at which the radiation from the radioactive labeling substance attenuates, the data having been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

The method of managing a biochemical analysis unit in accordance with the present invention should more preferably be modified such that the data concerning the time acting as the reference time for the calculation of the attenuation period, at which the radiation from the radioactive labeling substance attenuates, contains data concerning a date and hour of execution of the hybridization.

With the aforesaid modification of the method of managing a biochemical analysis unit in accordance with the present invention, the data concerning the time acting as the reference time for the calculation of the attenuation period, at which the radiation from the radioactive labeling substance attenuates, contains the data concerning the date and hour of execution of the hybridization. Therefore, in accordance with the data concerning the date and hour of execution of the hybridization, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Also, the method of managing a biochemical analysis unit in accordance with the present invention should more preferably be modified such that the data concerning the time acting as the reference time for the calculation of the attenuation period, at which the radiation from the radioactive labeling substance attenuates, contains data concerning a day of execution of the hybridization.

With the aforesaid modification of the method of managing a biochemical analysis unit in accordance with the present invention, the data concerning the time acting as the reference time for the calculation of the attenuation period, at which the radiation from the radioactive labeling substance attenuates, contains the data concerning the day of execution of the hybridization. Therefore, in accordance with the data concerning the day of execution of the hybridization, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Further, the method of managing a biochemical analysis unit in accordance with the present invention should preferably be modified such that, in cases where an organism-originating substance, which has been labeled with a radioactive labeling substance, is subjected by a hybridization apparatus to selective hybridization with the specific binding substances, each of which is contained in one of the plurality of the adsorptive regions of the biochemical analysis unit, and an exposure operation is thereafter executed with an exposure apparatus by superposing the biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective hybridization,

the management data that contains the data concerning the time acting as the reference time for the calculation of the attenuation period, at which the radiation from the radioactive labeling substance attenuates, is recorded on the biochemical analysis unit by the exposure apparatus when the exposure operation is executed.

With the aforesaid modification of the method of managing a biochemical analysis unit in accordance with the present invention, in cases where the organism-originating substance, which has been labeled with the radioactive labeling substance, is subjected by the hybridization apparatus to the selective hybridization with the specific binding substances, each of which is contained in one of the plurality of the adsorptive regions of the biochemical analysis unit, and the exposure operation is thereafter executed with the exposure apparatus by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective hybridization, the management data that contains the data concerning the time acting as the reference time for the calculation of the attenuation period, at which the radiation from the radioactive labeling substance attenuates, is recorded on the biochemical analysis unit by the exposure apparatus when the exposure operation is executed. Therefore, in accordance with the data concerning the time acting as the reference time for the calculation of the attenuation period, at which the radiation from the radioactive labeling substance attenuates, the data having been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

In such cases, the method of managing a biochemical analysis unit in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

With the aforesaid modification of the method of managing a biochemical analysis unit in accordance with the present invention, in accordance with the data concerning the date and hour of execution of the exposure operation, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

The method of managing a biochemical analysis unit in accordance with the present invention should more preferably be modified such that the data concerning the time acting as the reference time for the calculation of the attenuation period, at which the radiation from the radioactive labeling substance attenuates, contains data concerning a day of execution of the exposure operation.

With the aforesaid modification of the method of managing a biochemical analysis unit in accordance with the present invention, in accordance with the data concerning the day of execution of the exposure operation, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Furthermore, the method of managing a biochemical analysis unit in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

With the aforesaid modification of the method of managing a biochemical analysis unit in accordance with the present invention, the data concerning the date and hour of formation of the radioactive labeling substance is recorded on the biochemical analysis unit. Therefore, in accordance with the data concerning the date and hour of formation of the radioactive labeling substance, which data has been recorded on the biochemical analysis unit, and in accordance with the nuclide of the radioactive labeling substance used, the biochemical analysis unit having been used for the biochemical analysis is capable of being managed more reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Also, the method of managing a biochemical analysis unit in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

With the aforesaid modification of the method of managing a biochemical analysis unit in accordance with the present invention, the data concerning the day of formation of the radioactive labeling substance is recorded on the biochemical analysis unit. Therefore, in accordance with the data concerning the day of formation of the radioactive labeling substance, which data has been recorded on the biochemical analysis unit, and in accordance with the nuclide of the radioactive labeling substance used, the biochemical analysis unit having been used for the biochemical analysis is capable of being managed more reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

In the method of managing a biochemical analysis unit in accordance with the present invention, the management data may be recorded on the biochemical analysis unit with one of various recording techniques, such as printing and marking.

Further, the method of managing a biochemical analysis unit in accordance with the present invention should preferably be modified such that the management data is recorded as visible data on the biochemical analysis unit.

With the aforesaid modification of the method of managing a biochemical analysis unit in accordance with the present invention, the management data is recorded as the visible data on the biochemical analysis unit. Therefore, the user is capable of reading out the management data that contains the data concerning the time acting as the reference time for the calculation of the attenuation period, at which the radiation from the radioactive labeling substance attenuates, the management data having been recorded on the biochemical analysis unit. Also, in accordance with the management data having been read out, and in accordance with the nuclide of the radioactive labeling substance used, the user is capable of reliably managing the biochemical analysis unit, which has been used for the biochemical analysis, until the level of the radioactivity of the radioactive labeling substance attenuates to a level equal to at most a predetermined level. Further, after the level of the radioactivity of the radioactive labeling substance has attenuated to the level equal to at most the predetermined level, the biochemical analysis unit is capable of being scrapped. Furthermore, the management data that contains the data concerning the time acting as the reference time for the calculation of the attenuation period, at which the radiation from the radioactive labeling substance attenuates, is capable of being read out from the biochemical analysis unit by use of an optical data read-out apparatus. In accordance with the management data having been read out, and in accordance with the nuclide of the radioactive labeling substance used, the attenuation period, at which the level of the radioactivity of the radioactive labeling substance attenuates to the level equal to at most the predetermined level, is capable of being calculated automatically. The biochemical analysis unit may then be sorted in accordance with the attenuation period, at which the level of the radioactivity of the radioactive labeling substance attenuates to the level equal to at most the predetermined level. In this manner, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

Further, the method of managing a biochemical analysis unit in accordance with the present invention should preferably be modified such that the base plate of the biochemical analysis unit is provided with a magnetic recording layer, and the management data is magnetically recorded on the magnetic recording layer.

With the aforesaid modification of the method of managing a biochemical analysis unit in accordance with the present invention, the base plate of the biochemical analysis unit is provided with the magnetic recording layer, and the management data is magnetically recorded on the magnetic recording layer. Therefore, the management data that contains the data concerning the time acting as the reference time for the calculation of the attenuation period, at which the radiation from the radioactive labeling substance attenuates, is capable of being read out from the magnetic recording layer of the base plate of the biochemical analysis unit by use of a magnetic data read-out apparatus. Also, in accordance with the management data having been read out, and in accordance with the nuclide of the radioactive labeling substance used, the attenuation period, at which the level of the radioactivity of the radioactive labeling substance attenuates to the level equal to at most the predetermined level, is capable of being calculated automatically. The biochemical analysis unit may then be sorted in accordance with the attenuation period, at which the level of the radioactivity of the radioactive labeling substance attenuates to the level equal to at most the predetermined level. In this manner, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

Also, the method of managing a biochemical analysis unit in accordance with the present invention should preferably be modified such that, besides the data concerning the time acting as the reference time for the calculation of the attenuation period, at which the radiation from the radioactive labeling substance attenuates, the management data further contains data concerning a nuclide of the radioactive labeling substance.

With the aforesaid modification of the method of managing a biochemical analysis unit in accordance with the present invention, besides the data concerning the time acting as the reference time for the calculation of the attenuation period, at which the radiation from the radioactive labeling substance attenuates, the management data further contains the data concerning the nuclide of the radioactive labeling substance. Therefore, in cases where the management data is read out, the time required for the level of the radioactivity of the radioactive labeling substance to attenuate to a level equal to at most a predetermined level is capable of being calculated in accordance with the data concerning the time acting as the reference time for the calculation of the attenuation period, at which the radiation from the radioactive labeling substance attenuates, and in accordance with the data concerning the nuclide of the radioactive labeling substance. Accordingly, the biochemical analysis unit is capable of being sorted in accordance with the attenuation period, at which the level of the radioactivity of the radioactive labeling substance attenuates to the level equal to at most the predetermined level. In this manner, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

In such cases, the method of managing a biochemical analysis unit in accordance with the present invention should more preferably be modified such that a scrapping period, at which the biochemical analysis unit is capable of being scrapped, is determined in accordance with the data concerning the time acting as the reference time for the calculation of the attenuation period, at which the radiation from the radioactive labeling substance attenuates, and in accordance with the data concerning the nuclide of the radioactive labeling substance, and

the biochemical analysis unit is sorted and managed in accordance with the scrapping period, at which the biochemical analysis unit is capable of being scrapped.

With the aforesaid modification of the method of managing a biochemical analysis unit in accordance with the present invention, the scrapping period, at which the biochemical analysis unit is capable of being scrapped, is determined in accordance with the data concerning the time acting as the reference time for the calculation of the attenuation period, at which the radiation from the radioactive labeling substance attenuates, and in accordance with the data concerning the nuclide of the radioactive labeling substance. Also, the biochemical analysis unit is sorted and managed in accordance with the scrapping period, at which the biochemical analysis unit is capable of being scrapped. Therefore, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

The present invention still further provides a system for managing a biochemical analysis unit comprising a plurality of adsorptive regions located at a spacing from one another, each of the plurality of the adsorptive regions containing one of specific binding substances, whose structures or characteristics are known, the system comprising:

a management data recording apparatus for recording management data that contains data concerning time acting as reference time for a calculation of an attenuation period, at which radiation from a radioactive labeling substance attenuates, on the biochemical analysis unit.

With the system for managing a biochemical analysis unit in accordance with the present invention, wherein the biochemical analysis unit comprising the plurality of the adsorptive regions located at a spacing from one another, each of the plurality of the adsorptive regions containing one of specific binding substances, whose structures or characteristics are known, is managed, the system comprises the management data recording apparatus for recording the management data that contains the data concerning the time acting as the reference time for the calculation of the attenuation period, at which the radiation from the radioactive labeling substance attenuates, on the biochemical analysis unit. Therefore, in accordance with the data concerning the time acting as the reference time for the calculation of the attenuation period, at which the radiation from the radioactive labeling substance attenuates, the data having been recorded on the biochemical analysis unit by the management data recording apparatus, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

The system for managing a biochemical analysis unit in accordance with the present invention should preferably be modified such that the system further comprises a hybridization apparatus for subjecting an organism-originating substance, which has been labeled with a radioactive labeling substance, to selective hybridization with the specific binding substances, each of which is contained in one of the plurality of the adsorptive regions of the biochemical analysis unit, and

the hybridization apparatus is provided with the management data recording apparatus.

With the aforesaid modification of the system for managing a biochemical analysis unit in accordance with the present invention, the system further comprises the hybridization apparatus for subjecting the organism-originating substance, which has been labeled with the radioactive labeling substance, to the selective hybridization with the specific binding substances, each of which is contained in one of the plurality of the adsorptive regions of the biochemical analysis unit, and the hybridization apparatus is provided with the management data recording apparatus. Therefore, when the hybridization is executed, the data concerning the time acting as the reference time for the calculation of the attenuation period, at which the radiation from the radioactive labeling substance attenuates, is capable of being recorded on the biochemical analysis unit by the management data recording apparatus of the hybridization apparatus. Also, in accordance with the data concerning the time acting as the reference time for the calculation of the attenuation period, at which the radiation from the radioactive labeling substance attenuates, the data having been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Further, after the level of the radioactivity of the radioactive labeling substance has attenuated to the level equal to at most the predetermined level, the biochemical analysis unit is capable of being scrapped.

The system for managing a biochemical analysis unit in accordance with the present invention should more preferably be modified such that the data concerning the time acting as the reference time for the calculation of the attenuation period, at which the radiation from the radioactive labeling substance attenuates, contains data concerning a date and hour of execution of the hybridization.

With the aforesaid modification of the system for managing a biochemical analysis unit in accordance with the present invention, the data concerning the time acting as the reference time for the calculation of the attenuation period, at which the radiation from the radioactive labeling substance attenuates, contains the data concerning the date and hour of execution of the hybridization. Therefore, in accordance with the data concerning the date and hour of execution of the hybridization, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Also, the system for managing a biochemical analysis unit in accordance with the present invention should more preferably be modified such that the data concerning the time acting as the reference time for the calculation of the attenuation period, at which the radiation from the radioactive labeling substance attenuates, contains data concerning a day of execution of the hybridization.

With the aforesaid modification of the system for managing a biochemical analysis unit in accordance with the present invention, the data concerning the time acting as the reference time for the calculation of the attenuation period, at which the radiation from the radioactive labeling substance attenuates, contains the data concerning the day of execution of the hybridization. Therefore, in accordance with the data concerning the day of execution of the hybridization, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Further, the system for managing a biochemical analysis unit in accordance with the present invention should preferably be modified such that the system further comprises an exposure apparatus for:

receiving the biochemical analysis unit comprising the plurality of the adsorptive regions, each of the plurality of the adsorptive regions containing one of the specific binding substances, whose structures or characteristics are known, an organism-originating substance, which has been labeled with at least the radioactive labeling substance, having been subjected to selective hybridization with the specific binding substances, each of which is contained in one of the plurality of the adsorptive regions of the biochemical analysis unit, and

executing an exposure operation by superposing the received biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective hybridization, and

the exposure apparatus is provided with the management data recording apparatus.

With the aforesaid modification of the system for managing a biochemical analysis unit in accordance with the present invention, the system further comprises the exposure apparatus. The exposure apparatus receives the biochemical analysis unit comprising the plurality of the adsorptive regions, each of the plurality of the adsorptive regions containing one of the specific binding substances, whose structures or characteristics are known, the organism-originating substance, which has been labeled with at least the radioactive labeling substance, having been subjected to the selective hybridization with the specific binding substances, each of which is contained in one of the plurality of the adsorptive regions of the biochemical analysis unit. Also, the exposure apparatus performs the exposure operation by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective hybridization. Further, the exposure apparatus is provided with the management data recording apparatus. Therefore, when the exposure operation is executed, the data concerning the time acting as the reference time for the calculation of the attenuation period, at which the radiation from the radioactive labeling substance attenuates, is capable of being recorded on the biochemical analysis unit by the management data recording apparatus of the exposure apparatus. Also, in accordance with the data concerning the time acting as the reference time for the calculation of the attenuation period, at which the radiation from the radioactive labeling substance attenuates, the data having been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Further, after the level of the radioactivity of the radioactive labeling substance has attenuated to the level equal to at most the predetermined level, the biochemical analysis unit is capable of being scrapped.

In such cases, the system for managing a biochemical analysis unit in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

With the aforesaid modification of the system for managing a biochemical analysis unit in accordance with the present invention, in accordance with the data concerning the date and hour of execution of the exposure operation, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

The system for managing a biochemical analysis unit in accordance with the present invention should more preferably be modified such that the data concerning the time acting as the reference time for the calculation of the attenuation period, at which the radiation from the radioactive labeling substance attenuates, contains data concerning a day of execution of the exposure operation.

With the aforesaid modification of the system for managing a biochemical analysis unit in accordance with the present invention, in accordance with the data concerning the day of execution of the exposure operation, which data has been recorded on the biochemical analysis unit, and in accordance with the nuclide of the radioactive labeling substance used, the biochemical analysis unit having been used for the biochemical analysis is capable of being managed reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Furthermore, the system for managing a biochemical analysis unit in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

With the aforesaid modification of the system for managing a biochemical analysis unit in accordance with the present invention, the data concerning the date and hour of formation of the radioactive labeling substance is recorded on the biochemical analysis unit. Therefore, in accordance with the data concerning the date and hour of formation of the radioactive labeling substance, which data has been recorded on the biochemical analysis unit, and in accordance with the nuclide of the radioactive labeling substance used, the biochemical analysis unit having been used for the biochemical analysis is capable of being managed more reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

Also, the system for managing a biochemical analysis unit in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

With the aforesaid modification of the system for managing a biochemical analysis unit in accordance with the present invention, the data concerning the day of formation of the radioactive labeling substance is recorded on the biochemical analysis unit. Therefore, in accordance with the data concerning the day of formation of the radioactive labeling substance, which data has been recorded on the biochemical analysis unit, and in accordance with the nuclide of the radioactive labeling substance used, the biochemical analysis unit having been used for the biochemical analysis is capable of being managed more reliably until the level of the radioactivity of the radioactive labeling substance attenuates to a level equal to at most a predetermined level. Also, after the level of the radioactivity of the radioactive labeling substance has attenuated to the level equal to at most the predetermined level, the biochemical analysis unit is capable of being scrapped.

In the system for managing a biochemical analysis unit in accordance with the present invention, the management data may be recorded on the biochemical analysis unit with one of various recording techniques, such as printing and marking. Also, the management data recording apparatus may record the management data as visible data on the biochemical analysis unit. Further, the base plate of the biochemical analysis unit may be provided with a magnetic recording layer, and the management data recording apparatus may magnetically record the management data on the magnetic recording layer.

Also, the system for managing a biochemical analysis unit in accordance with the present invention should preferably be modified such that, besides the data concerning the time acting as the reference time for the calculation of the attenuation period, at which the radiation from the radioactive labeling substance attenuates, the management data further contains data concerning a nuclide of the radioactive labeling substance.

With the aforesaid modification of the system for managing a biochemical analysis unit in accordance with the present invention, besides the data concerning the time acting as the reference time for the calculation of the attenuation period, at which the radiation from the radioactive labeling substance attenuates, the management data further contains the data concerning the nuclide of the radioactive labeling substance. Therefore, in cases where the management data is read out, the time required for the level of the radioactivity of the radioactive labeling substance to attenuate to a level equal to at most a predetermined level is capable of being calculated in accordance with the data concerning the time acting as the reference time for the calculation of the attenuation period, at which the radiation from the radioactive labeling substance attenuates, and in accordance with the data concerning the nuclide of the radioactive labeling substance. Accordingly, the biochemical analysis unit is capable of being sorted in accordance with the attenuation period, at which the level of the radioactivity of the radioactive labeling substance attenuates to the level equal to at most the predetermined level. In this manner, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

In such cases, the system for managing a biochemical analysis unit in accordance with the present invention should more preferably be modified such that the system further comprises a biochemical analysis unit sorting apparatus for:

determining a scrapping period, at which the biochemical analysis unit is capable of being scrapped, in accordance with the data concerning the time acting as the reference time for the calculation of the attenuation period, at which the radiation from the radioactive labeling substance attenuates, and in accordance with the data concerning the nuclide of the radioactive labeling substance, and

sorting and managing the biochemical analysis unit in accordance with the scrapping period, at which the biochemical analysis unit is capable of being scrapped.

With the aforesaid modification of the system for managing a biochemical analysis unit in accordance with the present invention, the system further comprises the biochemical analysis unit sorting apparatus. The biochemical analysis unit sorting apparatus determines the scrapping period, at which the biochemical analysis unit is capable of being scrapped, in accordance with the data concerning the time acting as the reference time for the calculation of the attenuation period, at which the radiation from the radioactive labeling substance attenuates, and in accordance with the data concerning the nuclide of the radioactive labeling substance. Also, the biochemical analysis unit sorting apparatus sorts and manages the biochemical analysis unit in accordance with the scrapping period, at which the biochemical analysis unit is capable of being scrapped. Therefore, the biochemical analysis unit, which has been used for the biochemical analysis, is capable of being automatically sorted and managed in accordance with the scrapping period, at which the biochemical analysis unit is capable of being scrapped. Accordingly, the biochemical analysis unit, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit is capable of being scrapped.

The present invention also provides a method of managing a biochemical analysis medium, comprising the steps of:

i) recording management data on a biochemical analysis medium, the management data containing data concerning time acting as reference time for a calculation of an attenuation period, at which radiation from a radioactive labeling substance attenuates, and data concerning a nuclide of the radioactive labeling substance,

ii) executing a biochemical analysis by use of the biochemical analysis medium, on which the management data has been recorded,

iii) reading out the management data, which has been recorded on the biochemical analysis medium, from the biochemical analysis medium after the biochemical analysis has been executed,

iv) calculating a scrapping period, at which a level of radioactivity of the radioactive labeling substance contained in the biochemical analysis medium decreases to a level equal to at most a level that allows the biochemical analysis medium to be scrapped, in accordance with the management data having been read out,

v) sorting the biochemical analysis medium in accordance with the scrapping period,

vi) accommodating the biochemical analysis medium in a storage box in accordance with the scrapping period, and

vii) storing and managing the biochemical analysis medium, which has been accommodated in the storage box.

With the method of managing a biochemical analysis medium in accordance with the present invention, the management data is recorded on the biochemical analysis medium. The management data contains the data concerning the time acting as the reference time for the calculation of the attenuation period, at which the radiation from the radioactive labeling substance attenuates, and the data concerning the nuclide of the radioactive labeling substance. After the biochemical analysis has been executed by use of the biochemical analysis medium, on which the management data has been recorded, the management data, which has been recorded on the biochemical analysis medium, is readout from the biochemical analysis medium. The scrapping period, at which the level of the radioactivity of the radioactive labeling substance contained in the biochemical analysis medium decreases to the level equal to at most the level that allows the biochemical analysis medium to be scrapped, is then calculated in accordance with the management data having been read out. Also, the biochemical analysis medium is sorted in accordance with the scrapping period, accommodated in the storage box in accordance with the scrapping period, and stored and managed. Therefore, the biochemical analysis unit, which has been used for the biochemical analysis, or the stimulable phosphor sheet, which has been used for the biochemical analysis, is capable of being managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit or the stimulable phosphor sheet is capable of being scrapped without fail.

The method of managing a biochemical analysis medium in accordance with the present invention should preferably be modified such that the scrapping period of the biochemical analysis medium, which has been accommodated in the storage box, is indicated on the storage box.

With the aforesaid modification of the method of managing a biochemical analysis medium in accordance with the present invention, wherein the scrapping period of the biochemical analysis medium, which has been accommodated in the storage box, is indicated on the storage box, a biochemical analysis medium manager is capable of reliably managing the biochemical analysis unit, which has been used for the biochemical analysis, or the stimulable phosphor sheet, which has been used for the biochemical analysis, until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit or the stimulable phosphor sheet is capable of being scrapped without fail.

Also, the method of managing a biochemical analysis medium in accordance with the present invention should preferably be modified such that the biochemical analysis medium is constituted of a biochemical analysis unit comprising a plurality of adsorptive regions located at a spacing from one another, each of the plurality of the adsorptive regions containing one of specific binding substances, whose structures or characteristics are known,

an organism-originating substance, which has been labeled with at least the radioactive labeling substance, is subjected by a hybridization apparatus to hybridization with the specific binding substances, each of which is contained in one of the plurality of the adsorptive regions of the biochemical analysis unit, and

the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the hybridization apparatus when the hybridization is executed.

With the aforesaid modification of the method of managing a biochemical analysis medium in accordance with the present invention, when the organism-originating substance, which has been labeled with at least the radioactive labeling substance, is subjected by the hybridization apparatus to the hybridization with the specific binding substances, each of which is contained in one of the plurality of the adsorptive regions of the biochemical analysis unit, the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the hybridization apparatus. Therefore, in cases where the data concerning the time acting as the reference time for the calculation of the attenuation period, at which the radiation from the radioactive labeling substance attenuates, and the data concerning the nuclide of the radioactive labeling substance having been used for the hybridization, the data having been recorded on the biochemical analysis unit, are read out from the biochemical analysis unit, the scrapping period, at which the level of the radioactivity of the radioactive labeling substance contained in the biochemical analysis unit decreases to the level equal to at most the level that allows the biochemical analysis unit to be scrapped, is capable of being calculated. Also, the biochemical analysis unit is capable of being sorted reliably in accordance with the scrapping period and accommodated in the storage box in accordance with the scrapping period. In this manner, the biochemical analysis unit is capable of being stored and managed without fail until the level of the radioactivity of the radioactive labeling substance contained in the biochemical analysis unit decreases to the level equal to at most the level that allows the biochemical analysis unit to be scrapped.

The method of managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the hybridization.

Also, the method of managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the hybridization.

Further, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

Furthermore, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

Also, the method of managing a biochemical analysis medium in accordance with the present invention should preferably be modified such that the biochemical analysis medium is constituted of a biochemical analysis unit comprising a plurality of adsorptive regions located at a spacing from one another, each of the plurality of the adsorptive regions containing one of specific binding substances, whose structures or characteristics are known,

an organism-originating substance, which has been labeled with at least the radioactive labeling substance, is subjected to selective, specific binding to the specific binding substances, each of which is contained in one of the plurality of the adsorptive regions of the biochemical analysis unit,

an exposure operation is thereafter executed with an exposure apparatus by superposing the biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and

the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the exposure apparatus when the exposure operation is executed.

With the aforesaid modification of the method of managing a biochemical analysis medium in accordance with the present invention, when the exposure operation is executed with the exposure apparatus by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the exposure apparatus. Therefore, in cases where the data concerning the time acting as the reference time for the calculation of the attenuation period, at which the radiation from the radioactive labeling substance attenuates, and the data concerning the nuclide of the radioactive labeling substance having been used for the hybridization, the data having been recorded on the biochemical analysis unit, are read out from the biochemical analysis unit, the scrapping period, at which the level of the radioactivity of the radioactive labeling substance contained in the biochemical analysis unit decreases to the level equal to at most the level that allows the biochemical analysis unit to be scrapped, is capable of being calculated. Also, the biochemical analysis unit is capable of being sorted reliably in accordance with the scrapping period and accommodated in the storage box in accordance with the scrapping period. In this manner, the biochemical analysis unit is capable of being stored and managed without fail until the level of the radioactivity of the radioactive labeling substance contained in the biochemical analysis unit decreases to the level equal to at most the level that allows the biochemical analysis unit to be scrapped.

In such cases, the method of managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

Also, in such cases, the method of managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the exposure operation.

Also, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

Further, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

In the method of managing a biochemical analysis medium in accordance with the present invention, the management data may be recorded on the biochemical analysis unit with one of various recording techniques, such as printing and marking.

Also, the method of managing a biochemical analysis medium in accordance with the present invention should preferably be modified such that each of the adsorptive regions of the base plate of the biochemical analysis unit has an approximately circular shape.

Further, the method of managing a biochemical analysis medium in accordance with the present invention should preferably be modified such that each of the adsorptive regions of the base plate of the biochemical analysis unit has an approximately rectangular shape.

Furthermore, the method of managing a biochemical analysis medium in accordance with the present invention should preferably be modified such that the base plate of the biochemical analysis unit is provided with a data recording layer, and

the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the base plate of the biochemical analysis unit.

With the aforesaid modification of the method of managing a biochemical analysis medium in accordance with the present invention, the base plate of the biochemical analysis unit is provided with the data recording layer, and the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the base plate of the biochemical analysis unit. Therefore, in cases where the data concerning the time acting as the reference time for the calculation of the attenuation period, at which the radiation from the radioactive labeling substance attenuates, and the data concerning the nuclide of the radioactive labeling substance, the data having been recorded on the data recording layer of the base plate of the biochemical analysis unit, are read out from the data recording layer of the base plate of the biochemical analysis unit, the scrapping period, at which the level of the radioactivity of the radioactive labeling substance contained in the biochemical analysis unit decreases to the level equal to at most the level that allows the biochemical analysis unit to be scrapped, is capable of being calculated. Also, the biochemical analysis unit is capable of being sorted reliably in accordance with the scrapping period and accommodated in the storage box in accordance with the scrapping period. In this manner, the biochemical analysis unit is capable of being stored and managed without fail until the level of the radioactivity of the radioactive labeling substance contained in the biochemical analysis unit decreases to the level equal to at most the level that allows the biochemical analysis unit to be scrapped.

Also, with the aforesaid modification of the method of managing a biochemical analysis medium in accordance with the present invention, the management data that contains the data concerning the time acting as the reference time for the calculation of the attenuation period, at which the radiation from the radioactive labeling substance attenuates, and the data concerning the nuclide of the radioactive labeling substance is recorded on the data recording layer. Therefore, in cases where the biochemical analysis unit having been scrapped is collected, and the management data having been recorded on the data recording layer is erased, the biochemical analysis unit is capable of being recycled. As a result, cost reduction and efficient utilization of resources are capable of being achieved.

Further, the method of managing a biochemical analysis medium in accordance with the present invention should preferably be modified such that the biochemical analysis medium is constituted of a stimulable phosphor sheet comprising a support and a stimulable phosphor layer containing a stimulable phosphor, which stimulable phosphor layer is formed on or in the support,

a biochemical analysis unit comprising a plurality of adsorptive regions, each of the plurality of the adsorptive regions containing one of specific binding substances, whose structures or characteristics are known, is obtained, an organism-originating substance, which has been labeled with at least the radioactive labeling substance, having been subjected to selective, specific binding to the specific binding substances, each of which is contained in one of the plurality of the adsorptive regions of the biochemical analysis unit,

an exposure operation is executed with an exposure apparatus by superposing the biochemical analysis unit and the stimulable phosphor sheet one upon the other, the stimulable phosphor contained in the stimulable phosphor layer formed on or in the support of the stimulable phosphor sheet being thereby exposed to radiation radiated out from the radioactive labeling substance, which is contained selectively in at least one adsorptive region among the plurality of the adsorptive regions of the biochemical analysis unit as a result of the selective, specific binding, and

the management data that contains the data concerning the time acting as the reference time for the calculation of the attenuation period, at which the radiation from the radioactive labeling substance attenuates, and the data concerning the nuclide of the radioactive labeling substance is recorded on the support of the stimulable phosphor sheet by the exposure apparatus when the exposure operation is executed.

With the aforesaid modification of the method of managing a biochemical analysis medium in accordance with the present invention, the biochemical analysis unit comprising the plurality of the adsorptive regions, each of the plurality of the adsorptive regions containing one of the specific binding substances, whose structures or characteristics are known, is obtained, the organism-originating substance, which has been labeled with at least the radioactive labeling substance, having been subjected to the selective, specific binding to the specific binding substances, each of which is contained in one of the plurality of the adsorptive regions of the biochemical analysis unit, and the exposure operation is executed with the exposure apparatus by superposing the biochemical analysis unit and the stimulable phosphor sheet one upon the other, the stimulable phosphor contained in the stimulable phosphor layer formed on or in the support of the stimulable phosphor sheet being thereby exposed to the radiation radiated out from the radioactive labeling substance, which is contained selectively in at least one adsorptive region among the plurality of the adsorptive regions of the biochemical analysis unit as a result of the selective, specific binding. When the exposure operation is executed, the management data that contains the data concerning the time acting as the reference time for the calculation of the attenuation period, at which the radiation from the radioactive labeling substance attenuates, and the data concerning the nuclide of the radioactive labeling substance is recorded on the support of the stimulable phosphor sheet by the exposure apparatus. Therefore, in cases where the data concerning the time acting as the reference time for the calculation of the attenuation period, at which the radiation from the radioactive labeling substance attenuates, and the data concerning the nuclide of the radioactive labeling substance having been used in the exposure operation, the data having been recorded on the support of the stimulable phosphor sheet, are read out from the support of the stimulable phosphor sheet, the scrapping period, at which the level of the radioactivity of the radioactive labeling substance contained in the stimulable phosphor layer of the stimulable phosphor sheet decreases to the level equal to at most the level that allows the stimulable phosphor sheet to be scrapped, is capable of being calculated. Also, the stimulable phosphor sheet is capable of being sorted reliably in accordance with the scrapping period and accommodated in the storage box in accordance with the scrapping period. In this manner, the stimulable phosphor sheet is capable of being stored and managed without fail until the level of the radioactivity of the radioactive labeling substance contained in the stimulable phosphor layer of the stimulable phosphor sheet decreases to the level equal to at most the level that allows the stimulable phosphor sheet to be scrapped.

In such cases, the method of managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

Also, in such cases, the method of managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the exposure operation.

Further, in such cases, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

Furthermore, in such cases, the method of managing a biochemical analysis medium in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

Also, the method of managing a biochemical analysis medium in accordance with the present invention should preferably be modified such that the stimulable phosphor layer comprises a plurality of stimulable phosphor layer regions, which are located at a spacing from one another.

With the aforesaid modification of the method of managing a biochemical analysis medium in accordance with the present invention, the stimulable phosphor layer comprises the plurality of the stimulable phosphor layer regions, which are located at a spacing from one another. Therefore, a plurality of spot-shaped regions, each of which contains one of the specific binding substances, may be formed on or in the biochemical analysis unit in the pattern identical with the pattern of the plurality of the stimulable phosphor layer regions having been formed on or in the support of the stimulable phosphor sheet, and the organism-originating substance, which has been labeled with at least the radioactive labeling substance, may be subjected to the hybridization with the specific binding substances, each of which is contained in one of the plurality of the spot-shaped regions of the biochemical analysis unit. The exposure operation may then be executed with the exposure apparatus by superposing the biochemical analysis unit and the stimulable phosphor sheet one upon the other, such that each of the stimulable phosphor layer regions having been formed on or in the support of the stimulable phosphor sheet stand facing one of the spot-shaped regions of the biochemical analysis unit. The stimulable phosphor contained in each of the stimulable phosphor layer regions of the stimulable phosphor sheet may thus be exposed to the radiation radiated out from the radioactive labeling substance contained in the spot-shaped region of the biochemical analysis unit, which spot-shaped region stands facing the stimulable phosphor layer region. In this manner, the electron rays (the beta rays), which have been radiated out from the radioactive labeling substance contained in one of the spot-shaped regions of the biochemical analysis unit, are capable of selectively impinging upon the stimulable phosphor layer region, which stands facing the spot-shaped region of the biochemical analysis unit, and only the corresponding stimulable phosphor layer region is thus capable of being exposed to the electron rays (the beta rays). Therefore, in cases where the stimulable phosphor layer region having thus been exposed to the electron rays (the beta rays) radiated out from the radioactive labeling substance is thereafter scanned with the stimulating rays, and the light emitted by the stimulable phosphor layer region is photoelectrically detected, the data for a biochemical analysis having good quantitative characteristics is capable of being formed with a high resolution.

Further, the method of managing a biochemical analysis medium in accordance with the present invention should preferably be modified such that the support of the stimulable phosphor sheet is provided with a data recording layer, on which data is capable of being recorded, and

the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the support of the stimulable phosphor sheet by the exposure apparatus.

With the aforesaid modification of the method of managing a biochemical analysis medium in accordance with the present invention, the support of the stimulable phosphor sheet is provided with the data recording layer, on which data is capable of being recorded, and the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the support of the stimulable phosphor sheet by the exposure apparatus. Therefore, in cases where the data concerning the time acting as the reference time for the calculation of the attenuation period, at which the radiation from the radioactive labeling substance attenuates, and the data concerning the nuclide of the radioactive labeling substance having been used in the exposure operation, the data having been recorded on the data recording layer of the support of the stimulable phosphor sheet, are read out from the data recording layer of the support of the stimulable phosphor sheet, the scrapping period, at which the level of the radioactivity of the radioactive labeling substance contained in the stimulable phosphor layer of the stimulable phosphor sheet decreases to the level equal to at most the level that allows the stimulable phosphor sheet to be scrapped, is capable of being calculated. Also, the stimulable phosphor sheet is capable of being sorted reliably in accordance with the scrapping period and accommodated in the storage box in accordance with the scrapping period. In this manner, the stimulable phosphor sheet is capable of being stored and managed without fail until the level of the radioactivity of the radioactive labeling substance contained in the stimulable phosphor layer of the stimulable phosphor sheet decreases to the level equal to at most the level that allows the stimulable phosphor sheet to be scrapped.

Also, with the aforesaid modification of the method of managing a biochemical analysis medium in accordance with the present invention, the management data is recorded on the data recording layer of the support of the stimulable phosphor sheet. Therefore, in cases where the stimulable phosphor sheet having been scrapped is collected, and the management data having been recorded on the data recording layer is erased, the stimulable phosphor sheet is capable of being recycled. As a result, cost reduction and efficient utilization of resources are capable of being achieved.

The present invention still further provides a system for managing a biochemical analysis medium, comprising:

i) a radioactive label imparting apparatus for imparting a radioactive label to a biochemical analysis medium by use of a radioactive labeling substance, and

ii) a biochemical analysis medium sorting apparatus for sorting the biochemical analysis medium, which has been labeled with the radioactive labeling substance and has been used for a biochemical analysis,

wherein the radioactive label imparting apparatus is constituted to record management data on the biochemical analysis medium, the management data containing data concerning time acting as reference time for a calculation of an attenuation period, at which radiation from the radioactive labeling substance attenuates, and data concerning a nuclide of the radioactive labeling substance, and

the biochemical analysis medium sorting apparatus is constituted to perform the operations for:

reading out the management data, which has been recorded on the biochemical analysis medium, from the biochemical analysis medium,

calculating a scrapping period, at which a level of radioactivity of the radioactive labeling substance contained in the biochemical analysis medium decreases to a level equal to at most a level that allows the biochemical analysis medium to be scrapped, in accordance with the management data having been read out,

sorting the biochemical analysis medium in accordance with the scrapping period, and

accommodating the biochemical analysis medium in a storage box in accordance with the scrapping period.

With the system for managing a biochemical analysis medium in accordance with the present invention, the system comprises the radioactive label imparting apparatus for imparting the radioactive label to the biochemical analysis medium by use of the radioactive labeling substance, and the biochemical analysis medium sorting apparatus for sorting the biochemical analysis medium, which has been labeled with the radioactive labeling substance and has been used for a biochemical analysis. The radioactive label imparting apparatus is constituted to record the management data on the biochemical analysis medium, the management data containing the data concerning the time acting as the reference time for the calculation of the attenuation period, at which the radiation from the radioactive labeling substance attenuates, and the data concerning the nuclide of the radioactive labeling substance. Also, the biochemical analysis medium sorting apparatus is constituted to perform the operations for reading out the management data, which has been recorded on the biochemical analysis medium, from the biochemical analysis medium, calculating the scrapping period, at which the level of the radioactivity of the radioactive labeling substance contained in the biochemical analysis medium decreases to the level equal to at most the level that allows the biochemical analysis medium to be scrapped, in accordance with the management data having been read out, sorting the biochemical analysis medium in accordance with the scrapping period, and accommodating the biochemical analysis medium in the storage box in accordance with the scrapping period. Therefore, after the biochemical analysis has been executed by use of the biochemical analysis medium, the management data that contains the data concerning the time acting as the reference time for the calculation of the attenuation period, at which the radiation from the radioactive labeling substance attenuates, and the data concerning the nuclide of the radioactive labeling substance, the management data having been recorded on the biochemical analysis medium having been used for the biochemical analysis, is capable of being read out from the biochemical analysis medium by the biochemical analysis medium sorting apparatus. Also, the biochemical analysis unit, which has been used for the biochemical analysis, or the stimulable phosphor sheet, which has been used for the biochemical analysis, is capable of being sorted in accordance with the scrapping period and accommodated in the storage box in accordance with the scrapping period. Accordingly, the biochemical analysis unit, which has been used for the biochemical analysis, or the stimulable phosphor sheet, which has been used for the biochemical analysis, is capable of being stored and managed reliably until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit or the stimulable phosphor sheet is capable of being scrapped without fail.

The system for managing a biochemical analysis medium in accordance with the present invention should preferably be modified such that the scrapping period of the biochemical analysis medium, which has been accommodated in the storage box, is indicated on the storage box.

With the aforesaid modification of the system for managing a biochemical analysis medium in accordance with the present invention, wherein the scrapping period of the biochemical analysis medium, which has been accommodated in the storage box, is indicated on the storage box, a biochemical analysis medium manager is capable of reliably managing the biochemical analysis unit, which has been used for the biochemical analysis, or the stimulable phosphor sheet, which has been used for the biochemical analysis, until the level of the radioactivity of the radioactive labeling substance attenuates to the level equal to at most the predetermined level. Also, after the level of the radioactivity of the radioactive labeling substance has attenuated to the level equal to at most the predetermined level, the biochemical analysis unit or the stimulable phosphor sheet is capable of being scrapped without fail.

Also, the system for managing a biochemical analysis medium in accordance with the present invention should preferably be modified such that the biochemical analysis medium is constituted of a biochemical analysis unit comprising a plurality of adsorptive regions located at a spacing from one another, each of the plurality of the adsorptive regions containing one of specific binding substances, whose structures or characteristics are known,

the radioactive label imparting apparatus is constituted of a hybridization apparatus for subjecting an organism-originating substance, which has been labeled with at least the radioactive labeling substance, to hybridization with the specific binding substances, each of which is contained in one of the plurality of the adsorptive regions of the biochemical analysis unit, and

the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit when the hybridization of the organism-originating substance, which has been labeled with at least the radioactive labeling substance, with the specific binding substances, each of which is contained in one of the plurality of the adsorptive regions of the biochemical analysis unit, is executed.

With the aforesaid modification of the system for managing a biochemical analysis medium in accordance with the present invention, when the organism-originating substance, which has been labeled with at least the radioactive labeling substance, is subjected to the hybridization with the specific binding substances, each of which is contained in one of the plurality of the adsorptive regions of the biochemical analysis unit, the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit. Therefore, in cases where the data concerning the time acting as the reference time for the calculation of the attenuation period, at which the radiation from the radioactive labeling substance attenuates, and the data concerning the nuclide of the radioactive labeling substance having been used for the hybridization, the data having been recorded on the biochemical analysis unit, are read out from the biochemical analysis unit by the biochemical analysis medium sorting apparatus, the scrapping period, at which the level of the radioactivity of the radioactive labeling substance contained in the biochemical analysis unit decreases to the level equal to at most the level that allows the biochemical analysis unit to be scrapped, is capable of being calculated. Also, the biochemical analysis unit is capable of being sorted reliably in accordance with the scrapping period and accommodated in the storage box in accordance with the scrapping period. In this manner, the biochemical analysis unit is capable of being stored and managed without fail until the level of the radioactivity of the radioactive labeling substance contained in the biochemical analysis unit decreases to the level equal to at most the level that allows the biochemical analysis unit to be scrapped.

The system for managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the hybridization.

Also, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the hybridization.

Further, the system for managing a biochemical analysis medium in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

Furthermore, the system for managing a biochemical analysis medium in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

Also, the system for managing a biochemical analysis medium in accordance with the present invention should preferably be modified such that the biochemical analysis medium is constituted of a biochemical analysis unit comprising a plurality of adsorptive regions located at a spacing from one another, each of the plurality of the adsorptive regions containing one of specific binding substances, whose structures or characteristics are known,

the radioactive label imparting apparatus is constituted of an exposure apparatus for:

receiving the biochemical analysis unit comprising the plurality of the adsorptive regions, each of the plurality of the adsorptive regions containing one of the specific binding substances, whose structures or characteristics are known, an organism-originating substance, which has been labeled with at least the radioactive labeling substance, having been subjected to selective, specific binding to the specific binding substances, each of which is contained in one of the plurality of the adsorptive regions of the biochemical analysis unit, and

executing an exposure operation by superposing the received biochemical analysis unit and a stimulable phosphor sheet, which comprises a stimulable phosphor layer containing a stimulable phosphor, one upon the other, the stimulable phosphor contained in the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and

the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit when the exposure operation for exposing the stimulable phosphor contained in the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

With the aforesaid modification of the system for managing a biochemical analysis medium in accordance with the present invention, when the exposure operation for exposing the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed, the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit. Therefore, in cases where the data concerning the time acting as the reference time for the calculation of the attenuation period, at which the radiation from the radioactive labeling substance attenuates, and the data concerning the nuclide of the radioactive labeling substance having been used for the hybridization, the data having been recorded on the biochemical analysis unit, are read out from the biochemical analysis unit by the biochemical analysis medium sorting apparatus, the scrapping period, at which the level of the radioactivity of the radioactive labeling substance contained in the biochemical analysis unit decreases to the level equal to at most the level that allows the biochemical analysis unit to be scrapped, is capable of being calculated. Also, the biochemical analysis unit is capable of being sorted reliably in accordance with the scrapping period and accommodated in the storage box in accordance with the scrapping period. In this manner, the biochemical analysis unit is capable of being stored and managed without fail until the level of the radioactivity of the radioactive labeling substance contained in the biochemical analysis unit decreases to the level equal to at most the level that allows the biochemical analysis unit to be scrapped.

In such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

Also, in such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the exposure operation.

Further, in such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of formation of the radioactive labeling substance.

Furthermore, in such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a day of formation of the radioactive labeling substance.

In the system for managing a biochemical analysis medium in accordance with the present invention, the management data may be recorded on the biochemical analysis unit with one of various recording techniques, such as printing and marking.

Further, the system for managing a biochemical analysis medium in accordance with the present invention should preferably be modified such that the base plate of the biochemical analysis unit is provided with a data recording layer, and

the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the base plate of the biochemical analysis unit.

Also, the system for managing a biochemical analysis medium in accordance with the present invention should preferably be modified such that the biochemical analysis medium is constituted of a stimulable phosphor sheet comprising a support and a stimulable phosphor layer containing a stimulable phosphor, which stimulable phosphor layer is formed on or in the support,

the radioactive label imparting apparatus is constituted of an exposure apparatus for:

receiving a biochemical analysis unit comprising a plurality of adsorptive regions, each of the plurality of the adsorptive regions containing one of specific binding substances, whose structures or characteristics are known, an organism-originating substance, which has been labeled with at least the radioactive labeling substance, having been subjected to selective, specific binding to the specific binding substances, each of which is contained in one of the plurality of the adsorptive regions of the biochemical analysis unit, and

executing an exposure operation by superposing the received biochemical analysis unit and the stimulable phosphor sheet one upon the other, the stimulable phosphor contained in the stimulable phosphor layer formed on or in the support of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and

the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the support of the stimulable phosphor sheet when the exposure operation for exposing the stimulable phosphor contained in the stimulable phosphor layer formed on or in the support of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

With the aforesaid modification of the system for managing a biochemical analysis medium in accordance with the present invention, the exposure apparatus receives the biochemical analysis unit comprising the plurality of the adsorptive regions, each of the plurality of the adsorptive regions containing one of the specific binding substances, whose structures or characteristics are known, the organism-originating substance, which has been labeled with at least the radioactive labeling substance, having been subjected to the selective, specific binding to the specific binding substances, each of which is contained in one of the plurality of the adsorptive regions of the biochemical analysis unit. Also, the exposure apparatus executes the exposure operation by superposing the biochemical analysis unit and the stimulable phosphor sheet one upon the other, the stimulable phosphor contained in the stimulable phosphor layer formed on or in the support of the stimulable phosphor sheet being thereby exposed to the radiation radiated out from the radioactive labeling substance, which is contained selectively in at least one adsorptive region among the plurality of the adsorptive regions of the biochemical analysis unit as a result of the selective, specific binding. When the exposure operation is executed, the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the support of the stimulable phosphor sheet. Therefore, in cases where the data concerning the time acting as the reference time for the calculation of the attenuation period, at which the radiation from the radioactive labeling substance attenuates, and the data concerning the nuclide of the radioactive labeling substance having been used in the exposure operation, the data having been recorded on the support of the stimulable phosphor sheet having been used, are read out from the support of the stimulable phosphor sheet by the biochemical analysis medium sorting apparatus, the scrapping period, at which the level of the radioactivity of the radioactive labeling substance contained in the stimulable phosphor layer of the stimulable phosphor sheet decreases to the level equal to at most the level that allows the stimulable phosphor sheet to be scrapped, is capable of being calculated. Also, the stimulable phosphor sheet is capable of being sorted reliably in accordance with the scrapping period and accommodated in the storage box in accordance with the scrapping period. In this manner, the stimulable phosphor sheet is capable of being stored and managed without fail until the level of the radioactivity of the radioactive labeling substance contained in the stimulable phosphor layer of the stimulable phosphor sheet decreases to the level equal to at most the level that allows the stimulable phosphor sheet to be scrapped.

In such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a date and hour of execution of the exposure operation.

Also, in such cases, the system for managing a biochemical analysis medium in accordance with the present invention should more preferably be modified such that the data concerning the time acting as the reference time for the calculation of the attenuation period, at which the radiation from the radioactive labeling substance attenuates, contains data concerning a day of execution of the exposure operation.

Further, in such cases, the system for managing a biochemical analysis medium in accordance with the present invention should preferably be modified such that the data concerning the time acting as the reference time for the calculation of the attenuation period, at which the radiation from the radioactive labeling substance attenuates, contains data concerning a date and hour of formation of the radioactive labeling substance.

Furthermore, in such cases, the system for managing a biochemical analysis medium in accordance with the present invention should preferably be modified such that the data concerning the time acting as the reference time for the calculation of the attenuation period, at which the radiation from the radioactive labeling substance attenuates, contains data concerning a day of formation of the radioactive labeling substance.

Also, the system for managing a biochemical analysis medium in accordance with the present invention should preferably be modified such that the stimulable phosphor layer comprises a plurality of stimulable phosphor layer regions, which are located at a spacing from one another.

With the aforesaid modification of the system for managing a biochemical analysis medium in accordance with the present invention, the stimulable phosphor layer comprises the plurality of the stimulable phosphor layer regions, which are located at a spacing from one another. Therefore, a plurality of spot-shaped regions, each of which contains one of the specific binding substances, may be formed on or in the biochemical analysis unit in the pattern identical with the pattern of the plurality of the stimulable phosphor layer regions having been formed on or in the support of the stimulable phosphor sheet, and the organism-originating substance, which has been labeled with at least the radioactive labeling substance, may be subjected to the hybridization with the specific binding substances, each of which is contained in one of the plurality of the spot-shaped regions of the biochemical analysis unit. The exposure operation may then be executed with the exposure apparatus by superposing the biochemical analysis unit and the stimulable phosphor sheet one upon the other, such that each of the stimulable phosphor layer regions having been formed on or in the support of the stimulable phosphor sheet stand facing one of the spot-shaped regions of the biochemical analysis unit. The stimulable phosphor contained in each of the stimulable phosphor layer regions of the stimulable phosphor sheet may thus be exposed to the radiation radiated out from the radioactive labeling substance contained in the spot-shaped region of the biochemical analysis unit, which spot-shaped region stands facing the stimulable phosphor layer region. In this manner, the electron rays (the beta rays), which have been radiated out from the radioactive labeling substance contained in one of the spot-shaped regions of the biochemical analysis unit, are capable of selectively impinging upon the stimulable phosphor layer region, which stands facing the spot-shaped region of the biochemical analysis unit, and only the corresponding stimulable phosphor layer region is thus capable of being exposed to the electron rays (the beta rays). Therefore, in cases where the stimulable phosphor layer region having thus been exposed to the electron rays (the beta rays) radiated out from the radioactive labeling substance is thereafter scanned with the stimulating rays, and the light emitted by the stimulable phosphor layer region is photoelectrically detected, the data for a biochemical analysis having good quantitative characteristics is capable of being formed with a high resolution.

Further, the system for managing a biochemical analysis medium in accordance with the present invention should preferably be modified such that the support of the stimulable phosphor sheet is provided with a data recording layer, on which data is capable of being recorded, and

the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the data recording layer of the support of the stimulable phosphor sheet.

With the aforesaid modification of the system for managing a biochemical analysis medium in accordance with the present invention, the support of the stimulable phosphor sheet is provided with the data recording layer, on which data is capable of being recorded, and the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the data recording layer of the support of the stimulable phosphor sheet. Therefore, in cases where the data concerning the time acting as the reference time for the calculation of the attenuation period, at which the radiation from the radioactive labeling substance attenuates, and the data concerning the nuclide of the radioactive labeling substance having been used in the exposure operation, the data having been recorded on the support of the stimulable phosphor sheet having been used, are read out from the support of the stimulable phosphor sheet by the biochemical analysis medium sorting apparatus, the scrapping period, at which the level of the radioactivity of the radioactive labeling substance contained in the stimulable phosphor layer of the stimulable phosphor sheet decreases to the level equal to at most the level that allows the stimulable phosphor sheet to be scrapped, is capable of being calculated. Also, the stimulable phosphor sheet is capable of being sorted reliably in accordance with the scrapping period and accommodated in the storage box in accordance with the scrapping period. In this manner, the stimulable phosphor sheet is capable of being stored and managed without fail until the level of the radioactivity of the radioactive labeling substance contained in the stimulable phosphor layer of the stimulable phosphor sheet decreases to the level equal to at most the level that allows the stimulable phosphor sheet to be scrapped.

With the biochemical analysis system in accordance with the present invention, the organism-originating substance, which has been labeled with the at least one labeling substance selected from the group consisting of the radioactive labeling substance, the fluorescent labeling substance, and the labeling substance capable of producing chemiluminescence when being brought into contact with the chemiluminescence substrate, is subjected to the specific binding to the multiple kinds of the specific binding substances, each of which has been fixed to one of the spot-shaped regions of the biochemical analysis unit. Thereafter, the label signal is formed with the label signal forming means, which corresponds to the labeling substance having been used at the time of the specific binding, and the data for a biochemical analysis is formed by reading out the label signal. The thus obtained data for a biochemical analysis is utilized for analyzing the structure, the characteristics, and the like of the organism-originating substance. In such cases, the identification information of the biochemical analysis unit and the identification information of the organism-originating substance are acquired automatically, and the correspondence relationship between the two pieces of the identification information is specified. Therefore, without reliance upon memory of the operator, a memorandum made by the operator, or the like, being made, it is capable of being found accurately which organism-originating substance was subjected to the specific binding with which biochemical analysis unit. Accordingly, artificial mistakes are capable of being avoided, and correct results of analysis are capable of being obtained.

Also, with the biochemical analysis system in accordance with the present invention, the base plate of the biochemical analysis unit may be made from the material having the radiation attenuating properties and/or the light attenuating properties and may have the plurality of the holes, the adsorptive region may be formed in each of the plurality of the holes of the base plate, and each of the multiple kinds of the specific binding substances may be fixed to one of the adsorptive regions in order to form one of the spot-shaped regions. In such cases, the biochemical analysis unit is capable of being provided with the spot-shaped regions located at a high density, and the efficiency with which the analysis is made is capable of being enhanced. Also, when the label signal is formed by use of the biochemical analysis unit having been processed with the specific binding means, the radiation, the fluorescence, or the chemiluminescence produced from the spot-shaped region is capable of being prevented from scattering. Therefore, the occurrence of noise is capable of being suppressed, and good analysis results are capable of being obtained.

With the biochemical analysis system in accordance with the present invention, wherein the radioactive labeling substance is utilized as the labeling substance and the stimulable phosphor sheet is utilized, the identification information of the biochemical analysis unit and the identification information of the stimulable phosphor sheet are acquired, and the correspondence relationship between the two pieces of the identification information is specified. Therefore, it is capable of being found automatically which radiation information stored on which stimulable phosphor sheet was obtained from which biochemical analysis unit (in other words, which radiation information stored on which stimulable phosphor sheet was obtained with respect to which organism-originating substance). Accordingly, in the biochemical analysis system utilizing the stimulable phosphor sheet, artificial mistakes are capable of being avoided.

Also, in cases where the identification information of the stimulable phosphor sheet is acquired, the number of times of use of the stimulable phosphor sheet is capable of being counted. Therefore, the management of the scrapping period of the stimulable phosphor sheet, and the like, is capable of being performed easily in accordance with the number of times of use of the stimulable phosphor sheet. Accordingly, the problems are capable of being prevented from occurring in that a deteriorated stimulable phosphor sheet is further used.

Further, the biochemical analysis system in accordance with the present invention may be modified such that the stimulable phosphor sheet comprises the support and the plurality of the dot-shaped stimulable phosphor layer regions, which are located at a spacing from one another on or in the support, and the positions and the sizes of the plurality of the dot-shaped stimulable phosphor layer regions correspond respectively to the positions and the sizes of the spot-shaped regions of the biochemical analysis unit. In such cases, the stimulable phosphor sheet and the biochemical analysis unit having been processed with the specific binding means are capable of being superposed one upon the other, such that the position of each of the dot-shaped stimulable phosphor layer regions of the stimulable phosphor sheet coincides with the position of one of the spot-shaped regions of the biochemical analysis unit. Therefore, the radiation radiated out from each of the spot-shaped regions of the biochemical analysis unit impinges upon only the corresponding stimulable phosphor layer region of the stimulable phosphor sheet. Accordingly, the problems are capable of being prevented from occurring in that the radiation radiated out from a certain spot-shaped region of the biochemical analysis unit impinges upon a site on the stimulable phosphor sheet, which site corresponds to a spot-shaped region adjacent to the certain spot-shaped region of the biochemical analysis unit, and noise is thus caused to occur.

The stimulable phosphor sheet comprising the dot-shaped stimulable phosphor layer regions may be utilized in combination with the biochemical analysis unit, wherein the base plate is made from the material having the radiation attenuating properties and/or the light attenuating properties and has the plurality of the holes, the adsorptive region is formed in each of the plurality of the holes of the base plate, and each of the multiple kinds of the specific binding substances is fixed to one of the adsorptive regions in order to form one of the spot-shaped regions. In such cases, the positions and the sizes of the plurality of the dot-shaped stimulable phosphor layer regions of the stimulable phosphor sheet correspond respectively to the positions and the sizes of the holes of the biochemical analysis unit. Therefore, the occurrence of noise is capable of being prevented even further.

With the biochemical analysis system in accordance with the present invention, wherein the biochemical analysis system further comprises the spotting means for spotting each of the specific binding substances to one of different positions on or in the base plate in order to form the plurality of the spot-shaped regions, and thereby preparing the biochemical analysis unit, the identification information and the spot information of the thus formed biochemical analysis unit are capable of being acquired, and it is capable of being found automatically which specific binding substances have been formed in what location pattern on which biochemical analysis unit. Therefore, in cases where the commercially available biochemical analysis unit is not used and in cases where the biochemical analysis unit having been used for the biochemical analysis is reused, artificial mistakes are capable of being avoided.

With the biochemical analysis system in accordance with the present invention, wherein the identification information of the biochemical analysis unit contains the identification information of the base plate of the biochemical analysis unit, the number of times of use of the base plate is capable of being found, and the management of the scrapping period of the base plate, and the like, is capable of being performed easily. Therefore, the problems are capable of being prevented from occurring in that a deteriorated base plate is used again and the analysis is thus adversely affected.

With the biochemical analysis unit in accordance with the present invention, the data inherent to the biochemical analysis unit, such as the micro array or the macro array, is capable of being managed reliably, and the reliability of the biochemical analysis is capable of being enhanced markedly.

With the method and the system for managing a biochemical analysis unit in accordance with the present invention, the biochemical analysis unit having been used for the biochemical analysis, in which the organism-originating substance having been labeled with the radioactive labeling substance is subjected to the hybridization with the specific binding substances contained in the biochemical analysis unit, is capable of being managed reliably until the level of the radioactivity of the radioactive labeling substance attenuates to the level equal to at most the predetermined level.

With the method and system for managing a biochemical analysis medium in accordance with the present invention, the biochemical analysis unit having been used for the biochemical analysis, in which the organism-originating substance having been labeled with the radioactive labeling substance is subjected to the hybridization with the specific binding substances contained in the biochemical analysis unit, or the stimulable phosphor sheet having been used for the biochemical analysis, in which the stimulable phosphor layer of the stimulable phosphor sheet is exposed to the radiation radiated out from the radioactive labeling substance, is capable of being managed reliably until the level of the radioactivity of the radioactive labeling substance attenuates to the level equal to at most the predetermined level.

### BRIEF DESCRIPTION OF THE DRAWINGS

FIG. 1 is a block diagram showing an embodiment of the biochemical analysis system in accordance with the present invention,

FIG. 2 is a schematic perspective view showing a membrane utilized in the biochemical analysis system of FIG. 1,

FIG. 3 is an explanatory view showing a format of data stored in a data analyzing and control section of the biochemical analysis system of FIG. 1,

FIG. 4 is a schematic perspective view showing a first embodiment of the biochemical analysis unit in accordance with the present invention,

FIG. 5 is a schematic front view showing a spotting apparatus,

FIG. 6 is a schematic plan view showing the spotting apparatus,

FIG. 7 is a block diagram showing a control system, an input system, a driving system, a detecting system, and a recording system of the spotting apparatus,

FIG. 8 is a schematic side view showing a hybridization apparatus,

FIG. 9 is a schematic perspective view showing a cartridge,

FIG. 10 is a block diagram showing a control system, a detecting system, a driving system, an input system, and a displaying system of the hybridization apparatus,

FIG. 11 is a schematic perspective view showing a stimulable phosphor sheet,

FIG. 12 is a schematic perspective view showing an exposure apparatus for executing an exposure operation, wherein at least one stimulable phosphor layer region among a plurality of stimulable phosphor layer regions of the stimulable phosphor sheet is exposed to radiation radiated out from a radioactive labeling substance, which is contained selectively in at least one adsorptive region among a plurality of adsorptive regions of the biochemical analysis unit,

FIG. 13 is a partial schematic sectional view showing a mechanism for locking a cover member of the exposure apparatus for the stimulable phosphor sheet to a casing,

FIG. 14 is a block diagram showing a control system, a detecting system, a driving system, a displaying system, and an input system of the exposure apparatus for the stimulable phosphor sheet,

FIG. 15 is a schematic sectional view showing how at least one dot-shaped stimulable phosphor layer region among the plurality of the dot-shaped stimulable phosphor layer regions of the stimulable phosphor sheet is exposed to the radiation radiated out from the radioactive labeling substance, which is contained selectively in at least one adsorptive region among the plurality of the adsorptive regions of the biochemical analysis unit,

FIG. 16 is a schematic perspective view showing a scanner for reading out radiation information from the stimulable phosphor sheet in order to form data for a biochemical analysis, and reading out fluorescence information from the biochemical analysis unit in order to form data for a biochemical analysis,

FIG. 17 is a schematic perspective view showing a constitution in the vicinity of a photomultiplier in the scanner of FIG. 16,

FIG. 18 is a schematic sectional view taken on line I′-I of FIG. 17,

FIG. 19 is a schematic sectional view taken on line II′-II of FIG. 17,

FIG. 20 is a schematic sectional view taken on line III′-III of FIG. 17,

FIG. 21 is a schematic sectional view taken on line IV′-IV of FIG. 17,

FIG. 22 is a schematic plan view showing an optical head scanning mechanism,

FIG. 23 is a block diagram showing a control system, an input system, a driving system, and a detecting system of the scanner,

FIG. 24 is a schematic perspective view showing a second embodiment of the biochemical analysis unit in accordance with the present invention,

FIG. 25 is a schematic perspective view showing a third embodiment of the biochemical analysis unit in accordance with the present invention,

FIG. 26 is a schematic perspective view showing a fourth embodiment of the biochemical analysis unit in accordance with the present invention,

FIG. 27 is a schematic perspective view showing a fifth embodiment of the biochemical analysis unit in accordance with the present invention,

FIG. 28 is a schematic perspective view showing a biochemical analysis unit, which is managed by a first embodiment of the system for managing a biochemical analysis unit in accordance with the present invention,

FIG. 29 is a schematic side view showing a hybridization apparatus, which constitutes the first embodiment of the system for managing a biochemical analysis unit in accordance with the present invention,

FIG. 30 is a schematic perspective view showing a stimulable phosphor sheet,

FIG. 31 is a schematic sectional view showing how at least one stimulable phosphor layer region among the plurality of the stimulable phosphor layer regions of the stimulable phosphor sheet is exposed to the radiation radiated out from the radioactive labeling substance, which is contained selectively in at least one adsorptive region among the plurality of the adsorptive regions of the biochemical analysis unit,

FIG. 32 is a block diagram showing a control system, an input system, a driving system, and a detecting system of the scanner shown in FIG. 16,

FIG. 33 is a schematic plan view showing a biochemical analysis unit sorting apparatus, which constitutes the first embodiment of the system for managing a biochemical analysis unit in accordance with the present invention,

FIG. 34 is a block diagram showing a control system, a detecting system, a driving system, and an input system of the biochemical analysis unit sorting apparatus, which constitutes the first embodiment of the system for managing a biochemical analysis unit in accordance with the present invention,

FIG. 35 is a schematic perspective view showing a biochemical analysis unit, which is managed by a second embodiment of the system for managing a biochemical analysis unit in accordance with the present invention,

FIG. 36 is a schematic perspective view showing the biochemical analysis unit provided with a plurality of spot-shaped regions located at a spacing from one another, each of the plurality of the spot-shaped regions having been formed with a process for spotting a liquid containing one of specific binding substances onto an adsorptive plate of the biochemical analysis unit by use of the spotting apparatus,

FIG. 37 is a schematic side view showing a hybridization apparatus, which constitutes the second embodiment of the system for managing a biochemical analysis unit in accordance with the present invention,

FIG. 38 is a block diagram showing a control system, a detecting system, a driving system, an input system, and a displaying system of the hybridization apparatus, which constitutes the second embodiment of the system for managing a biochemical analysis unit in accordance with the present invention,

FIG. 39 is a schematic plan view showing a biochemical analysis unit sorting apparatus, which constitutes the second embodiment of the system for managing a biochemical analysis unit in accordance with the present invention,

FIG. 40 is a block diagram showing a control system, a detecting system, a driving system, and an input system of the biochemical analysis unit sorting apparatus, which constitutes the second embodiment of the system for managing a biochemical analysis unit in accordance with the present invention,

FIG. 41 is a schematic perspective view showing an exposure apparatus for executing an exposure operation, wherein a stimulable phosphor contained in at least one stimulable phosphor layer region among a plurality of stimulable phosphor layer regions of the stimulable phosphor sheet shown in FIG. 30 is exposed to the radiation radiated out from the radioactive labeling substance, which is contained selectively in at least one adsorptive region among the plurality of the adsorptive regions of the biochemical analysis unit,

FIG. 42 is a block diagram showing a control system, a detecting system, a driving system, a displaying system, and an input system of the exposure apparatus of FIG. 41 for the stimulable phosphor sheet,

FIG. 43 is a schematic perspective view showing a biochemical analysis unit, which is managed by a first embodiment of the system for managing a biochemical analysis medium in accordance with the present invention,

FIG. 44 is a schematic plan view showing a spotting apparatus,

FIG. 45 is a block diagram showing a control system, an input system, a driving system, and a detecting system of the spotting apparatus shown in FIG. 44,

FIG. 46 is a schematic plan view showing a biochemical analysis medium sorting apparatus,

FIG. 47 is a block diagram showing a control system, a detecting system, a driving system, and an input system of the biochemical analysis medium sorting apparatus,

FIG. 48 is a schematic front view showing a biochemical analysis unit collecting box for collecting the biochemical analysis unit,

FIG. 49 is a schematic front view showing a stimulable phosphor sheet collecting box for collecting the stimulable phosphor sheet,

FIG. 50 is a schematic perspective view showing a biochemical analysis unit, which is managed by a second embodiment of the system for managing a biochemical analysis medium in accordance with the present invention,

FIG. 51 is a schematic perspective view showing the biochemical analysis unit provided with a plurality of spot-shaped regions located at a spacing from one another, each of the plurality of the spot-shaped regions having been formed with a process for spotting a liquid containing one of specific binding substances, such as cDNA's, onto an adsorptive plate of the biochemical analysis unit by use of the spotting apparatus,

FIG. 52 is a schematic side view showing a hybridization apparatus, which constitutes the second embodiment of the system for managing a biochemical analysis medium in accordance with the present invention,

FIG. 53 is a block diagram showing a control system, a detecting system, a driving system, an input system, and a displaying system of the hybridization apparatus, which constitutes the second embodiment of the system for managing a biochemical analysis medium in accordance with the present invention,

FIG. 54 is a schematic perspective view showing a stimulable phosphor sheet, which is managed by the second embodiment of the system for managing a biochemical analysis medium in accordance with the present invention,

FIG. 55 is a schematic perspective view showing an exposure apparatus for executing an exposure operation, wherein a stimulable phosphor contained in a stimulable phosphor layer formed on a support of the stimulable phosphor sheet shown in FIG. 54 is exposed to the radiation radiated out from the radioactive labeling substance, which is contained selectively in at least one spot-shaped region among the plurality of the spot-shaped regions of the biochemical analysis unit,

FIG. 56 is a block diagram showing a control system, a detecting system, a driving system, and a displaying system, of the exposure apparatus of FIG. 55,

FIG. 57 is a schematic plan view showing a biochemical analysis medium sorting apparatus, which constitutes the second embodiment of the system for managing a biochemical analysis medium in accordance with the present invention, and

FIG. 58 is a block diagram showing a control system, a detecting system, a driving system, and an input system of the biochemical analysis medium sorting apparatus, which constitutes the second embodiment of the system for managing a biochemical analysis medium in accordance with the present invention.

### DESCRIPTION OF THE PREFERRED EMBODIMENTS

The present invention will hereinbelow be described in further detail with reference to the accompanying drawings.

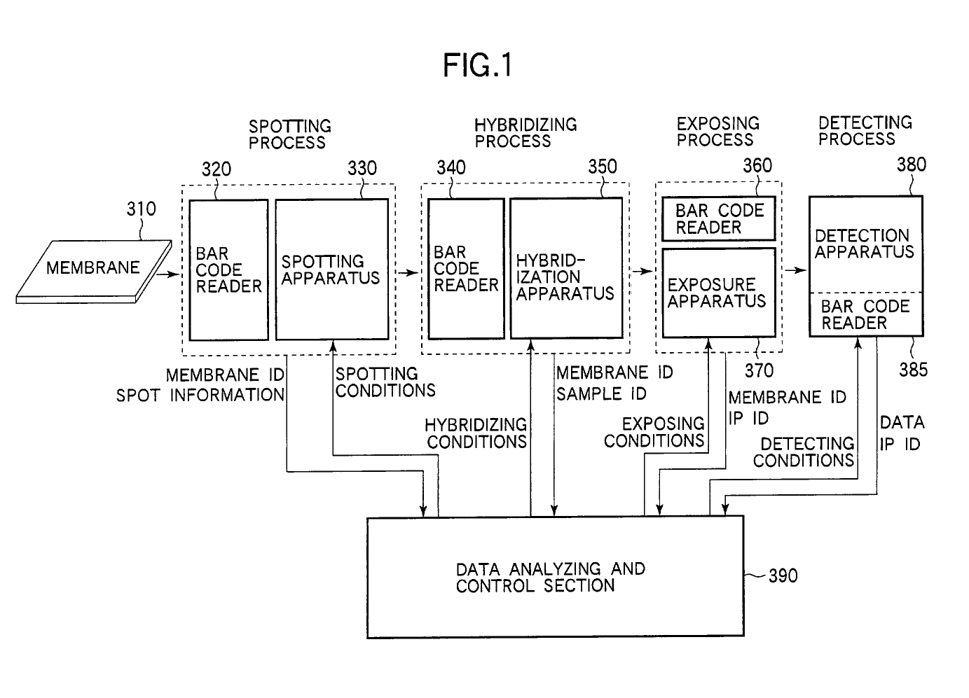

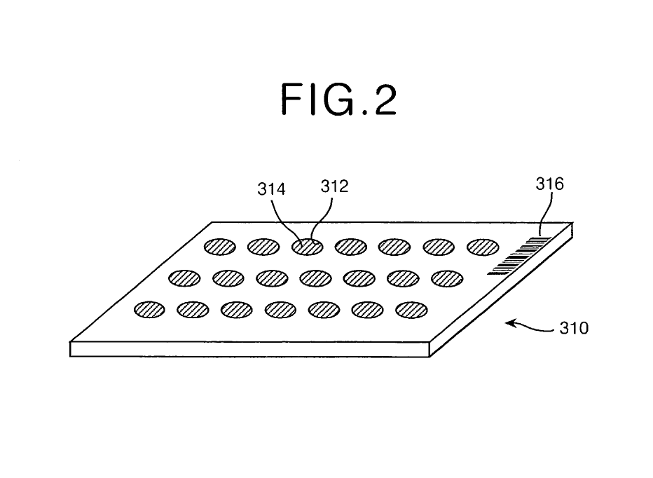

FIG. 1 is a block diagram showing an embodiment of the biochemical analysis system in accordance with the present invention. FIG. 2 is a schematic perspective view showing a base plate (a membrane 310) of a biochemical analysis unit utilized in the biochemical analysis system of FIG. 1.

As illustrated in FIG. 2, the membrane 310 utilized in this embodiment of the biochemical analysis system in accordance with the present invention comprises an aluminum metal plate, which has radiation attenuating properties, and a plurality of through-holes 312, 312, . . . which have approximately circular shapes and are formed at a high density in the aluminum metal plate. A porous material 314, such as cellulose acetate, is filled in each of the through holes 312, 312, . . . Also, a bar code 316, which represents information representing an identification (ID) number of the membrane 310, is attached to a marginal area of the membrane 310.

As illustrated in FIG. 1, this embodiment of the biochemical analysis system in accordance with the present invention comprises a bar code reader 320 for reading out the bar code 316 attached to the membrane 310 and acquiring the ID number of the membrane 310. The biochemical analysis system also comprises a spotting apparatus 330 for spotting specific binding substances respectively into the through-holes 312, 312, . . . of the membrane 310 in order to form spots and thereby preparing the biochemical analysis unit. The biochemical analysis system further comprises a bar code reader 340 for reading out the bar code 316 of the membrane 310 of the biochemical analysis unit immediately before the biochemical analysis unit is subjected to hybridization executed by a hybridization apparatus 350, which will be described later, and acquiring the ID number of the membrane 310, which is to be subjected to the hybridization. The biochemical analysis system still further comprises the hybridization apparatus 350 for applying a sample, which is to be analyzed and has been labeled with a radioactive isotope (RI) acting as a radioactive labeling substance, to the biochemical analysis unit, which has been prepared with the process for spotting the specific binding substances onto the membrane 310. With the hybridization apparatus 350, the labeled sample is thus subjected to selective binding (i.e., the hybridization) with the specific binding substances, which constitute the spots of the biochemical analysis unit. As a result of the hybridization, the labeled sample is selectively bound to at least one specific binding substance among the specific binding substances constituting the spots. The biochemical analysis system also comprises an exposure apparatus 370 for executing an exposure operation by superposing a stimulable phosphor sheet (hereinbelow referred to also as the IP sheet) and the biochemical analysis unit, which has been subjected to the hybridization, one upon the other. As a result of the exposure operation, the IP sheet is exposed to radiation radiated out from the radioactive labeling substance, with which the sample having been selectively bound to the at least one specific binding substance, has been labeled. The biochemical analysis system further comprises a bar code reader 360 for reading out a bar code attached to the IP sheet to be subjected to the exposure operation, and the bar code 316 of the membrane 310 of the biochemical analysis unit to be fed into the exposure apparatus 370, and thereby acquiring the ID number of the IP sheet and the ID number of the membrane 310 of the biochemical analysis unit, which are to be superposed one upon the other. The biochemical analysis system still further comprises a detection apparatus 380 provided with a bar code reader 385 for reading out the bar code of the IP sheet, which has been subjected to the exposure operation executed by the exposure apparatus 370, and thereby acquiring the ID number of the exposed IP sheet. In the detection apparatus 380, stimulating rays, which cause the IP sheet to emit light in proportion to the amount of energy stored on the IP sheet during the exposure of the IP sheet to the radiation, are irradiated to the IP sheet, and the light emitted by the IP sheet is photoelectrically detected. In this manner, data for a biochemical analysis is formed. The biochemical analysis system also comprises a data analyzing and control section 390. The data analyzing and control section 390 sends information representing spotting conditions to the spotting apparatus 330, sends information representing hybridizing conditions to the hybridization apparatus 350, sends information representing exposing conditions to the exposure apparatus 370, and sends information representing detecting conditions to the detection apparatus 380. Also, the data analyzing and control section 390 receives the ID information from each of the bar code readers 320, 340, 360, and 385, receives spot information from the spotting apparatus 330, receives the ID information of the sample from the hybridization apparatus 350, and receives data from the detection apparatus 380. The data analyzing and control section 390 stores the received pieces of information such that correspondence relationships among the received pieces of information are clear. Further, the data analyzing and control section 390 performs data analysis.

How this embodiment of the biochemical analysis system operates will be described hereinbelow.

When the membrane 310 having the structure shown in FIG. 2 is fed into the spotting process, the bar code reader 320 for the spotting process reads out the bar code 316 of the membrane 310 and acquires the ID number of the membrane 310. Thereafter, in accordance with the spotting conditions (i.e., the positions of the spots, the kinds of the specific binding substances, and the like), which have been received from the data analyzing and control section 390, the spotting apparatus 330 spots the specific binding substances respectively into the plurality of the through-holes 312, 312, . . . of the membrane 310 and thereby prepares the biochemical analysis unit. At the same time, the ID number and the spot information (in this case, the information representing the spotting conditions) of the membrane 310 of the biochemical analysis unit are sent to the data analyzing and control section 390.

The data analyzing and control section 390 stores the membrane ID number, which has been received from the bar code reader 320, and the spot information, which has been received from the spotting apparatus 330, in a storage section (not shown), such that it is clear which spot information corresponds to which membrane ID number. The membrane ID number and the corresponding spot information are utilized in the analysis, which will be performed later.

The biochemical analysis unit, which has been prepared with the spotting process performed by the spotting apparatus 330, is fed into the hybridizing process. Before the biochemical analysis unit is subjected to the hybridization with the hybridization apparatus 350, the bar code reader 340 for the hybridizing process reads out the bar code 316 of the membrane 310 of the biochemical analysis unit and acquires the membrane ID number. Thereafter, the biochemical analysis unit is subjected to the hybridization executed by the hybridization apparatus 350. The hybridization is executed in accordance with the hybridizing conditions (i.e., the ID number of the sample subjected to the hybridization, the time of hybridization, and the like) having been received from the data analyzing and control section 390. As a result of the hybridization, at least one spot among the plurality of the spots of the biochemical analysis unit is selectively labeled with the RI-labeled sample. At the same time, the membrane ID number of the biochemical analysis unit and the sample ID number are sent to the data analyzing and control section 390.

In accordance with the membrane ID number, which has been received from the bar code reader 340, the data analyzing and control section 390 stores the sample ID number, which has been received from the hybridization apparatus 350, in the storage section (not shown) such that it is clear which sample ID number corresponds to which spot information among the pieces of spot information having already been stored in association with membrane ID numbers. The sample ID number and the corresponding spot information are utilized in the analysis, which will be performed later.

The biochemical analysis unit, which has been subjected to the hybridization executed by the hybridization apparatus 350, is then fed into the exposing process. Before the biochemical analysis unit and the IP sheet are superposed one upon the other in the exposure apparatus 370, the bar code reader 360 for the exposing process reads out the bar code 316 of the membrane 310 of the biochemical analysis unit and the bar code of the IP sheet to be superposed upon the biochemical analysis unit and thereby acquires the membrane ID number and the IP sheet ID number. The information representing the membrane ID number and the information representing the IP sheet ID number are sent from the bar code reader 360 to the data analyzing and control section 390. Thereafter, the exposure apparatus 370 executes the exposure operation by superposing the biochemical analysis unit and the IP sheet one upon the other in accordance with the exposing conditions (i.e., the time of exposure, and the like), which have been received from the data analyzing and control section 390.

In accordance with the membrane ID number, which has been received from the bar code reader 360, the data analyzing and control section 390 stores the IP sheet ID number, which has been received from the bar code reader 360, in the storage section (not shown) such that it is clear which IP sheet ID number corresponds to which spot information and which sample ID number among the pieces of information having already been stored in association with the membrane ID numbers. The IP sheet ID number, the corresponding spot information, and the corresponding sample ID number are utilized in the analysis, which will be performed later.

The IP sheet, which has been subjected to the exposure operation, is then fed into the detecting process. Firstly, the bar code reader 385 of the detection apparatus 380 reads out the bar code of the IP sheet to be fed into the detecting process and acquires the IP sheet ID number. Thereafter, the detection apparatus 380 irradiates the stimulating rays to the IP sheet and photoelectrically detects the light emitted by the IP sheet. In this manner, digital data is obtained. The digital data is sent to the data analyzing and control section 390 together with the IP sheet ID number.

In accordance with the IP sheet ID number, which has been received from the detection apparatus 380, the data analyzing and control section 390 stores the digital data, which has been received from the detection apparatus 380, in the storage section (not shown) such that it is clear which digital data corresponds to which membrane ID number, which spot information, and which sample ID number among the pieces of information having already been stored in association with the IP sheet ID numbers. The digital data is utilized in the analysis, which will be performed later.

The biochemical analysis unit, which has been used in the exposure operation for the IP sheet, may be washed. The washed biochemical analysis unit is capable of being reused for the biochemical analysis.

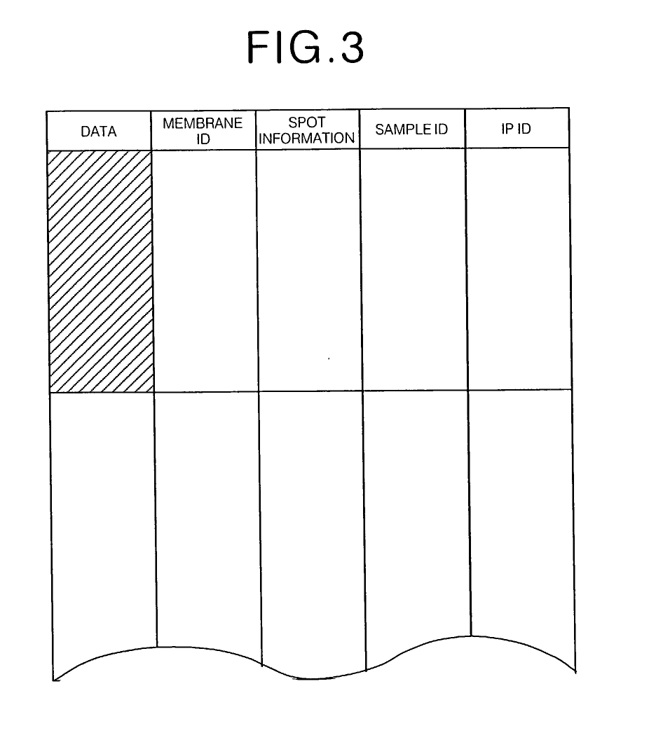

FIG. 3 shows a format, in which the digital data, the spot information, and ID numbers are stored in the storage section of the data analyzing and control section 390. As illustrated in FIG. 3, with respect to a single piece of digital data, the membrane ID number of the membrane having been used for acquiring the digital data, the spot information at the time of the preparation of the biochemical analysis unit by use of the membrane, the sample ID number of the sample having been used at the time of the hybridization utilizing the biochemical analysis unit, and the IP sheet ID number of the IP sheet having been subjected to the exposure operation utilizing the biochemical analysis unit, which has been subjected to the hybridization, are stored such that the correspondence relationships among the pieces of information are clear. In this embodiment, in the storage section (not shown) of the data analyzing and control section 390, information representing the hole location pattern of the membrane 310 has been inputted and stored previously in accordance with the membrane ID number, and sample specifying information has been inputted and stored previously in accordance with the sample ID number.

When the analysis is later performed on the digital, data, the data analyzing and control section 390 acquires the hole location pattern information of the membrane in accordance with the membrane ID number, which has been stored in association with the digital data. The data analyzing and control section 390 also acquires the sample specifying information corresponding to the sample ID number. In this manner, the analysis of the digital data is capable of being made in accordance with the hole location pattern information, the sample specifying information, and the spot information.

In this embodiment of the biochemical analysis system, the detection apparatus 380 is provided with the bar code reader 385, and the correspondence relationship between the digital data and the IP sheet ID number is made clear. Alternatively, in cases where the exposure apparatus 370 and the detection apparatus 380 are located at positions comparatively close to each other, and the IP sheets having been subjected to the exposure operations executed by the exposure apparatus 370 are fed into the detection apparatus 380 in the order, in which the IP sheets are subjected to the exposure operations, the bar code reader 385 of the detection apparatus 380 may be omitted. Also, in such cases, the order, in which the IP sheets are subjected to the exposure operations, and order, in which pieces of digital data are detected, may be collated with each other, and the correspondence relationship between the digital data and the IP sheet ID number may thereby be specified. In this manner, the biochemical analysis system may be kept simple.

Also, in this embodiment of the biochemical analysis system, the membrane 310 having the structure shown in FIG. 2 is employed. With the membrane 310, the spots are capable of being formed at a high density in the biochemical analysis unit, and the problems are capable of being prevented from occurring in that the electron rays, which have been radiated out from the radioactive labeling substance contained in at least one of the spots, are scattered within the membrane during the exposure operation performed by use of the biochemical analysis unit having been subjected to the hybridization and the IP sheet, and that the electron rays, which have been radiated out from the radioactive labeling substance contained in one of the spots, are scattered and impinge upon a region of the IP sheet, which region is to be exposed to the electron rays radiated out from an adjacent spot. However, in the biochemical analysis system in accordance with the present invention, the membrane is not limited to the membrane 310 having the structure shown in FIG. 2.

Further, in this embodiment of the biochemical analysis system, the membrane 310 having the through-holes is employed, such that the membrane 310 is capable of being reused easily through the washing and the biochemical analysis unit is capable of being prepared easily from the membrane 310. Alternatively, a membrane comprising a base plate, a plurality of holes, which are other than the through-holes and are formed in the base plate, and an adsorptive material, which has been filled in each of the recesses of the holes, may be employed.

Furthermore, in this embodiment of the biochemical analysis system, aluminum is employed as the material for the formation of the base plate. However, in cases where the radioactive labeling substance is employed as the labeling substance, the material for the formation of the base plate may be selected from a wide variety of materials having the radiation attenuating properties. The material for the formation of the base plate should preferably be selected from metal materials, ceramic materials, and plastic materials. Also, the material for the formation of the base plate may be selected from inorganic compound materials and organic compound materials.

Also, as the stimulable phosphor sheet (i.e., the IP sheet), a stimulable phosphor sheet comprising a support and dot-shaped stimulable phosphor layer regions, which are located at a spacing from one another on or in the support, may be employed. In such cases, the problems are capable of being prevented from occurring in that the radiation having been radiated out from the biochemical analysis unit is scattered within the stimulable phosphor sheet. Therefore, the occurrence of noise is capable of being suppressed even further.

As described above, with this embodiment of the biochemical analysis system, in the spotting process, the membrane ID number and the spot information are acquired such that the correspondence relationship between the two pieces of information is clear. Also, in the hybridizing process, the membrane ID number and the sample ID number are acquired such that the correspondence relationship between the two pieces of information is clear. Further, in the exposing process, the membrane ID number and the IP sheet ID number are acquired such that the correspondence relationship between the two pieces of information is clear. Therefore, without reliance upon memory of the operator, a memorandum made by the operator, or the like, being made, it is capable of being found accurately and automatically, for example, which data was acquired by use of which biochemical analysis unit (i.e., which membrane) and which sample. Accordingly, artificial mistakes are capable of being avoided, and correct results of analysis are capable of being obtained.

Also, with this embodiment of the biochemical analysis system, in cases where the identification information (i.e., the IP sheet ID number) of the stimulable phosphor sheet is acquired, the number of times of use of the stimulable phosphor sheet is capable of being counted. Therefore, the management of the scrapping period of the stimulable phosphor sheet, and the like, is capable of being performed easily in accordance with the number of times of use of the stimulable phosphor sheet. Accordingly, the problems are capable of being prevented from occurring in that a deteriorated stimulable phosphor sheet is further used.

Further, this embodiment of the biochemical analysis system is provided with the spotting apparatus 330, such that a membrane, on which the spots have not yet been formed, may be utilized, and such that a membrane having already been used for the biochemical analysis may be reused. Also, the membrane ID number and the spot information are stored such that the correspondence relationship between the two pieces of information is clear. Therefore, it is capable of being found automatically which specific binding substances have been formed in what location pattern on which biochemical analysis unit. Accordingly, in cases where the commercially available biochemical analysis unit is not used and in cases where the biochemical analysis unit having been used for the biochemical analysis is reused, artificial mistakes are capable of being avoided.

This embodiment of the biochemical analysis system is provided with the spotting apparatus 330, such that a membrane, on which the spots have not yet been formed, may be utilized, and such that a membrane having already been used for the biochemical analysis may be reused. Also, the membrane ID number, which has been acquired by reading out the bar code of the membrane 310 by use of the bar code reader 320 in the spotting process, is taken as the identification information of the biochemical analysis unit having been prepared by use of the membrane 310. However, in cases where a commercially available biochemical analysis unit having already been provided with the spots is utilized, the biochemical analysis system may be constituted such that the system is not provided with the spotting apparatus 330, the bar code reader 340 and the bar code reader 360 are located respectively for the hybridizing process and the exposing process, and the ID number of the biochemical analysis unit is acquired such that it is clear which ID number of the biochemical analysis unit corresponds to which sample ID number and which IP sheet ID number. With the thus constituted biochemical analysis system, as in the embodiment of the biochemical analysis system shown in FIG. 1, without reliance upon memory of the operator, a memorandum made by the operator, or the like, being made, it is capable of being found accurately and automatically, for example, which data was acquired by use of which biochemical analysis unit and which sample. Accordingly, artificial mistakes are capable of being avoided, and correct results of analysis are capable of being obtained.

Also, with the aforesaid embodiment of the biochemical analysis system, the membrane ID number is taken as the identification information of the biochemical analysis unit. Therefore, the number of times of use of the membrane is capable of being counted, and the management of the scrapping period of the base plate, and the like, is capable of being performed easily. Accordingly, the problems are capable of being prevented from occurring in that a deteriorated base plate is further used and the results of the analysis are adversely affected.

In the aforesaid embodiment of the biochemical analysis system, the radioactive labeling substance (i.e., the RI) is employed as the labeling substance, the exposure operation is executed by superposing the biochemical analysis unit having been subjected to the hybridization and the IP sheet one upon the other, and the data for a biochemical analysis is acquired by detecting the information from the IP sheet. However, the biochemical analysis system in accordance with the present invention is not limited to the use of the radioactive labeling substance as the labeling substance. For example, the biochemical analysis system in accordance with the present invention is also applicable to biochemical analysis systems, wherein the fluorescent labeling substance described above or the labeling substance described above, which is capable of producing the chemiluminescence when being brought into contact with a chemiluminescence substrate, may be employed as the labeling substance, and the system is provided with the label signal forming means and the read-out means corresponding to the kind of the labeling substance.

Also, in the aforesaid embodiment of the biochemical analysis system shown in FIG. 1, the data analyzing and control section 390 sends the pieces of information representing the processing conditions (i.e., the spotting conditions, the hybridizing conditions, the exposing conditions, and the detecting conditions) in the spotting process, the hybridizing process, the exposing process, and the detecting process into the corresponding apparatuses (i.e., the spotting apparatus 330, the hybridization apparatus 350, the exposure apparatus 370, and the detection apparatus 380). Also, appropriate adjustments are capable of being made. For example, in cases where a small amount of sample was used in the hybridizing process, and therefore the amount of the radioactive labeling substance contained in the biochemical analysis unit having been subjected to the hybridization with the sample is small, an adjustment is capable of being made such that the time of irradiation of the stimulating rays in the detecting process is set to be long. In this manner, the efficiency of the biochemical analysis system is capable of being enhanced. However, in cases where only a predetermined kind of sample or only a predetermined kind of biochemical analysis unit is utilized, the control for the adjustment of the processing conditions need not be performed, and only the predetermined processing conditions may be utilized.

Further, in the aforesaid embodiment of the biochemical analysis system, as the identification information of the membrane 310, the sample, and the IP sheet, the ID numbers are utilized, and the ID numbers are acquired by use of the bar code readers. However, the identification information is not limited to the ID number, and the means for acquiring the identification information is not limited to the bar code reader.

Embodiments of the biochemical analysis unit in accordance with the present invention will be described hereinbelow.

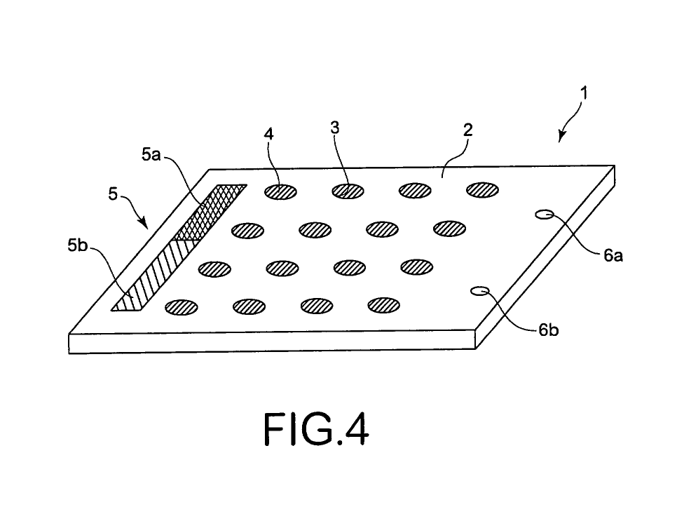

FIG. 4 is a schematic perspective view showing a first embodiment of the biochemical analysis unit in accordance with the present invention.

As illustrated in FIG. 4, a biochemical analysis unit 1, which is the first embodiment of the biochemical analysis unit in accordance with the present invention, comprises a base plate 2, which is made from aluminum and is provided with a plurality of through-holes 3, 3, . . . having an approximately circular shape. The through-holes 3, 3, . . . are located at a high density. Also, a 6,6-nylon is filled in each of the through-holes 3, 3, . . . . In this manner, a plurality of dot-shaped adsorptive regions 4, 4, . . . are formed.

Though not shown precisely in FIG. 4, in this embodiment, approximately 10,000 through-holes 3, 3, . . . , each of which has the approximately circular shape with a size of approximately 0.01 mm², are formed in a regular pattern at a density of approximately 5,000 holes/cm² in the base plate 2. The 6,6-nylon is filled in each of the through-holes 3, 3, . . . , and the dot-shaped adsorptive regions 4, 4, . . . are thereby formed such that the heights of the top surfaces of the adsorptive regions 4, 4, . . . may be identical with the height of the top surface of the base plate 2.

As illustrated in FIG. 4, the base plate 2 of the biochemical analysis unit 1 is provided with a magnetic recording layer 5 constituted of a magnetic recording medium. The base plate 2 is also provided with two circular position matching through-holes 6a and 6b.

The magnetic recording layer 5 comprises a first data recording region 5a, which is protected from data writing conducted by a user, and a second data recording region 5b, on which the data is capable of being written by the user.

In this embodiment of the biochemical analysis unit in accordance with the present invention, the adsorptive material is filled in each of the through-holes 3, 3, . . . of the base plate 2 of the biochemical analysis unit 1, and the adsorptive regions 4, 4, . . . are thereby formed. However, it is not always possible to form all of the through-holes 3, 3, . . . so as to have uniform size, and it is not always possible to fill the adsorptive material uniformly in the through-holes 3, 3, . . . . Therefore, as will be described later, in cases where radiation information or fluorescence information is recorded in at least one adsorptive region 4 among the plurality of the adsorptive regions 4, 4, . . . , and the data for a biochemical analysis is obtained by reading out the radiation information or fluorescence information from the adsorptive region 4, there is the risk that noise will occur in the data for a biochemical analysis due to nonuniformity in amount of the adsorptive material contained in the adsorptive regions 4, 4, . . . of the biochemical analysis unit 1.

Therefore, in this embodiment, a variation in size of the plurality of the adsorptive regions 4, 4, . . . of the base plate 2 of the biochemical analysis unit 1 is measured by use of an electron microscope, or the like. Also, the position of the top surface of the adsorptive material filled in each of the through-holes 3, 3, . . . is detected by use of a laser displacement meter. In this manner, the amount of the adsorptive material filled in each of the through-holes 3, 3, . . . is thereby measured. The thus obtained data concerning the amounts of the adsorptive material contained in the plurality of the adsorptive regions 4, 4, . . . is recorded on the first data recording region 5a of the magnetic recording layer 5 of the biochemical analysis unit 1 by use of a magnetic recording head (not shown).

Also, data concerning the kinds of the specific binding substances having been spotted by use of a spotting apparatus, data concerning the positions of the adsorptive regions 4, 4, . . . , to which positions the specific binding substances have been spotted respectively, data concerning the number of times of use of the biochemical analysis unit 1, and the like, are recorded on the first data recording region 5a of the magnetic recording layer 5.

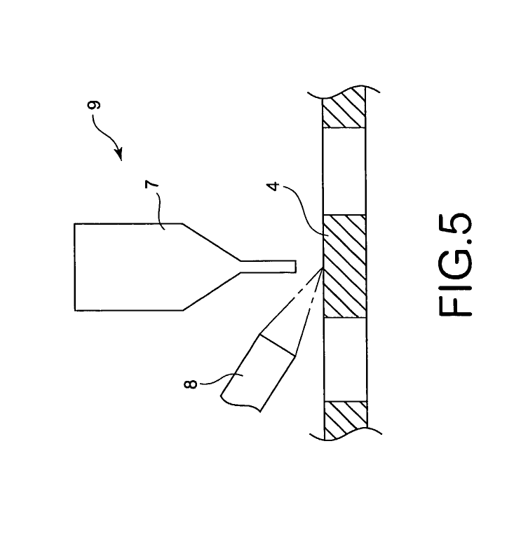

FIG. 5 is a schematic front view showing a spotting apparatus.

When the biochemical analysis is to be made, as illustrated in FIG. 5, for example, different kinds of cDNA's, whose base sequences are known, are spotted as the specific binding substances respectively onto the plurality of the adsorptive regions 4, 4, . . . , which have been formed in the regular pattern in the biochemical analysis unit 1, by use of the spotting apparatus.

As illustrated in FIG. 5, the spotting apparatus comprises a spotting head 9. The spotting head 9 is provided with an injector 7 for injecting a liquid containing a specific binding substance toward the biochemical analysis unit 1, and a CCD camera 8.

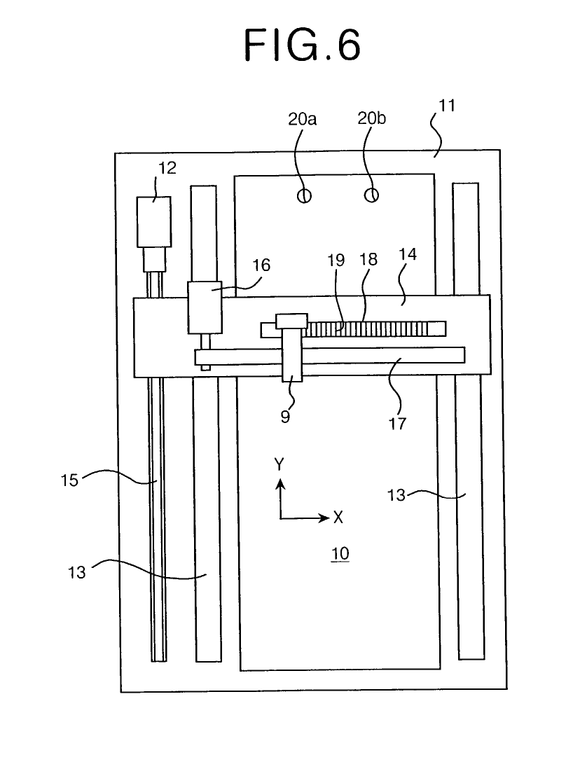

FIG. 6 is a schematic plan view showing the spotting apparatus.

As illustrated in FIG. 6, the spotting apparatus comprises a driving mechanism. The driving mechanism of the spotting apparatus is mounted on a frame 11 secured to a base 10, on which the biochemical analysis unit 1 to be spotted with the specific binding substances, such as the cDNA's, is placed.

As illustrated in FIG. 6, a sub-scanning pulse motor 12 and a pair of rails 13, 13 are secured to the frame 11. Also, a base 14 is located on the frame 11. The base 14 is capable of moving in a sub-scanning direction, which is indicated by the arrow Y in FIG. 6, along the pair of the rails 13, 13.

The movable base 14 has a threaded hole (not shown). A threaded rod 15, which is rotated by the sub-scanning pulse motor 12, is engaged with the threaded hole of the base 14.

A main scanning pulse motor 16 is secured to the movable base 14. The main scanning pulse motor 16 is capable of intermittently driving an endless belt 17 at a predetermined pitch.

The spotting head 9 of the spotting apparatus is secured to the endless belt 17. When the endless belt 17 is driven by the main scanning pulse motor 16, the spotting head 9 is moved by the endless belt 17 in a main scanning direction, which is indicated by the arrow X in FIG. 6.

In FIG. 6, reference numeral 18 represents a linear encoder for detecting the position of the spotting head 9 with respect to the main scanning direction. Also, reference numeral 19 represents slits of the linear encoder 18.

As illustrated in FIG. 6, two position matching pins 20a and 20b protrude from the base 10 of the spotting apparatus. The position matching pins 20a and 20b are located at the positions corresponding respectively to the two position matching through-holes 6a and 6b of the base plate 2 of the biochemical analysis unit 1. The biochemical analysis unit 1 is placed on the base 10 of the spotting apparatus, such that the two position matching through-holes 6a and 6b of the base plate 2 of the biochemical analysis unit 1 fit respectively onto the two position matching pins 20a and 20b of the base 10 of the spotting apparatus. In this manner, the biochemical analysis unit 1 is capable of being placed reliably at approximately the same position on the base 10 of the spotting apparatus.

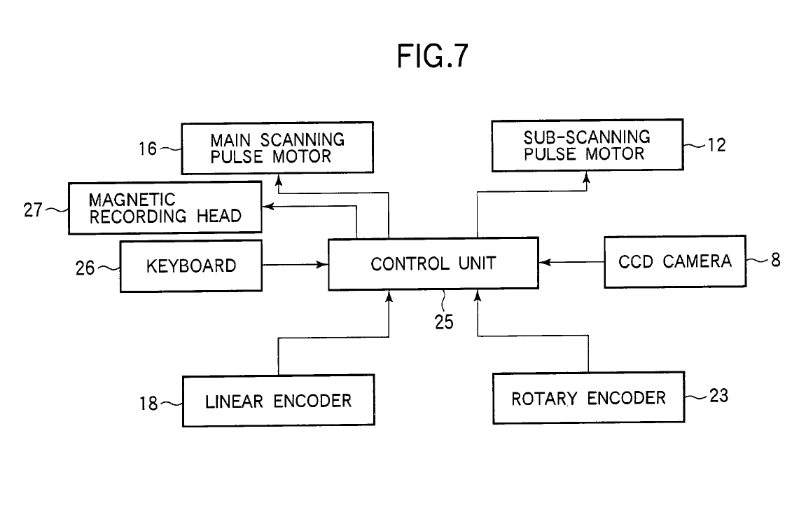

FIG. 7 is a block diagram showing a control system, an input system, a driving system, a detecting system, and a recording system of the spotting apparatus.

As illustrated in FIG. 7, the control system of the spotting apparatus comprises a control unit 25 for controlling the operations of the spotting apparatus. The input system of the spotting apparatus comprises a keyboard 26.

Also, the driving system of the spotting apparatus comprises the main scanning pulse motor 16 and the sub-scanning pulse motor 12. The detecting system of the spotting apparatus comprises the linear encoder 18 for detecting the position of the spotting head 9 with respect to the main scanning direction, a rotary encoder 23 for detecting the amount of rotation of the rod 15, and the CCD camera 8.

As illustrated in FIG. 7, the recording system of the spotting apparatus comprises a magnetic recording head 27.

With the spotting apparatus constituted in the manner described above, the specific binding substances, such as the cDNA's, are spotted respectively onto the plurality of the adsorptive regions 4, 4, . . . of the biochemical analysis unit 1.

Firstly, the biochemical analysis unit 1 is placed on the base 10 of the spotting apparatus, such that the two position matching through-holes 6a and 6b of the biochemical analysis unit 1 fit respectively onto the two position matching pins 20a and 20b of the base 10 of the spotting apparatus.

Thereafter, data concerning the kinds of the specific binding substances to be spotted and data concerning the positions of the adsorptive regions 4, 4, . . . , to which positions the specific binding substances are to be spotted respectively, are inputted by the operator from the keyboard 26. Also, in cases where the biochemical analysis unit 1 and a specific stimulable phosphor sheet are supplied as one biochemical analysis kit to the user, identification data for specifying the stimulable phosphor sheet, which constitutes the one biochemical analysis kit together with the biochemical analysis unit 1 and is to be used together with the biochemical analysis unit 1, is inputted by the operator from the keyboard 26. The data inputted from the keyboard 26 are fed into the control unit 25. The stimulable phosphor sheet will be described later.

In this embodiment of the biochemical analysis unit 1, the data concerning the kinds of the specific binding substances to be spotted and the data concerning the positions of the adsorptive regions 4, 4, . . . , to which positions the specific binding substances are to be spotted respectively, are stored in a memory (not shown) as predetermined patterns. Also, when a certain pattern of the data concerning the kinds of the specific binding substances to be spotted and the data concerning the positions of the adsorptive regions 4, 4, . . . , to which positions the specific binding substances are to be spotted respectively, is specified by the operator, the control unit 25 reads out the corresponding pattern from the memory and controls the operations of the spotting apparatus.

When the data concerning the kinds of the specific binding substances to be spotted and the data concerning the positions of the adsorptive regions 4, 4, . . . , to which positions the specific binding substances are to be spotted respectively, are inputted by the operator from the keyboard 26, and the identification data for specifying the stimulable phosphor sheet, which constitutes the one biochemical analysis kit together with the biochemical analysis unit 1 and is to be used together with the biochemical analysis unit 1, is inputted by the operator from the keyboard 26 in cases where the biochemical analysis unit 1 and the specific stimulable phosphor sheet are supplied as one biochemical analysis kit to the user, the control unit 25 feeds a writing signal to the magnetic recording head 27 in accordance with the inputted signal. The magnetic recording head 27 writes the data concerning the kinds of the specific binding substances to be spotted, the data concerning the positions of the adsorptive regions 4, 4, . . . , to which positions the specific binding substances are to be spotted respectively, and the identification data for specifying the stimulable phosphor sheet on the first data recording region 5a of the magnetic recording layer 5, which region is protected from data writing conducted by a user.

As described above, this embodiment is constituted such that the biochemical analysis unit 1 is placed approximately at the predetermined position on the base 10 of the spotting apparatus. However, in this embodiment, the size of each of the adsorptive regions 4, 4, . . . is approximately 0.01 mm². Therefore, it will not always be guaranteed that the center points of the plurality of the adsorptive regions 4, 4, . . . of the biochemical analysis unit 1 placed on the base 10 are accurately aligned along the main scanning direction and the sub-scanning direction of the spotting head 9.

Accordingly, in this embodiment, the spotting apparatus is constituted such that the relationship between the position of the biochemical analysis unit 1 placed on the base 10 and the position of movement of the spotting head 9 in the main scanning direction and the sub-scanning direction is detected previously, and the spotting head 9 is moved by the main scanning pulse motor 16 and the sub-scanning pulse motor 12 so as to be capable of injecting each specific binding substance accurately to each of the adsorptive regions 4, 4, . . . .

Thereafter, when a spotting start signal is inputted by the operator from the keyboard 26 into the control unit 25, the control unit 25 feeds a driving signal to the main scanning pulse motor 16, and the spotting head 9 being located at a reference position is moved in the main scanning direction, which is indicated by the arrow X in FIG. 6. The control unit 25 then feeds a driving signal to the sub-scanning pulse motor 12, and the spotting head 9 is moved in the sub-scanning direction, which is indicated by the arrow Y in FIG. 6.

While the spotting head 9 is being thus moved in the main scanning direction, which is indicated by the arrow X in FIG. 6, and in the sub-scanning direction, which is indicated by the arrow Y in FIG. 6, the control unit 25 monitors a detection signal, which is received from the CCD camera 8, and detects the positions of four corners of the biochemical analysis unit 1. Also, the control unit 25 calculates coordinate values of the four corners of the biochemical analysis unit 1 by taking the reference position of the spotting head 9 as an origin on a coordinate system. The control unit 25 stores the information, which represents the coordinate values of the four corners of the biochemical analysis unit 1, in a memory (not shown).

When the positions of the four corners of the biochemical analysis unit 1 have been detected, and the information representing the coordinate values of the four corners of the biochemical analysis unit 1 has been stored in the memory, the control unit 25 calculates the coordinate values of each of the adsorptive regions 4, 4, . . . of the biochemical analysis unit 1 in accordance with the coordinate values of the four corners of the biochemical analysis unit 1 and by taking the reference position of the spotting head 9 as the origin on the coordinate system. Also, the control unit 25 stores the information, which represents the coordinate values of each of the adsorptive regions 4, 4, . . . of the biochemical analysis unit 1, in the memory (not shown).

When the coordinate values of each of the adsorptive regions 4, 4, . . . of the biochemical analysis unit 1 have been calculated, and the information representing the calculated coordinate values has been stored in the memory, the control unit 25 feeds the driving signal into the main scanning pulse motor 16 and the sub-scanning pulse motor 12, and the spotting head 9 is returned to the original reference position.

In cases where the specific binding substance injected from the injector 7 of the spotting head 9 is spotted accurately onto the position, which stands facing the orifice of the injector 7, the specific binding substance may be injected from the injector 7 of the spotting head 9 in accordance with the coordinate values of each of the adsorptive regions 4, 4, . . . of the biochemical analysis unit 1, which coordinate values have been determined by taking the reference position of the spotting head 9 as the origin of the coordinate system. In this manner, the specific binding substance is capable of being spotted accurately to each of the adsorptive regions 4, 4, . . . of the biochemical analysis unit 1. However, in cases where the specific binding substance injected from the injector 7 of the spotting head 9 is spotted onto a position deviated from the position, which stands facing the orifice of the injector 7, in the direction indicated by the arrow X and/or in the direction indicated by the arrow Y, even if the specific binding substance is injected from the injector 7 of the spotting head 9 in accordance with the coordinate values of each of the adsorptive regions 4, 4, . . . of the biochemical analysis unit 1 in the manner described above, the specific binding substance cannot be spotted accurately to each of the adsorptive regions 4, 4, . . . of the biochemical analysis unit 1.

Therefore, in this embodiment, as illustrated in FIG. 7, the specific binding substance is injected from the injector 7 of the spotting head 9, which has been returned to the reference position, toward the surface of the biochemical analysis unit 1, and the position, to which the specific binding substance has thus been spotted, is detected by the CCD camera 8. In accordance with the detection signal obtained from the CCD camera 8, the control unit 25 calculates a deviation quantity δx of the position, to which the specific binding substance has been spotted, in the direction indicated by the arrow X and from a position O, which stands facing the orifice of the injector 7, and a deviation quantity δy of the position, to which the specific binding substance has been spotted, in the direction indicated by the arrow Y and from the position O, which stands facing the orifice of the injector 7. The information representing the calculated deviation quantities is stored in the memory.

The deviation quantity δx of the position, to which the specific binding substance has been spotted, in the direction indicated by the arrow X and from the position O, which stands facing the orifice of the injector 7, and the deviation quantity δy of the position, to which the specific binding substance has been spotted, in the direction indicated by the arrow Y and from the position O, which stands facing the orifice of the injector 7, are the values inherent to the injector 7 of each spotting head 9. Therefore, the spotting position of the specific binding substance, which is injected from the injector 7 toward the surface of the biochemical analysis unit 1 when the spotting head 9 is located at a position other than the reference position, is also deviated by δx in the direction indicated by the arrow X and from the position O, which stands facing the orifice of the injector 7, and deviated by δy in the direction indicated by the arrow Y and from the position O, which stands facing the orifice of the injector 7.

Thereafter, in accordance with the coordinate values of the four corners of the biochemical analysis unit 1, the coordinate values of each of the adsorptive regions 4, 4, . . . of the biochemical analysis unit 1, the deviation quantity δx of the spotting position of the specific binding substance in the direction indicated by the arrow X, and the deviation quantity δy of the spotting position of the specific binding substance in the direction indicated by the arrow Y, the control unit 25 calculates driving pulses to be given to the main scanning pulse motor 16 and the sub-scanning pulse motor 12 in order to move the spotting head 9 to the position, at which the orifice of the injector 7 of the spotting head 9 stands facing each of the adsorptive regions 4, 4, . . . . The driving pulse data is stored in the memory.

In this embodiment, the plurality of the adsorptive regions 4, 4, . . . of the biochemical analysis unit 1 are formed within the through-holes 3, 3, . . . , which are located in the regular pattern in the base plate 2. Therefore, the driving pulses to be given to the main scanning pulse motor 16 and the sub-scanning pulse motor 12 in order to move the spotting head 9 to the position, at which the orifice of the injector 7 of the spotting head 9 thirdly or thereafter stands facing an adsorptive region 4 to be spotted with the specific binding substance, may be identical with the driving pulses to be given to the main scanning pulse motor 16 and the sub-scanning pulse motor 12 in order to move the spotting head 9 from the position, at which the orifice of the injector 7 of the spotting head 9 firstly stands facing an adsorptive region 4 to be spotted with the specific binding substance, to the position, at which the orifice of the injector 7 of the spotting head 9 secondly stands facing an adsorptive region 4 to be spotted with the specific binding substance. Accordingly, it is sufficient for the calculations to be made to find only the driving pulses to be given to the main scanning pulse motor 16 and the sub-scanning pulse motor 12 in order to move the spotting head 9 to the position, at which the orifice of the injector 7 firstly stands facing an adsorptive region 4 to be spotted with the specific binding substance, and the driving pulses to be given to the main scanning pulse motor 16 and the sub-scanning pulse motor 12 in order to move the spotting head 9 from the position, at which the orifice of the injector 7 firstly stands facing an adsorptive region 4 to be spotted with the specific binding substance, to the position, at which the orifice of the injector 7 secondly stands facing an adsorptive region 4 to be spotted with the specific binding substance. The thus obtained driving pulse data may be stored in the memory.

In the manner described above, the driving pulses to be given to the main scanning pulse motor 16 and the sub-scanning pulse motor 12 in order to move the spotting head 9 to the position, at which the orifice of the injector 7 stands facing each of the adsorptive regions 4, 4, . . . , are calculated, and the thus obtained driving pulse data is stored in the memory. Thereafter, the control unit 25 gives the predetermined driving pulses to the main scanning pulse motor 16 and the sub-scanning pulse motor 12 in accordance with the driving pulse data having been stored in the memory, and the spotting head 9 is intermittently moved. When the orifice of the injector 7 arrives at the position, which stands facing each of the adsorptive regions 4, 4, . . . of the biochemical analysis unit 1, the control unit 25 feeds a driving stop signal to the main scanning pulse motor 16 and the sub-scanning pulse motor 12, and the spotting head 9 is stopped. Also, the control unit 25 feeds a spotting signal to the injector 7 of the spotting head 9 and causes the injector 7 to inject the specific binding substance.

In cases where the spotting head 9 is to be moved to the position, at which the orifice of the injector 7 of the spotting head 9 secondly or thereafter stands facing an adsorptive region 4 to be spotted with the specific binding substance, the spotting head 9 is moved at predetermined pitches in the main scanning direction indicated by the arrow X and in the sub-scanning direction indicated by the arrow Y.

In the same manner, the spotting head 9 is moved intermittently by the main scanning pulse motor 16 and the sub-scanning pulse motor 12. Also, in accordance with instruction signals inputted by the operator, predetermined specific binding substances are spotted respectively onto the plurality of the adsorptive regions 4, 4, . . . .

After the specific binding substances have been spotted respectively onto the plurality of the adsorptive regions 4, 4, . . . in the manner described above, the biochemical analysis unit 1 is delivered to the user.

The user receives the biochemical analysis unit 1 comprising the plurality of the adsorptive regions 4, 4, . . . , to which the specific binding substances have respectively been spotted. The user reads out the data, which has been recorded on the magnetic recording layer 5 of the biochemical analysis unit 1, by use of a reader (not shown) and confirms the kinds and the positions of the specific binding substances having been spotted and adsorbed to the plurality of the adsorptive regions 4, 4, . . . of the biochemical analysis unit 1. Thereafter, the user executes the hybridization, wherein an organism-originating substance having been labeled with a labeling substance is subjected to the selective hybridization with the specific binding substances, which have been spotted onto the plurality of the adsorptive regions 4, 4, . . . .

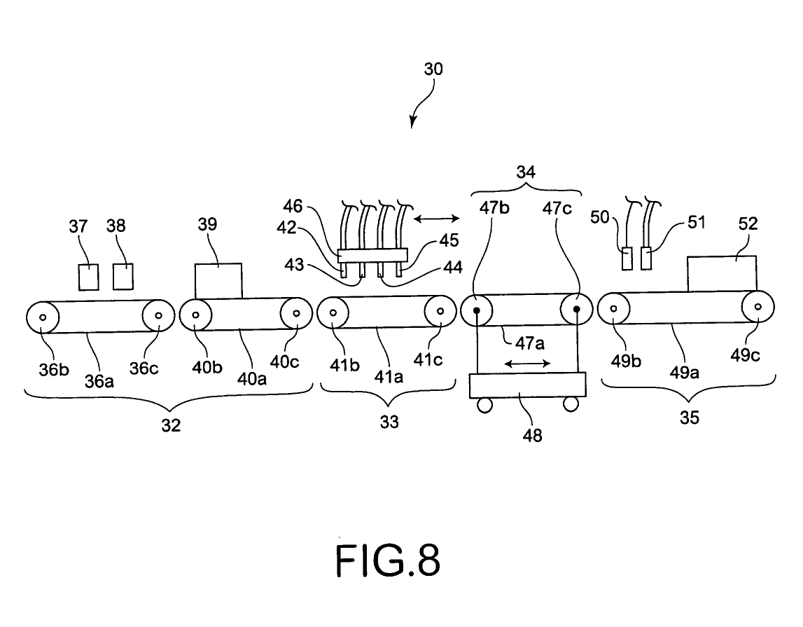

FIG. 8 is a schematic side view showing a hybridization apparatus.

As illustrated in FIG. 8, a hybridization apparatus 30 comprises a cartridge loading section 32 for loading the biochemical analysis unit 1 into a cartridge 31. The hybridization apparatus 30 also comprises a liquid injecting section 33 for selectively injecting a pre-treatment liquid, a hybridization liquid, a probe liquid containing the organism-originating substance having been labeled with the labeling substance, or a washing liquid into the cartridge 31, in which the biochemical analysis unit 1 has been accommodated. The hybridization apparatus 30 further comprises a reacting section 34 for shaking and vibrating the cartridge 31, in which the biochemical analysis unit 1 has been accommodated and which has been injected with the pre-treatment liquid, the hybridization liquid, a liquid prepared by the addition of the probe liquid to the hybridization liquid, or the washing liquid. The hybridization apparatus 30 still further comprises a biochemical analysis unit take-out section 35 for discharging the pre-treatment liquid, the liquid prepared by the addition of the probe liquid to the hybridization liquid, or the washing liquid from the cartridge 31, and taking out the biochemical analysis unit 1 from the cartridge 31.

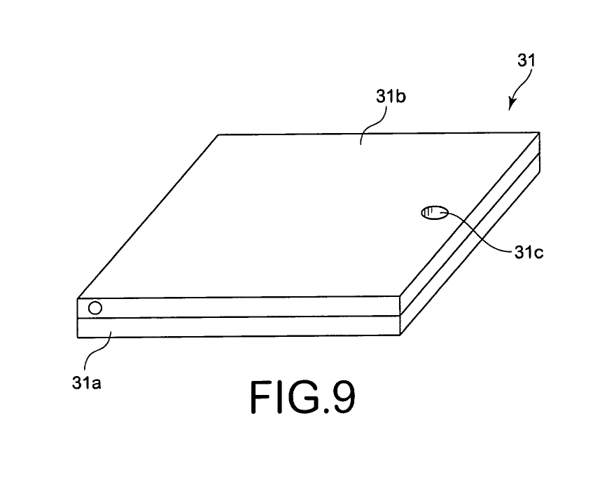

FIG. 9 is a schematic perspective view showing the cartridge 31.

As illustrated in FIG. 9, the cartridge 31 comprises a casing 31a and a cover 31b. The cover 31b has a liquid injecting and discharging port 31c, through which the pre-treatment liquid, the hybridization liquid, the probe liquid, and the washing liquid is capable of being injected into the cartridge 31 and discharged from the cartridge 31.

As illustrated in FIG. 8, the cartridge loading section 32 of the hybridization apparatus 30 comprises a first endless belt 36a, on which the biochemical analysis unit 1 is set. The cartridge loading section 32 also comprises a pair of pulleys 36b and 36c, over which the first endless belt 36a is threaded and which are capable of rotating selectively in the clockwise direction or the counter-clockwise direction in FIG. 8. The cartridge loading section 32 further comprises a read-out head 37 for reading out the data having been recorded on the first data recording region 5a of the magnetic recording layer 5 of the biochemical analysis unit 1, which has been set on the first endless belt 36a. The cartridge loading section 32 still further comprises a magnetic recording head 38 for writing data on the magnetic recording layer 5 of the biochemical analysis unit 1. The cartridge loading section 32 also comprises a loading mechanism 39 for opening the cover 31b of the cartridge 31, loading the biochemical analysis unit 1 into the cartridge 31, and closing the cover 31b of the cartridge 31. The cartridge loading section 32 further comprises a second endless belt 40a for conveying the cartridge 31, into which the biochemical analysis unit 1 has been loaded. The cartridge loading section 32 still further comprises a pair of pulleys 40b and 40c, over which the second endless belt 40a is threaded.

As illustrated in FIG. 8, the liquid injecting section 33 of the hybridization apparatus 30 comprises a third endless belt 41a for receiving the cartridge 31 from the second endless belt 40a of the cartridge loading section 32. The liquid injecting section 33 also comprises a pair of pulleys 41b and 41c, over which the third endless belt 41a is threaded. The liquid injecting section 33 further comprises a pre-treatment liquid injecting pin 42 for injecting the pre-treatment liquid through the liquid injecting and discharging port 31c into the cartridge 31, which is located at the position for liquid injection. The liquid injecting section 33 still further comprises a hybridization liquid injecting pin 43 for injecting the hybridization liquid through the liquid injecting and discharging port 31c into the cartridge 31, which is located at the position for liquid injection. The liquid injecting section 33 also comprises a probe liquid injecting pin 44 for injecting the probe liquid through the liquid injecting and discharging port 31c into the cartridge 31, which is located at the position for liquid injection, and thereby adding the probe liquid to the hybridization liquid. The liquid injecting section 33 further comprises a washing liquid injecting pin 45 for injecting the washing liquid through the liquid injecting and discharging port 31c into the cartridge 31, which is located at the position for liquid injection.

The pair of the pulleys 41b and 41c are capable of being rotated by a motor (not shown) selectively in the clockwise direction or the counter-clockwise direction in FIG. 8.

Also, as illustrated in FIG. 8, the pre-treatment liquid injecting pin 42, the hybridization liquid injecting pin 43, the probe liquid injecting pin 44, and the washing liquid injecting pin 45 are secured to a liquid pin head 46. The liquid pin head 46 is capable of being moved by a motor (not shown) along a pair or rails (not shown).

As illustrated in FIG. 8, the reacting section 34 of the hybridization apparatus 30 comprises a fourth endless belt 47a for receiving the cartridge 31 from the third endless belt 41a of the liquid injecting section 33, and transferring the cartridge 31 to the third endless belt 41a of the liquid injecting section 33. The reacting section 34 also comprises a pair of pulleys 47b and 47c, over which the fourth endless belt 47a is threaded and which are capable of rotating selectively in the clockwise direction or the counter-clockwise direction in FIG. 8. The reacting section 34 further comprises a vibrating table 48 for vibrating the fourth endless belt 47a.

As illustrated in FIG. 8, the biochemical analysis unit take-out section 35 of the hybridization apparatus 30 comprises a fifth endless belt 49a for receiving the cartridge 31 from the fourth endless belt 47a of the reacting section 34, and transferring the cartridge 31 to the fourth endless belt 47a of the reacting section 34. The biochemical analysis unit take-out section 35 also comprises a pair of pulleys 49b and 49c, over which the fifth endless belt 49a is threaded and which are capable of rotating selectively in the clockwise direction or the counter-clockwise direction in FIG. 8. The biochemical analysis unit take-out section 35 further comprises a radiation sensor 50 for detecting the concentration of the radioactive labeling substance contained in the washing liquid within the cartridge 31. The biochemical analysis unit take-out section 35 still further comprises a liquid discharging pin 51 for discharging the pre-treatment liquid, the liquid prepared by the addition of the probe liquid to the hybridization liquid, or the washing liquid from the cartridge 31. The biochemical analysis unit take-out section 35 also comprises a biochemical analysis unit take-out mechanism 52 for opening the cover 31b of the cartridge 31, and taking out the biochemical analysis unit 1 from the cartridge 31.

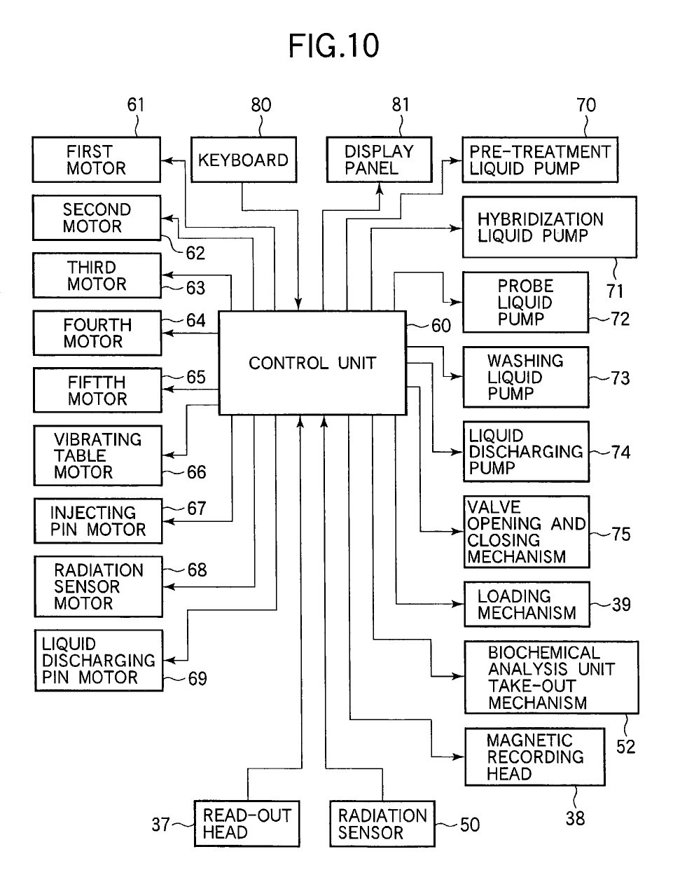

FIG. 10 is a block diagram showing a control system, a detecting system, a driving system, an input system, and a displaying system of the hybridization apparatus 30.

As illustrated in FIG. 10, the control system of the hybridization apparatus 30 comprises a control unit 60 for controlling the operations of the hybridization apparatus 30. The detecting system of the hybridization apparatus 30 comprises the read-out head 37 for reading out the data having been recorded on the magnetic recording layer 5 of the biochemical analysis unit 1, and the radiation sensor 50 for detecting the amount of the radioactive labeling substance contained in the washing liquid within the cartridge 31.

As illustrated in FIG. 10, the driving system of the hybridization apparatus 30 comprises a first motor 61 for rotating the pair of the pulleys 36b and 36c in order to drive the first endless belt 36a. The driving system of the hybridization apparatus 30 also comprises a second motor 62 for rotating the pair of the pulleys 40b and 40c in order to drive the second endless belt 40a. The driving system of the hybridization apparatus 30 further comprises a third motor 63 for rotating the pair of the pulleys 41b and 41c in order to drive the third endless belt 41a. The driving system of the hybridization apparatus 30 still further comprises a fourth motor 64 for rotating the pair of the pulleys 47b and 47c in order to drive the fourth endless belt 47a. The driving system of the hybridization apparatus 30 also comprises a fifth motor 65 for rotating the pulleys 49b and 49c in order to drive the fifth endless belt 49a. The driving system of the hybridization apparatus 30 further comprises a vibrating table motor 66 for driving the vibrating table 48. The driving system of the hybridization apparatus 30 still further comprises an injecting pin motor 67 for moving the liquid pin head 46 along the pair of the rails (not shown), such that the pre-treatment liquid injecting pin 42, the hybridization liquid injecting pin 43, the probe liquid injecting pin 44, or the washing liquid injecting pin 45 selectively stands facing the liquid injecting and discharging port 31c of the cartridge 31. The driving system of the hybridization apparatus 30 also comprises a radiation sensor motor 68 for moving the radiation sensor 50 between a position for detection within the cartridge 31, which is located at the position for liquid discharging, and a retreated position away from the cartridge 31. The driving system of the hybridization apparatus 30 further comprises a liquid discharging pin motor 69 for moving the liquid discharging pin 51 between a position for liquid suction within the cartridge 31, which is located at the position for liquid discharging, and are treated position away from the cartridge 31. The driving system of the hybridization apparatus 30 still further comprises a pre-treatment liquid pump 70 for supplying the pre-treatment liquid from a pre-treatment liquid tank (not shown), which accommodates the pre-treatment liquid, to the pre-treatment liquid injecting pin 42. The driving system of the hybridization apparatus 30 also comprises a hybridization liquid pump 71 for supplying the hybridization liquid from a hybridization liquid tank (not shown), which accommodates the hybridization liquid, to the hybridization liquid injecting pin 43. The driving system of the hybridization apparatus 30 further comprises a probe liquid pump 72 for supplying the probe liquid from a probe liquid tip (not shown), which accommodates the probe liquid containing the organism-originating substance having been labeled with the labeling substance, to the probe liquid injecting pin 44. The driving system of the hybridization apparatus 30 still further comprises a washing liquid pump 73 for supplying the washing liquid from a washing liquid tank (not shown), which accommodates the washing liquid, to the washing liquid injecting pin 45. The driving system of the hybridization apparatus 30 also comprises a liquid discharging pump 74 for discharging the pre-treatment liquid, the liquid prepared by the addition of the probe liquid to the hybridization liquid, or the washing liquid from the cartridge 31. The driving system of the hybridization apparatus 30 further comprises a valve opening and closing mechanism 75 for selectively opening and closing a valve (not shown) for communication of a pre-treatment liquid collecting tank (not shown) for collecting the pre-treatment liquid and the liquid discharging pin 51 with each other, a valve (not shown) for communication of a hybridization liquid collecting tank (not shown) for collecting the liquid, which has been prepared by the addition of the probe liquid to the hybridization liquid, and the liquid discharging pin 51 with each other, or a valve (not shown) for communication of a washing liquid collecting tank (not shown) for collecting the washing liquid and the liquid discharging pin 51 with each other. The driving system of the hybridization apparatus 30 still further comprises the loading mechanism 39 for opening the cover 31b of the cartridge 31, loading the biochemical analysis unit 1 into the cartridge 31, and closing the cover 31b of the cartridge 31. The driving system of the hybridization apparatus 30 also comprises the biochemical analysis unit take-out mechanism 52 for opening the cover 31b of the cartridge 31, and taking out the biochemical analysis unit 1 from the cartridge 31. The driving system of the hybridization apparatus 30 further comprises the magnetic recording head 38 for writing the data on the magnetic recording layer 5 of the biochemical analysis unit 1.

As illustrated in FIG. 10, the input system of the hybridization apparatus 30 comprises a keyboard 80. Also, the displaying system of the hybridization apparatus 30 comprises a displaying panel 81.

In the hybridization apparatus 30 constituted in the manner described above, the organism-originating substance having been labeled with the labeling substance is subjected to the selective hybridization with the specific binding substances, which have been adsorbed to the plurality of the adsorptive regions 4, 4, . . . of the base plate 2 of the biochemical analysis unit 1, in the manner described below.

Firstly, the hybridization liquid is prepared and accommodated in the hybridization liquid tank (not shown). Also, the washing liquid is prepared and accommodated in the washing liquid tank (not shown).

Further, the probe liquid, which contains the organism-originating substance having been labeled with the labeling substance, is prepared and accommodated in the probe liquid tip (not shown).

In cases where the specific binding substance, such as the cDNA, is to be labeled selectively with the radioactive labeling substance, the probe liquid containing the organism-originating substance, which has been labeled with the radioactive labeling substance and acts as a probe, is prepared and accommodated in the probe liquid tip.

In cases where the specific binding substance, such as the cDNA, is to be labeled selectively with the labeling substance capable of producing the chemiluminescence when being brought into contact with a chemiluminescence substrate, the probe liquid containing the organism-originating substance, which has been labeled with the labeling substance capable of producing the chemiluminescence when being brought into contact with a chemiluminescence substrate and which acts as the probe, is prepared and accommodated in the probe liquid tip.

In cases where the specific binding substance, such as the cDNA, is to be labeled selectively with the fluorescent labeling substance, such as the fluoro chrome, the probe liquid containing the organism-originating substance, which has been labeled with the fluorescent labeling substance, such as the fluoro chrome, and which acts as the probe, is prepared and accommodated in the probe liquid tip.

A probe liquid containing at least two organism-originating substances selected from among the organism-originating substance, which has been labeled with the radioactive labeling substance, the organism-originating substance, which has been labeled with the labeling substance capable of producing the chemiluminescence when being brought into contact with a chemiluminescence substrate, and the organism-originating substance, which has been labeled with the fluorescent labeling substance, such as the fluoro chrome, may be prepared and accommodated in the probe liquid tip. In this embodiment, the probe liquid containing the organism-originating substance, which has been labeled with the radioactive labeling substance, and the organism-originating substance, which has been labeled with the fluorescent labeling substance, is prepared and accommodated in the probe liquid tip.

When the hybridization is to be executed, the biochemical analysis unit 1 comprising the plurality of the adsorptive regions 4, 4, . . . , to which the specific binding substances, such as the cDNA's, have respectively been adsorbed, is set by the user on the first endless belt 36a of the cartridge loading section 32. Also, a start signal is inputted from the keyboard 80. At the same time, in cases where the organism-originating substance, which has been labeled with the radioactive labeling substance, is to be subjected to the hybridization with the specific binding substances contained in the plurality of the adsorptive regions 4, 4, . . . of the biochemical analysis unit 1, a radioactive label signal is inputted by the user from the keyboard 80.

The start signal and the radioactive label signal having been inputted from the keyboard 80 are fed into the control unit 60.

When the control unit 60 receives the start signal, the control unit 60 feeds a driving signal into the first motor 61. The first motor 61 rotates the pair of the pulleys 36b and 36c in order to rotate the first endless belt 36a clockwise in FIG. 8.

When the magnetic recording layer 5 of the biochemical analysis unit 1, which has been set on the first endless belt 36a, arrives at the position, which stands facing the read-out head 37, the control unit 60 feeds a driving stop signal to the first motor 61 in order to stop the first endless belt 36a. In this state, the data having been recorded on the first data recording region 5a of the magnetic recording layer 5, which region is protected from data writing conducted by a user, is read out by the read-out head 37.

In this embodiment, the data concerning the amount of the adsorptive material contained in each of the adsorptive regions 4, 4, . . . , the data concerning the kinds of the specific binding substances, the data concerning the positions of the adsorptive regions 4, 4, . . . , to which positions the specific binding substances have been spotted respectively, and the data concerning the number of times of use of the biochemical analysis unit 1 have been recorded on the first data recording region 5a of the magnetic recording layer 5. In cases where the biochemical analysis unit 1 and a specific stimulable phosphor sheet are supplied as one biochemical analysis kit to the user, the identification data for specifying the stimulable phosphor sheet, which constitutes the one biochemical analysis kit together with the biochemical analysis unit 1 and is to be used together with the biochemical analysis unit 1, has also been recorded on the first data recording region 5a of the magnetic recording layer 5.

The data having been read out by the read-out head 37 are fed into the control unit 60. In cases where it has been judged in accordance with the data, which have been received from the read-out head 37, that the biochemical analysis unit 1 has already been used N number of times, the control unit 60 feeds a reverse rotation signal into the first motor 61. The pulleys 36b and 36c are thus rotated counter-clockwise in FIG. 8, and the biochemical analysis unit 1 is sent back to the user. Also, a message instructing change-over of the biochemical analysis unit 1 is displayed on the displaying panel 81.

If the biochemical analysis unit 1 is used N number of times or more, the specific binding substances having been adsorbed to the adsorptive regions 4, 4, . . . will separate from the adsorptive regions 4, 4, . . . . As a result, the accuracy of the analysis will become markedly low, and reliable analysis results cannot be obtained. The value of N is set to be, for example, 2.

The biochemical analysis unit 1 having been returned to the user is collected by a maker and subjected to recycling.

In cases where it has been judged in accordance with the data, which have been received from the read-out head 37, that the number of times of use of the biochemical analysis unit 1 is smaller than N, the control unit 60 further feeds the driving signal into the first motor 61, and the biochemical analysis unit 1 is moved to the position, at which the magnetic recording layer 5 stands facing the magnetic recording head 38.

When the biochemical analysis unit 1 has been moved to the position, at which the magnetic recording layer 5 stands facing the magnetic recording head 38, the driving stop signal is fed from the control unit 60 into the first motor 61.

Thereafter, the control unit 60 feeds a writing signal to the magnetic recording head 38 in order to increase the number of times of use of the biochemical analysis unit 1 having been recorded on the first data recording region 5a of the magnetic recording layer 5, which region is protected from data writing conducted by a user, by a value of 1 and to write the data concerning the date and hour of execution of the hybridization on the first data recording region 5a of the magnetic recording layer 5 in accordance with a built-in clock. Also, in cases where the radioactive label signal has been inputted together with the start signal, information representing that the radioactive labeling substance has been used is written on the first data recording region 5a of the magnetic recording layer 5.

In cases where data, which is necessary for the user to manage the biochemical analysis unit 1, such as data for specifying a person who sampled the organism-originating substance to be subjected to the selective hybridization with the specific binding substances having been adsorbed to the adsorptive regions 4, 4, . . . of the biochemical analysis unit 1, has been inputted by the user from the keyboard 80, the control unit 60 feeds the writing signal to the magnetic recording head 38 in order to write the data on the second data recording region 5b, on which the data is capable of being written by the user.

When the data writing on the magnetic recording layer 5 is completed, the control unit 60 again feeds the driving signal to the first motor 61, and the pulleys 36b and 36c are rotated in order to convey the biochemical analysis unit 1 into the loading mechanism 39.

In the loading mechanism 39, the cartridge 31 is supported in a state in which the cover 31b is open. The biochemical analysis unit 1 is conveyed by the first endless belt 36a into the cartridge 31.

When the biochemical analysis unit 1 has been conveyed into the cartridge 31, the control unit 60 feeds the driving stop signal into the first motor 61 in order to stop the driving of the first endless belt 36a. Also, the control unit 60 feeds a loading signal to the loading mechanism 39 in order to close the cover 31b of the cartridge 31.

Thereafter, the control unit 60 feeds the driving signal into the second motor 62. The second motor 62 rotates the pair of the pulleys 40b and 40c in order to rotate the second endless belt 40a clockwise in FIG. 8. Also, the control unit 60 feeds the driving signal into the third motor 63. The third motor 63 rotates the pulleys 41b and 41c in order to rotate the third endless belt 41a clockwise in FIG. 8.

As a result, the cartridge 31, in which the biochemical analysis unit 1 has been accommodated, is transferred from the second endless belt 40a of the cartridge loading section 32 to the third endless belt 41a of the liquid injecting section 33.

When the cartridge 31 has been transferred to the third endless belt 41a of the liquid injecting section 33, the control unit 60 feeds the driving stop signal into the second motor 62 in order to stop the driving of the second endless belt 40a. Also, when the cartridge 31 has been moved by the third endless belt 41a to the position for liquid injection, the control unit 60 feeds the driving stop signal to the third motor 63 in order to stop the cartridge 31.

Thereafter, the control unit 60 feeds the driving signal into the injecting pin motor 67 in order to move the liquid pin head 46 along the pair of the rails (not shown) until the pre-treatment liquid injecting pin 42 arrives at the position, which stands facing the liquid injecting and discharging port 31c of the cartridge 31.

When the pre-treatment liquid injecting pin 42 has been moved to the position, which stands facing the liquid injecting and discharging port 31c of the cartridge 31, the control unit 60 feeds the driving signal into the pre-treatment liquid pump 70 in order to inject the pre-treatment liquid from the pre-treatment liquid tank (not shown) through the pre-treatment liquid injecting pin 42 and the liquid injecting and discharging port 31c into the cartridge 31.

When a predetermined length of time has elapsed, the control unit 60 feeds the driving stop signal into the pre-treatment liquid pump 70 in order to stop the injection of the pre-treatment liquid into the cartridge 31. Also, the control unit 60 feeds the driving signal into the third motor 63 in order to drive the third endless belt 41a.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately a center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and all of the adsorptive regions 4, 4, . . . of the biochemical analysis unit 1 accommodated in the cartridge 31 are wetted with the pre-treatment liquid.

When a predetermined length of time has elapsed, the control unit 60 feeds the driving stop signal into the vibrating table motor 66 in order to stop the vibration of the vibrating table 48. Also, the control unit 60 feeds the driving signal into the fourth motor 64. The fourth motor 64 rotates the pulleys 47b and 47c in order to rotate the fourth endless belt 47a clockwise in FIG. 8. Also, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is transferred from the fourth endless belt 47a of the reacting section 34 to the fifth endless belt 49a of the biochemical analysis unit take-out section 35.

When the cartridge 31 has been transferred to the fifth endless belt 49a of the biochemical analysis unit take-out section 35, the control unit 60 feeds the driving stop signal into the fourth motor 64 in order to stop the driving of the fourth endless belt 47a. When the cartridge 31 has been moved by the fifth endless belt 49a to the position for liquid discharging, the control unit 60 feeds the driving stop signal into the fifth motor 65 in order to stop the fifth endless belt 49a.

Thereafter, the control unit 60 feeds the driving signal to the valve opening and closing mechanism 75 in order to open the valve (not shown) for communication of the pre-treatment liquid collecting tank (not shown) for collecting the pre-treatment liquid and the liquid discharging pin 51 with each other. Also, the control unit 60 feeds the driving signal into the liquid discharging pin motor 69 in order to move the liquid discharging pin 51 to the position for liquid suction within the cartridge 31. Further, the control unit 60 feeds the driving signal into the liquid discharging pump 74. The liquid discharging pump 74 sucks up the pre-treatment liquid from the cartridge 31.

When the pre-treatment liquid has thus been sucked up by the liquid discharging pump 74 from the cartridge 31 and collected into the pre-treatment liquid collecting tank, the control unit 60 feeds the reverse driving signal into the fifth motor 65. The fifth motor 65 rotates the pulleys 49b and 49c in order to rotate the fifth endless belt 49a counter-clockwise in FIG. 8. Also, the control unit 60 feeds the reverse driving signal into the fourth motor 64. The fourth motor 64 rotates the pulleys 47b and 47c in order to rotate the fourth endless belt 47a counter-clockwise in FIG. 8.

As a result, the cartridge 31 is transferred from the fifth endless belt 49a of the biochemical analysis unit take-out section 35 to the fourth endless belt 47a of the reacting section 34.

When the cartridge 31 has been transferred to the fourth endless belt 47a of the reacting section 34, the control unit 60 feeds the driving stop signal into the fifth motor 65 in order to stop the driving of the fifth endless belt 49a. Also, the control unit 60 feeds the reverse driving signal into the third motor 63. The third motor 63 rotates the pulleys 41b and 41c in order to rotate the third endless belt 41a counter-clockwise in FIG. 8.

As a result, the cartridge 31 is transferred from the fourth endless belt 47a of the reacting section 34 to the third endless belt 41a of the liquid injecting section 33.

When the cartridge 31 has been transferred to the third endless belt 41a of the liquid injecting section 33, the control unit 60 feeds the driving stop signal into the fourth motor 64 in order to stop the driving of the fourth endless belt 47a. When the cartridge 31 has been moved by the third endless belt 41a to the position for liquid injection, the control unit 60 feeds the driving stop signal into the third motor 63 in order to stop the driving of the third endless belt 41a.

Thereafter, the control unit 60 feeds the driving signal into the injecting pin motor 67 in order to move the liquid pin head 46 along the pair of the rails (not shown) until the hybridization liquid injecting pin 43 arrives at the position, which stands facing the liquid injecting and discharging port 31c of the cartridge 31.

When the hybridization liquid injecting pin 43 has thus been moved to the position, which stands facing the liquid injecting and discharging port 31c of the cartridge 31, the control unit 60 feeds the driving signal into the hybridization liquid pump 71 in order to inject the hybridization liquid from the hybridization liquid tank (not shown) through the hybridization liquid injecting pin 43 and the liquid injecting and discharging port 31c into the cartridge 31.

When a predetermined length of time has elapsed, the control unit 60 feeds the driving stop signal into the hybridization liquid pump 71 in order to stop the injection of the hybridization liquid into the cartridge 31. Also, the control unit 60 feeds the driving signal into the third motor 63. The third motor 63 rotates the pulleys 41b and 41c in order to rotate the third endless belt 41a clockwise in FIG. 8.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the hybridization liquid is brought into uniform contact with all of the adsorptive regions 4, 4, . . . of the biochemical analysis unit 1 accommodated in the cartridge 31. In this manner, the hybridization is executed.

When a predetermined length of time has elapsed, the control unit 60 feeds the driving stop signal into the vibrating table motor 66 in order to stop the vibration of the vibrating table 48. Also, the control unit 60 feeds the reverse driving signal into the fourth motor 64. The fourth motor 64 rotates the pulleys 47b and 47c in order to rotate the fourth endless belt 47a counter-clockwise in FIG. 8. Also, the control unit 60 feeds the reverse driving signal into the third motor 63. The third motor 63 rotates the pulleys 41b and 41c in order to rotate the third endless belt 41a counter-clockwise in FIG. 8.

As a result, the cartridge 31 is transferred from the fourth endless belt 47a of the reacting section 34 to the third endless belt 41a of the liquid injecting section 33.

When the cartridge 31 has been transferred to the third endless belt 41a of the liquid injecting section 33, the control unit 60 feeds the driving stop signal into the fourth motor 64 in order to stop the driving of the fourth endless belt 47a. When the cartridge 31 has been moved by the third endless belt 41a to the position for liquid injection, the control unit 60 feeds the driving stop signal into the third motor 63 in order to stop the driving of the third endless belt 41a.

Thereafter, the control unit 60 feeds the driving signal into the injecting pin motor 67 in order to move the liquid pin head 46 along the pair of the rails (not shown) until the probe liquid injecting pin 44 arrives at the position, which stands facing the liquid injecting and discharging port 31c of the cartridge 31.

When the probe liquid injecting pin 44 has thus been moved to the position, which stands facing the liquid injecting and discharging port 31c of the cartridge 31, the control unit 60 feeds the driving signal into the probe liquid pump 72 in order to inject the probe liquid from the probe liquid tip (not shown) through the probe liquid injecting pin 44 and the liquid injecting and discharging port 31c into the cartridge 31.

As a result, the probe liquid, which contains the organism-originating substances having been labeled with the labeling substances, is added to the hybridization liquid having been accommodated in the cartridge 31.

When a predetermined length of time has elapsed, the control unit 60 feeds the driving stop signal into the probe liquid pump 72 in order to stop the injection of the probe liquid into the cartridge 31. Also, the control unit 60 feeds the driving signal into the third motor 63. The third motor 63 rotates the pulleys 41b and 41c in order to rotate the third endless belt 41a clockwise in FIG. 8.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the hybridization liquid is brought into uniform contact with all of the adsorptive regions 4, 4, . . . of the biochemical analysis unit 1 accommodated in the cartridge 31. Also, the organism-originating substance, which has been labeled with the radioactive labeling substance and contained in the hybridization liquid, and the organism-originating substance, which has been labeled with the fluorescent labeling substance and contained in the hybridization liquid, are subjected to the selective hybridization with the specific binding substances having been adsorbed to the plurality of the adsorptive regions 4, 4, . . . . In this manner, at least one specific binding substance among the plurality of the specific binding substances having been adsorbed to the plurality of the adsorptive regions 4, 4, . . . is selectively labeled with the radioactive labeling substance. Also, at least one specific binding substance is selectively labeled with the fluorescent labeling substance.

When a predetermined length of time has elapsed, the control unit 60 feeds the driving stop signal into the vibrating table motor 66 in order to stop the vibration of the vibrating table 48. Also, the control unit 60 feeds the driving signal into the fourth motor 64. The fourth motor 64 rotates the pulleys 47b and 47c in order to rotate the fourth endless belt 47a clockwise in FIG. 8. Also, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is transferred from the fourth endless belt 47a of the reacting section 34 to the fifth endless belt 49a of the biochemical analysis unit take-out section 35.

When the cartridge 31 has been transferred to the fifth endless belt 49a of the biochemical analysis unit take-out section 35, the control unit 60 feeds the driving stop signal into the fourth motor 64 in order to stop the driving of the fourth endless belt 47a. When the cartridge 31 has been moved by the fifth endless belt 49a to the position for liquid discharging, the control unit 60 feeds the driving stop signal into the fifth motor 65 in order to stop the fifth endless belt 49a.

Thereafter, the control unit 60 feeds the driving signal to the valve opening and closing mechanism 75 in order to open the valve (not shown) for communication of the hybridization liquid collecting tank (not shown) for collecting the liquid, which has been prepared by the addition of the probe liquid to the hybridization liquid, and the liquid discharging pin 51 with each other. Also, the control unit 60 feeds the driving signal into the liquid discharging pin motor 69 in order to move the liquid discharging pin 51 to the position for liquid suction within the cartridge 31. Further, the control unit 60 feeds the driving signal into the liquid discharging pump 74. The liquid discharging pump 74 sucks up the liquid, which has been prepared by the addition of the probe liquid to the hybridization liquid, from the cartridge 31.

When the liquid, which has been prepared by the addition of the probe liquid to the hybridization liquid, has thus been sucked up by the liquid discharging pump 74 from the cartridge 31 and collected into the hybridization liquid collecting tank, the control unit 60 feeds the reverse driving signal into the fifth motor 65. The fifth motor 65 rotates the pulleys 49b and 49c in order to rotate the fifth endless belt 49a counter-clockwise in FIG. 8. Also, the control unit 60 feeds the reverse driving signal into the fourth motor 64. The fourth motor 64 rotates the pulleys 47b and 47c in order to rotate the fourth endless belt 47a counter-clockwise in FIG. 8.

As a result, the cartridge 31 is transferred from the fifth endless belt 49a of the biochemical analysis unit take-out section 35 to the fourth endless belt 47a of the reacting section 34.

When the cartridge 31 has been transferred to the fourth endless belt 47a of the reacting section 34, the control unit 60 feeds the driving stop signal into the fifth motor 65 in order to stop the driving of the fifth endless belt 49a. Also, the control unit 60 feeds the reverse driving signal into the third motor 63. The third motor 63 rotates the pulleys 41b and 41c in order to rotate the third endless belt 41a counter-clockwise in FIG. 8.

As a result, the cartridge 31 is transferred from the fourth endless belt 47a of the reacting section 34 to the third endless belt 41a of the liquid injecting section 33.

When the cartridge 31 has been transferred to the third endless belt 41a of the liquid injecting section 33, the control unit 60 feeds the driving stop signal into the fourth motor 64 in order to stop the driving of the fourth endless belt 47a. When the cartridge 31 has been moved by the third endless belt 41a to the position for liquid injection, the control unit 60 feeds the driving stop signal into the third motor 63 in order to stop the driving of the third endless belt 41a.

Thereafter, the control unit 60 feeds the driving signal into the injecting pin motor 67 in order to move the liquid pin head 46 along the pair of the rails (not shown) until the washing liquid injecting pin 45 arrives at the position, which stands facing the liquid injecting and discharging port 31c of the cartridge 31.

When the washing liquid injecting pin 45 has thus been moved to the position, which stands facing the liquid injecting and discharging port 31c of the cartridge 31, the control unit 60 feeds the driving signal into the washing liquid pump 73 in order to inject the washing liquid from the washing liquid tank (not shown) through the washing liquid injecting pin 45 and the liquid injecting and discharging port 31c into the cartridge 31.

When a predetermined length of time has elapsed, the control unit 60 feeds the driving stop signal into the washing liquid pump 73 in order to stop the injection of the washing liquid into the cartridge 31. Also, the control unit 60 feeds the driving signal into the third motor 63. The third motor 63 rotates the pulleys 41b and 41c in order to rotate the third endless belt 41a clockwise in FIG. 8.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the washing liquid is brought into uniform contact with all of the adsorptive regions 4, 4, . . . of the biochemical analysis unit 1 accommodated in the cartridge 31. In this manner, the adsorptive regions 4, 4, . . . are washed with the washing liquid.

When a predetermined length of time has elapsed, the control unit 60 feeds the driving stop signal into the vibrating table motor 66 in order to stop the vibration of the vibrating table 48. Also, the control unit 60 feeds the driving signal into the fourth motor 64. The fourth motor 64 rotates the pulleys 47b and 47c in order to rotate the fourth endless belt 47a clockwise in FIG. 8. Also, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is transferred from the fourth endless belt 47a of the reacting section 34 to the fifth endless belt 49a of the biochemical analysis unit take-out section 35.

When the cartridge 31 has been transferred to the fifth endless belt 49a of the biochemical analysis unit take-out section 35, the control unit 60 feeds the driving stop signal into the fourth motor 64 in order to stop the driving of the fourth endless belt 47a. When the cartridge 31 has been moved by the fifth endless belt 49a to the position for liquid discharging, the control unit 60 feeds the driving stop signal into the fifth motor 65 in order to stop the driving of the fifth endless belt 49a.

Thereafter, the control unit 60 feeds the driving signal to the radiation sensor motor 68 in order to move the radiation sensor 50 to the position for detection within the cartridge 31. Also, the control unit 60 feeds the driving signal to the valve opening and closing mechanism 75 in order to open the valve (not shown) for communication of the washing liquid collecting tank (not shown) for collecting the washing liquid and the liquid discharging pin 51 with each other. Further, the control unit 60 feeds the driving signal into the liquid discharging pin motor 69 in order to move the liquid discharging pin 51 to the position for liquid suction within the cartridge 31.

In this manner, the concentration of the radioactive labeling substance in the washing liquid, which is accommodated in the cartridge 31, is detected by the radiation sensor 50. The detection signal obtained from the radiation sensor 50 is fed into the control unit 60.

Furthermore, the control unit 60 feeds the driving signal into the liquid discharging pump 74. The liquid discharging pump 74 sucks up the washing liquid from the cartridge 31. The washing liquid is collected into the washing liquid collecting tank (not shown).

When a predetermined length of time has elapsed, the control unit 60 feeds the driving stop signal into the liquid discharging pump 74 in order to stop the suction of the washing liquid from the cartridge 31. Also, the control unit 60 feeds the driving signal into the liquid discharging pin motor 69 in order to retreat the liquid discharging pin 51 to the retreated position away from the cartridge 31. Further, the control unit 60 feeds the driving signal into the radiation sensor motor 68 in order to retreat the radiation sensor 50 to the retreated position away from the cartridge 31.

In accordance with the detection signal having been received from the radiation sensor 50, the control unit 60 compares the concentration of the radioactive labeling substance in the washing liquid and a reference concentration of the radioactive labeling substance, which reference concentration has been stored in the memory (not shown), with each other. In cases where the concentration of the radioactive labeling substance in the washing liquid is higher than the reference concentration of the radioactive labeling substance, it is regarded that the washing of the adsorptive regions 4, 4, . . . is not sufficient, and that the washing operation should further be performed by injecting the washing liquid into the cartridge 31. Therefore, in such cases, at the time at which the washing liquid in the cartridge 31 has been sucked up by the liquid discharging pump 74 and collected into the washing liquid collecting tank, the control unit 60 feeds the reverse driving signal into the fifth motor 65. The fifth motor 65 rotates the pulleys 49b and 49c in order to rotate the fifth endless belt 49a counter-clockwise in FIG. 8. Also, the control unit 60 feeds the reverse driving signal into the fourth motor 64. The fourth motor 64 rotates the pulleys 47b and 47c in order to rotate the fourth endless belt 47a counter-clockwise in FIG. 8.

As a result, the cartridge 31 is transferred from the fifth endless belt 49a of the biochemical analysis unit take-out section 35 to the fourth endless belt 47a of the reacting section 34.

When the cartridge 31 has been transferred to the fourth endless belt 47a of the reacting section 34, the control unit 60 feeds the driving stop signal into the fifth motor 65 in order to stop the driving of the fifth endless belt 49a. Also, the control unit 60 feeds the reverse driving signal into the third motor 63. The third motor 63 rotates the pulleys 41b and 41c in order to rotate the third endless belt 41a counter-clockwise in FIG. 8.

As a result, the cartridge 31 is transferred from the fourth endless belt 47a of the reacting section 34 to the third endless belt 41a of the liquid injecting section 33.

When the cartridge 31 has been transferred to the third endless belt 41a of the liquid injecting section 33, the control unit 60 feeds the driving stop signal into the fourth motor 64 in order to stop the driving of the fourth endless belt 47a. When the cartridge 31 has been moved by the third endless belt 41a to the position for liquid injection, the control unit 60 feeds the driving stop signal into the third motor 63 in order to stop the driving of the third endless belt 41a.

When the cartridge 31 has thus been returned to the position for liquid injection, the control unit 60 again feeds the driving signal into the washing liquid pump 73 in order to inject the washing liquid from the washing liquid tank (not shown) through the washing liquid injecting pin 45 and the liquid injecting and discharging port 31c into the cartridge 31.

When a predetermined length of time has elapsed, the control unit 60 feeds the driving stop signal into the washing liquid pump 73 in order to stop the injection of the washing liquid into the cartridge 31. Also, the control unit 60 feeds the driving signal into the third motor 63. The third motor 63 rotates the pulleys 41b and 41c in order to rotate the third endless belt 41a clockwise in FIG. 8.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the washing liquid is brought into uniform contact with all of the adsorptive regions 4, 4, . . . of the biochemical analysis unit 1 accommodated in the cartridge 31. In this manner, the adsorptive regions 4, 4, . . . are washed with the washing liquid.

When a predetermined length of time has elapsed, the control unit 60 feeds the driving stop signal into the vibrating table motor 66 in order to stop the vibration of the vibrating table 48. Also, the control unit 60 feeds the driving signal into the fourth motor 64. The fourth motor 64 rotates the pulleys 47b and 47c in order to rotate the fourth endless belt 47a clockwise in FIG. 8. Also, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is transferred from the fourth endless belt 47a of the reacting section 34 to the fifth endless belt 49a of the biochemical analysis unit take-out section 35.

When the cartridge 31 has been transferred to the fifth endless belt 49a of the biochemical analysis unit take-out section 35, the control unit 60 feeds the driving stop signal into the fourth motor 64 in order to stop the driving of the fourth endless belt 47a. When the cartridge 31 has been moved by the fifth endless belt 49a to the position for liquid discharging, the control unit 60 feeds the driving stop signal into the fifth motor 65 in order to stop the driving of the fifth endless belt 49a.

Thereafter, the control unit 60 feeds the driving signal to the radiation sensor motor 68 in order to move the radiation sensor 50 to the position for detection within the cartridge 31. Also, the control unit 60 feeds the driving signal to the valve opening and closing mechanism 75 in order to open the valve (not shown) for communication of the washing liquid collecting tank (not shown) for collecting the washing liquid and the liquid discharging pin 51 with each other. Further, the control unit 60 feeds the driving signal into the liquid discharging pin motor 69 in order to move the liquid discharging pin 51 to the position for liquid suction within the cartridge 31.

In this manner, the concentration of the radioactive labeling substance in the washing liquid, which is accommodated in the cartridge 31, is detected by the radiation sensor 50. The detection signal obtained from the radiation sensor 50 is fed into the control unit 60.

Furthermore, the control unit 60 feeds the driving signal into the liquid discharging pump 74. The liquid discharging pump 74 sucks up the washing liquid from the cartridge 31. The washing liquid is collected into the washing liquid collecting tank (not shown).

When a predetermined length of time has elapsed, the control unit 60 feeds the driving stop signal into the liquid discharging pump 74 in order to stop the suction of the washing liquid from the cartridge 31. Also, the control unit 60 feeds the driving signal into the liquid discharging pin motor 69 in order to retreat the liquid discharging pin 51 to the retreated position away from the cartridge 31. Further, the control unit 60 feeds the driving signal into the radiation sensor motor 68 in order to retreat the radiation sensor 50 to the retreated position away from the cartridge 31.

In accordance with the detection signal having been received from the radiation sensor 50, the control unit 60 compares the concentration of the radioactive labeling substance in the washing liquid and the reference concentration of the radioactive labeling substance, which reference concentration has been stored in the memory (not shown), with each other. In cases where the concentration of the radioactive labeling substance in the washing liquid is higher than the reference concentration of the radioactive labeling substance, it is regarded that the washing of the adsorptive regions 4, 4, . . . is not sufficient, and that the washing operation should further be performed by injecting the washing liquid into the cartridge 31. Therefore, in such cases, at the time at which the washing liquid in the cartridge 31 has been sucked up by the liquid discharging pump 74 and collected into the washing liquid collecting tank, the control unit 60 feeds the reverse driving signal into the fifth motor 65. The fifth motor 65 rotates the pulleys 49b and 49c in order to rotate the fifth endless belt 49a counter-clockwise in FIG. 8. Also, the control unit 60 feeds the reverse driving signal into the fourth motor 64. The fourth motor 64 rotates the pulleys 47b and 47c in order to rotate the fourth endless belt 47a counter-clockwise in FIG. 8.

As a result, the cartridge 31 is transferred from the fifth endless belt 49a of the biochemical analysis unit take-out section 35 to the fourth endless belt 47a of the reacting section 34.

When the cartridge 31 has been transferred to the fourth endless belt 47a of the reacting section 34, the control unit 60 feeds the driving stop signal into the fifth motor 65 in order to stop the driving of the fifth endless belt 49a. Also, the control unit 60 feeds the reverse driving signal into the third motor 63. The third motor 63 rotates the pulleys 41b and 41c in order to rotate the third endless belt 41a counter-clockwise in FIG. 8.

As a result, the cartridge 31 is transferred from the fourth endless belt 47a of the reacting section 34 to the third endless belt 41a of the liquid injecting section 33.

When the cartridge 31 has been transferred to the third endless belt 41a of the liquid injecting section 33, the control unit 60 feeds the driving stop signal into the fourth motor 64 in order to stop the driving of the fourth endless belt 47a. When the cartridge 31 has been moved by the third endless belt 41a to the position for liquid injection, the control unit 60 feeds the driving stop signal into the third motor 63 in order to stop the driving of the third endless belt 41a.

When the cartridge 31 has thus been returned to the position for liquid injection, the control unit 60 again feeds the driving signal into the washing liquid pump 73 in order to inject the washing liquid from the washing liquid tank (not shown) through the washing liquid injecting pin 45 and the liquid injecting and discharging port 31c into the cartridge 31.

When a predetermined length of time has elapsed, the control unit 60 feeds the driving stop signal into the washing liquid pump 73 in order to stop the injection of the washing liquid into the cartridge 31. Also, the control unit 60 feeds the driving signal into the third motor 63. The third motor 63 rotates the pulleys 41b and 41c in order to rotate the third endless belt 41a clockwise in FIG. 8.

At the same time, the control unit 60 feeds the driving signal into the fourth motor 64. The fourth motor 64 rotates the pulleys 47b and 47c in order to rotate the fourth endless belt 47a clockwise in FIG. 8.

As a result, the cartridge 31, in which the biochemical analysis unit 1 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the washing liquid is brought into uniform contact with all of the adsorptive regions 4, 4, . . . of the biochemical analysis unit 1 accommodated in the cartridge 31. In this manner, the adsorptive regions 4, 4, . . . are washed with the washing liquid.

When a predetermined length of time has elapsed, the control unit 60 feeds the driving stop signal into the vibrating table motor 66 in order to stop the vibration of the vibrating table 48. Also, the control unit 60 feeds the driving signal into the fourth motor 64. The fourth motor 64 rotates the pulleys 47b and 47c in order to rotate the fourth endless belt 47a clockwise in FIG. 8. Also, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is transferred from the fourth endless belt 47a of the reacting section 34 to the fifth endless belt 49a of the biochemical analysis unit take-out section 35.

When the cartridge 31 has been transferred to the fifth endless belt 49a of the biochemical analysis unit take-out section 35, the control unit 60 feeds the driving stop signal into the fourth motor 64 in order to stop the driving of the fourth endless belt 47a. When the cartridge 31 has been moved by the fifth endless belt 49a to the position for liquid discharging, the control unit 60 feeds the driving stop signal into the fifth motor 65 in order to stop the driving of the fifth endless belt 49a.

Thereafter, the control unit 60 feeds the driving signal to the radiation sensor motor 68 in order to move the radiation sensor 50 to the position for detection within the cartridge 31. Also, the control unit 60 feeds the driving signal to the valve opening and closing mechanism 75 in order to open the valve (not shown) for communication of the washing liquid collecting tank (not shown) for collecting the washing liquid and the liquid discharging pin 51 with each other. Further, the control unit 60 feeds the driving signal into the liquid discharging pin motor 69 in order to move the liquid discharging pin 51 to the position for liquid suction within the cartridge 31.

In this manner, the concentration of the radioactive labeling substance in the washing liquid, which is accommodated in the cartridge 31, is detected by the radiation sensor 50. The detection signal obtained from the radiation sensor 50 is fed into the control unit 60.

Furthermore, the control unit 60 feeds the driving signal into the liquid discharging pump 74. The liquid discharging pump 74 sucks up the washing liquid from the cartridge 31. The washing liquid is collected into the washing liquid collecting tank (not shown).

When a predetermined length of time has elapsed, the control unit 60 feeds the driving stop signal into the liquid discharging pump 74 in order to stop the suction of the washing liquid from the cartridge 31. Also, the control unit 60 feeds the driving signal into the liquid discharging pin motor 69 in order to retreat the liquid discharging pin 51 to the retreated position away from the cartridge 31. Further, the control unit 60 feeds the driving signal into the radiation sensor motor 68 in order to retreat the radiation sensor 50 to the retreated position away from the cartridge 31.

In accordance with the detection signal having been received from the radiation sensor 50, the control unit 60 compares the concentration of the radioactive labeling substance in the washing liquid and the reference concentration of the radioactive labeling substance, which reference concentration has been stored in the memory (not shown), with each other.

In the manner described above, the washing operation with the washing liquid is repeated until the concentration of the radioactive labeling substance in the washing liquid decreases to a value at most equal to the reference concentration of the radioactive labeling substance. In cases where the concentration of the radioactive labeling substance in the washing liquid decreases to a value at most equal to the reference concentration of the radioactive labeling substance, the control unit 60 judges that the washing operation has been completed. In such cases, the control unit 60 feeds the driving signal into the fifth motor 65. The fifth motor 65 rotates the pulleys 49b and 49c in order to rotate the fifth endless belt 49a clockwise in FIG. 8.

As a result, the cartridge 31 is fed by the fifth endless belt 49a into the biochemical analysis unit take-out mechanism 52.

When the cartridge 31 has been fed into the biochemical analysis unit take-out mechanism 52, the control unit 60 feeds the driving stop signal into the fifth motor 65 in order to stop the driving of the fifth endless belt 49a. Also, the control unit 60 feeds the driving signal into the biochemical analysis unit take-out mechanism 52.

When the biochemical analysis unit take-out mechanism 52 receives the driving signal from the control unit 60, the biochemical analysis unit take-out mechanism 52 opens the cover 31b of the cartridge 31 and takes out the biochemical analysis unit 1 from the cartridge 31.

In the manner described above, the radiation information, which is formed with the radioactive labeling substance acting as the labeling substance, is recorded on at least one adsorptive region 4 among the plurality of the adsorptive regions 4, 4, . . . of the biochemical analysis unit 1. Also, the fluorescence information, which is formed with the fluorescent labeling substance, such as the fluoro chrome, is recorded on at least one adsorptive region 4 among the plurality of the adsorptive regions 4, 4, . . . of the biochemical analysis unit 1. The fluorescence information, which has been recorded on at least one adsorptive region 4 among the plurality of the adsorptive regions 4, 4, . . . , is read out by a scanner, which will be described later. In this manner, the data for a biochemical analysis is formed.

The radiation information, which is formed with the radioactive labeling substance, is transferred to a stimulable phosphor sheet. The radiation information, which has been transferred to the stimulable phosphor sheet, is read out by a scanner, which will be described later. In this manner, the data for a biochemical analysis is formed.

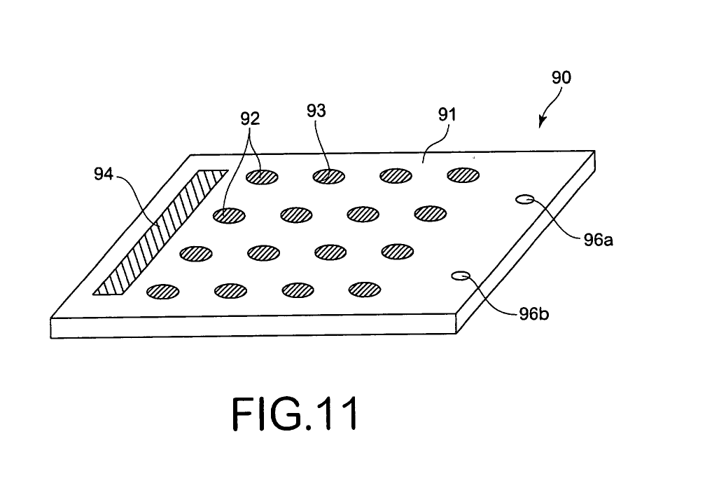

FIG. 11 is a schematic perspective view showing a stimulable phosphor sheet.

As illustrated in FIG. 11, a stimulable phosphor sheet 90 employed in this embodiment comprises a support 91, which is made from nickel and is provided with a plurality of through-holes 93, 93, . . . having an approximately circular shape. The through-holes 93, 93, . . . are located in a regular pattern. Also, a stimulable phosphor is filled in each of the through-holes 93, 93, . . . of the support 91. In this manner, a plurality of dot-shaped stimulable phosphor layer regions 92, 92, . . . are formed.

The plurality of the through-holes 93, 93, . . . of the support 91 are located in a pattern identical with the location pattern of the plurality of the adsorptive regions 4, 4, . . . of the base plate 2 of the biochemical analysis unit 1. Each of the stimulable phosphor layer regions 92, 92, . . . has a size identical with the side of each of the adsorptive regions 4, 4, . . . of the base plate 2 of the biochemical analysis unit 1.

Therefore, though not shown precisely in FIG. 11, in this embodiment, approximately 10,000 dot-shaped stimulable phosphor layer regions 92, 92, . . . , each of which has the approximately circular shape with a size of approximately 0.01 mm², are formed at a density of approximately 5,000 regions/cm² and in the regular pattern, which is identical with the location pattern of the plurality of the adsorptive regions 4, 4, . . . of the base plate 2 of the biochemical analysis unit 1, in the support 91 of the stimulable phosphor sheet 90.

As illustrated in FIG. 11, the support 91 is provided with two position matching through-holes 96a and 96b, which are located at the positions corresponding respectively to the position matching through-holes 6a and 6b of the base plate 2 of the biochemical analysis unit 1.

Also, in this embodiment, the stimulable phosphor is embedded in each of the through-holes 93, 93, . . . of the support 91, such that the heights of the top surfaces of the dot-shaped stimulable phosphor layer regions 92, 92, . . . may be identical with the height of the top surface of the support 91. In this manner, the stimulable phosphor sheet 90 is formed. Also, a magnetic recording layer 94 is formed on the surface of the support 91 and at the position corresponding to the magnetic recording layer 5 of the base plate 2 of the biochemical analysis unit 1.

In cases where the stimulable phosphor sheet 90 and a specific biochemical analysis unit 1 are supplied as one biochemical analysis kit to the user, the operator is capable of recording the identification data for specifying the biochemical analysis unit 1, which constitutes the one biochemical analysis kit together with the stimulable phosphor sheet 90, on the magnetic recording layer 94 of the stimulable phosphor sheet 90.

Byway of example, the data for a biochemical analysis may be obtained from an operation, wherein an organism-originating substance having been sampled from a person, who has an abnormality in genetic expression, and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. Also, the data for a biochemical analysis may be obtained from an operation, wherein an organism-originating substance having been sampled from a healthy person and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. An examination may then be executed by comparing the thus obtained two pieces of the data for a biochemical analysis. As described above, the identification data for specifying the biochemical analysis unit 1, which constitutes the one biochemical analysis kit together with the stimulable phosphor sheet 90, is capable of being recorded on the magnetic recording layer 94 of the stimulable phosphor sheet 90. Therefore, in cases where the examination is executed in the manner described above, the examination is capable of being executed reliably by using the same biochemical analysis unit 1 and the same stimulable phosphor sheet 90, and the accuracy of the examination is capable of being enhanced. Accordingly, in cases where the identification data for specifying the biochemical analysis unit 1 is recorded on the magnetic recording layer 94 of the stimulable phosphor sheet 90, the recorded identification data has a predetermined correspondence relationship to the identification data having been recorded on the magnetic recording layer 5 of the biochemical analysis unit 1, which constitutes the one biochemical analysis kit together with the stimulable phosphor sheet 90.

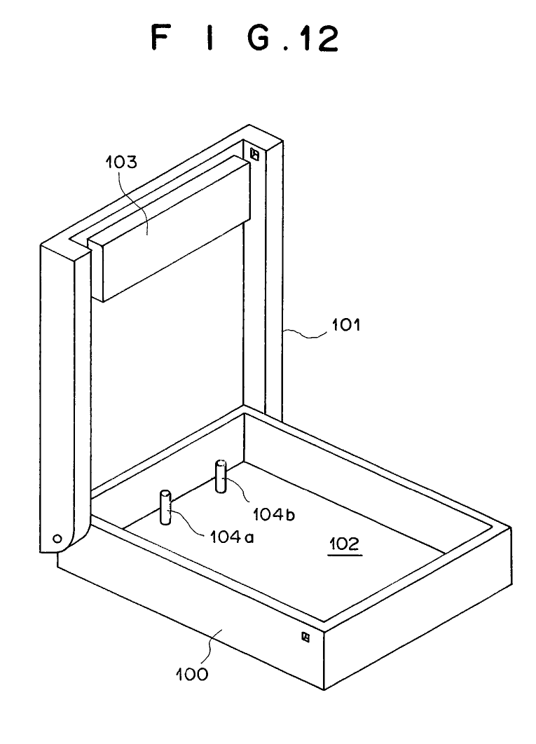

FIG. 12 is a schematic perspective view showing an exposure apparatus for executing an exposure operation, wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . of the biochemical analysis unit 1.

As illustrated in FIG. 12, the exposure apparatus for the stimulable phosphor sheet 90 comprises a casing 100 and a cover member 101. A base 102, on which the biochemical analysis unit 1 and the stimulable phosphor sheet 90 are to be placed, is located in the casing 100. the casing 100 and the cover member 101 are made from a metal, which has the radiation attenuating properties.

As illustrated in FIG. 12, the cover member 101 of the exposure apparatus is provided with a data read-out and recording section 103. The data read-out and recording section 103 comprises a read-out head (not shown) and a magnetic recording head (not shown).

As illustrated in FIG. 12, two position matching pins 104a and 104b protrude from the base 102 of the exposure apparatus.

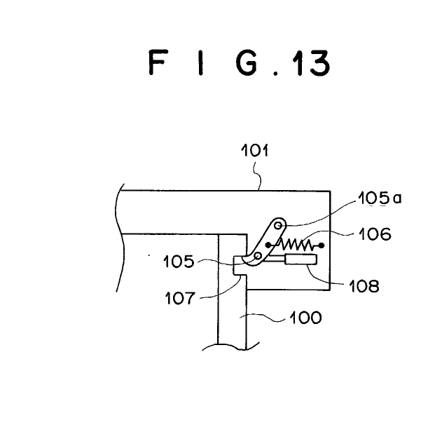

FIG. 13 is a partial schematic sectional view showing a mechanism for locking the cover member of the exposure apparatus for the stimulable phosphor sheet to the casing.

As illustrated in FIG. 13, the cover member locking mechanism for locking the cover member 101 to the casing 100 is located at each of two side regions of the cover member 101. One of the cover member locking mechanisms is shown in FIG. 13.

As illustrated in FIG. 13, the locking mechanism for the cover member 101 comprises a hook member 105, which is located within the side region of the cover member 101, and a compression spring 106, which urges the hook member 105 to the clockwise direction in FIG. 13 around a shaft 105a. The locking mechanism also comprises an engagement groove 107, which is located on a side plate of the casing 100.

The hook member 105 is constituted such that, when the cover member 101 is closed, the hook member 105 is urged by the spring force of the compression spring 106 to engage with the engagement groove 107 of the casing 100.

The cover member locking mechanism further comprises a solenoid 108, which is located within the side region of the cover member 101. The solenoid 108 swings the hook member 105 in the counter-clockwise direction in FIG. 13 around the shaft 105a against the urging force of the compression spring 106 and thereby releases the engagement of the hook member 105 with the engagement groove 107.

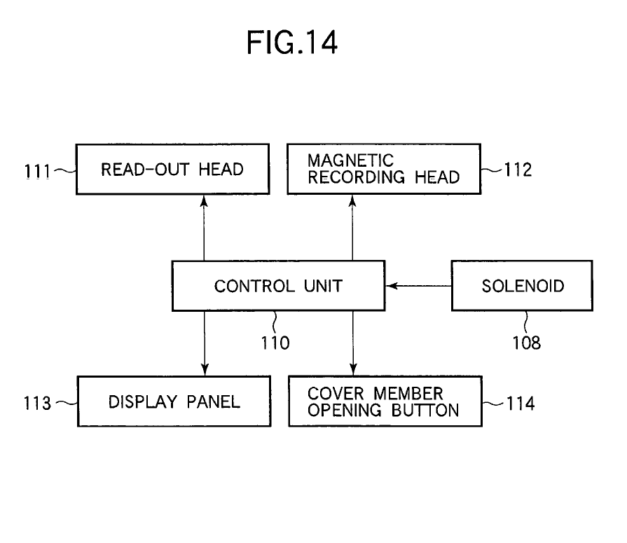

FIG. 14 is a block diagram showing a control system, a detecting system, a driving system, a displaying system, and an input system of the exposure apparatus for the stimulable phosphor sheet.

As illustrated in FIG. 14, the control system of the exposure apparatus for the stimulable phosphor sheet comprises a control unit 110 for controlling the entire exposure apparatus. The detecting system of the exposure apparatus for the stimulable phosphor sheet comprises a read-out head 111 of the data read-out and recording section 103. The read-out head 111 reads out the data having been recorded on the magnetic recording layer 5 of the biochemical analysis unit 1 and the magnetic recording layer 94 of the stimulable phosphor sheet 90. A read-out signal obtained from the read-out head 111 is fed into the control unit 110.

Also, as illustrated in FIG. 14, the driving system of the exposure apparatus for the stimulable phosphor sheet comprises a magnetic recording head 112 of the data read-out and recording section 103. The magnetic recording head 112 writes the data on the magnetic recording layer 94 of the stimulable phosphor sheet 90. The driving system also comprises the solenoid 108 for releasing the cover member locking mechanism. The displaying system of the exposure apparatus for the stimulable phosphor sheet comprises a display panel 113 constituted of the liquid crystal panel, or the like.

The input system of the exposure apparatus for the stimulable phosphor sheet comprises a cover member opening button 114.

When at least one dot-shaped stimulable phosphor layer region 92 among the plurality of the dot-shaped stimulable phosphor layer regions 92, 92, . . . of the stimulable phosphor sheet 90 is to be exposed to the radiation radiated out from the radioactive labeling substance, which is contained selectively in at least one dot-shaped adsorptive region 4 among the plurality of the dot-shaped adsorptive regions 4, 4, . . . of the biochemical analysis unit 1, the cover member opening button 114 is firstly operated.

When the cover member opening button 114 is operated, a cover member opening signal is fed into the control unit 110. The control unit 110 receives the cover member opening signal and feeds a driving signal into the solenoid 108.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

When the cover member 101 is opened, the biochemical analysis unit 1 is set on the base 102 of the exposure apparatus by the user, such that the two position matching through-holes 6a and 6b of the base plate 2 of the biochemical analysis unit 1 may fit respectively onto the two position matching pins 104a and 104b of the base 102 of the exposure apparatus. Thereafter, the cover member 101 is closed.

The data read-out and recording section 103 is secured to the cover member 101 such that, when the biochemical analysis unit 1 has been set on the base 102 and the cover member 101 has been closed, the data read-out and recording section 103 stands facing the magnetic recording layer 5 of the base plate 2 of the biochemical analysis unit 1. Firstly, the data having been recorded on the magnetic recording layer 5 of the base plate 2 of the biochemical analysis unit 1 is read out by the read-out head 111 of the data read-out and recording section 103. The thus obtained detection signal is fed into the control unit 110. In cases where the biochemical analysis unit 1 and the specific stimulable phosphor sheet 90 have been supplied as one biochemical analysis kit to the user, the identification data for specifying the stimulable phosphor sheet 90 has been recorded on the magnetic recording layer 5 of the biochemical analysis unit 1. The identification data having been read out by the read-out head 111 of the data read-out and recording section 103 is fed into the control unit 110.

The data having been fed into the control unit 110 are stored in a memory (not shown).

Thereafter, the cover member opening button 114 is operated, and the cover member 101 is opened. Also, the stimulable phosphor sheet 90 is set on the surface of the biochemical analysis unit 1 having been set on the base 102, such that the two position matching through-holes 96a and 96b of the support 91 of the stimulable phosphor sheet 90 may respectively fit onto the two position matching pins 104a and 104b of the base 102 of the exposure apparatus. Thereafter, the cover member 101 is closed.

The magnetic recording layer 94 is formed at the position on the support 91 of the stimulable phosphor sheet 90, which position corresponds to the position of the magnetic recording layer 5 of the biochemical analysis unit 1. Therefore, the read-out head 111 is capable of reading out the data having been recorded on the magnetic recording layer 94 of the stimulable phosphor sheet 90. The data having been recorded on the magnetic recording layer 94 of the stimulable phosphor sheet 90 is read out by the read-out head 111 of the data read-out and recording section 103. The thus obtained detection signal is fed into the control unit 110.

In cases where the stimulable phosphor sheet 90 is used repeatedly for the operation for exposing the stimulable phosphor layer regions 92, 92, . . . of the stimulable phosphor sheet 90 to the radiation radiated out from the radioactive labeling substance, irradiating the stimulating rays to the stimulable phosphor contained in the stimulable phosphor layer regions 92, 92, . . . , detecting the light emitted by the stimulable phosphor, and forming the data for a biochemical analysis, the accuracy of the analysis becomes markedly low, and reliable results of analysis cannot be obtained. Therefore, when the stimulable phosphor sheet 90 has been set in the exposure apparatus and exposed to the radiation radiated out from the radioactive labeling substance contained in at least one adsorptive region 4 among the plurality of the adsorptive regions 4, 4, . . . of the biochemical analysis unit 1, information representing the number of times of use for the exposure operation is recorded on the magnetic recording layer 94 of the stimulable phosphor sheet 90. The information representing the number of times of use for the exposure operation, which information has been recorded on the magnetic recording layer 94, is read out by the read-out head 111 of the data read-out and recording section 103 and fed into the control unit 110.

In cases where the stimulable phosphor sheet 90 and the specific biochemical analysis unit 1 have been supplied as one biochemical analysis kit to the user, the identification data for specifying the biochemical analysis unit 1 has been recorded on the magnetic recording layer 94 of the stimulable phosphor sheet 90. The identification data having been read out by the read-out head 111 of the data read-out and recording section 103 is fed into the control unit 110.

In cases where the control unit 110 receives the identification data of the biochemical analysis unit 1 and the identification data of the stimulable phosphor sheet 90 from the read-out head 111 of the data read-out and recording section 103, the control unit 110 makes a judgment in accordance with the received identification data as to whether the biochemical analysis unit 1 and the stimulable phosphor sheet 90 having been set in the exposure apparatus are or are not the ones which are allowed to be used together. In cases where it has been judged that the biochemical analysis unit 1 and the stimulable phosphor sheet 90 having been set in the exposure apparatus are not the ones which are allowed to be used together, the control unit 110 feeds an actuating signal to the solenoid 108 and feeds a displaying signal to the display panel 113.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

At the same time, the display panel 113 receives the displaying signal from the control unit 110 and displays a message indicating that the biochemical analysis unit 1 and the stimulable phosphor sheet 90 are not the ones which are allowed to be used together.

Also, in cases where the same stimulable phosphor sheet 90 is set repeatedly in the exposure apparatus by the user, the cover member 101 is opened.

Therefore, in cases where the user sets by mistake the biochemical analysis unit 1 and the stimulable phosphor sheet 90, which are not allowed to be used together, in the exposure apparatus, the cover member 101 cannot be closed. Accordingly, in such cases, the exposure operation for exposing the stimulable phosphor layer regions 92, 92, . . . of the stimulable phosphor sheet 90 to the radiation radiated out from the radioactive labeling substance contained selectively in the adsorptive regions 4, 4, . . . of the biochemical analysis unit 1 cannot be performed.

Further, in cases where it has been judged, in accordance with the data having been received from the read-out head 111 of the data read-out and recording section 103, that the stimulable phosphor layer regions 92, 92, . . . of the stimulable phosphor sheet 90 have already been subjected to the exposure operation M number of times, e.g. two times, the control unit 110 feeds the actuating signal to the solenoid 108 and feeds the displaying signal to the display panel 113.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

At the same time, the display panel 113 receives the displaying signal from the control unit 110 and displays a message indicating that the stimulable phosphor sheet 90 is the one which is not allowed to be used.

If the stimulable phosphor layer regions 92, 92, . . . of the stimulable phosphor sheet 90 are subjected to the exposure operation M number of times or more, and the data for a biochemical analysis is thereby formed, the accuracy of analysis will become markedly low, and reliable results of analysis cannot be obtained. The value of M is set to be, for example, 2.

Also, in cases where the same stimulable phosphor sheet 90 is set repeatedly in the exposure apparatus by the user, the cover member 101 is opened.

Therefore, in cases where the user sets by mistake the stimulable phosphor sheet 90, which has been subjected to the exposure operation M number of times and has thus been used for the formation of the data for a biochemical analysis, in the exposure apparatus, the cover member 101 cannot be closed. Accordingly, in such cases, the exposure operation for exposing the stimulable phosphor layer regions 92, 92, . . . of the stimulable phosphor sheet 90 to the radiation radiated out from the radioactive labeling substance contained selectively in the adsorptive regions 4, 4, . . . of the biochemical analysis unit 1 cannot be performed.

In cases where it has been judged that the identification data having been recorded on the magnetic recording layer 5 of the biochemical analysis unit 1 and the identification data having been recorded on the magnetic recording layer 94 of the stimulable phosphor sheet 90 have the correspondence relationship with each other, and that the number of times of use for the exposure operation of the stimulable phosphor sheet 90 is smaller than M, or in cases where the identification data has not been recorded on the magnetic recording layer 5 of the biochemical analysis unit 1 and the magnetic recording layer 94 of the stimulable phosphor sheet 90, and that the number of times of use for the exposure operation of the stimulable phosphor sheet 90 is smaller than M, the control unit 110 feeds the writing signal into the magnetic recording head 112 of the data read-out and recording section 103. In this manner, the number of times of use for the exposure operation of the stimulable phosphor sheet 90, which number of times has been recorded on the magnetic recording layer 94 of the stimulable phosphor sheet 90, is increased by 1, and the data concerning the date and hour of execution of the exposure operation is written on the magnetic recording layer 94 in accordance with a built-in clock.

Also, the control unit 110 reads out the data, which concerns the amount of the adsorptive material contained in each of the adsorptive regions 4, 4, . . . of the biochemical analysis unit 1, from among the pieces of data having been read out from the magnetic recording layer 5 of the biochemical analysis unit 1 and having been stored in the memory (not shown). The thus read-out data is fed into the magnetic recording head 112 of the data read-out and recording section 103 and written on the magnetic recording layer 94 of the stimulable phosphor sheet 90.

In such cases, since the cover member 101 of the exposure apparatus is locked, at least one dot-shaped stimulable phosphor layer region 92 among the plurality of the dot-shaped stimulable phosphor layer regions 92, 92, . . . of the stimulable phosphor sheet 90 is exposed to the radiation radiated out from the radioactive labeling substance, which is contained selectively in at least one dot-shaped adsorptive region 4 among the plurality of the dot-shaped adsorptive regions 4, 4, . . . of the biochemical analysis unit 1.

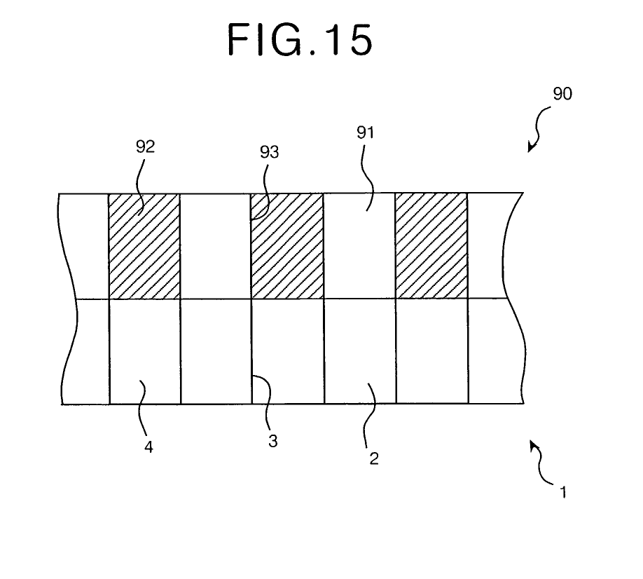

FIG. 15 is a schematic sectional view showing how at least one dot-shaped stimulable phosphor layer region 92 among the plurality of the dot-shaped stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . of the biochemical analysis unit 1.

In the exposure apparatus, the stimulable phosphor sheet 90 is superposed upon the surface of the biochemical analysis unit 1. As a result, at least one dot-shaped stimulable phosphor layer region 92 among the plurality of the dot-shaped stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . of the biochemical analysis unit 1. In this embodiment, the biochemical analysis unit 1 has been formed by filling a 6-nylon in each of the through-holes 3, 3, . . . of the base plate 2 made from aluminum. Therefore, in cases where the biochemical analysis unit 1 is subjected to treatment with the liquid for the hybridization, or the like, the biochemical analysis unit 1 suffers from little expansion or shrinkage. Accordingly, the stimulable phosphor sheet 90 is capable of being superposed upon the biochemical analysis unit 1, such that each of the adsorptive regions 4, 4, . . . of the biochemical analysis unit 1 accurately stands facing the corresponding dot-shaped stimulable phosphor layer region 92 among the dot-shaped stimulable phosphor layer regions 92, 92, . . . of the stimulable phosphor sheet 90. In this manner, the exposure operation for the dot-shaped stimulable phosphor layer regions 92, 92, . . . is capable of being executed.

In the manner described above, the biochemical analysis unit 1 and the stimulable phosphor sheet 90 are superposed one upon the other for a predetermined length of time, such that each of the adsorptive regions 4, 4, . . . of the biochemical analysis unit 1 stands facing the corresponding dot-shaped stimulable phosphor layer region 92 among the dot-shaped stimulable phosphor layer regions 92, 92, . . . of the stimulable phosphor sheet 90. As a result, at least one dot-shaped stimulable phosphor layer region 92 among the plurality of the dot-shaped stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . of the biochemical analysis unit 1.

At this time, electron rays (beta rays) are radiated out from the radioactive labeling substance having been adsorbed to at least one adsorptive region 4 among the plurality of the adsorptive regions 4, 4, . . . . However, the adsorptive regions 4, 4, . . . of the biochemical analysis unit 1 have been formed as the dot-shaped regions spaced from one another in the base plate 2, which is made from aluminum having the radiation attenuating properties. Therefore, the problems are capable of being efficiently prevented from occurring in that the electron rays (the beta rays), which have been radiated out from the radioactive labeling substance contained in one of the adsorptive regions 4, 4, . . . , are scattered within the base plate 2 of the biochemical analysis unit 1, mix with the electron rays (the beta rays) having been radiated out from an adjacent adsorptive region 4, and impinge upon the dot-shaped stimulable phosphor layer region 92, which stands facing the adjacent adsorptive region 4. Also, the dot-shaped stimulable phosphor layer regions 92, 92, . . . of the stimulable phosphor sheet 90 have been formed by embedding the stimulable phosphor in each of the through-holes 93, 93, . . . , which have been formed in the support 91 made from nickel having the radiation attenuating properties. Therefore, the problems are capable of being efficiently prevented from occurring in that the electron rays (the beta rays), which have been radiated out from the radioactive labeling substance contained in one of the adsorptive regions 4, 4, . . . , are scattered within the support 91 of the stimulable phosphor sheet 90 and impinge upon a stimulable phosphor layer region 92 adjacent to the stimulable phosphor layer region 92, which stands facing the adsorptive region 4. Accordingly, the electron rays (the beta rays), which have been radiated out from the radioactive labeling substance contained in the adsorptive region 4, are capable of selectively impinging upon the stimulable phosphor layer region 92, which stands facing the adsorptive region 4, and the problems are capable of being reliably prevented from occurring in that the electron rays (the beta rays), which have been radiated out from the radioactive labeling substance contained in the adsorptive region 4, impinge upon a stimulable phosphor layer region 92, which is to be exposed to the electron rays radiated out from an adjacent adsorptive region 4.

In the manner described above, the radiation information with the radioactive labeling substance is recorded on at least one dot-shaped stimulable phosphor layer region 92 among the plurality of the dot-shaped stimulable phosphor layer regions 92, 92, . . . of the stimulable phosphor sheet 90.

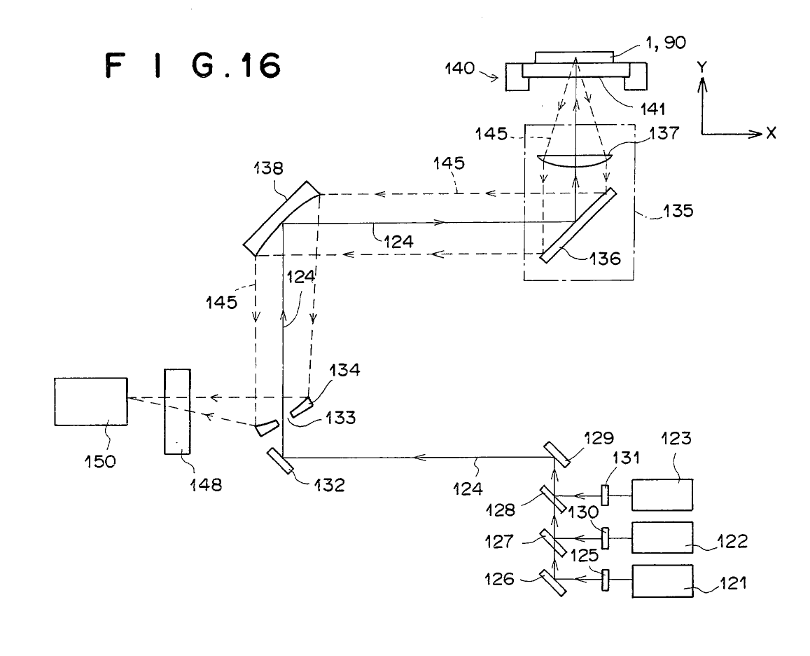

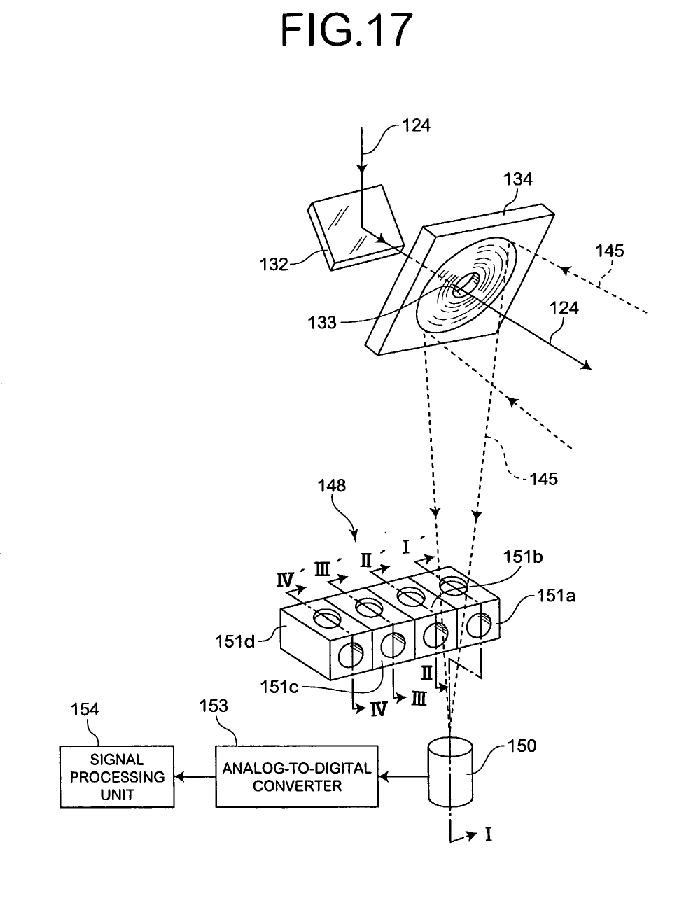

FIG. 16 is a schematic perspective view showing a scanner for reading out the radiation information from the stimulable phosphor sheet 90 in order to form the data for a biochemical analysis, and reading out the fluorescence information from the biochemical analysis unit 1 in order to form the data for a biochemical analysis. FIG. 17 is a schematic perspective view showing a constitution in the vicinity of a photomultiplier in the scanner of FIG. 16.

In this embodiment, the scanner is capable of reading out the radiation information formed with the radioactive labeling substance, which information has been recorded on the dot-shaped stimulable phosphor layer regions 92, 92, . . . of the stimulable phosphor sheet 90, and reading out the fluorescence information formed with the fluoro chrome, or the like, which information has been recorded on the adsorptive regions 4, 4, . . . of the biochemical analysis unit 1.

As illustrated in FIG. 16, the scanner comprises a first laser beam source 121 for producing a laser beam 124 having a wavelength of 640 nm. The scanner also comprises a second laser beam source 122 for producing a laser beam 124 having a wavelength of 532 nm. The scanner further comprises a third laser beam source 123 for producing a laser beam 124 having a wavelength of 473 nm.

In this embodiment, the first laser beam source 121 is constituted of a semiconductor laser beam source. Each of the second laser beam source 122 and the third laser beam source 123 is constituted of a second harmonic generating device.

The laser beam 124 having been produced by the first laser beam source 121 is collimated by a collimator lens 125, and the collimated laser beam 124 is reflected by a mirror 126. A first dichroic mirror 127 and a second dichroic mirror 128 are located in the optical path of the laser beam 124 having been reflected by the mirror 126. The first dichroic mirror 127 transmits only the laser beam 124 having a wavelength of 640 nm and reflects the light having a wavelength of 532 nm. The second dichroic mirror 128 transmits only the light having a wavelength of at least 532 nm and reflects the light having a wavelength of 473 nm. The laser beam 124 having been produced by the first laser beam source 121 passes through the first dichroic mirror 127 and the second dichroic mirror 128 and impinges upon a mirror 129.

The laser beam 124 having been produced by the second laser beam source 122 is collimated by a collimator lens 130, and the collimated laser beam 124 is reflected by the first dichroic mirror 127. The direction of the optical path of the laser beam 124 is thus changed by an angle of 90°. The laser beam 124 then passes through the second dichroic mirror 128 and impinges upon the mirror 129.

The laser beam 124 having been produced by the third laser beam source 123 is collimated by a collimator lens 131, and the collimated laser beam 124 is reflected by the second dichroic mirror 128. The direction of the optical path of the laser beam 124 is thus changed by an angle of 90°. The laser beam 124 then impinges upon the mirror 129.

The laser beam 124 impinging upon the mirror 129 is reflected by the mirror 129. The laser beam 124 then impinges upon a mirror 132 and is reflected by the mirror 132.

A perforated mirror 134 is located in the optical path of the laser beam 124, which has been reflected by the mirror 132. The perforated mirror 134 is constituted of a concave mirror having a hole 133 at a center area. The laser beam 124, which has been reflected by the mirror 132, passes through the hole 133 of the perforated mirror 134 and impinges upon a concave mirror 138.

The laser beam 124 impinging upon the concave mirror 138 is reflected by the concave mirror 138 and impinges upon an optical head 135.

The optical head 135 comprises a mirror 136 and an aspherical lens 137. The laser beam 124 impinging upon the optical head 135 is reflected by the mirror 136 and impinges upon the aspherical lens 137. The laser beam 124 is caused by the aspherical lens 137 to impinge upon the stimulable phosphor sheet 90 or the biochemical analysis unit 1, which is placed on a glass plate 141 of a stage 140.

In cases where the laser beam 124 thus impinges upon the stimulable phosphor sheet 90, the at least one dot-shaped stimulable phosphor layer region 92 of the support 91 of the stimulable phosphor sheet 90, which region has been exposed to the radiation radiated out from the radioactive labeling substance, is stimulated by the laser beam 124 to emit light 145 in proportion to the amount of energy stored on the region during exposure of the region to the radiation. In cases where the laser beam 124 impinges upon the biochemical analysis unit 1, the fluorescent labeling substance, such as the fluoro chrome, which is contained in the at least one adsorptive region 4, is excited by the laser beam 124 to produce fluorescence 145.

The light 145, which has been emitted by the at least one dot-shaped stimulable phosphor layer region 92 of the support 91 of the stimulable phosphor sheet 90, or the fluorescence 145, which has been produced by the fluorescent labeling substance contained in the at least one adsorptive region 4 of the biochemical analysis unit 1, is converged by the aspherical lens 137 of the optical head 135 onto the mirror 136. The emitted light 145 or the fluorescence 145 is reflected by the mirror 136 so as to follow reversely the same optical path as the optical path of the laser beam 124 and impinges as a collimated beam upon the concave mirror 138.

The emitted light 145 or the fluorescence 145 impinging upon the concave mirror 138 is reflected by the concave mirror 138 and impinges upon the perforated mirror 134.

As illustrated in FIG. 17, the emitted light 145 or the fluorescence 145 impinging upon the perforated mirror 134 is reflected downwardly by the perforated mirror 134 constituted of the concave mirror and impinges upon a filter unit 148. The filter unit 148 filters out light having predetermined wavelengths. The emitted light 145 or the fluorescence 145 having passed through the filter unit 148 impinges upon a photomultiplier 150 and is photoelectrically detected by the photomultiplier 150.

As illustrated in FIG. 17, the filter unit 148 comprises four filter members 151a, 151b, 151c, and 151d. The filter unit 148 is capable of being moved by a motor (not shown) horizontally in FIG. 17.

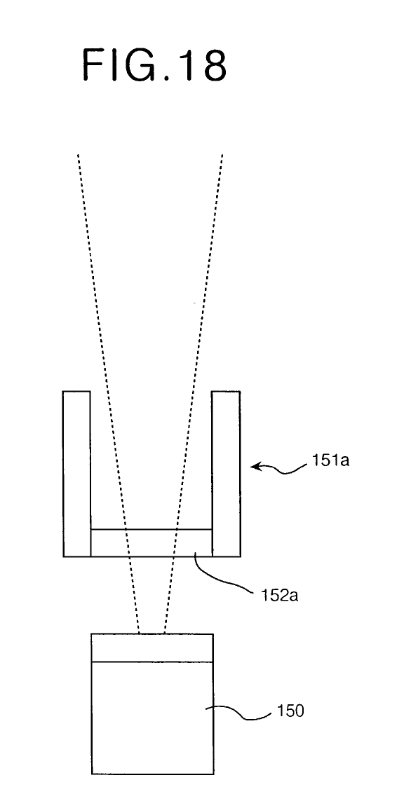

FIG. 18 is a schematic sectional view taken on line I′-I of FIG. 17.

As illustrated in FIG. 18, the filter member 151a comprises a filter 152a. The filter 152a is utilized in cases where the fluorescent labeling substance contained in the at least one adsorptive region 4 of the biochemical analysis unit 1 is excited by the laser beam 124 produced by the first laser beam source 121, and the thus produced fluorescence 145 is detected. The filter 152a has the properties for filtering out light having a wavelength of 640 nm and transmitting only light having wavelengths longer than 640 nm.

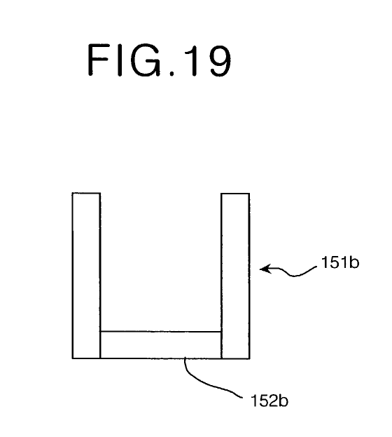

FIG. 19 is a schematic sectional view taken on line II′-II of FIG. 17.

As illustrated in FIG. 19, the filter member 151b comprises a filter 152b. The filter 152b is utilized in cases where the fluorescent labeling substance contained in the at least one adsorptive region 4 of the biochemical analysis unit 1 is excited by the laser beam 124 produced by the second laser beam source 122, and the thus produced fluorescence 145 is detected. The filter 152b has the properties for filtering out light having a wavelength of 532 nm and transmitting only light having wavelengths longer than 532 nm.

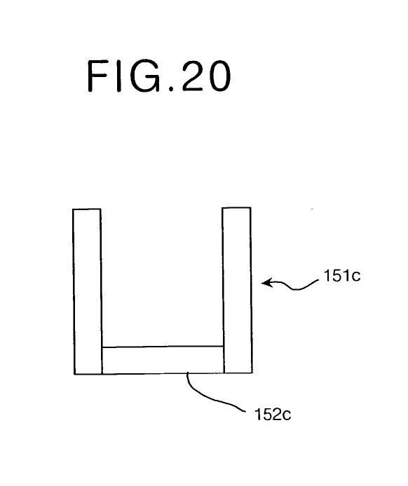

FIG. 20 is a schematic sectional view taken on line III′-III of FIG. 17.

As illustrated in FIG. 20, the filter member 151c comprises a filter 152c. The filter 152c is utilized in cases where the fluorescent labeling substance contained in the at least one adsorptive region 4 of the biochemical analysis unit 1 is excited by the laser beam 124 produced by the third laser beam source 123, and the thus produced fluorescence 145 is detected. The filter 152c has the properties for filtering out light having a wavelength of 473 nm and transmitting only light having wavelengths longer than 473 nm.

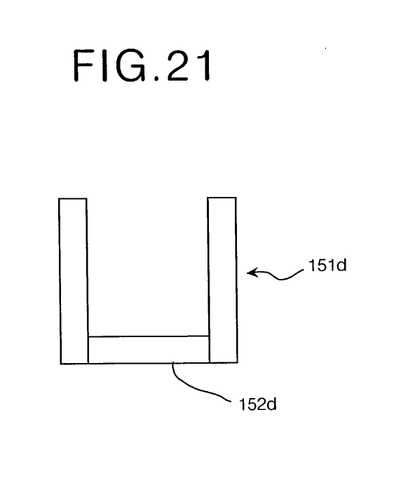

FIG. 21 is a schematic sectional view taken on line IV′-IV of FIG. 17.

As illustrated in FIG. 21, the filter member 151d comprises a filter 152d. The filter 152d is utilized in cases where the at least one dot-shaped stimulable phosphor layer region 92 of the stimulable phosphor sheet 90 is stimulated by the laser beam 124 produced by the first laser beam source 121, and the light 145 emitted by the dot-shaped stimulable phosphor layer region 92 is detected. The filter 152d has the properties for filtering out light having a wavelength of 640 nm and transmitting only light having wavelengths falling within the wavelength range of the light 145 emitted by the dot-shaped stimulable phosphor layer region 92.

The filter member 151a, 151b, 151c, or 151d is selectively located in front of the photomultiplier 150 in accordance with the laser beam source to be used. In this manner, the photomultiplier 150 is capable of photoelectrically detecting only the light which is to be detected.

The emitted light 145 is photoelectrically detected by the photomultiplier 150, and an analog signal is thereby obtained. The analog signal is fed into an analog-to-digital converter 153 and is converted into a digital signal. The digital signal is fed into a signal processing unit 154.

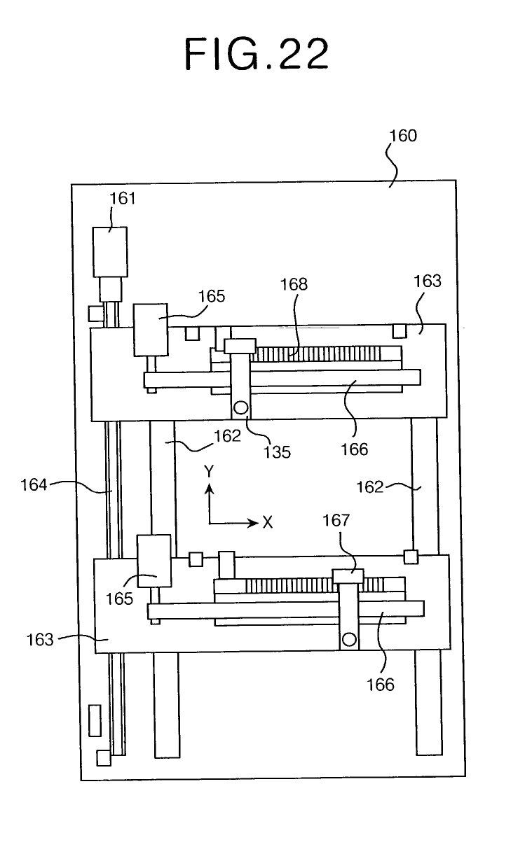

FIG. 22 is a schematic plan view showing a scanning mechanism for the optical head 135. In FIG. 22, as an aid in facilitating the explanation, the optical system other than the optical head 135, the optical path of the laser beam 124, and the optical path of the emitted light 145 or the fluorescence 145 are not shown.

As illustrated in FIG. 22, the scanning mechanism for scanning the optical head 135 comprises a base 160. A sub-scanning pulse motor 161 and a pair of rails 162, 162 are secured to the base 160. Also, a base 163, which is capable of moving in the sub-scanning direction indicated by the arrow Y in FIG. 22, is located on the base 160.

The movable base 163 has a threaded hole (not shown). A threaded rod 164, which is rotated by the sub-scanning pulse motor 161, is engaged with the threaded hole of the base 163.

A main scanning stepping motor 165 is secured to the movable base 163. The main scanning stepping motor 165 is capable of intermittently move an endless belt 166 at a pitch equal to the distance between two adjacent dot-shaped adsorptive regions 4, 4, of the biochemical analysis unit 1, i.e. at a pitch equal to the distance between two adjacent dot-shaped stimulable phosphor layer regions 92, 92 of the stimulable phosphor sheet 90. The optical head 135 is secured to the endless belt 166. When the endless belt 166 is moved by the main scanning stepping motor 165, the optical head 135 is moved by the endless belt 166 in the main scanning direction indicated by the arrow X in FIG. 22. In FIG. 22, reference numeral 167 represents a linear encoder for detecting the position of the optical head 135 with respect to the main scanning direction. Also, reference numeral 168 represents slits of the linear encoder 167.

The endless belt 166 is intermittently moved by the main scanning stepping motor 165 in the main scanning direction, and the scanning along one main scanning line is completed. Thereafter, the movable base 163 is intermittently moved by the sub-scanning pulse motor 161 in the sub-scanning direction. In this manner, the optical head 135 is moved in the main scanning direction indicated by the arrow X in FIG. 22 and in the sub-scanning direction indicated by the arrow Y in FIG. 22. As a result, all of the dot-shaped stimulable phosphor layer regions 92, 92, . . . of the stimulable phosphor sheet 90 or the entire surface of the biochemical analysis unit 1 is scanned with the laser beam 124.

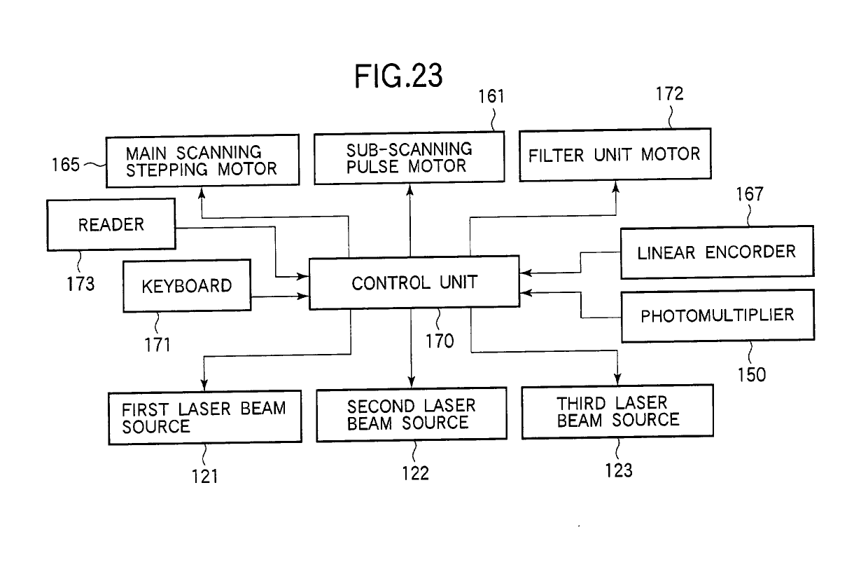

FIG. 23 is a block diagram showing a control system, an input system, a driving system, and a detecting system of the scanner.

As illustrated in FIG. 23, the control system of the scanner comprises a control unit 170. The input system of the scanner comprises a keyboard 171, which is operated by the user and is capable of inputting various instruction signals.

Also, as illustrated in FIG. 23, the driving system of the scanner comprises the main scanning stepping motor 165 for intermittently moving the optical head 135 in the main scanning direction. The driving system also comprises the sub-scanning pulse motor 161 for intermittently moving the optical head 135 in the sub-scanning direction. The driving system further comprises a filter unit motor 172 for moving the filter unit 148, which comprises the four filter members 151a, 151b, 151c, and 151d.

The control unit 170 is capable of feeding an actuating signal selectively into the first laser beam source 121, the second laser beam source 122, or the third laser beam source 123. Also, the control unit 170 is capable of feeding a driving signal into the filter unit motor 172.

Further, as illustrated in FIG. 23, the detecting system of the scanner comprises the photomultiplier 150, and the linear encoder 167 for detecting the position of the optical head 135 with respect to the main scanning direction. The detecting system also comprises a reader 173 for reading out the data having been recorded on the magnetic recording layer 5 of the biochemical analysis unit 1 and the magnetic recording layer 94 of the stimulable phosphor sheet 90.

In this embodiment, in accordance with a position detection signal, which represents the position of the optical head 135 and is received from the linear encoder 167, the control unit 170 performs on-off control of the first laser beam source 121, the second laser beam source 122, or the third laser beam source 123.

The scanner constituted in the manner described above reads out the radiation information, which has been recorded on the at least one dot-shaped stimulable phosphor layer region 92 of the stimulable phosphor sheet 90, in the manner described below and forms the data for a biochemical analysis.

Firstly, the stimulable phosphor sheet 90 is placed on the glass plate 141 of the stage 140.

Thereafter, an instruction signal for the scanning of the dot-shaped stimulable phosphor layer regions 92, 92, . . . of the stimulable phosphor sheet 90 with the laser beam 124 is inputted by the user from the keyboard 171.

The instruction signal having been inputted from the keyboard 171 is fed into the control unit 170. In accordance with the instruction signal, the control unit 170 feeds the driving signal into the filter unit motor 172 in order to move the filter unit 148. In this manner, the filter member 151d comprising the filter 152d, which has the properties for filtering out light having a wavelength of 640 nm and transmitting only light having wavelengths falling within the wavelength range of the light 145 emitted by the stimulable phosphor, is located in the optical path of the emitted light 145.

Also, when the stimulable phosphor sheet 90 is placed on the glass plate 141 of the stage 140, the reader 173, which is located above the stage 140, reads out the data having been recorded on the magnetic recording layer 94 of the stimulable phosphor sheet 90, which data concerns the amount of the adsorptive material contained in each of the adsorptive regions 4, 4, . . . of the biochemical analysis unit 1. The read-out data is fed from the reader 173 into the control unit 170. The control unit 170 stores the received data in a memory (not shown).

Further, the control unit 170 feeds the driving signal into the main scanning stepping motor 165 in order to move the optical head 135 in the main scanning direction. When it has been confirmed, in accordance with the position detection signal with respect to the optical head 135, which signal is received from the linear encoder 167, that the optical head 135 has moved to the position which is capable of irradiating the laser beam 124 to a first dot-shaped stimulable phosphor layer region 92, the control unit 170 feeds a stop signal into the main scanning stepping motor 165. Also, at this time, the control unit 170 feeds the actuating signal into the first laser beam source 121 in order to actuate the first laser beam source 121 to produce the laser beam 124 having a wavelength of 640 nm.

The laser beam 124 having been produced by the first laser beam source 121 is collimated by the collimator lens 125. The collimated laser beam 124 impinges upon the mirror 126 and is reflected by the mirror 126.

The laser beam 124 having been reflected by the mirror 126 passes through the first dichroic mirror 127 and the second dichroic mirror 128 and impinges upon the mirror 129.

The laser beam 124 impinging upon the mirror 129 is reflected by the mirror 129. The laser beam 124 then impinges upon the mirror 132 and is reflected by the mirror 132.

The laser beam 124 having been reflected by the mirror 132 passes through the hole 133 of the perforated mirror 134 and impinges upon the concave mirror 138.

The laser beam 124 impinging upon the concave mirror 138 is reflected by the concave mirror 138 and impinges upon the optical head 135.

The laser beam 124 impinging upon the optical head 135 is reflected by the mirror 136 and converged by the aspherical lens 137 onto the first dot-shaped stimulable phosphor layer region 92 of the stimulable phosphor sheet 90, which is placed on the glass plate 141 of the stage 140.

As a result, the stimulable phosphor contained in the first dot-shaped stimulable phosphor layer region 92 of the stimulable phosphor sheet 90 is stimulated by the laser beam 124 to emit the light 145.

As described above, the support 91 of the stimulable phosphor sheet 90 is made from nickel. Therefore, at this time, the problems are capable of being efficiently prevented from occurring in that the laser beam 124 is scattered within the support 91 and stimulates the stimulable phosphor, which is contained in a dot-shaped stimulable phosphor layer region 92 adjacent to the first dot-shaped stimulable phosphor layer region 92, to emit the light 145. Also, the problems are capable of being efficiently prevented from occurring in that the light 145 emitted by the first dot-shaped stimulable phosphor layer region 92 is scattered within the support 91 and cannot be detected by the photomultiplier 150.

The light 145 emitted by the first dot-shaped stimulable phosphor layer region 92 is converged by the aspherical lens 137 of the optical head 135. The emitted light 145 is then reflected by the mirror 136 so as to follow reversely the same optical path as the optical path of the laser beam 124. The emitted light 145 thus impinges as a collimated beam upon the concave mirror 138.

The emitted light 145 impinging upon the concave mirror 138 is reflected by the concave mirror 138 and impinges upon the perforated mirror 134. As illustrated in FIG. 17, the emitted light 145 impinging upon the perforated mirror 134 is reflected downwardly by the perforated mirror 134 constituted of the concave mirror. The emitted light 145 thus impinges upon the filter 152d of the filter unit 148.

The filter 152d has the properties for filtering out light having a wavelength of 640 nm and transmitting only light having wavelengths falling within the wavelength range of the light 145 emitted by the stimulable phosphor. Therefore, the light having a wavelength of 640 nm and acting as the stimulating rays is filtered out, and only the light having wavelengths falling within the wavelength range of the light 145 emitted by the dot-shaped stimulable phosphor layer region 92 passes through the filter 152d and is photoelectrically detected by the photomultiplier 150.

The analog signal obtained from the photomultiplier 150 is converted by the analog-to-digital converter 153 into the digital signal. The digital signal is fed into the signal processing unit 154.

When a predetermined length of time, e.g. several microseconds, has elapsed after the first laser beam source 121 was turned on, the control unit 170 feeds an actuation stop signal into the first laser beam source 121 in order to stop the actuation of the first laser beam source 121. Also, the control unit 170 feeds the driving signal into main scanning stepping motor 165 in order to move the optical head 135 by the pitch equal to the distance between two adjacent dot-shaped stimulable phosphor layer regions 92, 92 of the stimulable phosphor sheet 90.

The signal processing unit 154 receives the digital signal, which has been obtained from the photoelectric detection of the light 145 emitted by the first dot-shaped stimulable phosphor layer region 92 and the digitization of the thus obtained analog signal. Also, the signal processing unit 154 reads out the data, which concerns the amount of the adsorptive material contained in each of the adsorptive regions 4, 4, . . . of the biochemical analysis unit 1, from the memory (not shown). In accordance with the read-out data, the signal processing unit 154 corrects the digital signal and thereby forms the data for a biochemical analysis, which data corresponds to the radiation information having been recorded on the first dot-shaped stimulable phosphor layer region 92. In this manner, the problems are capable of being prevented from occurring in that noise due to variations in amount of the adsorptive material contained in the adsorptive regions 4, 4, . . . occurs in the data for a biochemical analysis.

When it has been confirmed, in accordance with the position detection signal with respect to the optical head 135, which signal is received from the linear encoder 167, that the optical head 135 has moved by one pitch equal to the distance between two adjacent dot-shaped stimulable phosphor layer regions 92, 92 of the stimulable phosphor sheet 90 and has arrived at the position which is capable of irradiating the laser beam 124 produced by the first laser beam source 121 to a second dot-shaped stimulable phosphor layer region 92 of the stimulable phosphor sheet 90, the control unit 170 feeds the actuating signal into the first laser beam source 121 and turns on the first laser beam source 121. In this manner, the stimulable phosphor contained in the second dot-shaped stimulable phosphor layer region 92 of the stimulable phosphor sheet 90 is stimulated by the laser beam 124.

In the same manner as that described above, the laser beam 124 produced by the first laser beam source 121 is irradiated for the predetermined length of time to the second dot-shaped stimulable phosphor layer region 92 of the stimulable phosphor sheet 90, and the stimulable phosphor contained in the second dot-shaped stimulable phosphor layer region 92 is stimulated to emit the light 145. The emitted light 145 is photoelectrically detected by the photomultiplier 150, and an analog signal is thereby obtained. The analog signal is digitized by the analog-to-digital converter 153. In this manner, the data for a biochemical analysis is formed from the radiation information having been recorded on the second dot-shaped stimulable phosphor layer region 92. The control unit 170 then feeds an off signal into the first laser beam source 121 in order to turn off the first laser beam source 121. Also, the control unit 170 feeds the driving signal into the main scanning stepping motor 165 in order to move the optical head 135 by one pitch equal to the distance between two adjacent dot-shaped stimulable phosphor layer regions 92, 92 of the stimulable phosphor sheet 90 .

The signal processing unit 154 receives the digital signal, which has been obtained from the photoelectric detection of the light 145 emitted by the second dot-shaped stimulable phosphor layer region 92 and the digitization of the thus obtained analog signal. Also, the signal processing unit 154 reads out the data, which concerns the amount of the adsorptive material contained in each of the adsorptive regions 4, 4, . . . of the biochemical analysis unit 1, from the memory (not shown). In accordance with the read-out data, the signal processing unit 154 corrects the digital signal and thereby forms the data for a biochemical analysis, which data corresponds to the radiation information having been recorded on the second dot-shaped stimulable phosphor layer region 92. In this manner, the problems are capable of being prevented from occurring in that noise due to variations in amount of the adsorptive material contained in the adsorptive regions 4, 4, . . . occurs in the data for a biochemical analysis.

In the manner described above, the on-off operations of the first laser beam source 121 are repeated in synchronization with the intermittent movement of the optical head 135. When it has been confirmed, in accordance with the position detection signal with respect to the optical head 135, which signal is received from the linear encoder 167, that the optical head 135 has been moved by the distance corresponding to the length of one line along the main scanning direction, and that the scanning of the dot-shaped stimulable phosphor layer regions 92, 92, . . . , which are located along the first main scanning line, with the laser beam 124 has been completed, the control unit 170 feeds the driving signal into the main scanning stepping motor 165 in order to return the optical head 135 to the original position. Also, the control unit 170 feeds the driving signal into the sub-scanning pulse motor 161 in order to move the movable base 163 in the sub-scanning direction by a distance corresponding to the width of one main scanning line.

When it has been confirmed, in accordance with the position detection signal with respect to the optical head 135, which signal is received from the linear encoder 167, that the optical head 135 has been returned to the original position, and that the movable base 163 has been moved in the sub-scanning direction by the distance corresponding to the width of one main scanning line, the control unit 170 operates in order to irradiate the laser beam 124 produced by the first laser beam source 121 successively to the dot-shaped stimulable phosphor layer regions 92, 92, . . . , which are located along the second main scanning line, in the same manner as that in the operation for irradiating the laser beam 124 produced by the first laser beam source 121 successively to the dot-shaped stimulable phosphor layer regions 92, 92, . . . , which are located along the first main scanning line. In this manner, the stimulable phosphor contained in each of the dot-shaped stimulable phosphor layer regions 92, 92, . . . is stimulated by the laser beam 124, and the light 145 emitted by each of the dot-shaped stimulable phosphor layer regions 92, 92, . . . is photoelectrically detected by the photomultiplier 150.

The analog signal obtained from the photomultiplier 150 is converted by the analog-to-digital converter 153 into the digital signal. In this manner, the data for a biochemical analysis is formed from the radiation information having been recorded on each of the dot-shaped stimulable phosphor layer regions 92, 92, . . . .

The signal processing unit 154 receives the digital signal, which has been obtained from the photoelectric detection of the light 145 emitted by each of the dot-shaped stimulable phosphor layer regions 92, 92, . . . and the digitization of the thus obtained analog signal. Also, the signal processing unit 154 reads out the data, which concerns the amount of the adsorptive material contained in each of the adsorptive regions 4, 4, . . . of the biochemical analysis unit 1, from the memory (not shown). In accordance with the read-out data, the signal processing unit 154 corrects the digital signal and thereby forms the data for a biochemical analysis, which data corresponds to the radiation information having been recorded on each of the dot-shaped stimulable phosphor layer regions 92, 92, . . . . In this manner, the problems are capable of being prevented from occurring in that noise due to variations in amount of the adsorptive material contained in the adsorptive regions 4, 4, . . . occurs in the data for a biochemical analysis.

In the manner described above, all of the dot-shaped stimulable phosphor layer regions 92, 92, . . . of the stimulable phosphor sheet 90 are scanned with the laser beam 124 having been produced by the first laser beam source 121. The stimulable phosphor contained in each of the dot-shaped stimulable phosphor layer regions 92, 92, . . . is thus stimulated by the laser beam 124, and the emitted light 145 is photoelectrically detected by the photomultiplier 150. The thus obtained analog signal is digitized by the analog-to-digital converter 153, and the data for a biochemical analysis is formed from the radiation information having been recorded on each of the dot-shaped stimulable phosphor layer regions 92, 92, . . . . The control unit 170 then feeds the actuation stop signal into the first laser beam source 121, and the actuation of the first laser beam source 121 is stopped.

In cases where the fluorescence information formed with the radioactive labeling substance, which information has been recorded on at least one adsorptive region 4 among the plurality of the adsorptive regions 4, 4, . . . of the biochemical analysis unit 1, is to be readout, and the data for a biochemical analysis is thus to be formed, the biochemical analysis unit 1 is firstly set on the glass plate 141 of the stage 140 by the user.

Thereafter, an instruction signal for specifying the kind of the fluorescent labeling substance and instructing the readout of the fluorescence information is inputted by the user from the keyboard 171.

The instruction signal having been inputted from the keyboard 171 is fed into the control unit 170. The control unit 170 receives the instruction signal, and determines the laser beam source, which is to be used, in accordance with a table stored in the memory (not shown). Also, the control unit 170 determines which filter among the filters 152a, 152b, 152c, and 152d is to be located in the optical path of the fluorescence 145.

For example, in cases where Rhodamine (trade name), which is capable of being excited most efficiently by the laser beam having a wavelength of 532 nm, is used as the fluorescent labeling substance, and information representing the use of Rhodamine is inputted from the keyboard 171, the control unit 170 selects the second laser beam source 122 and selects the filter 152b. Also, the control unit 170 feeds the driving signal into the filter unit motor 172 in order to move the filter unit 148. In this manner, the filter member 151b provided with the filter 152b, which has the properties for filtering out the light having a wavelength of 532 nm and transmitting only the light having wavelengths longer than 532 nm, is located in the optical path of the fluorescence 145, which is to be radiated out from the biochemical analysis unit 1.

Also, when the biochemical analysis unit 1 is placed on the glass plate 141 of the stage 140, the reader 173, which is located above the stage 140, reads out the data having been recorded on the magnetic recording layer 5 of the biochemical analysis unit 1, which data concerns the amount of the adsorptive material contained in each of the adsorptive regions 4, 4, . . . of the biochemical analysis unit 1. The read-out data is fed from the reader 173 into the control unit 170. The control unit 170 stores the received data in the memory (not shown).

Further, the control unit 170 feeds the driving signal into the main scanning stepping motor 165 in order to move the optical head 135 in the main scanning direction. When it has been confirmed, in accordance with the position detection signal with respect to the optical head 135, which signal is received from the linear encoder 167, that the optical head 135 has moved to the position which is capable of irradiating the laser beam 124 to a first adsorptive region 4 among the plurality of the adsorptive regions 4, 4, . . . of the biochemical analysis unit 1, the control unit 170 feeds a stop signal into the main scanning stepping motor 165. Also, at this time, the control unit 170 feeds the actuating signal into the second laser beam source 122 in order to actuate the second laser beam source 122 to produce the laser beam 124 having a wavelength of 532 nm.

The laser beam 124 having been produced by the second laser beam source 122 is collimated by the collimator lens 130. The collimated laser beam 124 impinges upon the first dichroic mirror 127 and is reflected by the first dichroic mirror 127.

The laser beam 124 having been reflected by the first dichroic mirror 127 passes through the second dichroic mirror 128 and impinges upon the mirror 129.

The laser beam 124 impinging upon the mirror 129 is reflected by the mirror 129. The laser beam 124 then impinges upon the mirror 132 and is reflected by the mirror 132.

The laser beam 124 having been reflected by the mirror 132 passes through the hole 133 of the perforated mirror 134 and impinges upon the concave mirror 138.

The laser beam 124 impinging upon the concave mirror 138 is reflected by the concave mirror 138 and impinges upon the optical head 135.

The laser beam 124 impinging upon the optical head 135 is reflected by the mirror 136 and converged by the aspherical lens 137 onto the biochemical analysis unit 1, which is placed on the glass plate 141 of the stage 140.

As a result, the fluorescent labeling substance, such as the fluoro chrome, e.g. Rhodamine, which is contained in the first adsorptive region 4 of the biochemical analysis unit 1, is excited by the laser beam 124 to produce the fluorescence.

As described above, in this embodiment, the adsorptive regions 4, 4, . . . of the biochemical analysis unit 1 have been formed by filling the adsorptive material in the plurality of the through-holes 3, 3, . . . , which have been formed at a spacing from one another in the aluminum base plate 2. The aluminum base plate 2 having the light attenuating properties is located around each of the adsorptive regions 4, 4, . . . . Therefore, the problems are capable of being reliably prevented from occurring in that the fluorescence 145, which has been produced by the fluorescent labeling substance contained in one adsorptive region 4, mixes with the fluorescence 145, which has been produced by the fluorescent labeling substance contained in an adjacent adsorptive region 4.

The fluorescence 145 produced by Rhodamine is converged by the aspherical lens 137 of the optical head 135. The fluorescence 145 is then reflected by the mirror 136 so as to follow reversely the same optical path as the optical path of the laser beam 124. The fluorescence 145 thus impinges as a collimated beam upon the concave mirror 138.

The fluorescence 145 impinging upon the concave mirror 138 is reflected by the concave mirror 138 and impinges upon the perforated mirror 134. As illustrated in FIG. 17, the fluorescence 145 impinging upon the perforated mirror 134 is reflected downwardly by the perforated mirror 134 constituted of the concave mirror. The fluorescence 145 thus impinges upon the filter 152b of the filter unit 148.

The filter 152b has the properties for filtering out light having a wavelength of 532 nm and transmitting only light having wavelengths longer than 532 nm. Therefore, the light having a wavelength of 532 nm and acting as the excitation light is filtered out, and only the light having wavelengths falling within the wavelength range of the fluorescence 145 produced by Rhodamine passes through the filter 152b and is photoelectrically detected by the photomultiplier 150.

The analog signal obtained from the photomultiplier 150 is converted by the analog-to-digital converter 153 into the digital signal. The digital signal is fed into the signal processing unit 154.

The signal processing unit 154 receives the digital signal, which has been obtained from the photoelectric detection of the fluorescence 145 radiated out from the first adsorptive region 4 and the digitization of the thus obtained analog signal. Also, the signal processing unit 154 reads out the data, which concerns the amount of the adsorptive material contained in each of the adsorptive regions 4, 4, . . . of the biochemical analysis unit 1, from the memory (not shown). In accordance with the read-out data, the signal processing unit 154 corrects the digital signal and thereby forms the data for a biochemical analysis, which data corresponds to the fluorescence information having been recorded on the first adsorptive region 4. In this manner, the problems are capable of being prevented from occurring in that noise due to variations in amount of the adsorptive material contained in the adsorptive regions 4, 4, . . . occurs in the data for a biochemical analysis.

When a predetermined length of time, e.g. several microseconds, has elapsed after the second laser beam source 122 was turned on, the control unit 170 feeds the actuation stop signal into the second laser beam source 122 in order to stop the actuation of the second laser beam source 122. Also, the control unit 170 feeds the driving signal into main scanning stepping motor 165 in order to move the optical head 135 by the pitch equal to the distance between two adjacent adsorptive regions 4, 4 of the biochemical analysis unit 1.

When it has been confirmed, in accordance with the position detection signal with respect to the optical head 135, which signal is received from the linear encoder 167, that the optical head 135 has moved by one pitch equal to the distance between two adjacent adsorptive regions 4, 4 of the biochemical analysis unit 1 and has arrived at the position which is capable of irradiating the laser beam 124 produced by the second laser beam source 122 to a second adsorptive region 4 of the biochemical analysis unit 1, the control unit 170 feeds the actuating signal into the second laser beam source 122 and turns on the second laser beam source 122. In this manner, the fluorescent labeling substance, e.g. Rhodamine, contained in the second adsorptive region 4 of the biochemical analysis unit 1 is excited by the laser beam 124.

In the same manner as that described above, the laser beam 124 produced by the second laser beam source 122 is irradiated for the predetermined length of time to the second adsorptive region 4 of the biochemical analysis unit 1. The fluorescence 145 radiated out from the second adsorptive region 4 is photoelectrically detected by the photomultiplier 150, and an analog signal is thereby obtained. The control unit 170 then feeds the off signal into the second laser beam source 122 in order to turn off the second laser beam source 122. Also, the control unit 170 feeds the driving signal into the main scanning stepping motor 165 in order to move the optical head 135 by one pitch equal to the distance between two adjacent adsorptive regions 4, 4 of the biochemical analysis unit 1.

In the manner described above, the on-off operations of the second laser beam source 122 are repeated in synchronization with the intermittent movement of the optical head 135. When it has been confirmed, in accordance with the position detection signal with respect to the optical head 135, which signal is received from the linear encoder 167, that the optical head 135 has been moved by the distance corresponding to the length of one line along the main scanning direction, and that the scanning of all of the adsorptive regions 4, 4, . . . , which are located along the first main scanning line on the biochemical analysis unit 1, with the laser beam 124 has been completed, the control unit 170 feeds the driving signal into the main scanning stepping motor 165 in order to return the optical head 135 to the original position. Also, the control unit 170 feeds the driving signal into the sub-scanning pulse motor 161 in order to move the movable base 163 in the sub-scanning direction by a distance corresponding to the width of one main scanning line.

When it has been confirmed, in accordance with the position detection signal with respect to the optical head 135, which signal is received from the linear encoder 167, that the optical head 135 has been returned to the original position, and that the movable base 163 has been moved in the sub-scanning direction by the distance corresponding to the width of one main scanning line, the control unit 170 operates in order to irradiate the laser beam 124 produced by the second laser beam source 122 successively to the adsorptive regions 4, 4, . . . , which are located along the second main scanning line on the biochemical analysis unit 1, in the same manner as that in the operation for irradiating the laser beam 124 produced by the second laser beam source 122 successively to the adsorptive regions 4, 4, . . . , which are located along the first main scanning line on the biochemical analysis unit 1. In this manner, Rhodamine contained in each of the adsorptive regions 4, 4, . . . is excited by the laser beam 124, and the fluorescence 145 radiated out from each of the adsorptive regions 4, 4, . . . is photoelectrically detected by the photomultiplier 150.

The analog signal obtained from the photomultiplier 150 is converted by the analog-to-digital converter 153 into the digital signal. The digital signal is fed into the signal processing unit 154.

The signal processing unit 154 receives the digital signal, which has been obtained from the photoelectric detection of the fluorescence 145 radiated out from each of the adsorptive regions 4, 4, . . . and the digitization of the thus obtained analog signal. Also, the signal processing unit 154 reads out the data, which concerns the amount of the adsorptive material contained in each of the adsorptive regions 4, 4, . . . of the biochemical analysis unit 1, from the memory (not shown). In accordance with the read-out data, the signal processing unit 154 corrects the digital signal and thereby forms the data for a biochemical analysis, which data corresponds to the fluorescence information having been recorded on each of the adsorptive regions 4, 4, . . . . In this manner, the problems are capable of being prevented from occurring in that noise due to variations in amount of the adsorptive material contained in the adsorptive regions 4, 4, . . . occurs in the data for a biochemical analysis.

In the manner described above, the entire surface of the biochemical analysis unit 1 is scanned with the laser beam 124 having been produced by the second laser beam source 122. Rhodamine contained in each of the adsorptive regions 4, 4, . . . is thus excited by the laser beam 124, and the fluorescence 145 is photoelectrically detected by the photomultiplier 150. The thus obtained analog signal is digitized by the analog-to-digital converter 153, and the digital signal is fed into the signal processing unit 154. The control unit 170 then feeds the actuation stop signal into the second laser beam source 122, and the actuation of the second laser beam source 122 is stopped.

In the manner described above, the data for a biochemical analysis is formed from the radiation information and the fluorescence information having been recorded on the adsorptive regions 4, 4, . . . of the biochemical analysis unit 1.

In cases where the biochemical analysis unit 1 has been subjected N number of times to the hybridization of the organism-originating substance, which has been labeled with the labeling substance, with the specific binding substances adsorbed to the adsorptive regions 4, 4, . . . , and the biochemical analysis unit 1 has thus been used for the formation of the data for a biochemical analysis, the biochemical analysis unit 1 is sent from the user to a maker and subjected to recycling. Also, in cases where the stimulable phosphor sheet 90 has been subjected M number of times to the exposure operation, in which the stimulable phosphor sheet 90 is exposed to the radiation radiated out from the radioactive labeling substance selectively contained in the at least one adsorptive region 4 of the biochemical analysis unit 1, and the stimulable phosphor sheet 90 has thus been used for the formation of the data for a biochemical analysis, the stimulable phosphor sheet 90 is sent from the user to the maker and subjected to the recycling.

When the maker receives the biochemical analysis unit 1 from the user for the purposes of the recycling, the maker reads out the data, which has been recorded on the magnetic recording layer 5 of the biochemical analysis unit 1, by use of a reader (not shown). In cases where the number of times of recycling recorded on the first data recording region 5a of the magnetic recording layer 5 is smaller than the predetermined number of times, a judgment is made as to the kind of the labeling substance used. In cases where the radioactive labeling substance has not been used as the labeling substance, the adsorptive regions 4, 4, . . . of the biochemical analysis unit 1 are washed, and the specific binding substances adsorbed to the adsorptive regions 4, 4, . . . are removed. Thereafter, new specific binding substances are spotted respectively onto the adsorptive regions 4, 4, . . . by use of the spotting apparatus. Also, the number of times of recycling, which has been recorded on the first data recording region 5a of the magnetic recording layer 5, is increased by 1, and the biochemical analysis unit 1 is delivered from the maker.

In cases where the radioactive labeling substance has been used as the labeling substance, the maker removes the adsorptive material from the through-holes 3, 3, . . . of the base plate 2 of the biochemical analysis unit 1 and fills a new adsorptive material in the adsorptive regions 4, 4, . . . . After the adsorptive regions 4, 4, . . . have thus been reproduced, new specific binding substances are spotted respectively onto the adsorptive regions 4, 4, . . . by use of the spotting apparatus. Also, the number of times of recycling, which has been recorded on the first data recording region 5a of the magnetic recording layer 5, is increased by 1, and the biochemical analysis unit 1 is delivered from the maker. The adsorptive material having been removed from the through-holes 3, 3, . . . of the biochemical analysis unit 1 is managed in accordance with the data concerning the date and hour of execution of the hybridization, which data has been recorded on the first data recording region 5a of the magnetic recording layer 5 of the biochemical analysis unit 1, until the adsorptive material becomes capable of being scrapped.

When the maker receives the stimulable phosphor sheet 90 from the user for the purposes of the recycling, the maker removes the stimulable phosphor having been filled in the through-holes 93, 93, . . . of the stimulable phosphor sheet 90, and fills a new stimulable phosphor in the through-holes 93, 93, . . . . After the dot-shaped stimulable phosphor layer regions 92, 92, . . . have thus been reproduced, the number of times of recycling, which has been recorded on the magnetic recording layer 94 of the stimulable phosphor sheet 90, is increased by 1, and the stimulable phosphor sheet 90 is delivered from the maker. The stimulable phosphor having been removed from the through-holes 93, 93, . . . of the stimulable phosphor sheet 90 is subjected to an erasing operation, in which erasing light is irradiated to the stimulable phosphor and energy remaining on the stimulable phosphor is erased. The stimulable phosphor is then scrapped.

With the embodiment described above, the base plate 2 of the biochemical analysis unit 1 is provided with the magnetic recording layer 5. Also, the data concerning the kinds of the specific binding substances having been spotted and the data concerning the positions of the adsorptive regions 4, 4, . . . , to which positions the specific binding substances have been spotted respectively, are recorded on the first data recording region 5a of the magnetic recording layer 5, which region is protected from data writing conducted by a user. Therefore, the user is capable of reading out the data from the first data recording region 5a of the magnetic recording layer 5 of the biochemical analysis unit 1 by use of the reader (not shown) and is capable of reliably executing the hybridization of the desired organism-originating substance with the specific binding substances adsorbed to the adsorptive regions 4, 4, . . . of the biochemical analysis unit 1. In this manner, the user is capable of reliably forming the data for a biochemical analysis. Accordingly, the efficiency and the accuracy of the biochemical analysis are capable of being enhanced markedly.

Also, with this embodiment, the base plate 2 of the biochemical analysis unit 1 is provided with the magnetic recording layer 5. Also, the data concerning the number of times of use of the biochemical analysis unit 1 is recorded on the first data recording region 5a of the magnetic recording layer 5, which region is protected from data writing conducted by a user. Therefore, in cases where the data having been recorded on the first data recording region 5a of the magnetic recording layer 5 is managed, the problems are capable of being reliably prevented from occurring in that the user executes the biochemical analysis by use of the biochemical analysis unit 1, which has been used the predetermined number of times and in which part of the specific binding substances adsorbed to the adsorptive regions 4, 4, . . . has separated from the adsorptive regions 4, 4, . . . . Accordingly, the efficiency and the accuracy of the biochemical analysis are capable of being enhanced markedly.

Further, with this embodiment, the hybridization apparatus is provided with the read-out head 37 for reading out the data from the first data recording region 5a of the magnetic recording layer 5 of the biochemical analysis unit 1. In cases where it has been judged, in accordance with the data having been read out by the read-out head 37, that the biochemical analysis unit 1 has been used N number of times, the control unit 60 operates such that the biochemical analysis unit 1 is returned to the user and the hybridization with the biochemical analysis unit 1 cannot be executed. Therefore, the problems are capable of being reliably prevented from occurring in that the user executes the biochemical analysis by use of the biochemical analysis unit 1, which has been used the predetermined number of times and in which part of the specific binding substances adsorbed to the adsorptive regions 4, 4, . . . has separated from the adsorptive regions 4, 4, . . . . Accordingly, the efficiency and the accuracy of the biochemical analysis are capable of being enhanced markedly.

Furthermore, with this embodiment, the adsorptive regions 4, 4, . . . are formed by filling the adsorptive material in the through-holes 3, 3, . . . of the base plate 2 of the biochemical analysis unit 1. In such cases, it is not always possible to form the through-holes 3, 3, . . . such that all of the through-holes 3, 3, . . . have uniform size. Also, it is not always possible to fill the adsorptive material uniformly in the through-holes 3, 3, . . . . In such cases, there is the risk that noise due to nonuniformity in amount of the adsorptive material contained in the adsorptive regions 4, 4, . . . of the biochemical analysis unit 1 will occur in the data for a biochemical analysis obtained by transferring the radiation information, which has been recorded on the adsorptive regions 4, 4, . . . of the biochemical analysis unit 1, to the dot-shaped stimulable phosphor layer regions 92, 92, . . . of the stimulable phosphor sheet 90, irradiating the laser beam 124 to the dot-shaped stimulable phosphor layer regions 92, 92, . . . , stimulating the stimulable phosphor contained in the dot-shaped stimulable phosphor layer regions 92, 92, . . . to emit the light 145, and photoelectrically detecting the emitted light 145. However, with this embodiment, the data concerning the amount of the adsorptive material contained in each of the adsorptive regions 4, 4, . . . , which data has been recorded on the magnetic recording layer 5 of the biochemical analysis unit 1, is read out by the read-out head 111 of the exposure apparatus and is written on the magnetic recording layer 94 of the stimulable phosphor sheet 90. At the time of formation of the data for a biochemical analysis, the data concerning the amount of the adsorptive material contained in each of the adsorptive regions 4, 4, . . . , which data has been recorded on the magnetic recording layer 94 of the stimulable phosphor sheet 90, is read out. In accordance with the data concerning the amount of the adsorptive material contained in each of the adsorptive regions 4, 4, . . . , the digital signal obtained from the photoelectric detection of the emitted light 145 is corrected. In this manner, the data for a biochemical analysis is formed. Therefore, the problems are capable of being efficiently prevented from occurring in that noise due to nonuniformity in amount of the adsorptive material contained in the adsorptive regions 4, 4, . . . of the biochemical analysis unit 1 occurs in the data for a biochemical analysis.

Also, with this embodiment, the adsorptive regions 4, 4, . . . are formed by filling the adsorptive material in the through-holes 3, 3, . . . of the base plate 2 of the biochemical analysis unit 1. In such cases, it is not always possible to form the through-holes 3, 3, . . . such that all of the through-holes 3, 3, . . . have uniform size. Also, it is not always possible to fill the adsorptive material uniformly in the through-holes 3, 3, . . . . In such cases, there is the risk that noise due to nonuniformity in amount of the adsorptive material contained in the adsorptive regions 4, 4, . . . of the biochemical analysis unit 1 will occur in the data for a biochemical analysis obtained by recording the fluorescence information on the adsorptive regions 4, 4, . . . of the biochemical analysis unit 1, irradiating the laser beam 124 to the adsorptive regions 4, 4, . . . of the biochemical analysis unit 1, exciting the fluorescent labeling substance, and photoelectrically detecting the fluorescence 145. However, with this embodiment, the data concerning the amount of the adsorptive material contained in each of the adsorptive regions 4, 4, . . . is recorded on the magnetic recording layer 5 of the biochemical analysis unit 1. At the time of formation of the data for a biochemical analysis, the data concerning the amount of the adsorptive material contained in each of the adsorptive regions 4, 4, . . . , which data has been recorded on the magnetic recording layer 5 of the biochemical analysis unit 1, is read out. In accordance with the data concerning the amount of the adsorptive material contained in each of the adsorptive regions 4, 4, . . . , the digital signal obtained from the photoelectric detection of the fluorescence 145 is corrected. In this manner, the data for a biochemical analysis is formed. Therefore, the problems are capable of being efficiently prevented from occurring in that noise due to nonuniformity in amount of the adsorptive material contained in the adsorptive regions 4, 4, . . . of the biochemical analysis unit 1 occurs in the data for a biochemical analysis.

Further, with this embodiment, such that a specific biochemical analysis unit 1 and a specific stimulable phosphor sheet 90 are capable of being used together by the user, in cases where the specific biochemical analysis unit 1 and the specific stimulable phosphor sheet 90 are supplied as one biochemical analysis kit to the user, the corresponding pieces of identification data are recorded respectively on the magnetic recording layer 5 of the biochemical analysis unit 1 and the magnetic recording layer 94 of the stimulable phosphor sheet 90. In this manner, it is guaranteed that only the specific biochemical analysis unit 1 and the specific stimulable phosphor sheet 90 are capable of being used together. Therefore, the effects described below are capable of being obtained. Specifically, by way of example, the data for a biochemical analysis may be obtained from an operation, wherein an organism-originating substance having been sampled from a person, who has an abnormality in genetic expression, and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. Also, the data for a biochemical analysis may be obtained from an operation, wherein an organism-originating substance having been sampled from a healthy person and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. An examination may then be executed by comparing the thus obtained two pieces of the data for a biochemical analysis. In such cases, the examination is capable of being executed reliably by using the same biochemical analysis unit 1 and the same stimulable phosphor sheet 90, and the accuracy of the examination is capable of being enhanced markedly.

Further, with this embodiment, such that a specific biochemical analysis unit 1 and a specific stimulable phosphor sheet 90 are capable of being used together by the user, in cases where the specific biochemical analysis unit 1 and the specific stimulable phosphor sheet 90 have been supplied as one biochemical analysis kit to the user, the identification data having been recorded on the magnetic recording layer 5 of the biochemical analysis unit 1 and the identification data having been recorded on the magnetic recording layer 94 of the stimulable phosphor sheet 90 are read out by the read-out head 111 of the exposure apparatus. In cases where the thus read-out pieces of identification data are not the ones corresponding to each other, the exposure operation for the stimulable phosphor sheet 90 by use of the biochemical analysis unit 1 cannot be executed. Therefore, the effects described below are capable of being obtained. Specifically, by way of example, the data for a biochemical analysis may be obtained from an operation, wherein an organism-originating substance having been sampled from a person, who has an abnormality in genetic expression, and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. Also, the data for a biochemical analysis may be obtained from an operation, wherein an organism-originating substance having been sampled from a healthy person and having been labeled with the radioactive labeling substance is subjected to the selective, specific binding to the specific binding substances adsorbed to the adsorptive regions 4, 4, . . . of the biochemical analysis unit 1, and wherein at least one stimulable phosphor layer region 92 among the plurality of the stimulable phosphor layer regions 92, 92, . . . of the stimulable phosphor sheet 90 is exposed to the radiation radiated out from the radioactive labeling substance, which is contained selectively in at least one adsorptive region 4 among the plurality of the adsorptive regions 4, 4, . . . as a result of the selective, specific binding. An examination may then be executed by comparing the thus obtained two pieces of the data for a biochemical analysis. In such cases, the examination is capable of being executed reliably by using the same biochemical analysis unit 1 and the same stimulable phosphor sheet 90, and the accuracy of the examination is capable of being enhanced markedly.

Furthermore, with this embodiment, at the time of the hybridization, the data concerning the date and hour of execution of the hybridization and the data representing whether the radioactive labeling substance has or has not been used are recorded on the magnetic recording layer 5 of the biochemical analysis unit 1. Therefore, in cases where the biochemical analysis unit 1 is to be recycled, appropriate processing is capable of being performed in accordance with the history of use of the biochemical analysis unit 1. Also, in cases where the radioactive labeling substance has been used, the adsorptive material having been removed from the adsorptive regions 4, 4, . . . is capable of being managed appropriately and scrapped at an appropriate period.

Also, with this embodiment, the data concerning the number of times of recycling is recorded on the magnetic recording layer 5 of the biochemical analysis unit 1 and the magnetic recording layer 94 of the stimulable phosphor sheet 90. Therefore, the base plate 2 of the biochemical analysis unit 1 and the support 91 of the stimulable phosphor sheet 90 are capable of being utilized efficiently, and the saving of resources is capable of being achieved.

Further, with this embodiment, the magnetic recording layer 5 of the biochemical analysis unit 1 is provided with the second data recording region 5b, on which the data is capable of being written by the user. Therefore, the user is capable of writing personally necessary data for the management of the biochemical analysis unit 1, such as data for specifying the person who sampled the organism-originating substance to be subjected to the selective hybridization with the specific binding substances adsorbed to the adsorptive regions 4, 4, . . . of the biochemical analysis unit 1, on the second data recording region 5b. Accordingly, the efficiency, with which the biochemical analysis is made, is capable of being enhanced markedly.

Furthermore, with this embodiment, by use of the hybridization apparatus, the organism-originating substance having been labeled with the radioactive labeling substance and the organism-originating substance having been labeled with the fluorescent labeling substance are subjected to the selective hybridization with the specific binding substances adsorbed to the adsorptive regions 4, 4, . . . of the biochemical analysis unit 1. Therefore, the labor is capable of being saved markedly. Also, it becomes possible for the user, who executes the hybridization, to minimize the occurrence of a variation in results of the hybridization.

Also, with this embodiment, the adsorptive regions 4, 4, . . . of the biochemical analysis unit 1 have been formed as the dot-shaped regions, which are located at a spacing from one another in the base plate 2 made from aluminum having the radiation attenuating properties. Therefore, the problems are capable of being efficiently prevented from occurring in that, at the time of the exposure operation, the electron rays (the beta rays), which have been radiated out from the radioactive labeling substance contained in one of the adsorptive regions 4, 4, . . . , are scattered within the base plate 2 of the biochemical analysis unit 1, mix with the electron rays (the beta rays) having been radiated out from an adjacent adsorptive region 4, and impinge upon the dot-shaped stimulable phosphor layer region 92, which stands facing the adjacent adsorptive region 4. Also, the dot-shaped stimulable phosphor layer regions 92, 92, . . . of the stimulable phosphor sheet 90 have been formed by embedding the stimulable phosphor in each of the through-holes 93, 93, . . . , which have been formed in the support 91 made from nickel having the radiation attenuating properties. Therefore, the problems are capable of being efficiently prevented from occurring in that the electron rays (the beta rays), which have been radiated out from the radioactive labeling substance contained in one of the adsorptive regions 4, 4, . . . , are scattered within the support 91 of the stimulable phosphor sheet 90 and impinge upon a stimulable phosphor layer region 92 adjacent to the stimulable phosphor layer region 92, which stands facing the adsorptive region 4. Accordingly, the electron rays (the beta rays), which have been radiated out from the radioactive labeling substance contained in the adsorptive region 4, are capable of selectively impinging upon the stimulable phosphor layer region 92, which stands facing the adsorptive region 4, and the problems are capable of being reliably prevented from occurring in that the electron rays (the beta rays), which have been radiated out from the radioactive labeling substance contained in the adsorptive region 4, impinge upon a stimulable phosphor layer region 92, which is to be exposed to the electron rays radiated out from an adjacent adsorptive region 4. As a result, the problems are capable of being prevented from occurring in that noise due to the exposure of the stimulable phosphor layer region 92, which is to be exposed to the electron rays (the beta rays) radiated out from an adsorptive region 4, to the electron rays (the beta rays) radiated out from the radioactive labeling substance contained in an adjacent adsorptive region 4 is formed in the data for a biochemical analysis, and the quantitative characteristics of the biochemical analysis are capable of being enhanced markedly.

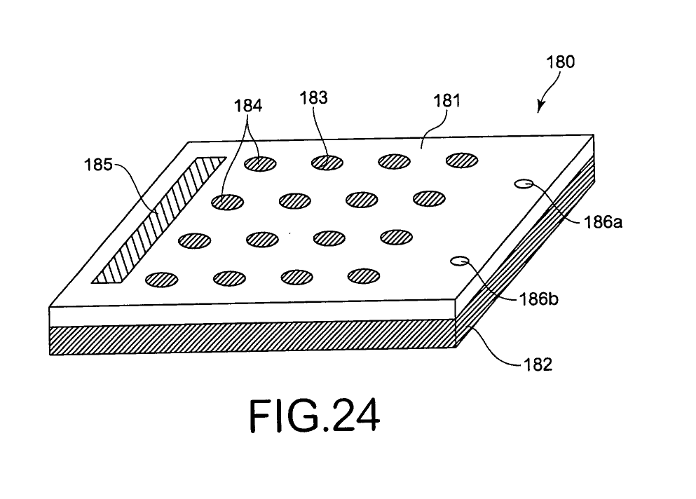

FIG. 24 is a schematic perspective view showing a second embodiment of the biochemical analysis unit in accordance with the present invention.

As illustrated in FIG. 24, a biochemical analysis unit 180, which constitutes the second embodiment of the biochemical analysis unit in accordance with the present invention, comprises a base plate 181, which is made from aluminum and is provided with a plurality of through-holes 183, 183, . . . having an approximately circular shape. The through-holes 183, 183, . . . are located as dot-shaped holes in a regular pattern. The biochemical analysis unit 180 also comprises an adsorptive film 182 made from a 6-nylon. The adsorptive film 182 is press-fitted into the through-holes 183, 183, . . . of the base plate 181 with a calendering apparatus (not shown). In this manner, a plurality of adsorptive regions 184, 184, . . . are formed. The adsorptive regions 184, 184, . . . are formed as the dot-shaped regions located in the regular pattern so as to correspond to the through-holes 183, 183, . . . of the base plate 181.

Though not shown precisely in FIG. 24, approximately 10,000 adsorptive regions 184, 184, . . . , each of which has the approximately circular shape with a size of approximately 0.01 mm², are formed in the regular pattern at a density of approximately 5,000 regions/cm² in the base plate 181 of the biochemical analysis unit 180.

In this embodiment, the adsorptive film 182 is press-fitted into the through-holes 183, 183, . . . of the base plate 181, such that the heights of the top surfaces of the adsorptive regions 184, 184, . . . may be identical with the height of the top surface of the base plate 181. The biochemical analysis unit 180 is formed in this manner.

As illustrated in FIG. 24, a magnetic recording layer 185 is embedded in the base plate 181. Also, the base plate 181 is provided with two circular position matching through-holes 186a and 186b.

In the second embodiment of the biochemical analysis unit in accordance with the present invention, as in the first embodiment of the biochemical analysis unit in accordance with the present invention, various kinds of data are capable of being recorded on the magnetic recording layer 185 of the biochemical analysis unit 180. Also, by use of the data having been recorded on the magnetic recording layer 185, the biochemical analysis unit 180 and the stimulable phosphor sheet 90 are capable of being utilized and managed appropriately, and the biochemical analysis is capable of being made accurately.

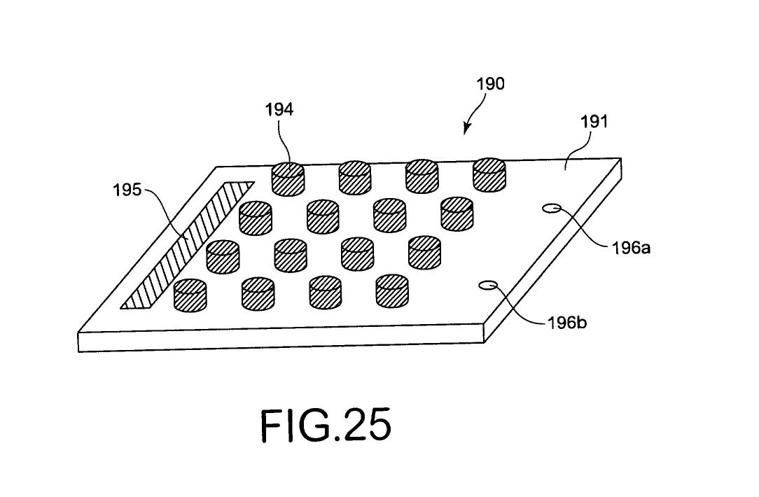

FIG. 25 is a schematic perspective view showing a third embodiment of the biochemical analysis unit in accordance with the present invention.

As illustrated in FIG. 25, a biochemical analysis unit 190, which constitutes the third embodiment of the biochemical analysis unit in accordance with the present invention, comprises a base plate 191, which is made from aluminum. Also, a plurality of adsorptive regions 194, 194, . . . made from a 6-nylon are formed in a regular pattern on the surface of the base plate 191.

Though not shown precisely in FIG. 25, approximately 10,000 adsorptive regions 194, 194, . . . , each of which has the approximately circular shape with a size of approximately 0.01 mm², are formed in the regular pattern at a density of approximately 5,000 regions/cm² on the surface of the base plate 191 made from aluminum.

As illustrated in FIG. 25, a magnetic recording layer 195 is embedded in the base plate 191. Also, the base plate 191 is provided with two circular position matching through-holes 196a and 196b.

Therefore, in the third embodiment of the biochemical analysis unit in accordance with the present invention, as in the first and second embodiments of the biochemical analysis unit in accordance with the present invention, various kinds of data are capable of being recorded on the magnetic recording layer 195 of the biochemical analysis unit 190. Also, by use of the data having been recorded on the magnetic recording layer 195, the biochemical analysis unit 190 and the stimulable phosphor sheet 90 are capable of being utilized and managed appropriately, and the biochemical analysis is capable of being made accurately.

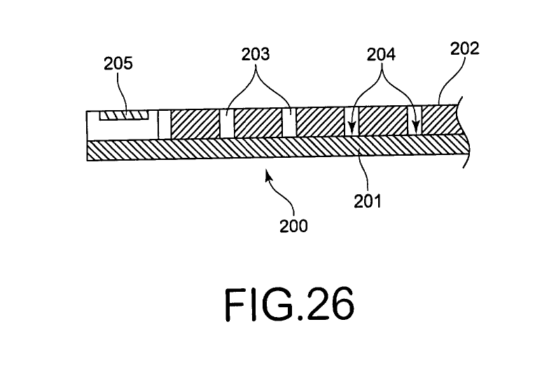

FIG. 26 is a schematic perspective view showing a fourth embodiment of the biochemical analysis unit in accordance with the present invention.

As illustrated in FIG. 26, a biochemical analysis unit 200, which constitutes the fourth embodiment of the biochemical analysis unit in accordance with the present invention, comprises an adsorptive plate 201 made from a 6-nylon. A perforated plate 202, which is made from aluminum and has a plurality of through-holes 203, 203, . . . , is located on one of two surfaces of the adsorptive plate 201, such that the perforated plate 202 is in close contact with the one surface of the adsorptive plate 201. Each of a plurality of adsorptive regions 204, 204, . . . is formed by a region of the adsorptive plate 201, which region is exposed within one of the through-holes 203, 203, . . . of the perforated plate 202.

Though not shown precisely in FIG. 26, approximately 10,000 through-holes 203, 203, . . . , each of which has the approximately circular shape with a size of approximately 0.01 mm², are formed in the regular pattern at a density of approximately 5,000 holes/cm² in the perforated plate 202 made from aluminum. Therefore, approximately 10,000 adsorptive regions 204, 204, . . . , each of which has the approximately circular shape with a size of approximately 0.01 mm², are formed in the regular pattern at a density of approximately 5,000 regions/cm² on the adsorptive plate 201.

As illustrated in FIG. 26, a magnetic recording layer 205 is embedded in the surface of the perforated plate 202.

Therefore, in the fourth embodiment of the biochemical analysis unit in accordance with the present invention, as in the first, second, and third embodiments of the biochemical analysis unit in accordance with the present invention, various kinds of data are capable of being recorded on the magnetic recording layer 205 of the biochemical analysis unit 200. Also, by use of the data having been recorded on the magnetic recording layer 205, the biochemical analysis unit 200 and the stimulable phosphor sheet 90 are capable of being utilized and managed appropriately, and the biochemical analysis is capable of being made accurately.

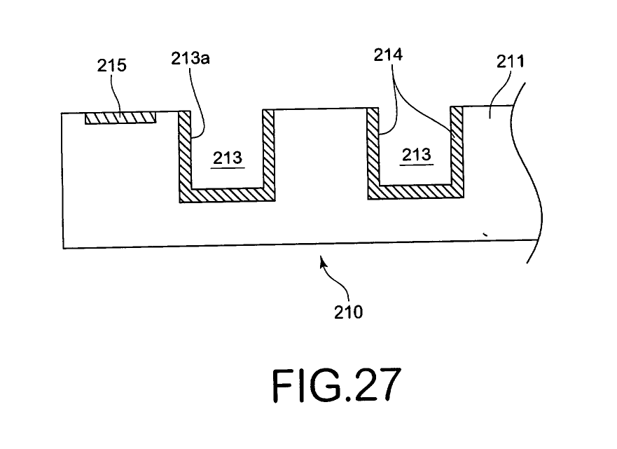

FIG. 27 is a schematic perspective view showing a fifth embodiment of the biochemical analysis unit in accordance with the present invention.

As illustrated in FIG. 27, a biochemical analysis unit 210, which constitutes the fifth embodiment of the biochemical analysis unit in accordance with the present invention, comprises a base plate 211, which is made from aluminum and is provided with a plurality of recesses 213, 213, . . . located in a regular pattern. An inner wall surface 213a of each of the recesses 213, 213, . . . is covered by a 6-nylon, and each of adsorptive regions 214, 214, . . . is thereby formed.

Though not shown precisely in FIG. 27, approximately 10,000 recesses 213, 213, . . . , each of which has the approximately circular shape with a size of approximately 0.01 mm², are formed in the regular pattern at a density of approximately 5,000 recesses/cm² in the base plate 211 made from aluminum.

As illustrated in FIG. 27, a magnetic recording layer 215 is embedded in the surface of the base plate 211.

Therefore, in the fifth embodiment of the biochemical analysis unit in accordance with the present invention, as in the first, second, third, and fourth embodiments of the biochemical analysis unit in accordance with the present invention, various kinds of data are capable of being recorded on the magnetic recording layer 215 of the biochemical analysis unit 210. Also, by use of the data having been recorded on the magnetic recording layer 215, the biochemical analysis unit 210 and the stimulable phosphor sheet 90 are capable of being utilized and managed appropriately, and the biochemical analysis is capable of being made accurately.

The biochemical analysis unit in accordance with the present invention is not limited to the embodiments described above and may be embodied in various other ways.

For example, in the aforesaid embodiments of the biochemical analysis unit in accordance with the present invention, the biochemical analysis units 1, 180, 190, 200, and 210 are provided respectively with the magnetic recording layers 5, 185, 195, 205, and 215. Alternatively, in lieu of the magnetic recording layers 5, 185, 195, 205, and 215, an optical recording layer may be employed. The biochemical analysis units 1, 180, 190, 200, and 210 may thus be provided with a data recording layer comprising a data-rewritable recording medium. No limitation is imposed upon the kind of the recording medium.

Also, in the aforesaid embodiments of the biochemical analysis unit in accordance with the present invention, various pieces of data are recorded on the magnetic recording layer 5, 185, 195, 205, or 215 of the biochemical analysis unit 1, 180, 190, 200, or 210. The pieces of data include the data concerning the amount of the adsorptive material contained in each of the adsorptive regions 4, 4, . . . , or the like, the data concerning the kinds of the specific binding substances having been spotted, the data concerning the positions of the adsorptive regions 4, 4, . . . , or the like, to which positions the specific binding substances have been spotted respectively, the data concerning the number of times of use of the biochemical analysis unit 1, or the like, the data concerning the date and hour of execution of the hybridization, the data representing whether the radioactive labeling substance has or has not been used, and the data concerning the number of times of recycling of the biochemical analysis unit 1, or the like. In cases where the biochemical analysis unit 1, or the like, and a specific stimulable phosphor sheet are supplied as one biochemical analysis kit to the user, the pieces of data also include the identification data for specifying the stimulable phosphor sheet, which constitutes the one biochemical analysis kit together with the biochemical analysis unit 1, or the like, and is to be used together with the biochemical analysis unit 1, or the like. The pieces of data further include the data which it is necessary for the user to utilize personally for the management of the biochemical analysis unit 1, or the like, such as the data for specifying the person who sampled the organism-originating substance to be subjected to the selective hybridization with the specific binding substances adsorbed to the adsorptive regions 4, 4, . . . of the biochemical analysis unit 1, or the like. However, all of the above-enumerated pieces of data need not necessarily be recorded on the magnetic recording layer 5, 185, 195, 205, or 215 of the biochemical analysis unit 1, 180, 190, 200, or 210. Several pieces of data among the above-enumerated pieces of data may not be recorded on the magnetic recording layer 5, 185, 195, 205, or 215 of the biochemical analysis unit 1, 180, 190, 200, or 210. Also, besides all of the above-enumerated pieces of data or several pieces of data among the above-enumerated pieces of data, different pieces of data may be recorded on the magnetic recording layer 5, 185, 195, 205, or 215 of the biochemical analysis unit 1, 180, 190, 200, or 210.

Further, in the aforesaid embodiments of the biochemical analysis unit in accordance with the present invention, by use of the hybridization apparatus, the organism-originating substance having been labeled with the radioactive labeling substance and the organism-originating substance having been labeled with the fluorescent labeling substance, which organism-originating substances are contained in the hybridization liquid, are automatically subjected to the selective hybridization with the specific binding substances adsorbed to the adsorptive regions 4, 4, . . . of the biochemical analysis unit 1, or the like. The hybridization apparatus is provided with the read-out head 37 and the magnetic recording head 38 for reading out the data from the magnetic recording layer 5 of the biochemical analysis unit 1, or the like, and recording the data on the magnetic recording layer 5 of the biochemical analysis unit 1, or the like. However, the hybridization apparatus need not necessarily be employed in order to execute the operation, wherein the organism-originating substance having been labeled with the radioactive labeling substance and the organism-originating substance having been labeled with the fluorescent labeling substance, which organism-originating substances are contained in the hybridization liquid, are automatically subjected to the selective hybridization with the specific binding substances adsorbed to the adsorptive regions 4, 4, . . . of the biochemical analysis unit 1, or the like. Alternatively, a different data read-out and recording apparatus having the functions for reading out and writing the data may be employed in order to read out the data from the magnetic recording layer 5 of the biochemical analysis unit 1, or the like, and to record the data on the magnetic recording layer 5 of the biochemical analysis unit 1, or the like. Also, a hybridization bag or a hybridization vessel containing the hybridization liquid may be employed in order to execute the operation, wherein the organism-originating substance having been labeled with the radioactive labeling substance and the organism-originating substance having been labeled with the fluorescent labeling substance, which organism-originating substances are contained in the hybridization liquid, are subjected to the selective hybridization with the specific binding substances adsorbed to the adsorptive regions 4, 4, . . . of the biochemical analysis unit 1, or the like.

Furthermore, in the aforesaid embodiments of the biochemical analysis unit in accordance with the present invention, the exposure apparatus is provided with the read-out head 111 and the magnetic recording head 112. The data having been recorded on the magnetic recording layer 5 of the biochemical analysis unit 1, or the like, and the data having been recorded on the magnetic recording layer 94 of the stimulable phosphor sheet 90 are read out with the read-out head 111. Also, the data is written on the magnetic recording layer 94 of the stimulable phosphor sheet 90 by use of the magnetic recording head 112. However, the exposure apparatus need not necessarily be provided with the read-out head 111 and the magnetic recording head 112. Alternatively, a different data read-out and recording apparatus having the functions for reading out and writing the data may be employed in order to read out the data having been recorded on the magnetic recording layer 5 of the biochemical analysis unit 1, or the like, and the data having been recorded on the magnetic recording layer 94 of the stimulable phosphor sheet 90, and to write the data on the magnetic recording layer 94 of the stimulable phosphor sheet 90.

Also, in the aforesaid embodiments of the biochemical analysis unit in accordance with the present invention, the electron microscope and the laser displacement meter are employed in order to form the data concerning the amount of the adsorptive material contained in each of the adsorptive regions 4, 4, . . . of the biochemical analysis unit 1, or the like. No limitation is imposed upon how the data concerning the amount of the adsorptive material contained in each of the adsorptive regions 4, 4, . . . of the biochemical analysis unit 1, or the like, is formed. The electron microscope and the laser displacement meter need not necessarily be employed for this purpose.

Further, in the aforesaid embodiments of the biochemical analysis unit in accordance with the present invention, the biochemical analysis unit 1, or the like, having been used N number of times and the stimulable phosphor sheet 90 having been used M number of times are collected by the maker and subjected to the recycling. However, the biochemical analysis unit 1, or the like, and the stimulable phosphor sheet 90 need not necessarily be recycled and may be processed arbitrarily by the user. In such cases, in cases where the radioactive labeling substance has been used, the biochemical analysis unit 1, or the like, having been used N number of times is managed in accordance with the data concerning the date and hour of execution of the hybridization, which data having been recorded on the magnetic recording layer 5 of the biochemical analysis unit 1, or the like. Also, the stimulable phosphor sheet 90 having been used M number of times is managed in accordance with the data concerning the date and hour of execution of the exposure operation, which data has been recorded on the magnetic recording layer 94 of the stimulable phosphor sheet 90. The biochemical analysis unit 1, or the like, and the stimulable phosphor sheet 90 are thereafter scrapped at an appropriate period.

Furthermore, in the aforesaid embodiments of the biochemical analysis unit in accordance with the present invention, the exposure apparatus is provided with the cover member locking mechanism comprising the hook member 105, the compression spring 106, the engagement groove 107, and the solenoid 108. Alternatively, the exposure apparatus may be provided with a different cover member locking mechanism.

Also, in the embodiment shown in FIGS. 4 to 23, the base plate 2 of the biochemical analysis unit 1 is provided with the two position matching through-holes 6a and 6b, and the support 91 of the stimulable phosphor sheet 90 is provided with the two position matching through-holes 96a and 96b at the positions corresponding to the position matching through-holes 6a and 6b. However, the base plate 2 of the biochemical analysis unit 1 need not necessarily be provided with the two position matching through-holes 6a and 6b, and the support 91 of the stimulable phosphor sheet 90 need not necessarily be provided with the two position matching through-holes 96a and 96b.

Further, in the aforesaid embodiments of the biochemical analysis unit in accordance with the present invention, approximately 10,000 adsorptive regions 4, 184, 194, 204, or 214, each of which has the approximately circular shape with a size of approximately 0.01 mm², are formed in a regular pattern at a density of approximately 5,000 regions/cm² in the base plate 2, 181, 191, 201, or 211 of the biochemical analysis unit 1, 180, 190, 200, or 210. However, the shape of each of the adsorptive regions 4, 184, 194, 204, or 214 is not limited to the approximately circular shape and may be set to be any of other shapes, such as a rectangular shape.

Furthermore, in the aforesaid embodiments of the biochemical analysis unit in accordance with the present invention, approximately 10,000 adsorptive regions 4, 184, 194, 204, or 214, each of which has the approximately circular shape with a size of approximately 0.01 mm², are formed in a regular pattern at a density of approximately 5,000 regions/cm² in the base plate 2, 181, 191, 201, or 211 of the biochemical analysis unit 1, 180, 190, 200, or 210. However, the number and the size of the adsorptive regions 4, 184, 194, 204, or 214 may be selected arbitrarily in accordance with the purposes of use. However, at least 10 adsorptive regions 4, 184, 194, 204, or 214, each of which has a size smaller than 5 mm², should preferably be formed at a density of at least 10 regions/cm² in the base plate 2, 181, 191, 201, or 211.

Also, in the aforesaid embodiments of the biochemical analysis unit in accordance with the present invention, approximately 10,000 adsorptive regions 4, 184, 194, 204, or 214, each of which has the approximately circular shape with a size of approximately 0.01 mm², are formed in a regular pattern at a density of approximately 5,000 regions/cm² in the base plate 2, 181, 191, 201, or 211 of the biochemical analysis unit 1, 180, 190, 200, or 210. However, the adsorptive regions 4, 184, 194, 204, or 214 need not necessarily be located in the regular pattern.

Further, in the aforesaid embodiments of the biochemical analysis unit in accordance with the present invention, approximately 10,000 dot-shaped stimulable phosphor layer regions 92, 92, . . . , each of which has the approximately circular shape with a size of approximately 0.01 mm², are formed at a density of approximately 5,000 regions/cm² and in the regular pattern, which is identical with the location pattern of the plurality of the adsorptive regions 4, 4, . . . of the base plate 2 of the biochemical analysis unit 1, or the like, in the support 91 of the stimulable phosphor sheet 90. However, the shape of each of the dot-shaped stimulable phosphor layer regions 92, 92, . . . is not limited to the approximately circular shape and may be set to be any of other shapes, such as a rectangular shape.

Further, in the aforesaid embodiments of the biochemical analysis unit in accordance with the present invention, approximately 10,000 dot-shaped stimulable phosphor layer regions 92, 92, . . . , each of which has the approximately circular shape with a size of approximately 0.01 mm², are formed at a density of approximately 5,000 regions/cm² and in the regular pattern, which is identical with the location pattern of the plurality of the adsorptive regions 4, 4, . . . of the base plate 2 of the biochemical analysis unit 1, or the like, in the support 91 of the stimulable phosphor sheet 90. However, the number and the size of the dot-shaped stimulable phosphor layer regions 92, 92, . . . may be selected arbitrarily in accordance with the purposes of use. However, at least 10 dot-shaped stimulable phosphor layer regions 92, 92, . . . , each of which has a size smaller than 5 mm², should preferably be formed at a density of at least 10 regions/cm² in the support 91.

Furthermore, in the aforesaid embodiments of the biochemical analysis unit in accordance with the present invention, approximately 10,000 dot-shaped stimulable phosphor layer regions 92, 92, . . . , each of which has the approximately circular shape with a size of approximately 0.01 mm², are formed at a density of approximately 5,000 regions/cm² and in the regular pattern, which is identical with the location pattern of the plurality of the adsorptive regions 4, 4, . . . of the base plate 2 of the biochemical analysis unit 1, or the like, in the support 91 of the stimulable phosphor sheet 90. However, the dot-shaped stimulable phosphor layer regions 92, 92, . . . need not necessarily be located in the regular pattern in the support 91 of the stimulable phosphor sheet 90. It is sufficient for the dot-shaped stimulable phosphor layer regions 92, 92, . . . to be located in the support 91 of the stimulable phosphor sheet 90 and in the pattern, which is identical with the location pattern of the plurality of the adsorptive regions 4, 184, 194, 204, or 214 of the biochemical analysis unit 1, 180, 190, 200, or 210.

Also, in the aforesaid embodiments of the biochemical analysis unit in accordance with the present invention, the size of each of the dot-shaped stimulable phosphor layer regions 92, 92, . . . of the stimulable phosphor sheet 90 is set to be identical with the size of each of the adsorptive regions 4, 184, 194, 204, or 214 of the biochemical analysis unit 1, 180, 190, 200, or 210. However, the size of each of the dot-shaped stimulable phosphor layer regions 92, 92, . . . of the stimulable phosphor sheet 90 need not necessarily be set to be identical with the size of each of the adsorptive regions 4, 184, 194, 204, or 214 of the biochemical analysis unit 1, 180, 190, 200, or 210. However, the size of each of the dot-shaped stimulable phosphor layer regions 92, 92, . . . of the stimulable phosphor sheet 90 should preferably be set at a value at least equal to the size of each of the adsorptive regions 4, 184, 194, 204, or 214 of the biochemical analysis unit 1, 180, 190, 200, or 210.

Further, in the aforesaid embodiments of the biochemical analysis unit in accordance with the present invention, the plurality of cDNA's are utilized. However, the specific binding substances, which may be utilized for the biochemical analysis unit in accordance with the present invention, are not limited to the cDNA's. The specific binding substances may be selected from a wide variety of specific binding substances, which are capable of specifically binding to organism-originating substances and whose base sequences, base lengths, compositions, and the like, are known. Examples of the specific binding substances include hormones, tumor markers, enzymes, antibodies, antigens, abzymes, other proteins, nucleic acids, cDNA's, DNA's, and RNA's.

Furthermore, in the aforesaid embodiments of the biochemical analysis unit in accordance with the present invention, the adsorptive regions 4, 184, 194, 204, or 214 of the biochemical analysis unit 1, 180, 190, 200, or 210 are formed by use of the 6-nylon. However, the adsorptive regions 4, 184, 194, 204, or 214 need not necessarily be formed by use of the 6-nylon. The adsorptive regions 4, 184, 194, 204, or 214 may be formed by use of other porous materials capable of forming a membrane filter. Examples of the porous materials other than the 6-nylon include nylons, such as a 6,6-nylon, and a 4,10-nylon; cellulose derivatives, such as nitrocellulose, cellulose acetate, and cellulose acetate butyrate; collagen; alginic acids, such as alginic acid, calcium alginate, and an alginic acid-polylysine polyion complex; polyolefins, such as a polyethylene and a polypropylene; polyvinyl chlorides; polyvinylidene chlorides; polyfluorides, such as a polyvinylidene fluoride and a polytetrafluoride; copolymers or composite materials of the above-enumerated materials; and porous carbon materials, such as active carbon. Also, the adsorptive regions 4, 184, 194, 204, or 214 of the biochemical analysis unit 1, 180, 190, 200, or 210 may be formed with inorganic porous materials. Examples of the inorganic porous materials, which may be utilized preferably, include metals, such as platinum, gold, iron, silver, nickel, and aluminum; metal oxides, such as alumina, silica, titania, and zeolite; metal salts, such as hydroxyapatite and calcium sulfate; and composite materials of the above-enumerated materials. Further, the adsorptive regions 4, 184, 194, 204, or 214 may be formed with fiber bundles.

Also, in the aforesaid embodiments of the biochemical analysis unit in accordance with the present invention, the base plate 2, 181, 191, 201, or 211 of the biochemical analysis unit 1, 180, 190, 200, or 210 is made from aluminum. However, the base plate 2, 181, 191, 201, or 211 of the biochemical analysis unit 1, 180, 190, 200, or 210 need not necessarily be made from aluminum and may be made from other materials. The base plate 2, 181, 191, 201, or 211 of the biochemical analysis unit 1, 180, 190, 200, or 210 should preferably have the radiation attenuating properties and/or the light attenuating properties. The material for the formation of the base plate may be selected from a wide variety of inorganic compound materials and organic compound materials, and should preferably be selected from metal materials, ceramic materials, and plastic materials.

Examples of the inorganic compound materials, which may be utilized preferably for the formation of the base plate 2, 181, 191, 201, or 211 of the biochemical analysis unit 1, 180, 190, 200, or 210, include metals, such as gold, silver, copper, zinc, aluminum, titanium, tantalum, chromium, iron, nickel, cobalt, lead, tin, and selenium; alloys, such as brass, stainless steel, and bronze; silicon materials, such as silicon, amorphous silicon, glass, quartz, silicon carbide, and silicon nitride; metal oxides, such as aluminum oxide, magnesium oxide, and zirconium oxide; and inorganic salts, such as tungsten carbide, calcium carbonate, calcium sulfate, hydroxyapatite, and gallium arsenide. The above-enumerated materials may have a single crystalline structure, an amorphous structure, or a structure of a polycrystalline sintered material, such as a ceramic material.

As the organic compound materials, which may be utilized for the formation of the base plate 2, 181, 191, 201, or 211 of the biochemical analysis unit 1, 180, 190, 200, or 210, high-molecular weight compounds are preferable. Examples of the high-molecular weight compounds, which may be utilized preferably, include polyolefins, such as a polyethylene and a polypropylene; acrylic resins, such as a polymethyl methacrylate and a butyl acrylate-methyl methacrylate copolymer; polyacrylonitriles; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; nylons, such as a 6-nylon, a 6,6-nylon, and a 4,10-nylon; polyimides; polysulfones; polyphenylene sulfides; silicon resins, such as a polydiphenyl siloxane; phenolic resins, such as novolak; epoxy resins; polyurethanes; polystyrenes; butadiene-styrene copolymers; polysaccharides, such as cellulose, cellulose acetate, nitrocellulose, starch, calcium alginate, and hydroxypropylmethylcellulose; chitin; chitosan; Japanese lacquer; polyamides, such as gelatin, collagen, and keratin; and copolymers of the above-enumerated high-molecular weight compounds. The above-enumerated organic compound materials may be composite materials. When necessary, the above-enumerated organic compound materials may be loaded with metal oxide particles, glass fibers, and the like. Also, the above-enumerated organic compound materials may be blended with organic compound materials.

In the embodiment of FIG. 24, the adsorptive film 182 is press-fitted into the through-holes 183, 183, . . . of the aluminum base plate 181 with the calendering apparatus (not shown). In this manner, the plurality of the adsorptive regions 184, 184, . . . of the biochemical analysis unit 180 are formed. Alternatively, the adsorptive film 182 may be press-fitted into the through-holes 183, 183, . . . of the base plate 181 with other means, such as a hot pressing apparatus. As another alternative, in lieu of the press fitting, other appropriate techniques may be utilized in order to embed the adsorptive film 182 in the through-holes 183, 183, . . . of the base plate 181 and to form the dot-shaped adsorptive regions 184, 184, . . . .

Further, in the aforesaid embodiments of the biochemical analysis unit in accordance with the present invention, the biochemical analysis unit 1, 180, 190, 200, or 210 is provided with the adsorptive regions 4, 184, 194, 204, or 214, which are spaced from one another. However, the biochemical analysis unit need not necessarily be provided with the adsorptive regions, which are spaced from one another.

Furthermore, in the embodiment shown in FIGS. 4 to 23, the adsorptive regions 4, 4, . . . of the biochemical analysis unit 1 are formed by filling the adsorptive material in each of the through-holes 3, 3, . . . of the base plate 2. Alternatively, instead of the through-holes 3, 3, . . . being formed, a plurality of recesses may be formed in the base plate 2, the adsorptive material may be filled in each of the recesses, and the adsorptive regions may thereby be formed.

Also, in the embodiment shown in FIG. 25, the adsorptive regions 194, 194, . . . of the biochemical analysis unit 190 are formed on the surface of the base plate 191. Alternatively, a plurality of protrusions may be formed in a regular pattern on the surface of the base plate 191 of the biochemical analysis unit 190, and each of the adsorptive regions may be formed on the top surface of each of the protrusions.

Further, in the aforesaid embodiments of the biochemical analysis unit in accordance with the present invention, the stimulable phosphor sheet 90 is provided with the support 91, which is made from nickel and is provided with the plurality of the through-holes 93, 93, . . . having the approximately circular shape. Also, the stimulable phosphor is filled in each of the through-holes 93, 93, . . . of the support 91, and the dot-shaped stimulable phosphor layer regions 92, 92, . . . are thereby formed. Alternatively, instead of the through-holes 93, 93, . . . being formed, a plurality of recesses, each of which has an approximately circular shape, may be formed in a regular pattern in the support 91, and the stimulable phosphor may be embedded in each of the recesses. In this manner, the dot-shaped stimulable phosphor layer regions 92, 92, . . . may be formed.

Furthermore, in the aforesaid embodiments of the biochemical analysis unit in accordance with the present invention, the stimulable phosphor sheet 90 is provided with the support 91, which is made from nickel. However, the support 91 of the stimulable phosphor sheet 90 need not necessarily be made from nickel and may be made from other materials. The support 91 of the stimulable phosphor sheet 90 should preferably have the radiation attenuating properties and/or the light attenuating properties. The material for the formation of the support may be selected from a wide variety of inorganic compound materials and organic compound materials, and should preferably be selected from metal materials, ceramic materials, and plastic materials.

Examples of the inorganic compound materials, which may be utilized preferably for the formation of the support 91 of the stimulable phosphor sheet 90, include metals, such as gold, silver, copper, zinc, aluminum, titanium, tantalum, chromium, iron, nickel, cobalt, lead, tin, and selenium; alloys, such as brass, stainless steel, and bronze; silicon materials, such as silicon, amorphous silicon, glass, quartz, silicon carbide, and silicon nitride; metal oxides, such as aluminum oxide, magnesium oxide, and zirconium oxide; and inorganic salts, such as tungsten carbide, calcium carbonate, calcium sulfate, hydroxyapatite, and gallium arsenide. The above-enumerated materials may have a single crystalline structure, an amorphous structure, or a structure of a polycrystalline sintered material, such as a ceramic material.

As the organic compound materials, which may be utilized for the formation of the support 91 of the stimulable phosphor sheet 90, high-molecular weight compounds are preferable. Examples of the high-molecular weight compounds, which may be utilized preferably, include polyolefins, such as a polyethylene and a polypropylene; acrylic resins, such as a polymethyl methacrylate and a butyl acrylate-methyl methacrylate copolymer; polyacrylonitriles; polyvinyl chlorides; polyvinylidene chlorides; polyvinylidene fluorides; polytetrafluoroethylenes; polychlorotrifluoroethylenes; polycarbonates; polyesters, such as a polyethylene naphthalate and a polyethylene terephthalate; nylons, such as a 6-nylon, a 6,6-nylon, and a 4,10-nylon; polyimides; polysulfones; polyphenylene sulfides; silicon resins, such as a polydiphenyl siloxane; phenolic resins, such as novolak; epoxy resins; polyurethanes; polystyrenes; butadiene-styrene copolymers; polysaccharides, such as cellulose, cellulose acetate, nitrocellulose, starch, calcium alginate, and hydroxypropylmethylcellulose; chitin; chitosan; Japanese lacquer; polyamides, such as gelatin, collagen, and keratin; and copolymers of the above-enumerated high-molecular weight compounds. The above-enumerated organic compound materials may be composite materials. When necessary, the above-enumerated organic compound materials may be loaded with metal oxide particles, glass fibers, and the like. Also, the above-enumerated organic compound materials may be blended with organic compound materials.

Also, in the aforesaid embodiments of the biochemical analysis unit in accordance with the present invention, the hybridization liquid containing the organism-originating substance, which has been labeled with the radioactive labeling substance, and the organism-originating substance, which has been labeled with the fluorescent labeling substance, such as the fluoro chrome, is prepared and subjected to the hybridization with the specific binding substances having been spotted onto the adsorptive regions 4, 4, . . . , or the like. However, the organism-originating substance need not necessarily be labeled with the radioactive labeling substance and the fluorescent labeling substance, such as the fluoro chrome. It is sufficient for the organism-originating substance to be labeled with at least one kind of labeling substance selected from the group consisting of the radioactive labeling substance, the fluorescent labeling substance, and the labeling substance capable of producing the chemiluminescence when being brought into contact with a chemiluminescence substrate.

Further, in the aforesaid embodiments of the biochemical analysis unit in accordance with the present invention, the organism-originating substance, which has been labeled with the radioactive labeling substance and the fluorescent labeling substance, such as the fluoro chrome, is subjected to the hybridization with the specific binding substances. However, the binding of the organism-originating substance to the at least one specific binding substance need not necessarily be effected through the hybridization. Alternatively, in lieu of the hybridization being employed, the organism-originating substance may be specifically bound to the at least one specific binding substance through an antigen-antibody reaction or a receptor-ligand reaction.

Furthermore, in the aforesaid embodiments of the biochemical analysis unit in accordance with the present invention, the exposure apparatus is constituted such that the stimulable phosphor sheet 90 is superposed upon the biochemical analysis unit 1, or the like, which has been set on the base 102. Alternatively, the stimulable phosphor sheet 90 may be set on the base 102, and the biochemical analysis unit 1, or the like, may be superposed upon the stimulable phosphor sheet 90.

Also, in the aforesaid embodiments of the biochemical analysis unit in accordance with the present invention, the scanner shown in FIGS. 16 to 23 is utilized in order to read the radiation information of the radioactive labeling substance, which information has been recorded on each of the dot-shaped stimulable phosphor layer regions 92, 92, . . . of the stimulable phosphor sheet 90, and the fluorescence information of the fluorescent labeling substance, which information has been recorded on the adsorptive regions 4, 4, . . . of the biochemical analysis unit 1, or the like. In this manner, the data for a biochemical analysis is formed. However, the single scanner need not necessarily be utilized in order to perform the readout of the radiation information of the radioactive labeling substance and the fluorescence information of the fluorescent labeling substance. Alternatively, the radiation information of the radioactive labeling substance and the fluorescence information of the fluorescent labeling substance may be read out by two independent scanners, and the data for a biochemical analysis may thereby be formed.

Further, in the aforesaid embodiments of the biochemical analysis unit in accordance with the present invention, the control unit 170 performs the on-off control of the first laser beam source 121 or the second laser beam source 122 in synchronization with the intermittent movement of the optical head 135. Alternatively, in cases where the movement speed of the optical head 135 in the main scanning direction is determined such that the laser beam 124 moves quickly between the two adjacent dot-shaped stimulable phosphor layer regions 92, 92 or between the two adjacent adsorptive regions 4, 4, or the like, in the main scanning direction, the first laser beam source 121 or the second laser beam source 122 may be kept in the on state, and the optical head 135 may merely be moved intermittently. The dot-shaped stimulable phosphor layer regions 92, 92, . . . or the adsorptive regions 4, 4, . . . , or the like, may thus be successively scanned with the laser beam 124, and the light 145 emitted by the dot-shaped stimulable phosphor layer regions 92, 92, . . . or the fluorescence 145 produced by the adsorptive regions 4, 4, . . . , or the like, may be detected photoelectrically. In this manner, the data for a biochemical analysis may be formed.

The scanner shown in FIGS. 16 to 23 is provided with the first laser beam source 121, the second laser beam source 122, and the third laser beam source 123. However, the scanner need not necessarily be provided with the three laser beam sources.

Also, the scanner shown in FIGS. 16 to 23 is provided with the first laser beam source 121 for producing the laser beam 124 having a wavelength of 640 nm, the second laser beam source 122 for producing the laser beam 124 having a wavelength of 532 nm, and the third laser beam source 123 for producing the laser beam 124 having a wavelength of 473 nm. However, the laser beam sources need not necessarily be utilized as the light sources for stimulation or excitation. In lieu of the laser beam sources, LED light sources may be utilized as the light sources for stimulation or excitation. Also, a halogen lamp may be utilized as the light sources for stimulation or excitation, and wavelength components which do not contribute to the stimulation or excitation may be filtered out by a spectral filter.

Furthermore, in the aforesaid embodiments of the biochemical analysis unit in accordance with the present invention, the optical head 135 is moved with the scanning mechanism in the main scanning direction indicated by the arrow X in FIG. 22 and in the sub-scanning direction indicated by the arrow Y in FIG. 22. In this manner, all of the dot-shaped stimulable phosphor layer regions 92, 92, . . . of the stimulable phosphor sheet 90 or all of the adsorptive regions 4, 4, . . . of the biochemical analysis unit 1, or the like, are scanned with the laser beam 124. The stimulable phosphor may thus be stimulated, or the fluorescent labeling substance may thus be excited. Alternatively, the optical head 135 may be kept stationary, and the stage 140 may be moved in the main scanning direction indicated by the arrow X in FIG. 22 and in the sub-scanning direction indicated by the arrow Y in FIG. 22. In this manner, all of the dot-shaped stimulable phosphor layer regions 92, 92, . . . of the stimulable phosphor sheet 90 or all of the adsorptive regions 4, 4, . . . of the biochemical analysis unit 1, or the like, are scanned with the laser beam 124. The stimulable phosphor may thus be stimulated, or the fluorescent labeling substance may thus be excited. As another alternative, the optical head 135 may be moved in the main scanning direction indicated by the arrow X in FIG. 22 or in the sub-scanning direction indicated by the arrow Y in FIG. 22, and the stage 140 may be moved in the sub-scanning direction indicated by the arrow Y or in the main scanning direction indicated by the arrow X.

Also, in the scanner shown in FIGS. 16 to 23, the photomultiplier 150 is utilized as the photodetector, and the emitted light 145 or the fluorescence 145 is detected photoelectrically. The photodetector is not limited to the photomultiplier 150 and may be constituted of one of various devices capable of photoelectrically detecting the emitted light 145 or the fluorescence 145. For example, a photodiode, a line CCD, or a two-dimensional CCD may be utilized as the photodetector.

Further, in the aforesaid embodiments of the biochemical analysis unit in accordance with the present invention, the spotting head 9 of the spotting apparatus is moved with the driving mechanism in the main scanning direction indicated by the arrow X in FIG. 6 and in the sub-scanning direction indicated by the arrow Y in FIG. 6. In this manner, the specific binding substances are spotted onto the adsorptive regions 4, 4, . . . of the base plate 2 of the biochemical analysis unit 1, or the like. Alternatively, the spotting head 9 of the spotting apparatus may be kept stationary, and the biochemical analysis unit 1 may be moved in the main scanning direction indicated by the arrow X in FIG. 6 and in the sub-scanning direction indicated by the arrow Y in FIG. 6. In this manner, the specific binding substances may be spotted onto the adsorptive regions 4, 4, . . . of the base plate 2 of the biochemical analysis unit 1. As another alternative, the spotting head 9 of the spotting apparatus may be moved in the main scanning direction indicated by the arrow X in FIG. 6 or in the sub-scanning direction indicated by the arrow Y in FIG. 6, and the biochemical analysis unit 1 may be moved in the sub-scanning direction indicated by the arrow Y in FIG. 6 or in the main scanning direction indicated by the arrow X in FIG. 6. In this manner, the specific binding substances may be spotted onto the adsorptive regions 4, 4, . . . of the base plate 2 of the biochemical analysis unit 1.

Furthermore, in the aforesaid embodiments of the biochemical analysis unit in accordance with the present invention, the spotting apparatus provided with the driving mechanism is employed in order to spot the specific binding substances onto the adsorptive regions 4, 4, . . . of the base plate 2 of the biochemical analysis unit 1, or the like. Alternatively, a spotting apparatus provided with the injector 7 and the CCD camera 8 may be utilized, the orifice of the injector 7 and the adsorptive region 4, onto which the specific binding substance, such as the cDNA, is to be spotted, may be monitored with the CCD camera 8, and the specific binding substance, such as the cDNA, may be spotted from the injector 7 when the position of the orifice of the injector 7 and the center position of the adsorptive region 4, onto which the specific binding substance, such as the cDNA, is to be spotted, coincide with each other.

Embodiments of the system for managing a biochemical analysis unit in accordance with the present invention will be described hereinbelow.

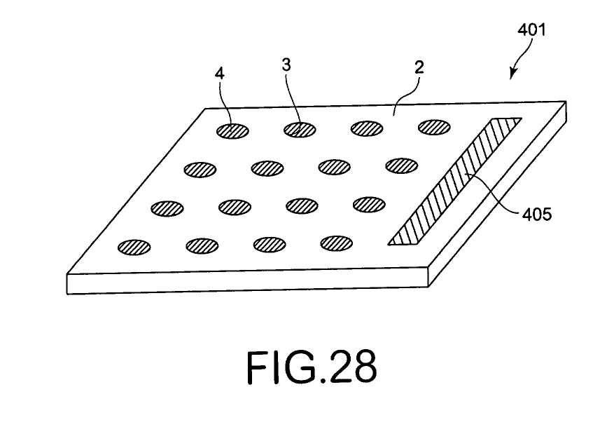

FIG. 28 is a schematic perspective view showing a biochemical analysis unit, which is managed by a first embodiment of the system for managing a biochemical analysis unit in accordance with the present invention.

As illustrated in FIG. 28, a biochemical analysis unit 401, which is to be managed by the first embodiment of the system for managing a biochemical analysis unit in accordance with the present invention, comprises the base plate 2, which is made from aluminum and is provided with the plurality of the through-holes 3, 3, . . . having an approximately circular shape. The through-holes 3, 3, . . . are located at a high density. Also, the 6-nylon is filled in each of the through-holes 3, 3, . . . . In this manner, the plurality of the dot-shaped adsorptive regions 4, 4, . . . are formed.

The base plate 2 of the biochemical analysis unit 401 is provided with a magnetic recording layer 405 constituted of a magnetic recording medium. When the biochemical analysis unit 401 is delivered, the ID data inherent to the biochemical analysis unit 401 is written on the magnetic recording layer 405. Also, when the biochemical analysis unit 401 is utilized for the hybridization, pieces of data concerning the hybridization are written on the magnetic recording layer 405 by the hybridization apparatus, which will be described later. The pieces of data include the data concerning the date and hour of execution of the hybridization, the data concerning the number of times of use for the hybridization, a radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance.

Though not shown precisely in FIG. 28, in this embodiment, approximately 10,000 through-holes 3, 3, . . . , each of which has the approximately circular shape with a size of approximately 0.01 mm², are formed in a regular pattern at a density of approximately 5,000 holes/cm² in the base plate 2. The 6-nylon is filled in each of the through-holes 3, 3, . . . , and the dot-shaped adsorptive regions 4, 4, . . . are thereby formed such that the heights of the top surfaces of the adsorptive regions 4, 4, . . . may be identical with the height of the top surface of the base plate 2.

FIG. 5 is a schematic front view showing a spotting apparatus.

When the biochemical analysis is to be made, as illustrated in FIG. 5, for example, different kinds of cDNA's, whose base sequences are known, are spotted as the specific binding substances respectively onto the plurality of the adsorptive regions 4, 4, . . . , which have been formed in the regular pattern in the biochemical analysis unit 401, by use of the spotting apparatus.

As illustrated in FIG. 5, the spotting apparatus comprises the spotting head 9. The spotting head 9 is provided with the injector 7 for injecting a liquid containing a specific binding substance toward the biochemical analysis unit 401, and the CCD camera 8. The spotting apparatus is constituted such that the orifice of the injector 7 and the adsorptive region 4, onto which the specific binding substance, such as the cDNA, is to be spotted, are monitored with the CCD camera 8, and the specific binding substance, such as the cDNA, is spotted from the injector 7 when the position of the orifice of the injector 7 and the center position of the adsorptive region 4, onto which the specific binding substance, such as the cDNA, is to be spotted, coincide with each other. In this manner, the cDNA is capable of being spotted accurately onto each of the adsorptive regions 4, 4, . . . having been formed respectively in the through-holes 3, 3, . . . .

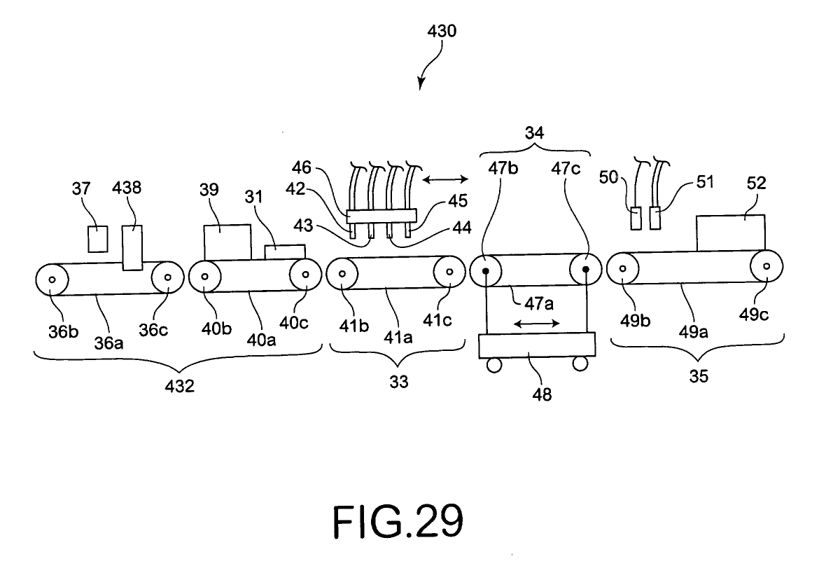

FIG. 29 is a schematic side view showing a hybridization apparatus, which constitutes the first embodiment of the system for managing a biochemical analysis unit in accordance with the present invention. In FIG. 29, similar elements are numbered with the same reference numerals with respect to FIG. 8.

As illustrated in FIG. 29, a hybridization apparatus 430 comprises a cartridge loading section 432 for loading the biochemical analysis unit 401 into the cartridge 31, which is shown in FIG. 9. The hybridization apparatus 430 also comprises the liquid injecting section 33 for selectively injecting the pre-treatment liquid, the hybridization liquid, the probe liquid containing the organism-originating substance having been labeled with the labeling substance, or the washing liquid into the cartridge 31, in which the biochemical analysis unit 401 has been accommodated. The hybridization apparatus 430 further comprises the reacting section 34 for shaking and vibrating the cartridge 31, in which the biochemical analysis unit 401 has been accommodated and which has been injected with the pre-treatment liquid, the hybridization liquid, the liquid prepared by the addition of the probe liquid to the hybridization liquid, or the washing liquid. The hybridization apparatus 430 still further comprises the biochemical analysis unit take-out section 35 for discharging the pre-treatment liquid, the liquid prepared by the addition of the probe liquid to the hybridization liquid, or the washing liquid from the cartridge 31, and taking out the biochemical analysis unit 401 from the cartridge 31.

Though not shown in FIG. 29, the hybridization apparatus 430 is provided with a temperature controller. The temperature of the region within the hybridization apparatus 430 is set at a value falling within a predetermined temperature range.

As illustrated in FIG. 29, the cartridge loading section 432 of the hybridization apparatus 430 comprises the first endless belt 36a, on which the biochemical analysis unit 401 is set. The cartridge loading section 432 also comprises the pair of the pulleys 36b and 36c, over which the first endless belt 36a is threaded and which are capable of rotating selectively in the clockwise direction or the counter-clockwise direction in FIG. 29. The cartridge loading section 432 further comprises the read-out head 37 for reading out the data having been recorded on the magnetic recording layer 405 of the biochemical analysis unit 401, which has been set on the first endless belt 36a. The cartridge loading section 432 still further comprises a magnetic recording head 438 for writing data on the magnetic recording layer 405 of the biochemical analysis unit 401. The cartridge loading section 432 also comprises the loading mechanism 39 for opening the cover 31b of the cartridge 31, loading the biochemical analysis unit 401 into the cartridge 31, and closing the cover 31b of the cartridge 31. The cartridge loading section 432 further comprises the second endless belt 40a for conveying the cartridge 31, into which the biochemical analysis unit 401 has been loaded. The cartridge loading section 432 still further comprises the pair of the pulleys 40b and 40c, over which the second endless belt 40a is threaded.

As illustrated in FIG. 29, the liquid injecting section 33, the reacting section 34, and the biochemical analysis unit take-out section 35 of the hybridization apparatus 430 have the same constitutions as those in the hybridization apparatus 30 shown in FIG. 8.

A control system, a detecting system, a driving system, an input system, and a displaying system of the hybridization apparatus 430 have basically the same constitutions as those shown in FIG. 10.

As illustrated in FIG. 10, the control system of the hybridization apparatus 430 comprises the control unit 60 for controlling the operations of the hybridization apparatus 430. The detecting system of the hybridization apparatus 430 comprises the read-out head 37 for reading out the data having been recorded on the magnetic recording layer 405 of the biochemical analysis unit 401, and the radiation sensor 50 for detecting the amount of the radioactive labeling substance contained in the washing liquid within the cartridge 31.

The driving system, the input system, and the displaying system of the hybridization apparatus 430 are constituted in the same manners as those shown in FIG. 10.

In the hybridization apparatus 430 constituted in the manner described above, the organism-originating substance having been labeled with the labeling substance is subjected to the selective hybridization with the specific binding substances, which have been adsorbed to the plurality of the adsorptive regions 4, 4, . . . of the base plate 2 of the biochemical analysis unit 401, in the manner described below.

Firstly, the hybridization liquid is prepared and accommodated in the hybridization liquid tank (not shown). Also, the washing liquid is prepared and accommodated in the washing liquid tank (not shown).

Further, the probe liquid, which contains the organism-originating substance having been labeled with the labeling substance, is prepared and accommodated in the probe liquid tip (not shown).

In cases where the specific binding substance, such as the cDNA, is to be labeled selectively with the radioactive labeling substance, the probe liquid containing the organism-originating substance, which has been labeled with the radioactive labeling substance and acts as a probe, is prepared and accommodated in the probe liquid tip.

In cases where the specific binding substance, such as the cDNA, is to be labeled selectively with the labeling substance capable of producing the chemiluminescence when being brought into contact with a chemiluminescence substrate, the probe liquid containing the organism-originating substance, which has been labeled with the labeling substance capable of producing the chemiluminescence when being brought into contact with a chemiluminescence substrate and which acts as the probe, is prepared and accommodated in the probe liquid tip.

In cases where the specific binding substance, such as the cDNA, is to be labeled selectively with the fluorescent labeling substance, such as the fluoro chrome, the probe liquid containing the organism-originating substance, which has been labeled with the fluorescent labeling substance, such as the fluoro chrome, and which acts as the probe, is prepared and accommodated in the probe liquid tip.

A probe liquid containing at least two organism-originating substances selected from among the organism-originating substance, which has been labeled with the radioactive labeling substance, the organism-originating substance, which has been labeled with the labeling substance capable of producing the chemiluminescence when being brought into contact with a chemiluminescence substrate, and the organism-originating substance, which has been labeled with the fluorescent labeling substance, such as the fluoro chrome, may be prepared and accommodated in the probe liquid tip. In this embodiment, the probe liquid containing the organism-originating substance, which has been labeled with the radioactive labeling substance, and the organism-originating substance, which has been labeled with the fluorescent labeling substance, is prepared and accommodated in the probe liquid tip.

When the hybridization is to be executed, the biochemical analysis unit 401 comprising the plurality of the adsorptive regions 4, 4, . . . , to which the specific binding substances, such as the cDNA's, have respectively been adsorbed, is set by the user on the first endless belt 36a of the cartridge loading section 432. Also, a start signal is inputted from the keyboard 80. At the same time, in cases where the organism-originating substance, which has been labeled with the radioactive labeling substance, is to be subjected to the hybridization with the specific binding substances contained in the plurality of the adsorptive regions 4, 4, . . . of the biochemical analysis unit 401, a radioactive label signal and a nuclide specifying signal for specifying the nuclide of the radioactive labeling substance are inputted by the user from the keyboard 80. In this embodiment, wherein the probe liquid containing the organism-originating substance having been labeled with the radioactive labeling substance has been prepared and accommodated in the probe liquid tip, the radioactive label signal and the nuclide specifying signal for specifying the nuclide of the radioactive labeling substance are inputted by the user from the keyboard 80.

The start signal, the radioactive label signal, and the nuclide specifying signal having been inputted from the keyboard 80 are fed into the control unit 60.

When the control unit 60 receives the start signal, the control unit 60 feeds a driving signal into the first motor 61. The first motor 61 rotates the pair of the pulleys 36b and 36c in order to rotate the first endless belt 36a clockwise in FIG. 29.

When the magnetic recording layer 405 of the biochemical analysis unit 401, which has been set on the first endless belt 36a, arrives at the position, which stands facing the read-out head 37, the control unit 60 feeds a driving stop signal to the first motor 61 in order to stop the first endless belt 36a. In this state, the data having been recorded on the magnetic recording layer 405 is read out by the read-out head 37.

In this embodiment, the ID data inherent to the biochemical analysis unit 401, the data concerning the date and hour of execution of the hybridization using the biochemical analysis unit 401, and the data concerning the number of times of use of the biochemical analysis unit 401 for the hybridization have been recorded on the magnetic recording layer 405. In cases where the radioactive labeling substance has been used as the labeling substance at the time of the hybridization, the radioactive label data representing the use of the radioactive labeling substance and the data concerning the nuclide of the radioactive labeling substance have also been recorded on the magnetic recording layer 405.

The data having been read out by the read-out head 37 are fed into the control unit 60. In cases where it has been judged in accordance with the data concerning the number of times of use for the hybridization, which data has been received from the read-out head 37, that the biochemical analysis unit 401 has already been used N number of times, the control unit 60 feeds a reverse rotation signal into the first motor 61. The pulleys 36b and 36c are thus rotated counter-clockwise in FIG. 29, and the biochemical analysis unit 401 is sent back to the user. Also, a message instructing change-over of the biochemical analysis unit 401 is displayed on the displaying panel 81.

If the biochemical analysis unit 401 is used N number of times or more, the specific binding substances having been adsorbed to the adsorptive regions 4, 4, . . . will separate from the adsorptive regions 4, 4, . . . . As a result, the accuracy of the analysis will become markedly low, and reliable analysis results cannot be obtained. The value of N is set to be, for example, 2.

The biochemical analysis unit 401 having been returned to the user is managed in accordance with the data having been recorded on the magnetic recording layer 405 of the biochemical analysis unit 401, as will be described later, and is then scrapped.

In cases where it has been judged in accordance with the data concerning the number of times of use for the hybridization, which data has been received from the read-out head 37, that the number of times of use of the biochemical analysis unit 401 is smaller than N, the control unit 60 further feeds the driving signal into the first motor 61, and the biochemical analysis unit 401 is moved to the position, at which the magnetic recording layer 405 stands facing the magnetic recording head 438.

When the biochemical analysis unit 401 has been moved to the position, at which the magnetic recording layer 405 stands facing the magnetic recording head 438, the driving stop signal is fed from the control unit 60 into the first motor 61.

Thereafter, the control unit 60 forms the data concerning the date and hour of execution of the hybridization in accordance with a built-in clock. Also, in accordance with the radioactive label signal and the nuclide specifying signal having been received, the control unit 60 feeds a writing signal to the magnetic recording head 438 in order to increase the number of times of use of the biochemical analysis unit 401, which is contained in the data concerning the number of times of use for the hybridization, by a value of 1 and to write the data concerning the date and hour of execution of the hybridization, the radioactive label data, and the data concerning the nuclide on the magnetic recording layer 405 of the biochemical analysis unit 401.

When the data writing on the magnetic recording layer 405 is completed, the control unit 60 again feeds the driving signal to the first motor 61, and the pulleys 36b and 36c are rotated in order to convey the biochemical analysis unit 401 into the loading mechanism 39.

Thereafter, the operations ranging from the operation, in which the biochemical analysis unit 401 is conveyed into the cartridge 31, to the operation, in which the probe liquid containing the organism-originating substances having been labeled with the labeling substances is added to the hybridization liquid having been accommodated in the cartridge 31, are performed in the same manner as that described above with reference to FIGS. 8, 9, and 10.

When a predetermined length of time has elapsed, the control unit 60 feeds the driving stop signal into the probe liquid pump 72 in order to stop the injection of the probe liquid into the cartridge 31. Also, the control unit 60 feeds the driving signal into the third motor 63. The third motor 63 rotates the pulleys 41b and 41c in order to rotate the third endless belt 41a clockwise in FIG. 29.

At the same time, the control unit 60 feeds the driving signal into the fourth motor 64. The fourth motor 64 rotates the pulleys 47b and 47c in order to rotate the fourth endless belt 47a clockwise in FIG. 29.

As a result, the cartridge 31, in which the biochemical analysis unit 401 has been accommodated, is transferred from the third endless belt 41a of the liquid injecting section 33 to the fourth endless belt 47a of the reacting section 34.

When the cartridge 31 has been transferred to the fourth endless belt 47a of the reacting section 34, the control unit 60 feeds the driving stop signal into the third motor 63 in order to stop the driving of the third endless belt 41a. When the cartridge 31 has been moved by the fourth endless belt 47a to approximately the center position of the reacting section 34, the control unit 60 feeds the driving stop signal into the fourth motor 64 in order to stop the cartridge 31.

Thereafter, the control unit 60 feeds the driving signal into the vibrating table motor 66 in order to vibrate the vibrating table 48.

As a result, the cartridge 31 is vibrated, and the hybridization liquid, to which the probe liquid containing the organism-originating substances having been labeled with the labeling substances has been added, is brought into uniform contact with all of the adsorptive regions 4, 4, . . . of the biochemical analysis unit 401 accommodated in the cartridge 31. Also, the organism-originating substance, which has been labeled with the radioactive labeling substance, and the organism-originating substance, which has been labeled with the fluorescent labeling substance, are subjected to the selective hybridization with the specific binding substances having been adsorbed to the plurality of the adsorptive regions 4, 4, . . . . In this manner, at least one specific binding substance among the plurality of the specific binding substances having been adsorbed to the plurality of the adsorptive regions 4, 4, . . . is selectively labeled with the radioactive labeling substance. Also, at least one specific binding substance is selectively labeled with the fluorescent labeling substance.

Thereafter, the operations up to the operation, in which the cartridge 31 is fed by the fifth endless belt 49a into the biochemical analysis unit take-out mechanism 52, are performed in the same manner as that described above with reference to FIGS. 8, 9, and 10.

In this embodiment, wherein the organism-originating substance contained in the probe liquid has been labeled with the radioactive labeling substance, in accordance with the radioactive label signal having been inputted from the keyboard 80, the radiation sensor 50 detects the concentration of the radioactive labeling substance in the washing liquid, and the washing operation with the washing liquid is repeated until the concentration of the radioactive labeling substance in the washing liquid decreases to a value at most equal to the reference concentration of the radioactive labeling substance. However, in cases where the organism-originating substance contained in the probe liquid has not been labeled with the radioactive labeling substance, the control unit 60 operates to finish the washing operation at the time at which it is judged that the injection of the washing liquid has been performed a predetermined number of times and the washing operation has thus been executed.

When the cartridge 31 has been fed into the biochemical analysis unit take-out mechanism 52, the control unit 60 feeds the driving stop signal into the fifth motor 65 in order to stop the driving of the fifth endless belt 49a. Also, the control unit 60 feeds the driving signal into the biochemical analysis unit take-out mechanism 52.

When the biochemical analysis unit take-out mechanism 52 receives the driving signal from the control unit 60, the biochemical analysis unit take-out mechanism 52 opens the cover 31b of the cartridge 31 and takes out the biochemical analysis unit 401 from the cartridge 31.

In the manner described above, the radiation information, which is formed with the radioactive labeling substance acting as the labeling substance, is recorded on at least one adsorptive region 4 among the plurality of the adsorptive regions 4, 4, . . . of the biochemical analysis unit 401. Also, the fluorescence information, which is formed with the fluorescent labeling substance, such as the fluoro chrome, is recorded on at least one adsorptive region 4 among the plurality of the adsorptive regions 4, 4, . . . of the biochemical analysis unit 401. The fluorescence information, which has been recorded on at least one adsorptive region 4 among the plurality of the adsorptive regions 4, 4, . . . , is read out by a scanner, which will be described later. In this manner, the data for a biochemical analysis is formed.

The radiation information, which is formed with the radioactive labeling substance, is transferred to a stimulable phosphor sheet. The radiation information, which has been transferred to the stimulable phosphor sheet, is read out by a scanner, which will be described later. In this manner, the data for a biochemical analysis is formed.

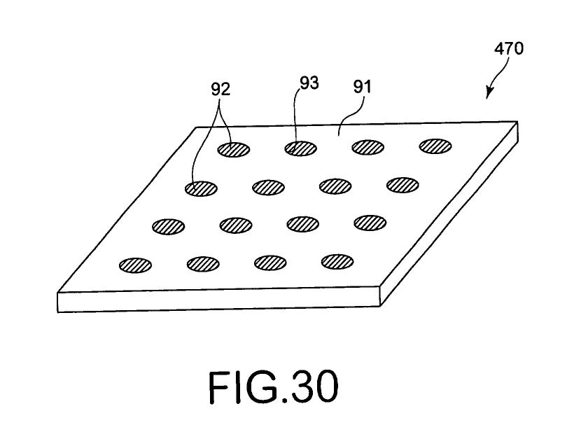

FIG. 30 is a schematic perspective view showing a stimulable phosphor sheet.

As illustrated in FIG. 30, a stimulable phosphor sheet 470 employed in this embodiment comprises the support 91, which is made from nickel and is provided with the plurality of the through-holes 93, 93, . . . having an approximately circular shape. The through-holes 93, 93, . . . are located in a regular pattern. Also, a stimulable phosphor is filled in each of the through-holes 93, 93, . . . of the support 91. In this manner, the plurality of the dot-shaped stimulable phosphor layer regions 92, 92, . . . are formed.

The plurality of the through-holes 93, 93, . . . of the support 91 are located in a pattern identical with the location pattern of the plurality of the adsorptive regions 4, 4, . . . of the base plate 2 of the biochemical analysis unit 401. Each of the stimulable phosphor layer regions 92, 92, . . . has a size identical with the side of each of the adsorptive regions 4, 4, . . . of the base plate 2 of the biochemical analysis unit 401.

Therefore, though not shown precisely in FIG. 30, in this embodiment, approximately 10,000 dot-shaped stimulable phosphor layer regions 92, 92, . . . , each of which has the approximately circular shape with a size of approximately 0.01 mm², are formed at a density of approximately 5,000 regions/cm² and in the regular pattern, which is identical with the location pattern of the plurality of the adsorptive regions 4, 4, . . . of the base plate 2 of the biochemical analysis unit 401, in the support 91 of the stimulable phosphor sheet 470.

Also, in this embodiment, the stimulable phosphor is embedded in each of the through-holes 93, 93, . . . of the support 91, such that the heights of the top surfaces of the dot-shaped stimulable phosphor layer regions 92, 92, . . . may be identical with the height of the top surface of the support 91. In this manner, the stimulable phosphor sheet 470 is formed.

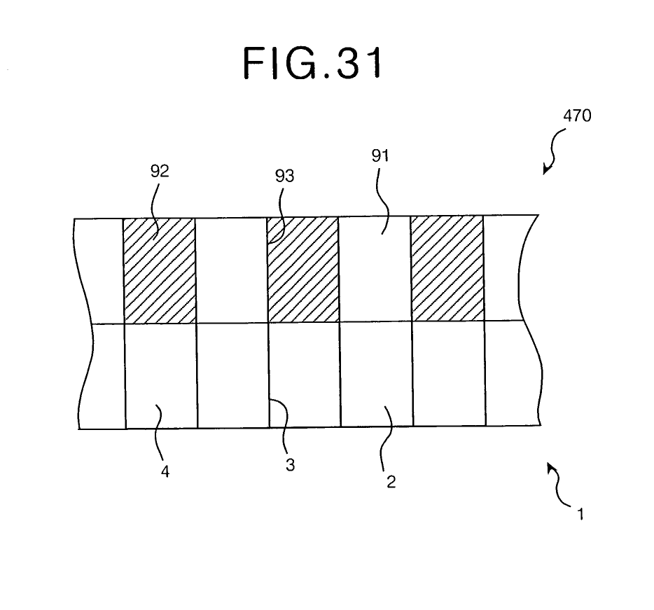

FIG. 31 is a schematic sectional view showing how at least one stimulable phosphor layer region 92 among the plurality of the dot-shaped stimulable phosphor layer regions 92, 92, . . . of the stimulable phosphor sheet 470 is exposed to the radiation radiated out from the radioactive labeling substance, which is contained selectively in at least one adsorptive region 4 among the plurality of the adsorptive regions 4, 4, . . . of the biochemical analysis unit 401.

In the exposure operation, the stimulable phosphor sheet 470 is superposed upon the surface of the biochemical analysis unit 401. As a result, at least one dot-shaped stimulable phosphor layer region 92 among the plurality of the dot-shaped stimulable phosphor layer regions 92, 92, . . . of the stimulable phosphor sheet 470 is exposed to the radiation radiated out from the radioactive labeling substance, which is contained selectively in at least one adsorptive region 4 among the plurality of the adsorptive regions 4, 4, . . . of the biochemical analysis unit 401. In this embodiment, the biochemical analysis unit 401 has been formed by filling a 6-nylon in each of the through-holes 3, 3, . . . of the base plate 2 made from aluminum. Therefore, in cases where the biochemical analysis unit 401 is subjected to treatment with the liquid for the hybridization, or the like, the biochemical analysis unit 401 suffers from little expansion or shrinkage. Accordingly, the stimulable phosphor sheet 470 is capable of being superposed upon the biochemical analysis unit 401, such that each of the adsorptive regions 4, 4, . . . of the biochemical analysis unit 401 accurately stands facing the corresponding dot-shaped stimulable phosphor layer region 92 among the dot-shaped stimulable phosphor layer regions 92, 92, . . . of the stimulable phosphor sheet 470. In this manner, the exposure operation for the dot-shaped stimulable phosphor layer regions 92, 92, . . . is capable of being executed.

In the manner described above, the biochemical analysis unit 401 and the stimulable phosphor sheet 470 are superposed one upon the other for a predetermined length of time, such that each of the adsorptive regions 4, 4, . . . of the biochemical analysis unit 401 stands facing the corresponding dot-shaped stimulable phosphor layer region 92 among the dot-shaped stimulable phosphor layer regions 92, 92, . . . of the stimulable phosphor sheet 470. As a result, at least one dot-shaped stimulable phosphor layer region 92 among the plurality of the dot-shaped stimulable phosphor layer regions 92, 92, . . . of the stimulable phosphor sheet 470 is exposed to the radiation radiated out from the radioactive labeling substance, which is contained selectively in at least one adsorptive region 4 among the plurality of the adsorptive regions 4, 4, . . . of the biochemical analysis unit 401.

At this time, electron rays (beta rays) are radiated out from the radioactive labeling substance having been adsorbed to at least one adsorptive region 4 among the plurality of the adsorptive regions 4, 4, . . . . However, the adsorptive regions 4, 4, . . . of the biochemical analysis unit 401 have been formed as the dot-shaped regions spaced from one another in the base plate 2, which is made from aluminum having the radiation attenuating properties. Therefore, the problems are capable of being efficiently prevented from occurring in that the electron rays (the beta rays), which have been radiated out from the radioactive labeling substance contained in one of the adsorptive regions 4, 4, . . . , are scattered within the base plate 2 of the biochemical analysis unit 401, mix with the electron rays (the beta rays) having been radiated out from an adjacent adsorptive region 4, and impinge upon the dot-shaped stimulable phosphor layer region 92, which stands facing the adjacent adsorptive region 4. Also, the dot-shaped stimulable phosphor layer regions 92, 92, . . . of the stimulable phosphor sheet 470 have been formed by embedding the stimulable phosphor in each of the through-holes 93, 93, . . . , which have been formed in the support 91 made from nickel having the radiation attenuating properties. Therefore, the problems are capable of being efficiently prevented from occurring in that the electron rays (the beta rays), which have been radiated out from the radioactive labeling substance contained in one of the adsorptive regions 4, 4, . . . , are scattered within the support 91 of the stimulable phosphor sheet 470 and impinge upon a stimulable phosphor layer region 92 adjacent to the stimulable phosphor layer region 92, which stands facing the adsorptive region 4. Accordingly, the electron rays (the beta rays), which have been radiated out from the radioactive labeling substance contained in the adsorptive region 4, are capable of selectively impinging upon the stimulable phosphor layer region 92, which stands facing the adsorptive region 4, and the problems are capable of being reliably prevented from occurring in that the electron rays (the beta rays), which have been radiated out from the radioactive labeling substance contained in the adsorptive region 4, impinge upon a stimulable phosphor layer region 92, which is to be exposed to the electron rays radiated out from an adjacent adsorptive region 4.

In the manner described above, the radiation information with the radioactive labeling substance is recorded on at least one dot-shaped stimulable phosphor layer region 92 among the plurality of the dot-shaped stimulable phosphor layer regions 92, 92, . . . of the support 91 of the stimulable phosphor sheet 470.

The scanner for reading out the radiation information from the stimulable phosphor sheet 470 in order to form the data for a biochemical analysis, and reading out the fluorescence information from the biochemical analysis unit 401 in order to form the data for a biochemical analysis is constituted in the same manner as that in the scanner described above with reference to FIGS. 16 to 22.

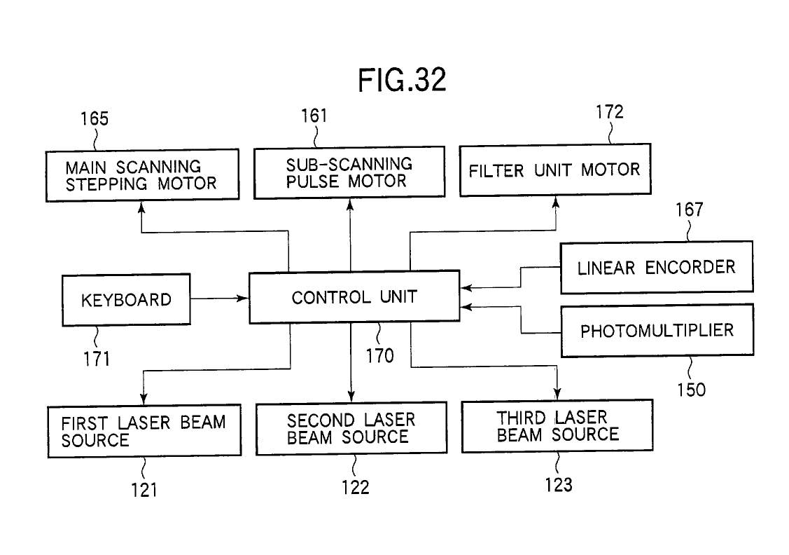

FIG. 32 is a block diagram showing a control system, an input system, a driving system, and a detecting system of the scanner. The constitution shown in FIG. 32 is identical with the constitution shown in FIG. 23, except that the reader 173 of the detecting system is omitted, and the processing for preventing noise due to the variation in amount of the adsorptive material contained in the adsorptive regions 4, 4, . . . from occurring in the data for a biochemical analysis is omitted.

Basically in the same manner as that described above with reference to FIG. 23, the data for a biochemical analysis is formed from the radiation information and the fluorescence information having been recorded on the adsorptive regions 4, 4, . . . of the biochemical analysis unit 401.

In cases where the biochemical analysis unit 401 has been subjected N number of times to the hybridization of the organism-originating substance, which has been labeled with the labeling substance, with the specific binding substances contained in the adsorptive regions 4, 4, . . . , and the biochemical analysis unit 401 has thus been used for the formation of the data for a biochemical analysis, the biochemical analysis unit 401 is sorted in accordance with the data concerning the date and hour of execution of the hybridization, the radioactive label data, and the data concerning the nuclide of the radioactive labeling substance, which data have been recorded on the magnetic recording layer 405 of the base plate 2 of the biochemical analysis unit 401. The sorted biochemical analysis unit 401 is stored and managed.

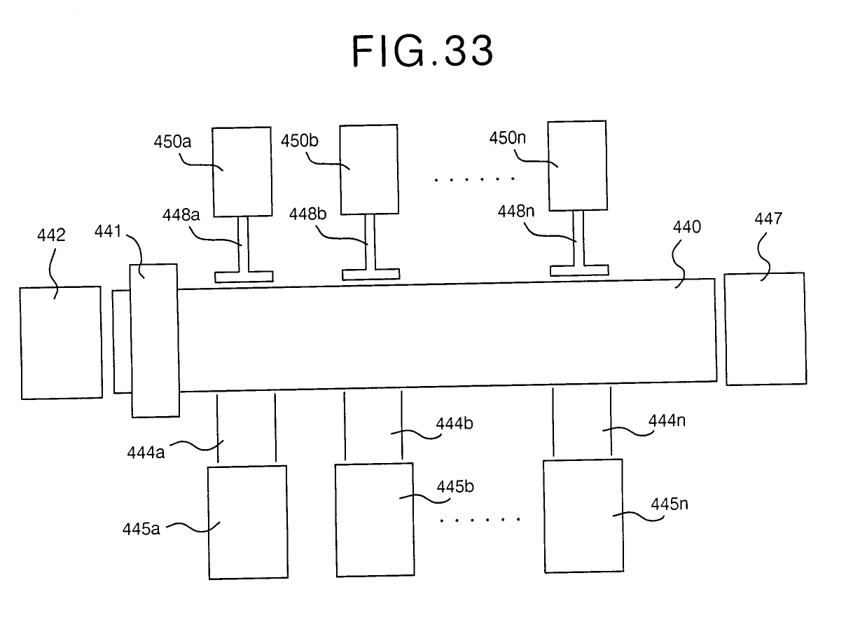

FIG. 33 is a schematic plan view showing a biochemical analysis unit sorting apparatus, which constitutes the first embodiment of the system for managing a biochemical analysis unit in accordance with the present invention.

As illustrated in FIG. 33, the biochemical analysis unit sorting apparatus comprises an endless belt 440 capable of conveying the biochemical analysis unit 401, and a read-out head 441 for reading out the magnetic data having been recorded on the magnetic recording layer 405 of the base plate 2 of the biochemical analysis unit 401, which is conveyed by the endless belt 440. The biochemical analysis unit sorting apparatus also comprises a biochemical analysis unit loading box 442, which is capable of being loaded with biochemical analysis units 401, 401, . . . and is provided with a biochemical analysis unit feeding mechanism 462 for feeding biochemical analysis units 401, 401, . . . one after another onto the endless belt 440. A first chute 444a, a second chute 444b, . . . , and an n-th chute 444n are located along one side of the endless belt 440.

Each of the first chute 444a, the second chute 444b, . . . , and the n-th chute 444n is capable of being selectively connected to a biochemical analysis unit collecting box 445a, 445b, . . . , or 445n for collecting the biochemical analysis unit 401 in accordance with a scrapping period. A scrapping box 447 is located at a downstream end of the endless belt 440. The scrapping box 447 collects a biochemical analysis unit 401, which has been subjected N number of times to the hybridization of the organism-originating substance having been labeled with the fluorescent labeling substance and/or the labeling substance capable of producing the chemiluminescence when being brought into contact with a chemiluminescence substrate, instead of being subjected to the hybridization of the organism-originating substance having been labeled with the radioactive labeling substance.

As illustrated in FIG. 33, a first sorting member 448a, a second sorting member 448b, . . . , and an n-th sorting member 448n are located on the other side of the endless belt 440 and at positions, which stand facing the first chute 444a, the second chute 444b, . . . , and the n-th chute 444n, respectively. Each of the first sorting member 448a, the second sorting member 448b, . . . , and the n-th sorting member 448n is capable of projecting onto the endless belt 440 in order to push and feed the biochemical analysis unit 401, which is conveyed on the top surface of the endless belt 440, to the first chute 444a, the second chute 444b, . . . , and the n-th chute 444n.

The first sorting member 448a, the second sorting member 448b, . . . , and the n-th sorting member 448n are driven respectively by a first solenoid 450a, a second solenoid 450b, . . . , and an n-th solenoid 450n so as to project onto the endless belt 440.

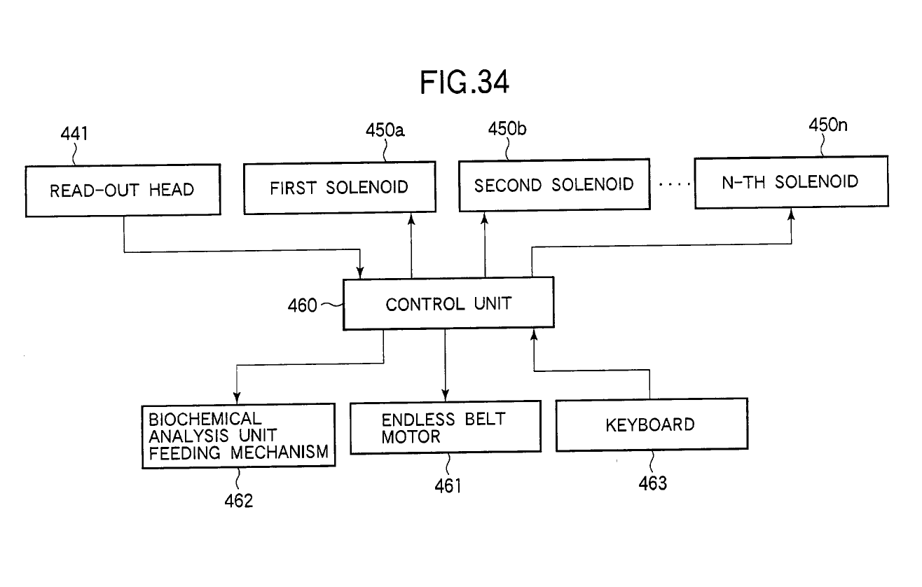

FIG. 34 is a block diagram showing a control system, a detecting system, a driving system, and an input system of the biochemical analysis unit sorting apparatus, which constitutes the first embodiment of the system for managing a biochemical analysis unit in accordance with the present invention.

As illustrated in FIG. 34, the control system of the biochemical analysis unit sorting apparatus comprises a control unit 460 for controlling the operations of the entire biochemical analysis unit sorting apparatus. The detecting system comprises the read-out head 441 for reading out the magnetic data having been recorded on the magnetic recording layer 405 of the base plate 2 of the biochemical analysis unit 401, which is placed on the endless belt 440.

Also, as illustrated in FIG. 34, the driving system of the biochemical analysis unit sorting apparatus comprises an endless belt motor 461 for driving the endless belt 440, and the biochemical analysis unit feeding mechanism 462 for feeding the biochemical analysis units 401, 401, . . . , which have been loaded into the biochemical analysis unit loading box 442, one after another onto the endless belt 440. The driving system also comprises the first solenoid 450a for driving the first sorting member 448a, the second solenoid 450b for driving the second sorting member 448b, . . . , and the n-th solenoid 450n for driving the n-th sorting member 448n. The input system of the biochemical analysis unit sorting apparatus comprises a keyboard 463.

The biochemical analysis unit sorting apparatus operates in the manner described below in order to sort the biochemical analysis unit 401 having been used, and to collect the biochemical analysis unit 401 into the biochemical analysis unit collecting box 445a, 445b, . . . , or 445n via the first chute 444a, the second chute 444b, . . . , or the n-th chute 444n, or into the scrapping box 447.

Firstly, the biochemical analysis unit collecting boxes 445a, 445b, . . . , and 445n are connected respectively to the first chute 444a, the second chute 444b, . . . , and the n-th chute 444n. In this embodiment, the biochemical analysis unit 401 is collected into the biochemical analysis unit collecting box 445a, 445b, . . . , or 445n in accordance with a month containing the date and hour, at which the biochemical analysis unit 401 is capable of being scrapped.

When the biochemical analysis unit collecting boxes 445a, 445b, . . . , and 445n have been connected respectively to the first chute 444a, the second chute 444b, . . . , and the n-th chute 444n, the user inputs data concerning a year and a month of scrapping and for specifying the year and month, at which the biochemical analysis unit 401 to be collected into one of the biochemical analysis unit collecting box 445a, 445b, . . . , or 445n is capable of being scrapped, from the keyboard 463.

Thereafter, a predetermined number of the biochemical analysis units 401, 401, . . . having been used are loaded into the biochemical analysis unit loading box 442, and an operation start signal is inputted from the keyboard 463.

The operation start signal having been inputted from the keyboard 463 is fed into the control unit 460. The control unit 460 feeds a driving signal into the endless belt motor 461 in order to drive the endless belt 440. Also, the control unit 460 feeds the driving signal into the biochemical analysis unit feeding mechanism 462 in order to feed the biochemical analysis units 401, 401, . . . , which have been loaded into the biochemical analysis unit loading box 442, one after another onto the endless belt 440.

The biochemical analysis unit 401 having been fed onto the endless belt 440 is conveyed by the endless belt 440 to the read-out head 441.

When the magnetic recording layer 405 of the base plate 2 of the biochemical analysis unit 401 arrives at the position which stands facing the read-out head 441, the control unit 460 feeds a driving stop signal into the endless belt motor 461 in order to stop the endless belt 440. In this state, the magnetic data having been recorded on the magnetic recording layer 405 of the base plate 2 of the biochemical analysis unit 401 is read out by the read-out head 441.

The data having been read out by the read-out head 441 is fed into the control unit 460.

When the control unit 460 receives the magnetic data from the read-out head 441, the control unit 460 makes a judgment as to whether the radioactive label data is or is not contained in the data having been received from the read-out head 441.

In cases where it has been judged that the radioactive label data is not contained in the data having been received from the read-out head 441 and that the radioactive labeling substance has not been used at the time of the hybridization, it is regarded that the biochemical analysis unit 401 is capable of being scrapped immediately. Therefore, in such cases, the control unit 460 feeds the driving signal into the endless belt motor 461 in order to drive the endless belt 440 and to collect the biochemical analysis unit 401 into the scrapping box 447, which is located at the downstream end of the endless belt 440.

In cases where it has been judged that the radioactive label data is contained in the data having been received from the read-out head 441 and that the radioactive labeling substance has been used at the time of the hybridization, the control unit 460 reads out reference data with respect to each nuclide of the radioactive labeling substance from a memory (not shown). Also, the control unit 460 calculates a scrapping period of the biochemical analysis unit 401, at which the level of the radioactivity of the radioactive labeling substance contained in the adsorptive region 4 of the biochemical analysis unit 401 attenuates to a level equal to at most the level that allows the biochemical analysis unit 401 to be scrapped. The calculation is made in accordance with the data concerning the date and hour of execution of the hybridization of the biochemical analysis unit 401, the radioactive label data, and the data concerning the nuclide of the radioactive labeling substance, which data have been received from the read-out head 441. In accordance with the thus calculated scrapping period of the biochemical analysis unit 401 and the data concerning the year and month of scrapping, which data is stored in the memory, the control unit 460 determines which sorting member among the first sorting member 448a, the second sorting member 448b, . . . , and the n-th sorting member 448n is to be driven. Also, the control unit 460 calculates the time of driving of the endless belt 440 required to feed the biochemical analysis unit 401 to the position which stands facing the determined sorting member.

Thereafter, the control unit 460 feeds the driving signal into the endless belt motor 461 in order to drive the endless belt 440. When the calculated time of driving of the endless belt 440 has elapsed, the control unit 460 feeds the driving stop signal into the endless belt motor 461 in order to stop the endless belt motor 461. Also, the control unit 460 feeds an actuating signal into the first solenoid 450a, the second solenoid 450b, . . . , or the n-th solenoid 450n for driving the first sorting member 448a, the second sorting member 448b, . . . , or the n-th sorting member 448n having been determined in accordance with the scrapping period of the biochemical analysis unit 401. The first sorting member 448a, the second sorting member 448b, . . . , or the n-th sorting member 448n is thus driven in order to feed the biochemical analysis unit 401 to the first chute 444a, the second chute 444b, . . . , or the n-th chute 444n, which stands facing the sorting member. The biochemical analysis unit 401 is thus collected into the biochemical analysis unit collecting box 445a, 445b, . . . , or 445n, which is connected to the chute.

In the same manner, the control unit 460 operates in order to feed the biochemical analysis units 401, 401, . . . , which have been loaded into the biochemical analysis unit loading box 442, one after another onto the endless belt 440. Also, the control unit 460 calculates the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. The calculation is made in accordance with the data concerning the date and hour of execution of the hybridization of the biochemical analysis unit 401 and the data concerning the nuclide of the radioactive labeling substance, which data have been recorded on the magnetic recording layer 405 of the biochemical analysis unit 401. In accordance with the thus calculated scrapping period of the biochemical analysis unit 401 and the data concerning the year and month of scrapping, which data is stored in the memory, the control unit 460 determines the biochemical analysis unit collecting box 445a, 445b, . . . , or 445n which is to be used for collecting the biochemical analysis unit 401. Further, the control unit 460 drives the corresponding sorting member among the first sorting member 448a, the second sorting member 448b, . . . , and the n-th sorting member 448n in order to feed the biochemical analysis unit 401 into the determined biochemical analysis unit collecting box 445a, 445b, . . . , or 445n via the first chute 444a, the second chute 444b, . . . , or the n-th chute 444n. The biochemical analysis unit 401 is thus sorted and collected.

In the manner described above, all of the biochemical analysis units 401, 401, . . . having been loaded into the biochemical analysis unit loading box 442 are sorted and collected into the biochemical analysis unit collecting box 445a, 445b, . . . , or 445n or into the scrapping box 447. Thereafter, an operation end signal is fed from the keyboard 463, the driving of the endless belt motor 461 is stopped, and the sorting and collection of the biochemical analysis units 401, 401, . . . are completed.

In the manner described above, in accordance with the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped, the biochemical analysis unit 401 is collected into the biochemical analysis unit collecting box 445a, 445b, . . . , or 445n. The biochemical analysis unit 401 is stored in the biochemical analysis unit collecting box 445a, 445b, . . . , or 445n until the calculated scrapping period of the biochemical analysis unit 401. The biochemical analysis unit 401 is then scrapped at the scrapping period.

In this embodiment, with the magnetic recording head 438 of the hybridization apparatus 430, the pieces of data are written on the magnetic recording layer 405 of the base plate 2 of the biochemical analysis unit 401. The pieces of data include the data concerning the date and hour of execution of the hybridization of the biochemical analysis unit 401, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. In cases where it has been judged, in accordance with the data concerning the number of times of use for the hybridization, that the biochemical analysis unit 401 has been subjected N number of times to the hybridization, the biochemical analysis unit sorting apparatus calculates the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. The calculation is made in accordance with the data concerning the date and hour of execution of the hybridization, the radioactive label data, and the data concerning the nuclide of the radioactive labeling substance, which data have been recorded on the magnetic recording layer 405 of the biochemical analysis unit 401. The biochemical analysis unit 401 is sorted in accordance with the calculated scrapping period, stored, and managed. Therefore, the biochemical analysis unit 401 is capable of being stored and managed reliably until the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. Also, the biochemical analysis unit 401 is capable of being scrapped after the level of the radioactivity of the radioactive labeling substance contained in the adsorptive region 4 of the biochemical analysis unit 401 has attenuated to the level equal to at most the level that allows the biochemical analysis unit 401 to be scrapped.

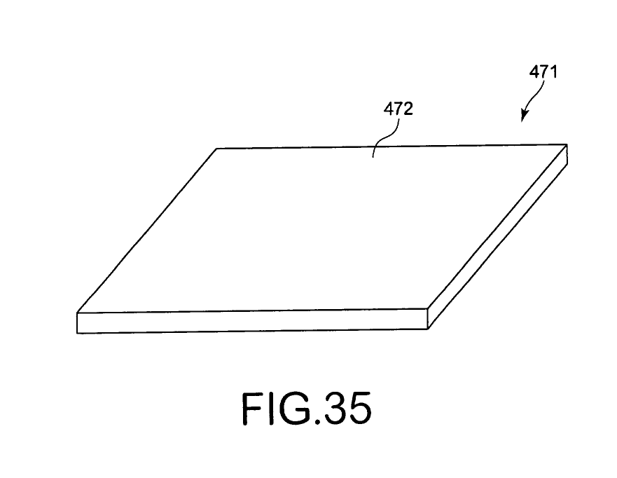

FIG. 35 is a schematic perspective view showing a biochemical analysis unit, which is managed by a second embodiment of the system for managing a biochemical analysis unit in accordance with the present invention.

As illustrated in FIG. 35, a biochemical analysis unit 471, which is managed by the second embodiment of the system for managing a biochemical analysis unit in accordance with the present invention, comprises an adsorptive plate 472 made from the 6-nylon.

At the time of the biochemical analysis, by use of the spotting apparatus shown in FIG. 5, liquids containing the specific binding substances, such as the cDNA's, are spotted onto the adsorptive plate 472 of the biochemical analysis unit 471. In this manner, a plurality of spot-shaped regions, which are spaced from one another, are formed.

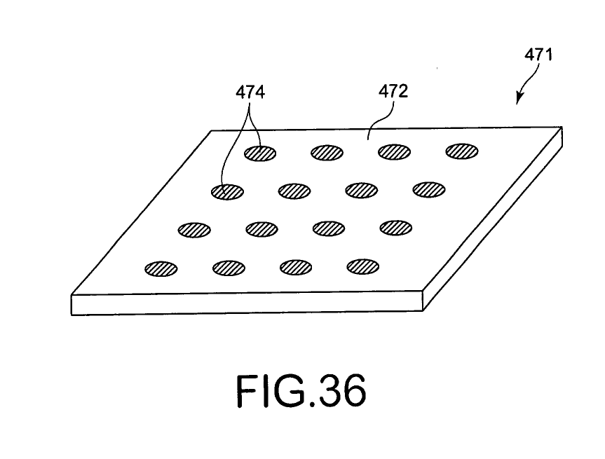

FIG. 36 is a schematic perspective view showing the biochemical analysis unit 471 provided with a plurality of spot-shaped regions located at a spacing from one another, each of the plurality of the spot-shaped regions having been formed with a process for spotting a liquid containing one of specific binding substances, such as cDNA's, onto the adsorptive plate 472 of the biochemical analysis unit 471 by use of the spotting apparatus.

As illustrated in FIG. 36, the liquids containing the specific binding substances, such as the cDNA's, are spotted onto the adsorptive plate 472 of the biochemical analysis unit 471. In this manner, a plurality of spot-shaped regions 474, 474, . . . , which are spaced from one another, are formed.

Though not shown precisely in FIG. 36, approximately 10,000 spot-shaped regions 474, 474, . . . , each of which has an approximately circular shape with a size of approximately 0.01 mm², are formed in a regular pattern at a density of approximately 5,000 regions/cm² on the adsorptive plate 472 of the biochemical analysis unit 471.

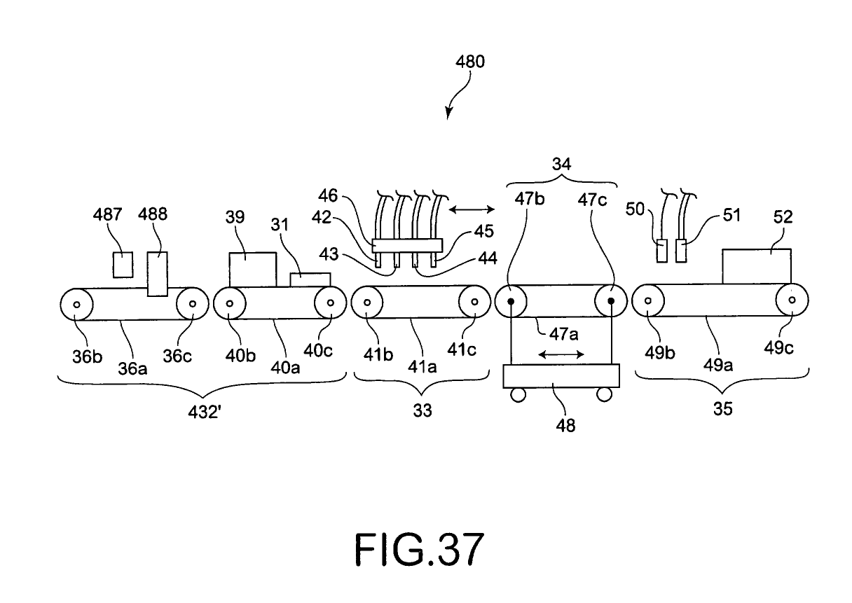

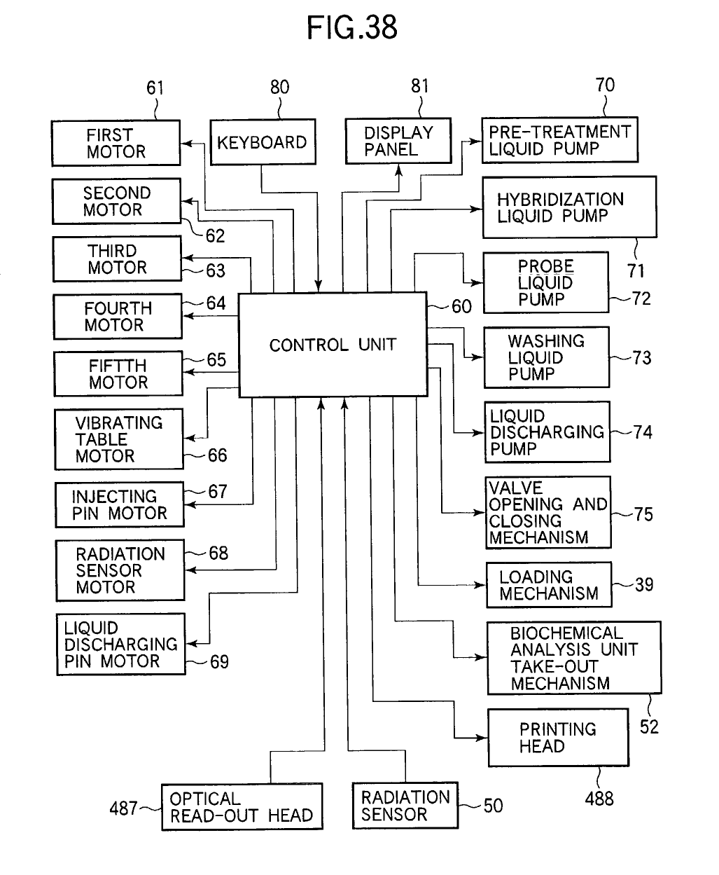

FIG. 37 is a schematic side view showing a hybridization apparatus, which constitutes the second embodiment of the system for managing a biochemical analysis unit in accordance with the present invention. FIG. 38 is a block diagram showing a control system, a detecting system, a driving system, an input system, and a displaying system of the hybridization apparatus, which constitutes the second embodiment of the system for managing a biochemical analysis unit in accordance with the present invention.

As illustrated in FIGS. 37 and 38, a hybridization apparatus 480, which constitutes the second embodiment of the system for managing a biochemical analysis unit in accordance with the present invention, has basically the same constitution as the constitution of the hybridization apparatus 430 described above with reference to FIGS. 29 and 10, except that a printing head 488 is utilized in lieu of the magnetic recording head 438 for writing the magnetic data on the magnetic recording layer 405 of the base plate 2 of the biochemical analysis unit 401, and an optical read-out head 487 is utilized in lieu of the read-out head 37 for reading out the magnetic data from the magnetic recording layer 405 of the base plate 2 of the biochemical analysis unit 401. At a cartridge loading section 432′, the printing head 488 prints the pieces of data on the adsorptive plate 472 of the biochemical analysis unit 471. The pieces of data include the data concerning the date and hour of execution of the hybridization, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. Also, the optical read-out head 487 reads out the data having been printed on the adsorptive plate 472 of the biochemical analysis unit 471.

In the hybridization apparatus 480, the organism-originating substance having been labeled with the radioactive labeling substance and the organism-originating substance having been labeled with the fluorescent labeling substance, which organism-originating substances are contained in the probe liquid, are subjected to the selective hybridization with the specific binding substances contained in the spot-shaped regions 474, 474, . . . of the adsorptive plate 472 of the biochemical analysis unit 471 basically in the same manner as that in the hybridization apparatus 430 described above with reference to FIGS. 29 and 10, except that the aforesaid pieces of data are printed on the adsorptive plate 472 of the biochemical analysis unit 471 by the printing head 488 and are read out optically by the optical read-out head 487.

In the manner described above, the radiation information, which is formed with the radioactive labeling substance acting as the labeling substance, is recorded on at least one spot-shaped region 474 among the plurality of the spot-shaped regions 474, 474, . . . of the biochemical analysis unit 471. Also, the fluorescence information, which is formed with the fluorescent labeling substance, such as the fluoro chrome, is recorded on at least one spot-shaped region 474. The fluorescence information, which has been recorded on at least one spot-shaped region 474, is read out by the scanner shown in FIGS. 16 to 22 and FIG. 32 in the same manner as that described above. In this manner, the data for a biochemical analysis is formed.

In the same manner as that described above, the radiation information, which is formed with the radioactive labeling substance, is transferred to the stimulable phosphor sheet 470 shown in FIG. 30. The radiation information, which has been transferred to the stimulable phosphor sheet 470, is read out by the scanner shown in FIGS. 16 to 22 and FIG. 32. In this manner, the data for a biochemical analysis is formed.

In cases where the biochemical analysis unit 471 has been subjected N number of times to the hybridization of the organism-originating substance, which has been labeled with the labeling substance, with the specific binding substances contained in the spot-shaped regions 474, 474, . . . , and the biochemical analysis unit 471 has thus been used for the formation of the data for a biochemical analysis, the biochemical analysis unit 471 is sorted in accordance with the data concerning the date and hour of execution of the hybridization, the radioactive label data, and the data concerning the nuclide of the radioactive labeling substance, which data have been printed on the adsorptive plate 472 of the biochemical analysis unit 471. The sorted biochemical analysis unit 471 is stored and managed.

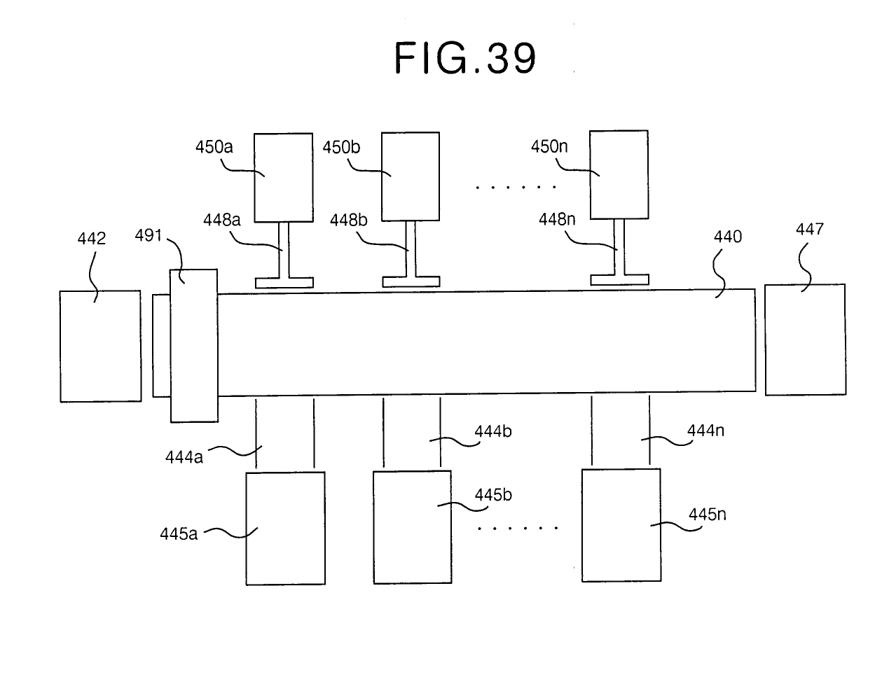

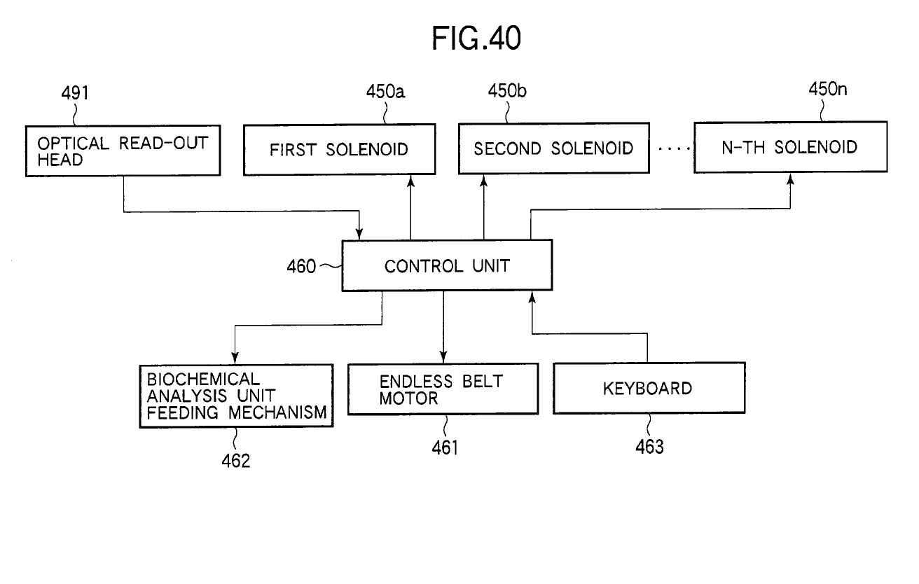

FIG. 39 is a schematic plan view showing a biochemical analysis unit sorting apparatus, which constitutes the second embodiment of the system for managing a biochemical analysis unit in accordance with the present invention. FIG. 40 is a block diagram showing a control system, a detecting system, a driving system, and an input system of the biochemical analysis unit sorting apparatus, which constitutes the second embodiment of the system for managing a biochemical analysis unit in accordance with the present invention.

As illustrated in FIGS. 39 and 40, the biochemical analysis unit sorting apparatus, which constitutes the second embodiment of the system for managing a biochemical analysis unit in accordance with the present invention, has basically the same constitution as the constitution of the biochemical analysis unit sorting apparatus shown in FIGS. 33 and 34, except that an optical read-out head 491 is utilized in lieu of the read-out head 441 for reading out the magnetic data from the magnetic recording layer 405 of the base plate 2 of the biochemical analysis unit 401. The optical read-out head 491 reads out the data concerning the date and hour of execution of the hybridization, the radioactive label data, and the data concerning the nuclide of the radioactive labeling substance from the adsorptive plate 472 of the biochemical analysis unit 471.

The biochemical analysis unit sorting apparatus operates basically in the same manner as that in the biochemical analysis unit sorting apparatus shown in FIGS. 33 and 34, except that, in lieu of the magnetic data being read out from the magnetic recording layer 405 of the base plate 2 of the biochemical analysis unit 401 by the read-out head 441, the data concerning the date and hour of execution of the hybridization, the radioactive label data, and the data concerning the nuclide of the radioactive labeling substance are read out from the adsorptive plate 472 of the biochemical analysis unit 471 by the optical read-out head 491. In this manner, in accordance with the scrapping period of the biochemical analysis unit 471, at which the level of the radioactivity of the radioactive labeling substance contained in the spot-shaped region 474 of the biochemical analysis unit 471 attenuates to the level equal to at most the level that allows the biochemical analysis unit 471 to be scrapped, the biochemical analysis unit 471 is sorted and is collected into the biochemical analysis unit collecting box 445a, 445b, . . . , or 445n.

In this embodiment, with the printing head 488 of the hybridization apparatus 480, the pieces of data are printed on the adsorptive plate 472 of the biochemical analysis unit 471. The pieces of data include the data concerning the date and hour of execution of the hybridization of the biochemical analysis unit 471, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. In cases where it has been judged, in accordance with the data concerning the number of times of use for the hybridization, that the biochemical analysis unit 471 has been subjected N number of times to the hybridization, the biochemical analysis unit sorting apparatus calculates the scrapping period of the biochemical analysis unit 471, at which the level of the radioactivity of the radioactive labeling substance contained in the spot-shaped region 474 of the biochemical analysis unit 471 attenuates to the level equal to at most the level that allows the biochemical analysis unit 471 to be scrapped. The calculation is made in accordance with the data concerning the date and hour of execution of the hybridization, the radioactive label data, and the data concerning the nuclide of the radioactive labeling substance, which data have been printed on the adsorptive plate 472 of the biochemical analysis unit 471 and have been read out by the optical read-out head 491 of the biochemical analysis unit sorting apparatus. The biochemical analysis unit 471 is sorted in accordance with the calculated scrapping period, stored, and managed. Therefore, the biochemical analysis unit 471 is capable of being stored and managed reliably until the scrapping period of the biochemical analysis unit 471, at which the level of the radioactivity of the radioactive labeling substance contained in the spot-shaped region 474 of the biochemical analysis unit 471 attenuates to the level equal to at most the level that allows the biochemical analysis unit 471 to be scrapped. Also, the biochemical analysis unit 471 is capable of being scrapped after the level of the radioactivity of the radioactive labeling substance contained in the spot-shaped region 474 of the biochemical analysis unit 471 has attenuated to the level equal to at most the level that allows the biochemical analysis unit 471 to be scrapped.

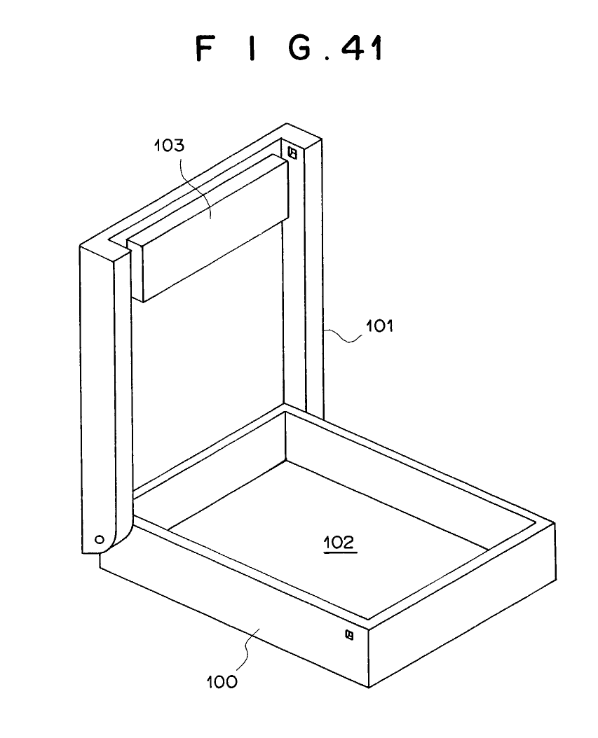

FIG. 41 is a schematic perspective view showing an exposure apparatus for executing an exposure operation, wherein a stimulable phosphor contained in at least one stimulable phosphor layer region 92 among the plurality of the dot-shaped stimulable phosphor layer regions 92, 92, . . . of the stimulable phosphor sheet 470 shown in FIG. 30 is exposed to the radiation radiated out from the radioactive labeling substance, which is contained selectively in at least one adsorptive region 4 among the plurality of the adsorptive regions 4, 4, . . . of the biochemical analysis unit 401.

As illustrated in FIG. 41, the exposure apparatus for the stimulable phosphor sheet 470 has the same constitution as that of the exposure apparatus for the stimulable phosphor sheet 90 shown in FIGS. 12 and 13, except that the two position matching pins 104a and 104b are not provided.

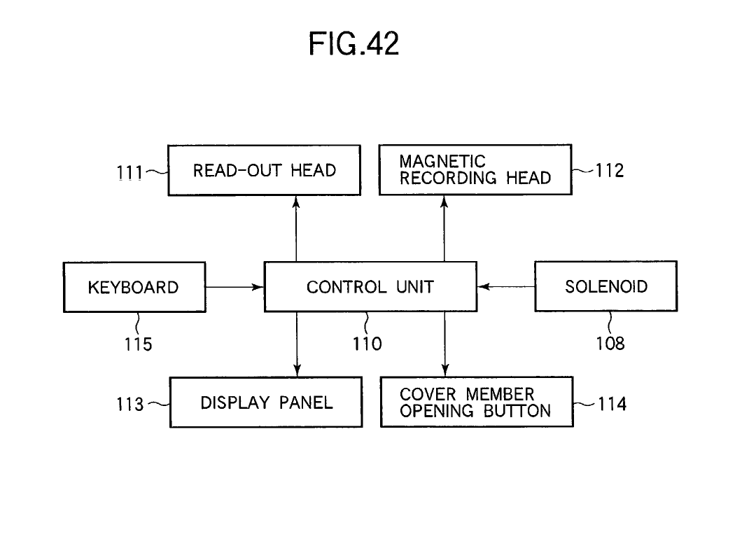

FIG. 42 is a block diagram showing a control system, a detecting system, a driving system, a displaying system, and an input system of the exposure apparatus of FIG. 41 for the stimulable phosphor sheet.

As illustrated in FIG. 42, the control system of the exposure apparatus for the stimulable phosphor sheet comprises the control unit 110 for controlling the entire exposure apparatus. The detecting system of the exposure apparatus for the stimulable phosphor sheet comprises the read-out head 111 of the data read-out and recording section 103. The read-out head 111 reads out the data having been recorded on the magnetic recording layer 405 of the biochemical analysis unit 401. The read-out signal obtained from the read-out head 111 is fed into the control unit 110.

Also, as illustrated in FIG. 42, the driving system of the exposure apparatus for the stimulable phosphor sheet comprises the magnetic recording head 112 of the data read-out and recording section 103. The magnetic recording head 112 writes the data on the magnetic recording layer 405 of the biochemical analysis unit 401. The driving system also comprises the solenoid 108 for releasing the cover member locking mechanism. The displaying system of the exposure apparatus for the stimulable phosphor sheet comprises the display panel 113 constituted of the liquid crystal panel, or the like.

Further, as illustrated in FIG. 42, the input system of the exposure apparatus for the stimulable phosphor sheet comprises a keyboard 115 and the cover member opening button 114.

When at least one dot-shaped stimulable phosphor layer region 92 among the plurality of the dot-shaped stimulable phosphor layer regions 92, 92, . . . of the stimulable phosphor sheet 470 is to be exposed to the radiation radiated out from the radioactive labeling substance, which is contained selectively in at least one dot-shaped adsorptive region 4 among the plurality of the dot-shaped adsorptive regions 4, 4, . . . of the biochemical analysis unit 401, the data concerning the nuclide of the radioactive labeling substance used is firstly inputted from the keyboard 115, and the cover member opening button 114 is then operated.

When the cover member opening button 114 is operated, the cover member opening signal is fed into the control unit 110. The control unit 110 receives the cover member opening signal and feeds the driving signal into the solenoid 108.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

When the cover member 101 is opened, the biochemical analysis unit 401 is set on the base 102 of the exposure apparatus by the user. Thereafter, the cover member 101 is closed.

In this embodiment, instead of the magnetic data being written on the magnetic recording layer 405 of the base plate 2 of the biochemical analysis unit 401 by the hybridization apparatus 430, the magnetic data is written on the magnetic recording layer 405 of the base plate 2 of the biochemical analysis unit 401 by the exposure apparatus. Therefore, the data read-out and recording section 103 is secured to the cover member 101 such that, when the cover member 101 has been closed, the data read-out and recording section 103 stands facing the magnetic recording layer 405 of the base plate 2 of the biochemical analysis unit 401 having been set on the base 102. Accordingly, the magnetic data having been recorded on the magnetic recording layer 405 of the base plate 2 of the biochemical analysis unit 401 is capable of being read out by the read-out head 111, and the magnetic data is capable of being written on the magnetic recording layer 405 of the base plate 2 of the biochemical analysis unit 401 by the magnetic recording head 112.

When the cover member 101 has been closed, if necessary, the read-out head 111 of the data read-out and recording section 103 read out the ID data, which has been recorded on the magnetic recording layer 405 of the base plate 2 of the biochemical analysis unit 401. The thus obtained detection signal is fed into the control unit 110.

Thereafter, the control unit 110 forms the data concerning the date and hour of execution of the exposure operation and the radioactive label data in accordance with the built-in clock and feeds the thus formed data to the magnetic recording head 112 in order to write the data on the magnetic recording layer 405 of the base plate 2 of the biochemical analysis unit 401. Also, the data concerning the nuclide of the radioactive labeling substance, which data has been inputted from the keyboard 115, is written on the magnetic recording layer 405.

When the writing of the magnetic data is finished, the cover member 101 is opened. The stimulable phosphor sheet 470 is then superposed upon the biochemical analysis unit 401, and the cover member 101 is closed.

In this manner, the biochemical analysis unit 401 and the stimulable phosphor sheet 470 are superposed one upon the other for a predetermined length of time. As a result, the stimulable phosphor contained in at least one dot-shaped stimulable phosphor layer region 92 among the plurality of the dot-shaped stimulable phosphor layer regions 92, 92, . . . of the stimulable phosphor sheet 470 is exposed to the radiation radiated out from the radioactive labeling substance, which is contained selectively in at least one adsorptive region 4 among the plurality of the adsorptive regions 4, 4, . . . of the biochemical analysis unit 401.

In the manner described above, the radiation information is recorded on the at least one dot-shaped stimulable phosphor layer region 92 of the stimulable phosphor sheet 470.

Thereafter, in the same manner as that in the first embodiment of the system for managing a biochemical analysis unit in accordance with the present invention, the radiation information having been recorded on the at least one dot-shaped stimulable phosphor layer region 92 of the stimulable phosphor sheet 470 is read out by the scanner shown in FIGS. 16 to 22 and FIG. 32. The data for a biochemical analysis is thus formed.

The biochemical analysis unit 401 is sorted and collected by the biochemical analysis unit sorting apparatus shown in FIGS. 33 and 34.

In this embodiment, in lieu of the data concerning the date and hour of execution of the hybridization, the radioactive label data, and the data concerning the nuclide of the radioactive labeling substance being read out, the data concerning the date and hour of execution of the exposure operation, the radioactive label data, and the data concerning the nuclide of the radioactive labeling substance are read out by the read-out head 441 and fed into the control unit 460.

In accordance with the data concerning the date and hour of execution of the exposure operation, the radioactive label data, and the data concerning the nuclide of the radioactive labeling substance, the control unit 460 calculates the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. In accordance with the calculated scrapping period of the biochemical analysis unit 401, the control unit 460 controls the endless belt motor 461, the first solenoid 450a, the second solenoid 450b, . . . , the and n-th solenoid 450n in order to sort and collect the biochemical analysis unit 401.

With this embodiment, the data concerning the date and hour of execution of the exposure operation, the radioactive label data, and the data concerning the nuclide of the radioactive labeling substance are recorded on the magnetic recording layer 405 of the base plate 2 of the biochemical analysis unit 401 by the magnetic recording head 112 of the exposure apparatus. Also, in accordance with the data concerning the date and hour of execution of the exposure operation, the radioactive label data, and the data concerning the nuclide of the radioactive labeling substance, the biochemical analysis unit sorting apparatus calculates the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. In accordance with the calculated scrapping period of the biochemical analysis unit 401, the biochemical analysis unit 401 is sorted, stored, and managed. Therefore, the biochemical analysis unit 401 is capable of being stored and managed reliably until the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. Also, the biochemical analysis unit 401 is capable of being scrapped after the level of the radioactivity of the radioactive labeling substance contained in the adsorptive region 4 of the biochemical analysis unit 401 has attenuated to the level equal to at most the level that allows the biochemical analysis unit 401 to be scrapped.

The system for managing a biochemical analysis unit in accordance with the present invention may be embodied in various other ways.

For example, in the embodiment shown in FIGS. 28 to 34, the data concerning the nuclide of the radioactive labeling substance is recorded on the magnetic recording layer 405 of the base plate 2 of the biochemical analysis unit 401. Also, in the embodiment shown in FIGS. 35 to 40, the data concerning the nuclide of the radioactive labeling substance is printed on the adsorptive plate 472 of the biochemical analysis unit 471. The data concerning the date and hour of execution of the hybridization and the data concerning the nuclide of the radioactive labeling substance having been recorded on the magnetic recording layer 405 of the base plate 2 of the biochemical analysis unit 401 are read out by the read-out head 441 of the biochemical analysis unit sorting apparatus, or the data concerning the date and hour of execution of the hybridization and the data concerning the nuclide of the radioactive labeling substance having been printed on the adsorptive plate 472 of the biochemical analysis unit 471 are read out by the optical read-out head 491 of the biochemical analysis unit sorting apparatus. In accordance with the read-out data, the calculation is made to find the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped. In accordance with the calculated scrapping period of the biochemical analysis unit 401, the biochemical analysis unit 401 is sorted. However, in cases where the nuclide of the radioactive labeling substance used for labeling the biochemical analysis unit is known, the data concerning the nuclide of the radioactive labeling substance need not be recorded on the magnetic recording layer 405 of the base plate 2 of the biochemical analysis unit 401 or on the adsorptive plate 472 of the biochemical analysis unit 471. In such cases, the information representing the nuclide of the radioactive labeling substance having been used for the labeling of the biochemical analysis unit 401 may be inputted from the keyboard 463 of the biochemical analysis unit sorting apparatus, the scrapping period of the biochemical analysis unit 401, at which the level of the radioactivity of the radioactive labeling substance contained in the adsorptive region 4 of the biochemical analysis unit 401 attenuates to the level equal to at most the level that allows the biochemical analysis unit 401 to be scrapped, may be calculated in accordance with the data concerning the date and hour of execution of the hybridization and the data concerning the nuclide of the radioactive labeling substance, and the biochemical analysis unit 401 may thus be sorted. Alternatively, the user may read out the data concerning the date and hour of execution of the hybridization, which data has been printed on the adsorptive plate 472 of the biochemical analysis unit 471, may calculate the scrapping period of the biochemical analysis unit 471, at which the level of the radioactivity of the radioactive labeling substance contained in the adsorptive plate 472 of the biochemical analysis unit 471 attenuates to the level equal to at most the level that allows the biochemical analysis unit 471 to be scrapped, in accordance with the data concerning the date and hour of execution of the hybridization, and the data concerning the nuclide of the radioactive labeling substance having been used for the labeling of the biochemical analysis unit 471, and may thus sort the biochemical analysis unit 471.

In the embodiment shown in FIGS. 37 to 40, with the printing head 488 of the hybridization apparatus 480, the pieces of data are printed on the adsorptive plate 472 of the biochemical analysis unit 471 provided with the spot-shaped regions 474, 474, . . . containing the specific binding substances. The pieces of data include the data concerning the date and hour of execution of the hybridization of the biochemical analysis unit 471, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. The data concerning the date and hour of execution of the hybridization, the radioactive label data, and the data concerning the nuclide of the radioactive labeling substance, which data have been printed on the adsorptive plate 472 of the biochemical analysis unit 471, are read out by the optical read-out head 491 of the biochemical analysis unit sorting apparatus. In accordance with the read-out data, the scrapping period of the biochemical analysis unit 471, at which the level of the radioactivity of the radioactive labeling substance contained in the spot-shaped region 474 of the biochemical analysis unit 471 attenuates to the level equal to at most the level that allows the biochemical analysis unit 471 to be scrapped, is calculated. The biochemical analysis unit 471 is sorted in accordance with the calculated scrapping period, stored, and managed. Alternatively, the data concerning the date and hour of execution of the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance may be printed on the base plate 2 of the biochemical analysis unit 401 shown in FIG. 28, which is provided with the adsorptive regions 4, 4, . . . formed at a high density in the through-holes 3, 3, . . . having the approximately circular shape. Also, the data concerning the date and hour of execution of the hybridization, the radioactive label data, and the data concerning the nuclide of the radioactive labeling substance, which data have been printed on the base plate 2 of the biochemical analysis unit 401, may be read out with the optical read-out head 491 of the biochemical analysis unit sorting apparatus, and the biochemical analysis unit 401 may be sorted, stored, and managed in accordance with the read-out data.

Also, in the embodiment shown in FIGS. 37 to 40, with the printing head 488 of the hybridization apparatus 480, the pieces of data are printed on the adsorptive plate 472 of the biochemical analysis unit 471 provided with the spot-shaped regions 474, 474, . . . containing the specific binding substances. The pieces of data include the data concerning the date and hour of execution of the hybridization of the biochemical analysis unit 471, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. The data concerning the date and hour of execution of the hybridization, the radioactive label data, and the data concerning the nuclide of the radioactive labeling substance, which data have been printed on the adsorptive plate 472 of the biochemical analysis unit 471, are read out by the optical read-out head 491 of the biochemical analysis unit sorting apparatus. In accordance with the read-out data, the scrapping period of the biochemical analysis unit 471, at which the level of the radioactivity of the radioactive labeling substance contained in the spot-shaped region 474 of the biochemical analysis unit 471 attenuates to the level equal to at most the level that allows the biochemical analysis unit 471 to be scrapped, is calculated. The biochemical analysis unit 471 is sorted, stored, and managed in accordance with the calculated scrapping period. Alternatively, instead of the biochemical analysis unit sorting apparatus being used, the user may sort, store, and manage the biochemical analysis unit 471 in accordance with the data concerning the date and hour of execution of the hybridization, the radioactive label data, and the data concerning the nuclide of the radioactive labeling substance, which data have been printed on the adsorptive plate 472 of the biochemical analysis unit 471.

Further, in the embodiment shown in FIGS. 37 to 40, with the printing head 488 of the hybridization apparatus 480, the pieces of data are printed on the adsorptive plate 472 of the biochemical analysis unit 471 provided with the spot-shaped regions 474, 474, . . . containing the specific binding substances. The pieces of data include the data concerning the date and hour of execution of the hybridization of the biochemical analysis unit 471, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. Alternatively, the pieces of data described above may be recorded on the adsorptive plate 472 of the biochemical analysis unit 471 as visible data and with a technique other than the printing, such as marking.

In the embodiment shown in FIGS. 28 to 34, the base plate 2 of the biochemical analysis unit 401 is provided with the magnetic recording layer 405, and the ID data, the data concerning the date and hour of execution of the hybridization, the data representing whether the radioactive labeling substance has or has not been used, and the data concerning the nuclide of the radioactive labeling substance are recorded on the magnetic recording layer 405. Alternatively, in lieu of the magnetic recording layer 405, an optical recording layer may be provided, and the pieces of data described above, and the like, may be recorded on the optical recording layer. As another alternative, a data recording layer, on which the data is capable of being recorded with a recording technique other than the magnetic recording and the optical recording, may be employed.

In the embodiment shown in FIGS. 28 to 34, the data concerning the number of times of use for the hybridization is magnetically recorded on the magnetic recording layer 405 of the base plate 2 of the biochemical analysis unit 401. Also, in the embodiment shown in FIGS. 35 to 40, the data concerning the number of times of use for the hybridization is printed on the adsorptive plate 472 of the biochemical analysis unit 471. However, the data concerning the number of times of use for the hybridization need not necessarily be magnetically recorded on the magnetic recording layer 405 of the base plate 2 of the biochemical analysis unit 401 or printed on the adsorptive plate 472 of the biochemical analysis unit 471, and the number of times of use for the hybridization may be detected with other means.

Also, in the embodiment shown in FIGS. 28 to 34, the data concerning the number of times of use for the hybridization is magnetically recorded on the magnetic recording layer 405 of the base plate 2 of the biochemical analysis unit 401. Also, in the embodiment shown in FIGS. 35 to 40, the data concerning the number of times of use for the hybridization is printed on the adsorptive plate 472 of the biochemical analysis unit 471. In cases where it has been judged, in accordance with the data concerning the number of times of use for the hybridization, that the biochemical analysis unit 401 or 471 has been used N number of times for the hybridization, the hybridization with the biochemical analysis unit 401 or 471 cannot be executed. However, the number of times of use of the biochemical analysis unit 401 or 471 for the hybridization need not necessarily be limited.

In the embodiments of the system for managing a biochemical analysis unit in accordance with the present invention, the biochemical analysis unit 401 is accommodated in the cartridge 31 shown in FIG. 9. Also, by use of the hybridization apparatus 430 shown in FIGS. 29 and 10 or the hybridization apparatus 480 shown in FIGS. 37 and 38, the organism-originating substance having been labeled with the radioactive labeling substance and the organism-originating substance having been labeled with the fluorescent labeling substance are subjected to the selective hybridization with the specific binding substances contained in the adsorptive regions 4, 4, . . . of the biochemical analysis unit 401 or the spot-shaped regions 474, 474, . . . of the biochemical analysis unit 471. Alternatively, the selective hybridization may be executed with one of other hybridization apparatuses, which are capable of recording the data concerning the date and hour of execution of the hybridization, the radioactive label data, and the data concerning the nuclide of the radioactive labeling substance on the biochemical analysis unit 401 or 471.

Also, in the embodiments of the system for managing a biochemical analysis unit in accordance with the present invention, the organism-originating substance having been labeled with the radioactive labeling substance and the organism-originating substance having been labeled with the fluorescent labeling substance are subjected to the selective hybridization with the specific binding substances contained in the adsorptive regions 4, 4, . . . of the biochemical analysis unit 401 or the spot-shaped regions 474, 474, . . . of the biochemical analysis unit 471. However, the selective hybridization need not necessarily be executed in this manner, and it is sufficient for the organism-originating substance having been labeled with at least the radioactive labeling substance to be subjected to the selective hybridization. Besides the organism-originating substance having been labeled with the fluorescent labeling substance, or in lieu of the organism-originating substance having been labeled with the fluorescent labeling substance, an organism-originating substance having been labeled with the labeling substance capable of producing the chemiluminescence when being brought into contact with a chemiluminescence substrate may be subjected to the selective hybridization. Alternatively, only the organism-originating substance having been labeled with the radioactive labeling substance may be subjected to the selective hybridization.

In the embodiment shown in FIGS. 28 to 34, when the hybridization is executed, the data concerning the date and hour of execution of the hybridization is recoded on the magnetic recording layer 405 of the base plate 2 of the biochemical analysis unit 401. Alternatively, when the hybridization is executed, the data concerning the date and hour of execution of the hybridization may be overwritten on the magnetic recording layer 405 of the base plate 2 of the biochemical analysis unit 401.

In the embodiment shown in FIGS. 28 to 34, when the hybridization is executed, the data concerning the date and hour of execution of the hybridization is recoded on the magnetic recording layer 405 of the base plate 2 of the biochemical analysis unit 401. Also, in the embodiment shown in FIGS. 35 to 40, when the hybridization is executed, the data concerning the date and hour of execution of the hybridization is printed on the adsorptive plate 472 of the biochemical analysis unit 471. Alternatively, when the hybridization is executed, the data concerning the day of execution of the hybridization may be recorded on the magnetic recording layer 405 of the base plate 2 of the biochemical analysis unit 401 or may be printed on the adsorptive plate 472 of the biochemical analysis unit 471. Also, the biochemical analysis unit 401 or 471 may be managed in accordance with the data concerning the day of execution of the hybridization.

In the embodiment shown in FIGS. 28 to 34, before the hybridization is executed, the data concerning the date and hour of execution of the hybridization, the radioactive label data, and the data concerning the nuclide of the radioactive labeling substance are recoded on the magnetic recording layer 405 of the base plate 2 of the biochemical analysis unit 401 by use of the hybridization apparatus 430. Also, in the embodiment shown in FIGS. 35 to 40, before the hybridization is executed, the data concerning the date and hour of execution of the hybridization, the radioactive label data, and the data concerning the nuclide of the radioactive labeling substance are printed on the adsorptive plate 472 of the biochemical analysis unit 471 by use of the hybridization apparatus 480. Alternatively, the recording or the printing of the data described above may be performed after the hybridization is executed.

In the embodiment shown in FIGS. 41 and 42, by use of the exposure apparatus, the exposure operation is executed for the stimulable phosphor sheet 470, and the data concerning the date and hour of execution of the exposure operation, the radioactive label data, and the data concerning the nuclide of the radioactive labeling substance are recorded on the magnetic recording layer 405 of the base plate 2 of the biochemical analysis unit 401. Alternatively, one of other exposure apparatuses may be employed, which are capable of recording the data described above on the magnetic recording layer 405 of the base plate 2 of the biochemical analysis unit 401.

Also, in the embodiment shown in FIGS. 41 and 42, by use of the magnetic recording head 112 of the exposure apparatus, the data concerning the date and hour of execution of the exposure operation, the radioactive label data, and the data concerning the nuclide of the radioactive labeling substance are recorded on the magnetic recording layer 405 of the base plate 2 of the biochemical analysis unit 401. Alternatively, in lieu of the exposure apparatus provided with the magnetic recording head 112, an exposure apparatus provided with an optical recording head may be employed. Also, the exposure operation may be performed with respect to the stimulable phosphor sheet 470 by use of the biochemical analysis unit 471 shown in FIG. 35 in lieu of the biochemical analysis unit 401 shown in FIG. 28. Further, the data concerning the date and hour of execution of the exposure operation, the radioactive label data, and the data concerning the nuclide of the radioactive labeling substance may be printed on the adsorptive plate 472 of the biochemical analysis unit 471, and the biochemical analysis unit 471 may be sorted and collected by use of the biochemical analysis unit sorting apparatus shown in FIGS. 39 and 40.

Further, in the embodiment shown in FIGS. 41 and 42, before the exposure operation is executed, the data concerning the date and hour of execution of the exposure operation, the radioactive label data, and the data concerning the nuclide of the radioactive labeling substance are recorded on the magnetic recording layer 405 of the base plate 2 of the biochemical analysis unit 401 by use of the exposure apparatus. Alternatively, after the exposure operation has been performed, the data described above may be recorded on the magnetic recording layer 405 of the base plate 2 of the biochemical analysis unit 401 by use of the exposure apparatus.

In the embodiment shown in FIGS. 28 to 34, the data concerning the date and hour of execution of the hybridization, the radioactive label data, and the data concerning the nuclide of the radioactive labeling substance are recoded on the magnetic recording layer 405 of the base plate 2 of the biochemical analysis unit 401 by use of the hybridization apparatus 430. The biochemical analysis unit 401 is sorted and collected by the biochemical analysis unit sorting apparatus shown in FIGS. 33 and 34 and in accordance with the data having been recorded on the magnetic recording layer 405. Also, in the embodiment shown in FIGS. 35 to 40, the data concerning the date and hour of execution of the hybridization, the radioactive label data, and the data concerning the nuclide of the radioactive labeling substance are printed on the adsorptive plate 472 of the biochemical analysis unit 471 by use of the hybridization apparatus 480. The biochemical analysis unit 471 is sorted and collected by the biochemical analysis unit sorting apparatus shown in FIGS. 39 and 40 and in accordance with the data having been printed on the adsorptive plate 472 of the biochemical analysis unit 471. Alternatively, in lieu of the data concerning the date and hour of execution of the hybridization, the data concerning the date and hour, or the day, of formation of the radioactive labeling substance may be inputted from the keyboard 80 of the hybridization apparatus 430 and may be recorded on the magnetic recording layer 405 of the base plate 2 of the biochemical analysis unit 401. The biochemical analysis unit 401 may then be sorted and collected by the biochemical analysis unit sorting apparatus shown in FIGS. 33 and 34 and in accordance with the data concerning the date and hour, or the day, of formation of the radioactive labeling substance, the radioactive label data, and the data concerning the nuclide of the radioactive labeling substance, which data have been recorded on the magnetic recording layer 405. Also, in lieu of the data concerning the date and hour of execution of the hybridization, the data concerning the date and hour, or the day, of formation of the radioactive labeling substance may be inputted from the keyboard 80 of the hybridization apparatus 480 and may be printed on the adsorptive plate 472 of the biochemical analysis unit 471. The biochemical analysis unit 471 may then be sorted and collected by the biochemical analysis unit sorting apparatus shown in FIGS. 39 and 40 and in accordance with the data concerning the date and hour, or the day, of formation of the radioactive labeling substance, the radioactive label data, and the data concerning the nuclide of the radioactive labeling substance, which data have been printed on the adsorptive plate 472 of the biochemical analysis unit 471.

In the embodiment shown in FIGS. 41 and 42, by use of the exposure apparatus, by use of the exposure apparatus, the data concerning the date and hour of execution of the exposure operation, the radioactive label data, and the data concerning the nuclide of the radioactive labeling substance are recorded on the magnetic recording layer 405 of the base plate 2 of the biochemical analysis unit 401. The biochemical analysis unit 401 is sorted and collected by the biochemical analysis unit sorting apparatus shown in FIGS. 33 and 34 and in accordance with the data having thus been recorded on the magnetic recording layer 405. Alternatively, in lieu of the data concerning the date and hour of execution of the hybridization or the data concerning the date and hour of execution of the exposure operation, the data concerning the date and hour, or the day, of formation of the radioactive labeling substance may be inputted from the keyboard 115 of the exposure apparatus and may be recorded on the magnetic recording layer 405 of the base plate 2 of the biochemical analysis unit 401. The biochemical analysis unit 401 may then be sorted and collected by the biochemical analysis unit sorting apparatus shown in FIGS. 33 and 34 and in accordance with the concerning the date and hour, or the day, of formation of the radioactive labeling substance, the radioactive label data, and the data concerning the nuclide of the radioactive labeling substance, which data have been recorded on the magnetic recording layer 405.

In the embodiment shown in FIGS. 28 to 34, each of the adsorptive regions 4, 4, . . . need not necessarily have the approximately circular shape and may have one of other shapes, such as a rectangular shape. Also, in the embodiment shown in FIGS. 35 to 40, each of the spot-shaped regions 474, 474, . . . need not necessarily have the approximately circular shape and may have one of other shapes, such as a rectangular shape.

In the embodiment shown in FIGS. 28 to 34 and in the embodiment shown in FIGS. 35 to 40, the number and the size of the adsorptive regions 4, 4, . . . or the spot-shaped regions 474, 474, . . . may be set arbitrarily in accordance with the purposes of use. However, at least 10 adsorptive regions 4, 4, . . . or spot-shaped regions 474, 474, . . . , each of which has a size smaller than 5 mm², should preferably be formed at a density of at least 10 regions/cm² in the base plate 2 or the adsorptive plate 472. Also, the adsorptive regions 4, 4, . . . or the spot-shaped regions 474, 474, . . . need not necessarily be located in the regular pattern.

Embodiments of the system for managing a biochemical analysis medium in accordance with the present invention will be described hereinbelow.

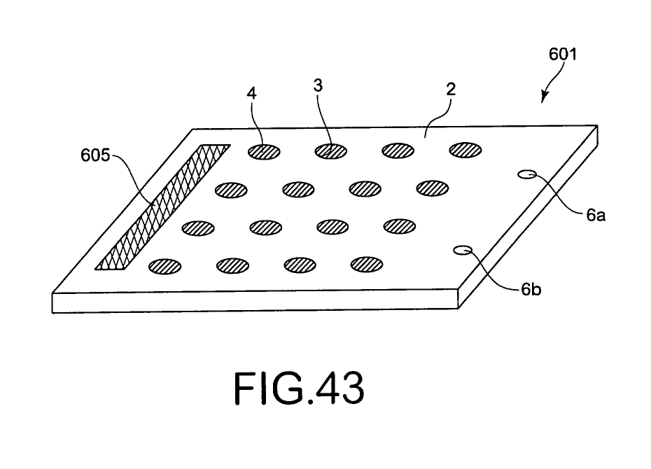

FIG. 43 is a schematic perspective view showing a biochemical analysis unit, which is managed by a first embodiment of the system for managing a biochemical analysis medium in accordance with the present invention.

In FIG. 43, similar elements are numbered with the same reference numerals with respect to FIG. 28. As illustrated in FIG. 43, a biochemical analysis unit 601, which is to be managed by the first embodiment of the system for managing a biochemical analysis medium in accordance with the present invention, is constituted basically in the same manner as that in the biochemical analysis unit 401 shown in FIG. 28, except that the base plate 2 is provided with a magnetic recording layer 605 in lieu of the magnetic recording layer 405 and is provided with the two circular position matching through-holes 6a and 6b.

When the biochemical analysis unit 601 is delivered, the ID data inherent to the biochemical analysis unit 601 is written on the magnetic recording layer 605. Also, when the biochemical analysis unit 601 is utilized for the hybridization, pieces of data concerning the hybridization are written on the magnetic recording layer 605 by the hybridization apparatus, which will be described later. The pieces of data include the data concerning the date and hour of execution of the hybridization, the data concerning the number of times of use for the hybridization, a radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance.

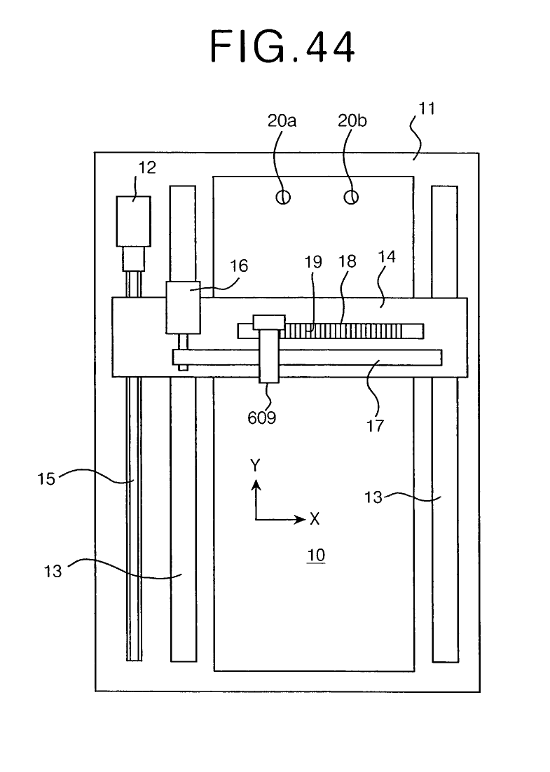

FIG. 44 is a schematic front view showing a spotting apparatus.

As illustrated in FIG. 44, the spotting apparatus comprises a spotting head 609. The spotting head 609 is provided with an injector for injecting a liquid containing a specific binding substance toward the biochemical analysis unit 601. The spotting head 609 is capable of being moved by a driving mechanism in the main scanning direction indicated by the arrow X in FIG. 44 and in the sub-scanning direction indicated by the arrow Y in FIG. 44.

The driving mechanism of the spotting apparatus is mounted on the frame 11 secured to the base 10, on which the biochemical analysis unit 601 to be spotted with the specific binding substances is placed.

As illustrated in FIG. 44, the sub-scanning pulse motor 12 and the pair of the rails 13, 13 are secured to the frame 11. Also, the base 14 is located on the frame 11. The base 14 is capable of moving in the sub-scanning direction, which is indicated by the arrow Y in FIG. 44, along the pair of the rails 13, 13.

The movable base 14 has a threaded hole (not shown). The threaded rod 15, which is rotated by the sub-scanning pulse motor 12, is engaged with the threaded hole of the base 14.

The main scanning pulse motor 16 is secured to the movable base 14. The main scanning pulse motor 16 is capable of intermittently driving the endless belt 17 at a predetermined pitch.

The spotting head 609 of the spotting apparatus is secured to the endless belt 17. When the endless belt 17 is driven by the main scanning pulse motor 16, the spotting head 9 is moved by the endless belt 17 in the main scanning direction, which is indicated by the arrow X in FIG. 44.

In FIG. 44, reference numeral 18 represents the linear encoder for detecting the position of the spotting head 609 with respect to the main scanning direction. Also, reference numeral 19 represents slits of the linear encoder 18.

As illustrated in FIG. 44, the two position matching pins 20a and 20b protrude from the base 10 of the spotting apparatus. The position matching pins 20a and 20b are located at the positions corresponding respectively to the two position matching through-holes 6a and 6b of the base plate 2 of the biochemical analysis unit 601. The biochemical analysis unit 601 is placed on the base 10 of the spotting apparatus, such that the two position matching through-holes 6a and 6b of the base plate 2 of the biochemical analysis unit 601 fit respectively onto the two position matching pins 20a and 20b of the base 10 of the spotting apparatus. In this manner, the biochemical analysis unit 601 is capable of being placed reliably at approximately the same position on the base 10 of the spotting apparatus.

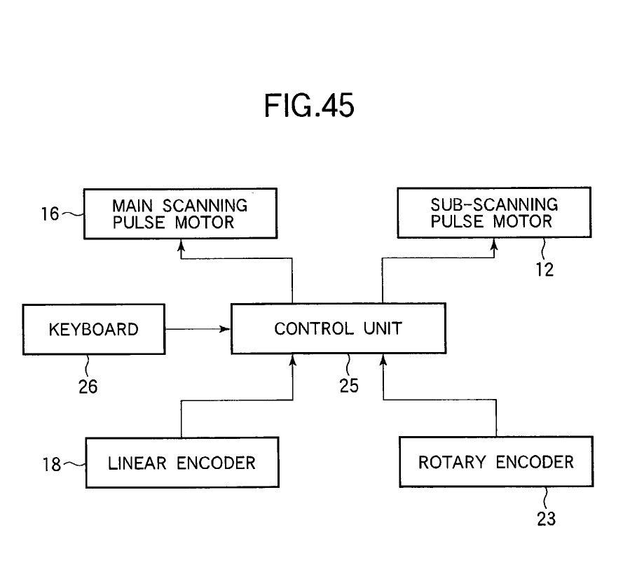

FIG. 45 is a block diagram showing a control system, an input system, a driving system, and a detecting system of the spotting apparatus shown in FIG. 44.

As illustrated in FIG. 45, the control system of the spotting apparatus comprises the control unit 25 for controlling the operations of the spotting apparatus. The input system of the spotting apparatus comprises the keyboard 26.

Also, the driving system of the spotting apparatus comprises the main scanning pulse motor 16 and the sub-scanning pulse motor 12. The detecting system of the spotting apparatus comprises the linear encoder 18 for detecting the position of the spotting head 609 with respect to the main scanning direction, and the rotary encoder 23 for detecting the amount of rotation of the rod 15.

With the spotting apparatus constituted in the manner described above, the specific binding substances, such as the cDNA's, are spotted respectively onto the plurality of the adsorptive regions 4, 4, . . . of the biochemical analysis unit 601.

Firstly, the biochemical analysis unit 601 is placed on the base 10 of the spotting apparatus, such that the two position matching through-holes 6a and 6b of the biochemical analysis unit 601 fit respectively onto the two position matching pins 20a and 20b of the base 10 of the spotting apparatus.

Thereafter, spotting data concerning the positions of the adsorptive regions 4, 4, . . . of the base plate 2 of the biochemical analysis unit 601 is inputted from the keyboard 26.

In this embodiment, approximately 10,000 adsorptive regions 4, 4, . . . , each of which has a size of approximately 0.01 mm², are formed at a density of approximately 5,000 regions/cm² on the base plate 2 of the biochemical analysis unit 601. Each of the specific binding substances is spotted onto one of the adsorptive regions 4, 4, . . . of the biochemical analysis unit 601.

The spotting data having been inputted from the keyboard 26 is fed into the control unit 25. The control unit 25 receives the spotting data and calculates the driving pulses to be given to the main scanning pulse motor 16 and the sub-scanning pulse motor 12 in order to move the spotting head 609 to the position of each of the adsorptive regions 4, 4, . . . of the base plate 2 of the biochemical analysis unit 601. The driving pulse data is stored in the memory (not shown).

In this embodiment, the plurality of the adsorptive regions 4, 4, . . . of the biochemical analysis unit 601 are located at predetermined intervals and in the regular pattern in the base plate 2. Therefore, the driving pulses to be given to the main scanning pulse motor 16 and the sub-scanning pulse motor 12 in order to move the spotting head 609 to the position, at which the spotting head 609 thirdly or thereafter stands facing an adsorptive region 4 to be spotted with the specific binding substance, may be identical with the driving pulses to be given to the main scanning pulse motor 16 and the sub-scanning pulse motor 12 in order to move the spotting head 609 from the position, at which the spotting head 609 firstly stands facing an adsorptive region 4 to be spotted with the specific binding substance, to the position, at which the spotting head 609 secondly stands facing an adsorptive region 4 to be spotted with the specific binding substance. Accordingly, it is sufficient for the calculations to be made to find only the driving pulses to be given to the main scanning pulse motor 16 and the sub-scanning pulse motor 12 in order to move the spotting head 609 to the position, at which the spotting head 609 firstly stands facing an adsorptive region 4 to be spotted with the specific binding substance, and the driving pulses to be given to the main scanning pulse motor 16 and the sub-scanning pulse motor 12 in order to move the spotting head 609 from the position, at which the spotting head 609 firstly stands facing an adsorptive region 4 to be spotted with the specific binding substance, to the position, at which the spotting head 609 secondly stands facing an adsorptive region 4 to be spotted with the specific binding substance. The thus obtained driving pulse data may be stored in the memory.

In the manner described above, the driving pulses to be given to the main scanning pulse motor 16 and the sub-scanning pulse motor 12 in order to move the spotting head 609 to the position, at which the spotting head 609 stands facing each of the adsorptive regions 4, 4, . . . , are calculated, and the thus obtained driving pulse data is stored in the memory. Thereafter, the control unit 25 gives the predetermined driving pulses to the main scanning pulse motor 16 and the sub-scanning pulse motor 12 in accordance with the driving pulse data having been stored in the memory, and the spotting head 609 is intermittently moved. When the spotting head 609 arrives at the position, which stands facing the adsorptive region 4 to be spotted with the specific binding substance, the control unit 25 feeds the driving stop signal to the main scanning pulse motor 16 and the sub-scanning pulse motor 12, and the spotting head 609 is stopped. Also, the control unit 25 feeds the spotting signal to the spotting head 609 and causes the injector to inject the specific binding substance.

In cases where the spotting head 609 is to be moved to the position, at which the spotting head 609 secondly or thereafter stands facing an adsorptive region 4 to be spotted with the specific binding substance, the spotting head 609 is moved at the predetermined pitches in the main scanning direction indicated by the arrow X and in the sub-scanning direction indicated by the arrow Y.

In the same manner, the spotting head 609 is moved intermittently by the main scanning pulse motor 16 and the sub-scanning pulse motor 12. Also, in accordance with the inputted spotting data, predetermined specific binding substances are spotted respectively onto the plurality of the adsorptive regions 4, 4, . . . .

In this embodiment, a hybridization apparatus having basically the same constitution as that of the hybridization apparatus 30 shown in FIG. 8 is employed. In this embodiment, the read-out head 37 shown in FIG. 8 reads out the data having been recorded on the magnetic recording layer 605 of the biochemical analysis unit 601, which is set on the first endless belt 36a. Also, in the same manner as that described above, the biochemical analysis unit 601 is loaded into the cartridge 31 shown in FIG. 9. Further, in this embodiment, the control system, the detecting system, the driving system, the input system, and the displaying system of the hybridization apparatus 30 are constituted basically in the same manner as that shown in FIG. 10.

In this embodiment, in the hybridization apparatus 30, the organism-originating substance having been labeled with the labeling substance is subjected to the selective hybridization with the specific binding substances, which have been adsorbed to the plurality of the adsorptive regions 4, 4, . . . of the base plate 2 of the biochemical analysis unit 601, in the manner described below.

Firstly, the hybridization liquid is prepared and accommodated in the hybridization liquid tank (not shown). Also, the washing liquid is prepared and accommodated in the washing liquid tank (not shown).

Further, the probe liquid, which contains the organism-originating substance having been labeled with the labeling substance, is prepared and accommodated in the probe liquid tip (not shown). In this embodiment, the probe liquid containing the organism-originating substance, which has been labeled with the radioactive labeling substance, and the organism-originating substance, which has been labeled with the fluorescent labeling substance, is prepared and accommodated in the probe liquid tip.

When the hybridization is to be executed, the biochemical analysis unit 601 comprising the plurality of the adsorptive regions 4, 4, . . . , to which the specific binding substances, such as the cDNA's, have respectively been adsorbed, is set by the user on the first endless belt 36a of the cartridge loading section 32. Also, a start signal is inputted from the keyboard 80. At the same time, in cases where the organism-originating substance, which has been labeled with the radioactive labeling substance, is to be subjected to the hybridization with the specific binding substances contained in the plurality of the adsorptive regions 4, 4, . . . of the biochemical analysis unit 601, a radioactive label signal and a nuclide specifying signal for specifying the nuclide of the radioactive labeling substance are inputted by the user from the keyboard 80. In this embodiment, wherein the probe liquid contains the organism-originating substance having been labeled with the radioactive labeling substance, the radioactive label signal and the nuclide specifying signal for specifying the nuclide of the radioactive labeling substance are inputted by the user from the keyboard 80.

The start signal, the radioactive label signal, and the nuclide specifying signal having been inputted from the keyboard 80 are fed into the control unit 60.

When the control unit 60 receives the start signal, the control unit 60 feeds a driving signal into the first motor 61. The first motor 61 rotates the pair of the pulleys 36b and 36c in order to rotate the first endless belt 36a clockwise in FIG. 8.

When the magnetic recording layer 605 of the biochemical analysis unit 601, which has been set on the first endless belt 36a, arrives at the position, which stands facing the read-out head 37, the control unit 60 feeds a driving stop signal to the first motor 61 in order to stop the first endless belt 36a. In this state, the magnetic data having been recorded on the magnetic recording layer 605 is read out by the read-out head 37.

In this embodiment, at the time of the delivery of the biochemical analysis unit 601, the ID data inherent to the biochemical analysis unit 601 has been recorded on the magnetic recording layer 605 of the biochemical analysis unit 601. Also, the data concerning the date and hour of execution of the hybridization and the data concerning the number of times of use of the biochemical analysis unit 601 for the hybridization are capable of being recorded on the magnetic recording layer 605 by the magnetic recording head 38 of the hybridization apparatus 30. In cases where the radioactive labeling substance has been used as the labeling substance at the time of the hybridization, the radioactive label data representing the use of the radioactive labeling substance and the data concerning the nuclide of the radioactive labeling substance are also capable of being recorded on the magnetic recording layer 605 by the magnetic recording head 38 of the hybridization apparatus 30.

The data having been read out by the read-out head 37 are fed into the control unit 60. In cases where it has been judged in accordance with the data concerning the number of times of use for the hybridization, which data has been received from the read-out head 37, that the biochemical analysis unit 601 has already been used N number of times, the control unit 60 feeds a reverse rotation signal into the first motor 61. The pulleys 36b and 36c are thus rotated counter-clockwise in FIG. 8, and the biochemical analysis unit 601 is sent back to the user. Also, a message instructing change-over of the biochemical analysis unit 601 is displayed on the displaying panel 81.

If the biochemical analysis unit 601 is used N number of times or more, the specific binding substances having been adsorbed to the adsorptive regions 4, 4, . . . will separate from the adsorptive regions 4, 4, . . . . As a result, the accuracy of the analysis will become markedly low, and reliable analysis results cannot be obtained. The value of N is set to be, for example, 2.

The biochemical analysis unit 601 having been returned to the user is sorted by a biochemical analysis medium sorting apparatus and in accordance with a scrapping period, as will be described later, and is managed until the scrapping period.

In cases where it has been judged in accordance with the data concerning the number of times of use for the hybridization, which data has been received from the read-out head 37, that the number of times of use of the biochemical analysis unit 601 is smaller than N, the control unit 60 further feeds the driving signal into the first motor 61, and the biochemical analysis unit 601 is moved to the position, at which the magnetic recording layer 605 stands facing the magnetic recording head 38.

When the biochemical analysis unit 601 has been moved to the position, at which the magnetic recording layer 605 stands facing the magnetic recording head 38, the driving stop signal is fed from the control unit 60 into the first motor 61.

Thereafter, the control unit 60 feeds a writing signal to the magnetic recording head 38 in order to increase the number of times of use of the biochemical analysis unit 601, which number has been recorded on the magnetic recording layer 605, by a value of 1 and to write the data concerning the date and hour of execution of the hybridization on the magnetic recording layer 605 in accordance with the built-in clock. Further, in cases where the radioactive label signal and the nuclide specifying signal have been inputted from the keyboard 80, the radioactive label data representing the use of the radioactive labeling substance and the data concerning the nuclide of the radioactive labeling substance are written on the magnetic recording layer 605.

When the data writing on the magnetic recording layer 605 is completed, the control unit 60 again feeds the driving signal to the first motor 61, and the pulleys 36b and 36c are rotated in order to convey the biochemical analysis unit 601 into the loading mechanism 39.

Thereafter, the operations ranging from the operation, in which the biochemical analysis unit 601 is conveyed into the cartridge 31, to the operation, in which the cartridge 31 is fed by the fifth endless belt 49a into the biochemical analysis unit take-out mechanism 52, are performed in the same manner as that described above with reference to FIGS. 8, 9, and 10.

In this embodiment, wherein the organism-originating substance contained in the probe liquid has been labeled with the radioactive labeling substance, in accordance with the radioactive label signal having been inputted from the keyboard 80, the radiation sensor 50 detects the concentration of the radioactive labeling substance in the washing liquid, and the washing operation with the washing liquid is repeated until the concentration of the radioactive labeling substance in the washing liquid decreases to a value at most equal to the reference concentration of the radioactive labeling substance. However, in cases where the organism-originating substance contained in the probe liquid has not been labeled with the radioactive labeling substance, the control unit 60 operates to finish the washing operation at the time at which it is judged that the injection of the washing liquid has been performed a predetermined number of times and the washing operation has thus been executed.

When the cartridge 31 has been fed into the biochemical analysis unit take-out mechanism 52, the control unit 60 feeds the driving stop signal into the fifth motor 65 in order to stop the driving of the fifth endless belt 49a. Also, the control unit 60 feeds the driving signal into the biochemical analysis unit take-out mechanism 52.

When the biochemical analysis unit take-out mechanism 52 receives the driving signal from the control unit 60, the biochemical analysis unit take-out mechanism 52 opens the cover 31b of the cartridge 31 and takes out the biochemical analysis unit 601 from the cartridge 31.

In the manner described above, the radiation information, which is formed with the radioactive labeling substance acting as the labeling substance, is recorded on at least one adsorptive region 4 among the plurality of the adsorptive regions 4, 4, . . . of the biochemical analysis unit 601. Also, the fluorescence information, which is formed with the fluorescent labeling substance, such as the fluoro chrome, is recorded on at least one adsorptive region 4 among the plurality of the adsorptive regions 4, 4, . . . of the biochemical analysis unit 601. The fluorescence information, which has been recorded on at least one adsorptive region 4 among the plurality of the adsorptive regions 4, 4, . . . , is read out by a scanner, which will be described later. In this manner, the data for a biochemical analysis is formed.

A stimulable phosphor sheet, which is managed by the first embodiment of the system for managing a biochemical analysis medium in accordance with the present invention, is constituted basically in the same manner as that in the stimulable phosphor sheet 90 shown in FIG. 11.

In this embodiment, when the stimulable phosphor sheet 90 is delivered, the ID data inherent to the stimulable phosphor sheet 90 is written on the magnetic recording layer 94 of the support 91 of the stimulable phosphor sheet 90. Also, when the exposure operation is executed with respect to the stimulable phosphor sheet 90, the data concerning the exposure operation is written on the magnetic recording layer 94 by the exposure apparatus described below. The data concerning the exposure operation includes the data concerning the date and hour of execution of the exposure operation, the data concerning the number of times of use for the exposure operation, and the data concerning the nuclide of the radioactive labeling substance.

The exposure apparatus utilized in this embodiment has basically the same constitution as that of the exposure apparatus shown in FIGS. 12 and 13.

A control system, a detecting system, a driving system, a displaying system, and an input system of the exposure apparatus employed in this embodiment will be described hereinbelow with reference to FIG. 42.

In this embodiment, as illustrated in FIG. 42, the control system of the exposure apparatus for the stimulable phosphor sheet comprises the control unit 110 for controlling the entire exposure apparatus. The detecting system of the exposure apparatus for the stimulable phosphor sheet comprises the read-out head 111 of the data read-out and recording section 103. The read-out head 111 reads out the data having been recorded on the magnetic recording layer 605 of the biochemical analysis unit 601 and the magnetic recording layer 94 of the stimulable phosphor sheet 90. A read-out signal obtained from the read-out head 111 is fed into the control unit 110.

Also, as illustrated in FIG. 42, the driving system of the exposure apparatus for the stimulable phosphor sheet comprises the magnetic recording head 112 of the data read-out and recording section 103. The magnetic recording head 112 writes the data on the magnetic recording layer 94 of the stimulable phosphor sheet 90. The driving system also comprises the solenoid 108 for releasing the cover member locking mechanism. The displaying system of the exposure apparatus for the stimulable phosphor sheet comprises the display panel 113 constituted of the liquid crystal panel, or the like.

The input system of the exposure apparatus for the stimulable phosphor sheet comprises the keyboard 115 and the cover member opening button 114.

When at least one dot-shaped stimulable phosphor layer region 92 among the plurality of the dot-shaped stimulable phosphor layer regions 92, 92, . . . of the stimulable phosphor sheet 90 is to be exposed to the radiation radiated out from the radioactive labeling substance, which is contained selectively in at least one dot-shaped adsorptive region 4 among the plurality of the dot-shaped adsorptive regions 4, 4, . . . of the biochemical analysis unit 601, the data concerning the nuclide of the radioactive labeling substance used is firstly inputted from the keyboard 115, and the cover member opening button 114 is then operated.

When the cover member opening button 114 is operated, the cover member opening signal is fed into the control unit 110. The control unit 110 receives the cover member opening signal and feeds the driving signal into the solenoid 108.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

When the cover member 101 is opened, the biochemical analysis unit 601 is set on the base 102 of the exposure apparatus by the user, such that the two position matching through-holes 6a and 6b of the base plate 2 of the biochemical analysis unit 601 may fit respectively onto the two position matching pins 104a and 104b of the base 102 of the exposure apparatus.

Thereafter, the stimulable phosphor sheet 90 is set on the surface of the biochemical analysis unit 601, which has been set on the base 102, such that the two position matching through-holes 96a and 96b of the support 91 of the stimulable phosphor sheet 90 may fit respectively onto the two position matching pins 104a and 104b of the base 102 of the exposure apparatus. The cover member 101 is then closed.

In this embodiment, the data read-out and recording section 103 is secured to the cover member 101 such that, when the stimulable phosphor sheet 90 has been set on the surface of the biochemical analysis unit 601, which has been set on the base 102, and the cover member 101 has been closed, the data read-out and recording section 103 stands facing the magnetic recording layer 94 of the support 91 of the stimulable phosphor sheet 90. Accordingly, the magnetic data having been recorded on the magnetic recording layer 94 of the support 91 of the stimulable phosphor sheet 90 is capable of being read out by the read-out head 111, and the magnetic data is capable of being written on the magnetic recording layer 94 of the support 91 of the stimulable phosphor sheet 90 by the magnetic recording head 112.

When the cover member 101 has been closed, the data having been recorded on the magnetic recording layer 94 of the support 91 of the stimulable phosphor sheet 90 is read out by the read-out head 111 of the data read-out and recording section 103. The thus obtained detection signal is fed into the control unit 110.

In cases where the stimulable phosphor sheet 90 is used repeatedly for the operation for exposing the stimulable phosphor layer regions 92, 92, . . . of the stimulable phosphor sheet 90 to the radiation radiated out from the radioactive labeling substance, irradiating the stimulating rays to the stimulable phosphor contained in the stimulable phosphor layer regions 92, 92, . . . , detecting the light emitted by the stimulable phosphor, and forming the data for a biochemical analysis, the accuracy of the analysis becomes markedly low, and reliable results of analysis cannot be obtained. Therefore, before the stimulable phosphor sheet 90 is exposed to the radiation radiated out from the radioactive labeling substance contained in at least one adsorptive region 4 among the plurality of the adsorptive regions 4, 4, . . . of the biochemical analysis unit 601 in the exposure apparatus, the data concerning the number of times of use for the exposure operation is recorded on the magnetic recording layer 94 of the stimulable phosphor sheet 90 by the magnetic recording head 112 of the data read-out and recording section 103. The data concerning the number of times of use for the exposure operation, which data has been recorded on the magnetic recording layer 94, is read out by the read-out head 111 of the data read-out and recording section 103 and fed into the control unit 110.

In cases where it has been judged, in accordance with the magnetic data having been received from the read-out head 111 of the data read-out and recording section 103, that the stimulable phosphor layer regions 92, 92, . . . of the stimulable phosphor sheet 90 have already been subjected to the exposure operation M number of times, the control unit 110 feeds the actuating signal to the solenoid 108 and feeds the displaying signal to the display panel 113.

As a result, the solenoid 108 is actuated in order to swing the hook member 105 in the counter-clockwise direction in FIG. 13 around the shaft 105a against the urging force of the compression spring 106. In this manner, the engagement of the hook member 105 with the engagement groove 107 is released, and the cover member 101 of the exposure apparatus is opened.

At the same time, the display panel 113 receives the displaying signal from the control unit 110 and displays a message indicating that the stimulable phosphor sheet 90 is the one which is not allowed to be used.

If the stimulable phosphor layer regions 92, 92, . . . of the stimulable phosphor sheet 90 are subjected to the exposure operation M number of times or more, and the data for a biochemical analysis is thereby formed, the accuracy of analysis will become markedly low, and reliable results of analysis cannot be obtained. The value of M is set to be, for example, 2.

Also, in cases where the same stimulable phosphor sheet 90 is set repeatedly in the exposure apparatus by the user, the cover member 101 is opened.

Therefore, in cases where the user sets by mistake the stimulable phosphor sheet 90, which has been subjected to the exposure operation M number of times and has thus been used for the formation of the data for a biochemical analysis, in the exposure apparatus, the cover member 101 cannot be closed. Accordingly, in such cases, the exposure operation for exposing the stimulable phosphor layer regions 92, 92, . . . of the stimulable phosphor sheet 90 to the radiation radiated out from the radioactive labeling substance contained selectively in the adsorptive regions 4, 4, . . . of the biochemical analysis unit 601 cannot be performed.

In such cases, the user takes out the stimulable phosphor sheet 90 from the exposure apparatus. The stimulable phosphor sheet 90 having been taken out from the exposure apparatus is sorted in accordance with the scrapping period by a biochemical analysis medium sorting apparatus, which will be described later. The thus sorted stimulable phosphor sheet 90 is stored and managed until the scrapping period.

In cases where it has been judged that the number of times of use for the exposure operation of the stimulable phosphor sheet 90 is smaller than M, the control unit 110 feeds the writing signal into the magnetic recording head 112 of the data read-out and recording section 103. In this manner, the number of times of use for the exposure operation of the stimulable phosphor sheet 90, which number of times has been recorded on the magnetic recording layer 94 of the support 91 of the stimulable phosphor sheet 90, is increased by 1.

At the same time, in accordance with the built-in clock, the control unit 110 forms the data concerning the date and hour of execution of the exposure operation. The thus formed data is written on the magnetic recording layer 94 of the support 91 of the stimulable phosphor sheet 90 by the magnetic recording head 112. The data concerning the nuclide of the radioactive labeling substance, which has been inputted from the keyboard 115, is also written on the magnetic recording layer 94.

In such cases, since the cover member 101 of the exposure apparatus is locked, the stimulable phosphor contained in at least one dot-shaped stimulable phosphor layer region 92 among the plurality of the dot-shaped stimulable phosphor layer regions 92, 92, . . . of the stimulable phosphor sheet 90 is exposed to the radiation radiated out from the radioactive labeling substance, which is contained selectively in at least one dot-shaped adsorptive region 4 among the plurality of the dot-shaped adsorptive regions 4, 4, . . . of the biochemical analysis unit 601.

In the manner described above, the radiation information with the radioactive labeling substance is recorded on at least one dot-shaped stimulable phosphor layer region 92 among the plurality of the dot-shaped stimulable phosphor layer regions 92, 92, . . . of the support 91 of the stimulable phosphor sheet 90.

The scanner for reading out the radiation information from the stimulable phosphor sheet 90 in order to form the data for a biochemical analysis, and reading out the fluorescence information from the biochemical analysis unit 601 in order to form the data for a biochemical analysis is constituted in the same manner as that in the scanner described above with reference to FIGS. 16 to 22.

The control system, the input system, the driving system, and the detecting system of the scanner are constituted in the same manner as that shown in FIG. 32.

As in the constitution shown in FIG. 32, basically in the same manner as that described above with reference to FIG. 23, the data for a biochemical analysis is formed from the radiation information and the fluorescence information having been recorded on the adsorptive regions 4, 4, . . . of the biochemical analysis unit 601.

In cases where the biochemical analysis unit 601 has been subjected N number of times to the hybridization of the organism-originating substance, which has been labeled with the labeling substance, with the specific binding substances contained in the adsorptive regions 4, 4, . . . , and the biochemical analysis unit 601 has thus been used for the formation of the data for a biochemical analysis, the biochemical analysis unit 601 is sorted in accordance with the scrapping period by the biochemical analysis medium sorting apparatus. Also, in cases where the stimulable phosphor sheet 90 has been subjected M number of times to the exposure operation, the stimulable phosphor sheet 90 is sorted in accordance with the scrapping period by the biochemical analysis medium sorting apparatus. The sorted biochemical analysis unit 601 and the sorted stimulable phosphor sheet 90 are stored and managed until the scrapping period.

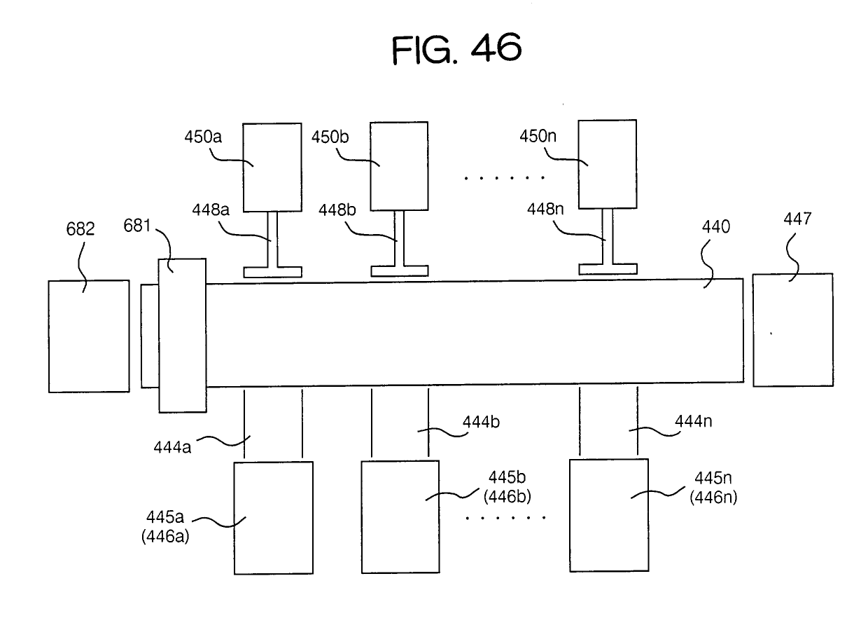

FIG. 46 is a schematic plan view showing the biochemical analysis medium sorting apparatus.

As illustrated in FIG. 46, the biochemical analysis medium sorting apparatus comprises the endless belt 440 capable of conveying the biochemical analysis unit 601 or the stimulable phosphor sheet 90, and a read-out head 681 for reading out the magnetic data having been recorded on the magnetic recording layer 605 of the base plate 2 of the biochemical analysis unit 601, which is placed on the endless belt 440, and for reading out the magnetic data having been recorded on the magnetic recording layer 94 of the support 91 of the stimulable phosphor sheet 90, which is placed on the endless belt 440. The biochemical analysis medium sorting apparatus also comprises a biochemical analysis medium loading box 682, which is capable of being loaded with biochemical analysis units 601, 601, . . . or stimulable phosphor sheets 90, 90, . . . and is provided with a biochemical analysis medium feeding mechanism 662 for feeding the biochemical analysis units 601, 601, . . . or the stimulable phosphor sheets 90, 90, . . . one after another onto the endless belt 440. The first chute 444a, the second chute 444b, . . . , and the n-th chute 444n are located along one side of the endless belt 440.

Each of the first chute 444a, the second chute 444b, . . . , and the n-th chute 444n is capable of being selectively connected to the biochemical analysis unit collecting box 445a, 445b, . . . , or 445n for collecting the biochemical analysis unit 601 in accordance with the scrapping period or to a stimulable phosphor sheet collecting box 446a, 446b, . . . , or 446n for collecting the stimulable phosphor sheet 90 in accordance with the scrapping period. The scrapping box 447 is located at the downstream end of the endless belt 440. The scrapping box 447 collects a biochemical analysis unit 601, which has been subjected N number of times to the hybridization of the organism-originating substance having been labeled with the fluorescent labeling substance and/or the labeling substance capable of producing the chemiluminescence when being brought into contact with a chemiluminescence substrate, instead of being subjected to the hybridization of the organism-originating substance having been labeled with the radioactive labeling substance.

As illustrated in FIG. 46, the first sorting member 448a, the second sorting member 448b, . . . , and the n-th sorting member 448n are located on the other side of the endless belt 440 and at positions, which stand facing the first chute 444a, the second chute 444b, . . . , and the n-th chute 444n, respectively. Each of the first sorting member 448a, the second sorting member 448b, . . . , and the n-th sorting member 448n is capable of projecting onto the endless belt 440 in order to push and feed the biochemical analysis unit 601 or the stimulable phosphor sheet 90, which is conveyed on the top surface of the endless belt 440, to the first chute 444a, the second chute 444b, . . . , and the n-th chute 444n.

The first sorting member 448a, the second sorting member 448b, . . . , and the n-th sorting member 448n are driven respectively by the first solenoid 450a, the second solenoid 450b, . . . , and the n-th solenoid 450n so as to project onto the endless belt 440.

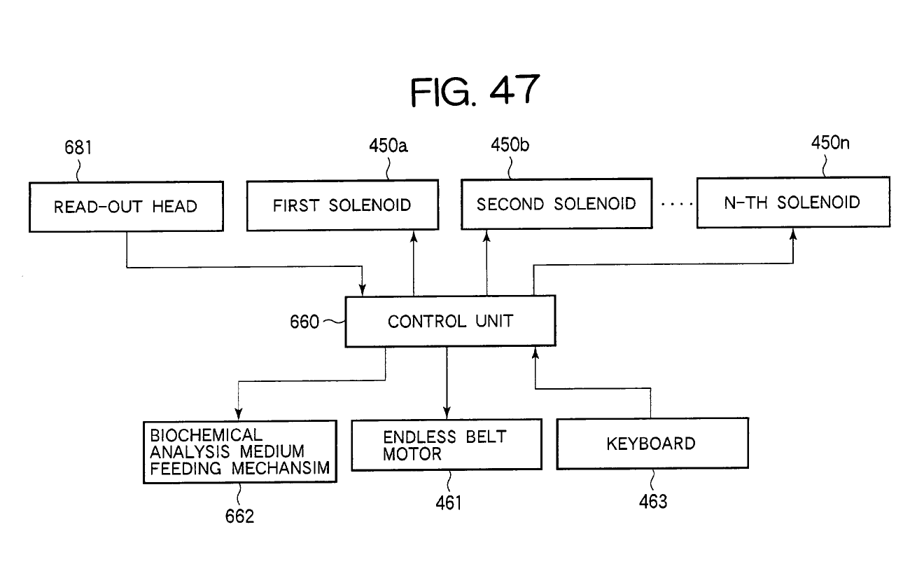

FIG. 47 is a block diagram showing a control system, a detecting system, a driving system, and an input system of the biochemical analysis medium sorting apparatus.

As illustrated in FIG. 47, the control system of the biochemical analysis medium sorting apparatus comprises a control unit 660 for controlling the operations of the entire biochemical analysis medium sorting apparatus. The detecting system comprises the read-out head 681 for reading out the magnetic data having been recorded on the magnetic recording layer 605 of the base plate 2 of the biochemical analysis unit 601, which is placed on the endless belt 440, and for reading out the magnetic data having been recorded on the magnetic recording layer 94 of the support 91 of the stimulable phosphor sheet 90, which is placed on the endless belt 440.

Also, as illustrated in FIG. 47, the driving system of the biochemical analysis medium sorting apparatus comprises the endless belt motor 461 for driving the endless belt 440, and the biochemical analysis medium feeding mechanism 662 for feeding the biochemical analysis units 601, 601, . . . or the stimulable phosphor sheets 90, 90, . . . , which have been loaded into the biochemical analysis medium loading box 682, one after another onto the endless belt 440. The driving system also comprises the first solenoid 450a for driving the first sorting member 448a, the second solenoid 450b for driving the second sorting member 448b, . . . , and the n-th solenoid 450n for driving the n-th sorting member 448n. The input system of the biochemical analysis medium sorting apparatus comprises the keyboard 463.

The biochemical analysis medium sorting apparatus operates in the manner described below in order to sort the biochemical analysis unit 601 having been used, and to collect the biochemical analysis unit 601 into the biochemical analysis unit collecting box 445a, 445b, . . . , or 445n via the first chute 444a, the second chute 444b, . . . , or the n-th chute 444n.

Firstly, the biochemical analysis unit collecting boxes 445a, 445b, . . . , and 445n are connected respectively to the first chute 444a, the second chute 444b, . . . , and the n-th chute 444n. In this embodiment, the biochemical analysis unit 601 is collected into the biochemical analysis unit collecting box 445a, 445b, . . . , or 445n in accordance with a month containing the date and hour, at which the biochemical analysis unit 601 is capable of being scrapped.

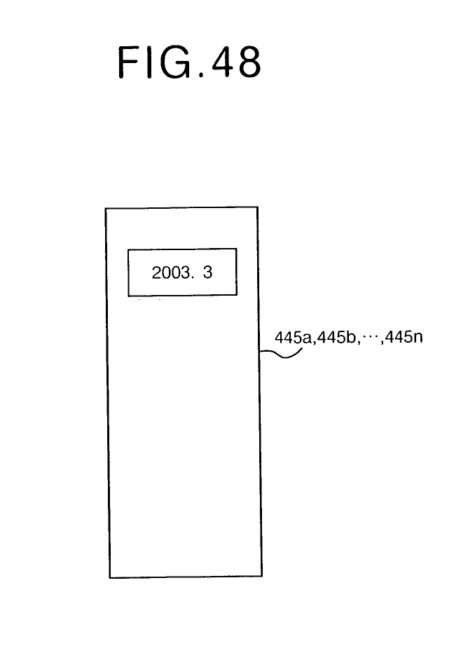

FIG. 48 is a schematic front view showing each of the biochemical analysis unit collecting boxes 445a, 445b, . . . , and 445n for collecting the biochemical analysis unit 601.

As illustrated in FIG. 48, the year and the month, at the end of which month the accommodated biochemical analysis unit 601 is capable of being scrapped, are indicated on each of the biochemical analysis unit collecting boxes 445a, 445b, . . . , and 445n.

When the biochemical analysis unit collecting boxes 445a, 445b, . . . , and 445n have been connected respectively to the first chute 444a, the second chute 444b, . . . , and the n-th chute 444n, the user inputs data concerning a year and a month of scrapping and for specifying the year and month, at which the biochemical analysis unit 601 to be collected into one of the biochemical analysis unit collecting box 445a, 445b, . . . , or 445n is capable of being scrapped, from the keyboard 463.

Thereafter, a predetermined number of the biochemical analysis units 601, 601, . . . having been used are loaded into the biochemical analysis medium loading box 682, and an operation start signal is inputted from the keyboard 463.

The operation start signal having been inputted from the keyboard 463 is fed into the control unit 660. The control unit 660 feeds a driving signal into the endless belt motor 461 in order to drive the endless belt 440. Also, the control unit 660 feeds the driving signal into the biochemical analysis medium feeding mechanism 662 in order to feed the biochemical analysis units 601, 601, . . . , which have been loaded into the biochemical analysis medium loading box 682, one after another onto the endless belt 440.

The biochemical analysis unit 601 having been fed onto the endless belt 440 is conveyed by the endless belt 440 to the read-out head 681.

When the magnetic recording layer 605 of the base plate 2 of the biochemical analysis unit 601 arrives at the position which stands facing the read-out head 681, the control unit 660 feeds a driving stop signal into the endless belt motor 461 in order to stop the endless belt 440. In this state, the magnetic data having been recorded on the magnetic recording layer 605 of the base plate 2 of the biochemical analysis unit 601 is read out by the read-out head 681.

The data having been read out by the read-out head 681 is fed into the control unit 660.

When the control unit 660 receives the magnetic data from the read-out head 681, the control unit 660 makes a judgment as to whether the radioactive label data is or is not contained in the data having been received from the read-out head 681.

In cases where it has been judged that the radioactive label data is not contained in the data having been received from the read-out head 681 and that the radioactive labeling substance has not been used at the time of the hybridization, the control unit 660 feeds the driving signal into the endless belt motor 461 in order to drive the endless belt 440 and to collect the biochemical analysis unit 601 into the scrapping box 447, which is located at the downstream end of the endless belt 440.

In cases where it has been judged that the radioactive label data is contained in the data having been received from the read-out head 681 and that the radioactive labeling substance has been used at the time of the hybridization, the control unit 660 calculates the scrapping period of the biochemical analysis unit 601, at which the level of the radioactivity of the radioactive labeling substance contained in the adsorptive region 4 of the biochemical analysis unit 601 attenuates to the level equal to at most the level that allows the biochemical analysis unit 601 to be scrapped. The calculation is made in accordance with the data concerning the date and hour of execution of the hybridization of the biochemical analysis unit 601 and the data concerning the nuclide of the radioactive labeling substance, which data have been received from the read-out head 681. In accordance with the thus calculated scrapping period of the biochemical analysis unit 601 and the data concerning the year and month of scrapping, which data is stored in the memory, the control unit 660 determines which sorting member among the first sorting member 448a, the second sorting member 448b, . . . , and the n-th sorting member 448n is to be driven. Also, the control unit 660 calculates the time of driving of the endless belt 440 required to feed the biochemical analysis unit 601 to the position which stands facing the determined sorting member.

Thereafter, the control unit 660 feeds the driving signal into the endless belt motor 461 in order to drive the endless belt 440. When the calculated time of driving of the endless belt 440 has elapsed, the control unit 660 feeds the driving stop signal into the endless belt motor 461 in order to stop the endless belt motor 461. Also, the control unit 660 feeds an actuating signal into the first solenoid 450a, the second solenoid 450b, . . . , or the n-th solenoid 450n for driving the first sorting member 448a, the second sorting member 448b, . . . , or the n-th sorting member 448n having been determined in accordance with the scrapping period of the biochemical analysis unit 601. The first sorting member 448a, the second sorting member 448b, . . . , or the n-th sorting member 448n is thus driven in order to feed the biochemical analysis unit 601 to the first chute 444a, the second chute 444b, . . . , or the n-th chute 444n, which stands facing the sorting member. The biochemical analysis unit 601 is thus collected into the biochemical analysis unit collecting box 445a, 445b, . . . , or 445n, which is connected to the chute.

In the same manner, the control unit 660 operates in order to feed the biochemical analysis units 601, 601, . . . , which have been loaded into the biochemical analysis medium loading box 682, one after another onto the endless belt 440. Also, the control unit 660 calculates the scrapping period of the biochemical analysis unit 601, at which the level of the radioactivity of the radioactive labeling substance contained in the adsorptive region 4 of the biochemical analysis unit 601 attenuates to the level equal to at most the level that allows the biochemical analysis unit 601 to be scrapped. The calculation is made in accordance with the data concerning the date and hour of execution of the hybridization of the biochemical analysis unit 601 and the data concerning the nuclide of the radioactive labeling substance, which data have been recorded on the magnetic recording layer 605 of the biochemical analysis unit 601. In accordance with the thus calculated scrapping period of the biochemical analysis unit 601 and the data concerning the year and month of scrapping, which data is stored in the memory, the control unit 660 determines the biochemical analysis unit collecting box 445a, 445b, . . . , or 445n which is to be used for collecting the biochemical analysis unit 601. Further, the control unit 660 drives the corresponding sorting member among the first sorting member 448a, the second sorting member 448b, . . . , and the n-th sorting member 448n in order to feed the biochemical analysis unit 601 into the determined biochemical analysis unit collecting box 445a, 445b, . . . , or 445n via the first chute 444a, the second chute 444b, . . . , or the n-th chute 444n. The biochemical analysis unit 601 is thus sorted and collected.

In the manner described above, all of the biochemical analysis units 601, 601, . . . having been loaded into the biochemical analysis medium loading box 682 are sorted and collected into the biochemical analysis unit collecting box 445a, 445b, . . . , or 445n or into the scrapping box 447. Thereafter, an operation end signal is fed from the keyboard 463, the driving of the endless belt motor 461 is stopped, and the sorting and collection of the stimulable phosphor sheets 601, 601, . . . are completed.

In cases where the stimulable phosphor sheet 90 having been used is to be sorted and collected, firstly, the stimulable phosphor sheet collecting boxes 446a, 446b, . . . , and 446n are connected respectively to the first chute 444a, the second chute 444b, . . . , and the n-th chute 444n. In this embodiment, the stimulable phosphor sheet 90 is collected into the stimulable phosphor sheet collecting box 446a, 446b, . . . , or 446n in accordance with a month containing the date and hour, at which the stimulable phosphor sheet 90 is capable of being scrapped.

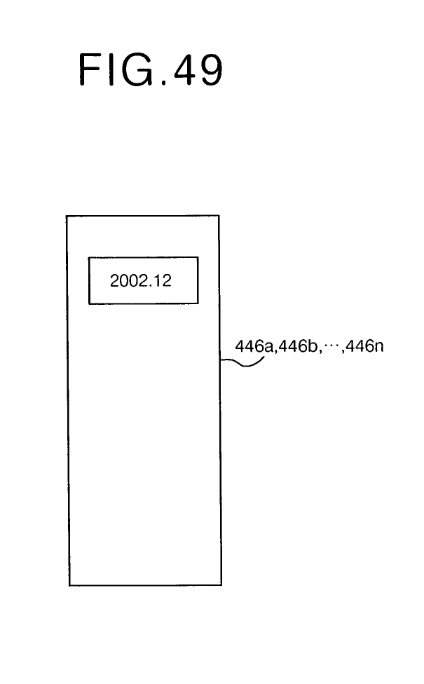

FIG. 49 is a schematic front view showing each of the stimulable phosphor sheet collecting boxes 446a, 446b, . . . , and 446n for collecting the stimulable phosphor sheet 90.

As illustrated in FIG. 49, the year and the month, at the end of which month the accommodated stimulable phosphor sheet 90 is capable of being scrapped, are indicated on each of the stimulable phosphor sheet collecting boxes 446a, 446b, . . . , and 446n.

When the stimulable phosphor sheet collecting boxes 446a, 446b, . . . , and 446n have been connected respectively to the first chute 444a, the second chute 444b, . . . , and the n-th chute 444n, the user inputs data concerning a year and a month of scrapping and for specifying the year and month, at which the stimulable phosphor sheet 90 to be collected into one of the stimulable phosphor sheet collecting box 446a, 446b, . . . , or 446n is capable of being scrapped, from the keyboard 463.

When the operation start signal is inputted from the keyboard 463, the operation start signal is fed into the control unit 660. The control unit 660 feeds a driving signal into the endless belt motor 461 in order to drive the endless belt 440. Also, the control unit 660 feeds the driving signal into the biochemical analysis medium feeding mechanism 662 in order to feed the stimulable phosphor sheets 90, 90, . . . , which have been loaded into the biochemical analysis medium loading box 682, one after another onto the endless belt 440.

The stimulable phosphor sheet 90 having been fed onto the endless belt 440 is conveyed by the endless belt 440 to the read-out head 681.

When the magnetic recording layer 94 of the support 91 of the stimulable phosphor sheet 90 arrives at the position which stands facing the read-out head 681, the control unit 660 feeds a driving stop signal into the endless belt motor 461 in order to stop the endless belt 440. In this state, the magnetic data having been recorded on the magnetic recording layer 94 of the support 91 of the stimulable phosphor sheet 90 is read out by the read-out head 681.

The data having been read out by the read-out head 681 is fed into the control unit 660.

The control unit 660 calculates the scrapping period of the stimulable phosphor sheet 90, at which the level of the radioactivity of the radioactive labeling substance contained in the stimulable phosphor layer region 92 of the stimulable phosphor sheet 90 attenuates to the level equal to at most the level that allows the stimulable phosphor sheet 90 to be scrapped. The calculation is made in accordance with the data concerning the date and hour of execution of the exposure operation for the stimulable phosphor sheet 90 and the data concerning the nuclide of the radioactive labeling substance, which data have been received from the read-out head 681. In accordance with the thus calculated scrapping period of the stimulable phosphor sheet 90 and the data concerning the year and month of scrapping, which data is stored in the memory, the control unit 660 determines which sorting member among the first sorting member 448a, the second sorting member 448b, . . . , and the n-th sorting member 448n is to be driven. Also, the control unit 660 calculates the time of driving of the endless belt 440 required to feed the stimulable phosphor sheet 90 to the position which stands facing the determined sorting member.

Thereafter, the control unit 660 feeds the driving signal into the endless belt motor 461 in order to drive the endless belt 440. When the calculated time of driving of the endless belt 440 has elapsed, the control unit 660 feeds the driving stop signal into the endless belt motor 461 in order to stop the endless belt motor 461. Also, the control unit 660 feeds an actuating signal into the first solenoid 450a, the second solenoid 450b, . . . , or the n-th solenoid 450n for driving the first sorting member 448a, the second sorting member 448b, . . . , or the n-th sorting member 448n having been determined in accordance with the scrapping period of the stimulable phosphor sheet 90. The first sorting member 448a, the second sorting member 448b, . . . , or the n-th sorting member 448n is thus driven in order to feed the stimulable phosphor sheet 90 to the first chute 444a, the second chute 444b, . . . , or the n-th chute 444n, which stands facing the sorting member. The stimulable phosphor sheet 90 is thus collected into the stimulable phosphor sheet collecting box 446a, 446b, . . . , or 446n, which is connected to the chute.

In the same manner, the control unit 660 operates in order to feed the stimulable phosphor sheets 90, 90, . . . , which have been loaded into the biochemical analysis medium loading box 682, one after another onto the endless belt 440. Also, the control unit 660 calculates the scrapping period of the stimulable phosphor sheet 90, at which the level of the radioactivity of the radioactive labeling substance contained in the stimulable phosphor layer region 92 of the stimulable phosphor sheet 90 attenuates to the level equal to at most the level that allows the stimulable phosphor sheet 90 to be scrapped. The calculation is made in accordance with the data concerning the date and hour of execution of the exposure operation for the stimulable phosphor sheet 90 and the data concerning the nuclide of the radioactive labeling substance, which data have been recorded on the magnetic recording layer 94 of the stimulable phosphor sheet 90. In accordance with the thus calculated scrapping period of the stimulable phosphor sheet 90 and the data concerning the year and month of scrapping, which data is stored in the memory, the control unit 660 determines the stimulable phosphor sheet collecting box 446a, 446b, . . . , or 446n which is to be used for collecting the stimulable phosphor sheet 90. Further, the control unit 660 drives the corresponding sorting member among the first sorting member 448a, the second sorting member 448b, . . . , and the n-th sorting member 448n in order to feed the stimulable phosphor sheet 90 into the determined stimulable phosphor sheet collecting box 446a, 446b, . . . , or 446n via the first chute 444a, the second chute 444b, . . . , or the n-th chute 444n. The stimulable phosphor sheet 90 is thus sorted and collected.

In the manner described above, all of the stimulable phosphor sheets 90, 90, . . . having bee loaded into the biochemical analysis medium loading box 682 are sorted and collected into the stimulable phosphor sheet collecting box 446a, 446b, . . . , or 446n or into the scrapping box 447. Thereafter, an operation end signal is fed from the keyboard 463, the driving of the endless belt motor 461 is stopped, and the sorting and collection of the stimulable phosphor sheets 90, 90, . . . are completed.

The biochemical analysis units 601, 601, . . . , which have been collected into the biochemical analysis unit collecting box 445a, 445b, . . . , or 445n in the manner described above, are stored and managed in the biochemical analysis unit collecting box 445a, 445b, . . . , or 445n until the year and the month indicated on the collecting box. At the end of the month indicated on the collecting box, the biochemical analysis units 601, 601, . . . having been stored in the collecting box are scrapped collectively.

The biochemical analysis unit 601, which has been collected into the scrapping box 447 by the biochemical analysis medium sorting apparatus, is scrapped immediately.

The stimulable phosphor sheets 90, 90, . . . , which have been collected into the stimulable phosphor sheet collecting box 446a, 446b, . . . , or 446n in the manner described above, are stored and managed in the stimulable phosphor sheet collecting box 446a, 446b, . . . , or 446n until the year and the month indicated on the collecting box. At the end of the month indicated on the collecting box, the stimulable phosphor sheets 90, 90, . . . having been stored in the collecting box are scrapped collectively.

In this embodiment, when the hybridization is executed, with the magnetic recording head 38 of the hybridization apparatus 30 shown in FIG. 8, the pieces of data are written on the magnetic recording layer 605 of the base plate 2 of the biochemical analysis unit 601. The pieces of data include the data concerning the date and hour of execution of the hybridization of the biochemical analysis unit 601, the radioactive label data, and the data concerning the nuclide of the radioactive labeling substance. The biochemical analysis unit 601 having been subjected N number of times to the hybridization is fed into the biochemical analysis medium sorting apparatus. The biochemical analysis medium sorting apparatus calculates the scrapping period of the biochemical analysis unit 601, at which the level of the radioactivity of the radioactive labeling substance contained in the adsorptive region 4 of the biochemical analysis unit 601 attenuates to the level equal to at most the level that allows the biochemical analysis unit 601 to be scrapped. The calculation is made in accordance with the data concerning the date and hour of execution of the hybridization and the data concerning the nuclide of the radioactive labeling substance, which data have been recorded on the magnetic recording layer 605 of the biochemical analysis unit 601. The biochemical analysis unit 601 is sorted in accordance with the calculated scrapping period and collected into one of the biochemical analysis unit collecting box 445a, 445b, . . . , or 445n. Therefore, the biochemical analysis unit 601 is capable of being managed reliably until the scrapping period of the biochemical analysis unit 601, at which the level of the radioactivity of the radioactive labeling substance contained in the adsorptive region 4 of the biochemical analysis unit 601 attenuates to the level equal to at most the level that allows the biochemical analysis unit 601 to be scrapped. Also, the biochemical analysis unit 601 is capable of being scrapped after the level of the radioactivity of the radioactive labeling substance contained in the adsorptive region 4 of the biochemical analysis unit 601 has attenuated to the level equal to at most the level that allows the biochemical analysis unit 601 to be scrapped.

Also, with this embodiment, in cases where the radioactive labeling substance has not been used as the labeling substance, the radioactive label data is not recorded on the magnetic recording layer 605 of the base plate 2 of the biochemical analysis unit 601. The biochemical analysis medium sorting apparatus is constituted such that, in cases where radioactive label data is not contained in the magnetic data having been recorded on the magnetic recording layer 605 of the base plate 2 of the biochemical analysis unit 601, the biochemical analysis unit 601 is collected immediately into the scrapping box 447. Therefore, the biochemical analysis unit 601 is capable of being sorted efficiently, and the problems are capable of being reliably prevented from occurring in that the biochemical analysis unit 601, which is capable of being scrapped immediately, is stored and managed by mistake.

Further, with this embodiment, when the stimulable phosphor sheet 90 is subjected to the exposure operation, with the magnetic recording head 112 of the exposure apparatus shown in FIG. 42, the data concerning the date and hour of execution of the exposure operation and the data concerning the nuclide of the radioactive labeling substance are recorded on the magnetic recording layer 94 of the support 91 of the stimulable phosphor sheet 90. The stimulable phosphor sheet 90, which has been subjected M number of times to the exposure operation, is fed into the biochemical analysis medium sorting apparatus. The biochemical analysis medium sorting apparatus calculates the scrapping period of the stimulable phosphor sheet 90, at which the level of the radioactivity of the radioactive labeling substance contained in the dot-shaped stimulable phosphor layer region 92 of the stimulable phosphor sheet 90 attenuates to the level equal to at most the level that allows the stimulable phosphor sheet 90 to be scrapped. The calculation is made in accordance with the data concerning the date and hour of execution of the exposure operation and the data concerning the nuclide of the radioactive labeling substance, which data have been recorded on the magnetic recording layer 94 of the support 91 of the stimulable phosphor sheet 90. The stimulable phosphor sheet 90 is sorted in accordance with the calculated scrapping period and collected into one of the stimulable phosphor sheet collecting box 446a, 446b, . . . , or 446n. Therefore, the stimulable phosphor sheet 90 is capable of being managed reliably until the scrapping period of the stimulable phosphor sheet 90, at which the level of the radioactivity of the radioactive labeling substance contained in the dot-shaped stimulable phosphor layer region 92 of the stimulable phosphor sheet 90 attenuates to the level equal to at most the level that allows the stimulable phosphor sheet 90 to be scrapped. Also, the stimulable phosphor sheet 90 is capable of being scrapped after the level of the radioactivity of the radioactive labeling substance contained in dot-shaped stimulable phosphor layer region 92 of the stimulable phosphor sheet 90 has attenuated to the level equal to at most the level that allows the stimulable phosphor sheet 90 to be scrapped.

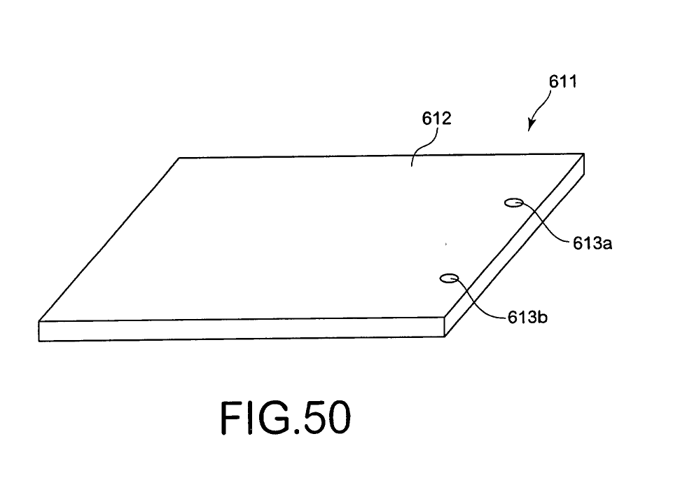

FIG. 50 is a schematic perspective view showing a biochemical analysis unit, which is managed by a second embodiment of the system for managing a biochemical analysis medium in accordance with the present invention.

As illustrated in FIG. 50, a biochemical analysis unit 611, which is managed by the second embodiment of the system for managing a biochemical analysis medium in accordance with the present invention, comprises an adsorptive plate 612 made from the 6-nylon.

As illustrated in FIG. 50, the adsorptive plate 612 of the biochemical analysis unit 611 is provided with two circular position matching through-holes 613a and 613b.

At the time of the biochemical analysis, by use of the spotting apparatus shown in FIG. 44, liquids containing the specific binding substances, such as the cDNA's, are spotted onto the adsorptive plate 612 of the biochemical analysis unit 611. In this manner, a plurality of spot-shaped regions, which are spaced from one another, are formed.

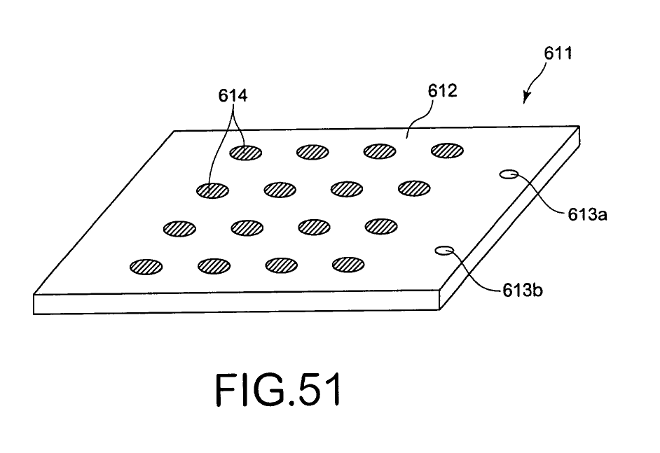

FIG. 51 is a schematic perspective view showing the biochemical analysis unit 611 provided with a plurality of spot-shaped regions located at a spacing from one another, each of the plurality of the spot-shaped regions having been formed with a process for spotting a liquid containing one of specific binding substances, such as cDNA's, onto the adsorptive plate 612 of the biochemical analysis unit 611 by use of the spotting apparatus.

As illustrated in FIG. 51, the liquids containing the specific binding substances, such as the cDNA's, are spotted onto the adsorptive plate 612 of the biochemical analysis unit 611. In this manner, a plurality of spot-shaped regions 614, 614, . . . , which are spaced from one another, are formed.

Though not shown precisely in FIG. 51, approximately 10,000 spot-shaped regions 614, 614, . . . , each of which has an approximately circular shape with a size of approximately 0.01 mm², are formed in a regular pattern at a density of approximately 5,000 regions/cm² on the adsorptive plate 612 of the biochemical analysis unit 611.

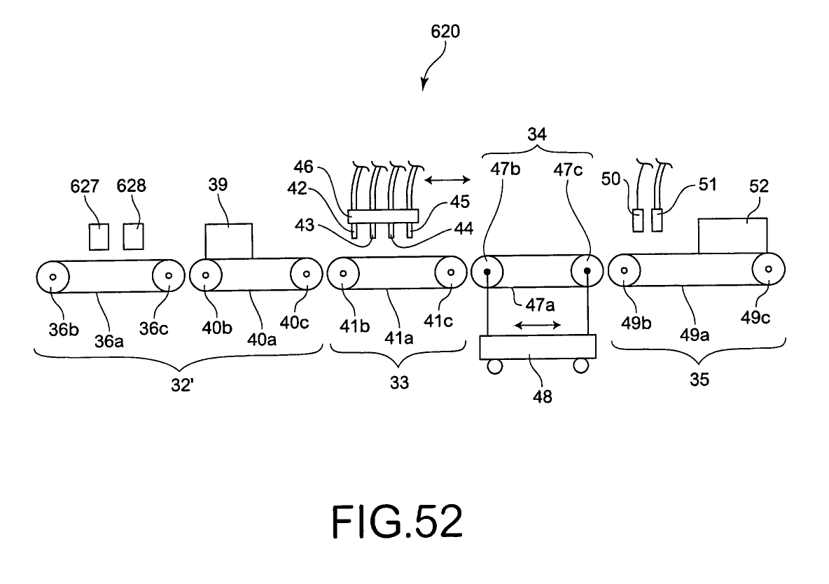

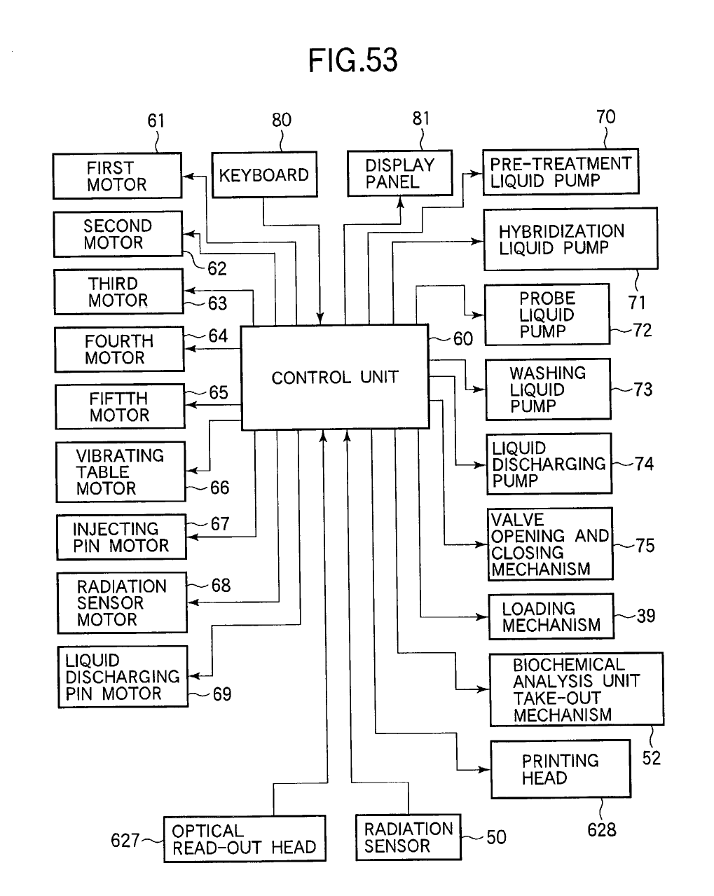

FIG. 52 is a schematic side view showing a hybridization apparatus, which constitutes the second embodiment of the system for managing a biochemical analysis medium in accordance with the present invention. FIG. 53 is a block diagram showing a control system, a detecting system, a driving system, an input system, and a displaying system of the hybridization apparatus, which constitutes the second embodiment of the system for managing a biochemical analysis medium in accordance with the present invention.

As illustrated in FIGS. 52 and 53, a hybridization apparatus 620, which constitutes the second embodiment of the system for managing a biochemical analysis medium in accordance with the present invention, has basically the same constitution as the constitution of the hybridization apparatus 30, which has been described above with reference to FIGS. 8 and 10 and is employed in the first embodiment of the system for managing a biochemical analysis medium in accordance with the present invention, except that a printing head 628 is utilized in lieu of the magnetic recording head 38 for writing the magnetic data on the magnetic recording layer 605 of the base plate 2 of the biochemical analysis unit 601, and an optical read-out head 627 is utilized in lieu of the read-out head 37 for reading out the magnetic data from the magnetic recording layer 605 of the base plate 2 of the biochemical analysis unit 601. At a cartridge loading section 32′, the printing head 628 prints the pieces of data on the adsorptive plate 612 of the biochemical analysis unit 611. The pieces of data include the data concerning the date and hour of execution of the hybridization, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. Also, the optical read-out head 627 reads out the data having been printed on the adsorptive plate 612 of the biochemical analysis unit 611.

In the hybridization apparatus 620, the organism-originating substance having been labeled with the radioactive labeling substance and the organism-originating substance having been labeled with the fluorescent labeling substance, which organism-originating substances are contained in the probe liquid, are subjected to the selective hybridization with the specific binding substances contained in the spot-shaped regions 614, 614, . . . of the adsorptive plate 612 of the biochemical analysis unit 611 basically in the same manner as that in the hybridization apparatus 30, which has been described above with reference to FIGS. 8 and 10 and is employed in the first embodiment of the system for managing a biochemical analysis medium in accordance with the present invention, except that the aforesaid pieces of data are printed on the adsorptive plate 612 of the biochemical analysis unit 611 by the printing head 628 and are read out optically by the optical read-out head 627.

In the manner described above, the radiation information, which is formed with the radioactive labeling substance acting as the labeling substance, is recorded on at least one spot-shaped region 614 among the plurality of the spot-shaped regions 614, 614, . . . of the biochemical analysis unit 611. Also, the fluorescence information, which is formed with the fluorescent labeling substance, such as the fluoro chrome, is recorded on at least one spot-shaped region 614. The fluorescence information, which has been recorded on at least one spot-shaped region 614, is read out by the scanner shown in FIGS. 16 to 22 and FIG. 32 in the same manner as that described above. In this manner, the data for a biochemical analysis is formed.

In the same manner as that described above, the radiation information, which is formed with the radioactive labeling substance, is transferred to a stimulable phosphor sheet.

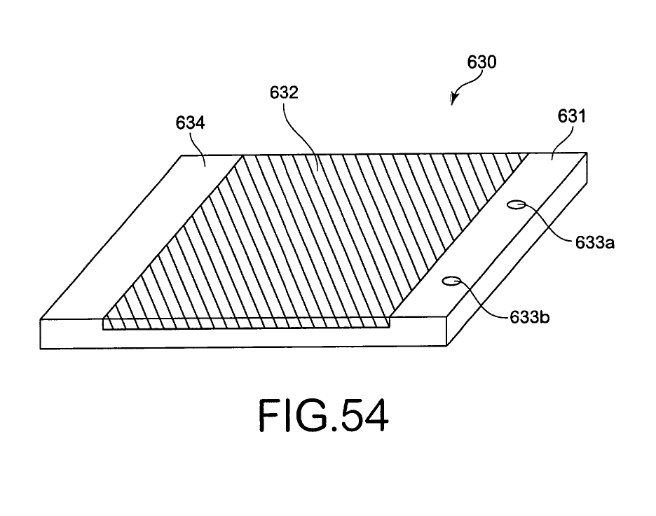

FIG. 54 is a schematic perspective view showing a stimulable phosphor sheet, which is managed by the second embodiment of the system for managing a biochemical analysis medium in accordance with the present invention.

As illustrated in FIG. 54, a stimulable phosphor sheet 630, which is managed by the second embodiment of the system for managing a biochemical analysis medium in accordance with the present invention, comprises a support 631 and a stimulable phosphor layer 632, which is formed on the surface of the support 631.

Also, as illustrated in FIG. 54, the support 631 is provided with position matching through-holes 633a and 633b. The support 631 is further provided with a printable region 634 at a position corresponding to the position of the magnetic recording layer 94 of the support 91 of the stimulable phosphor sheet 90 shown in FIG. 11.

In this embodiment, the data concerning the number of times of use for the exposure operation, the data concerning the date and hour of execution of the exposure operation, and the data concerning the nuclide of the radioactive labeling substance are capable of being printed on the printable region 634 of the support 631 by an exposure apparatus, which is described below.

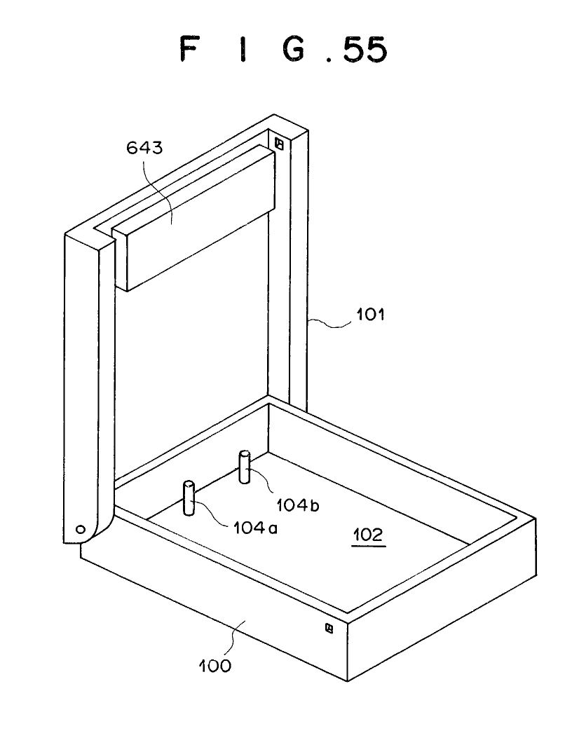

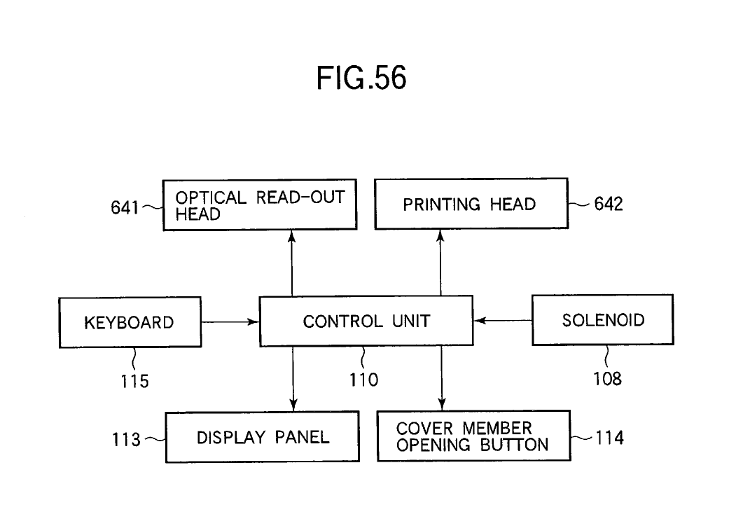

FIG. 55 is a schematic perspective view showing an exposure apparatus for executing an exposure operation, wherein the stimulable phosphor contained in the stimulable phosphor layer 632 formed on the support 631 of the stimulable phosphor sheet 630 is exposed to the radiation radiated out from the radioactive labeling substance, which is contained selectively in at least one spot-shaped region 614 among the plurality of the spot-shaped regions 614, 614, . . . of the adsorptive plate 612 of the biochemical analysis unit 611. FIG. 56 is a block diagram showing a control system, a detecting system, a driving system, and a displaying system, of the exposure apparatus of FIG. 55.

As illustrated in FIGS. 55 and 56, the exposure apparatus has basically the same constitution as the constitution of the exposure apparatus shown in FIGS. 12, 13 and FIG. 42, except that the cover member 101 is provided with a data read-out and recording section 643, which comprises an optical read-out head 641 and a printing head 642.

In cases where the stimulable phosphor layer 632 formed on the support 631 of the stimulable phosphor sheet 630 is to be exposed to the radiation radiated out from the radioactive labeling substance, which is contained selectively in at least one spot-shaped region 614 among the plurality of the spot-shaped regions 614, 614, . . . of the adsorptive plate 612 of the biochemical analysis unit 611, firstly, the data concerning the nuclide of the radioactive labeling substance is inputted from the keyboard 115, and the cover member 101 is opened.

Thereafter, the biochemical analysis unit 611 is set on the base 102 of the exposure apparatus by the user, such that the two position matching through-holes 613a and 613b of the adsorptive plate 612 of the biochemical analysis unit 611 may fit respectively onto the two position matching pins 104a and 104b of the base 102 of the exposure apparatus. Thereafter, the cover member 101 is closed.

The stimulable phosphor sheet 630 is set on the surface of the biochemical analysis unit 611 having been set on the base 102, such that the two position matching through-holes 633a and 633b of the support 631 of the stimulable phosphor sheet 630 may respectively fit onto the two position matching pins 104a and 104b of the base 102 of the exposure apparatus. Thereafter, the cover member 101 is closed.

When the cover member 101 has been closed, the optical read-out head 641 of the data read-out and recording section 643 reads out the data having been printed on the printable region 634 of the support 631 of the stimulable phosphor sheet 630. The thus obtained detection signal is fed into the control unit 110. Also, in the same manner as that in the first embodiment of the system for managing a biochemical analysis medium in accordance with the present invention, a judgment is made as to whether the stimulable phosphor layer 632 of the stimulable phosphor sheet 630 has or has not been subjected M number of times to the exposure operation.

As in the first embodiment of the system for managing a biochemical analysis medium in accordance with the present invention, in cases where it has been judged that the stimulable phosphor sheet 630 has been subjected M number of times to the exposure operation, the stimulable phosphor sheet 630 is removed from the exposure apparatus by the user.

In cases where it has been judged that the number of times of use for the exposure operation of the stimulable phosphor sheet 630 is smaller than M, with the printing head 642 of the data read-out and recording section 643, the number of times of use for the exposure operation of the stimulable phosphor sheet 630, which number of times has been recorded on the printable region 634 of the support 631 of the stimulable phosphor sheet 630, is increased by 1. At the same time, in accordance with the built-in clock, the control unit 110 forms the data concerning the date and hour of execution of the exposure operation. Also, the data concerning the nuclide of the radioactive labeling substance is formed in accordance with the information representing the data concerning the nuclide of the radioactive labeling substance, which information has been inputted from the keyboard 115. The thus formed data are written on the printable region 634 of the support 631.

In this manner, the biochemical analysis unit 611 and the stimulable phosphor sheet 630 are superposed one upon the other for a predetermined length of time. As a result, the stimulable phosphor layer 632 formed on the support 631 of the stimulable phosphor sheet 630 is exposed to the radiation radiated out from the radioactive labeling substance, which is contained selectively in at least one spot-shaped region 614 among the plurality of the spot-shaped regions 614, 614, . . . of the adsorptive plate 612 of the biochemical analysis unit 611.

In the manner described above, the radiation information with the radioactive labeling substance is recorded on the stimulable phosphor layer 632 formed on the support 631 of the stimulable phosphor sheet 630.

The radiation information having been recorded on the stimulable phosphor layer 632 formed on the support 631 of the stimulable phosphor sheet 630 is read out by the scanner described above with reference to FIGS. 16 to 22 and FIG. 32.

In cases where the biochemical analysis unit 611 has been subjected N number of times to the hybridization of the organism-originating substance, which has been labeled with the labeling substance, with the specific binding substances contained in the spot-shaped regions 614, 614, . . . , and the biochemical analysis unit 611 has thus been used for the formation of the data for a biochemical analysis, the biochemical analysis unit 611 is sorted in accordance with the scrapping period by the biochemical analysis medium sorting apparatus. Also, in cases where the stimulable phosphor sheet 630 has been subjected M number of times to the exposure operation, the stimulable phosphor sheet 630 is sorted in accordance with the scrapping period by the biochemical analysis medium sorting apparatus. The sorted biochemical analysis unit 611 and the sorted stimulable phosphor sheet 630 are stored and managed until the scrapping period.

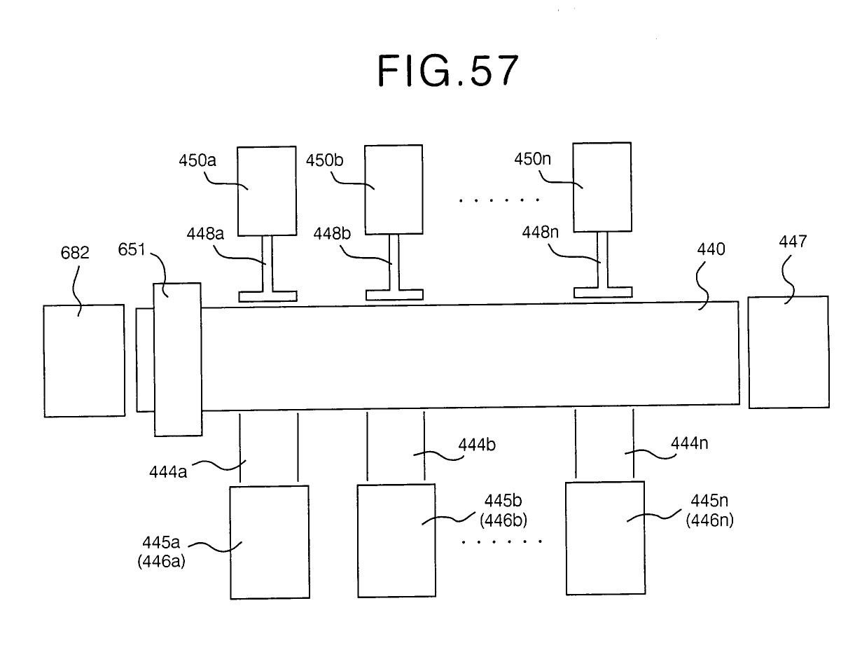

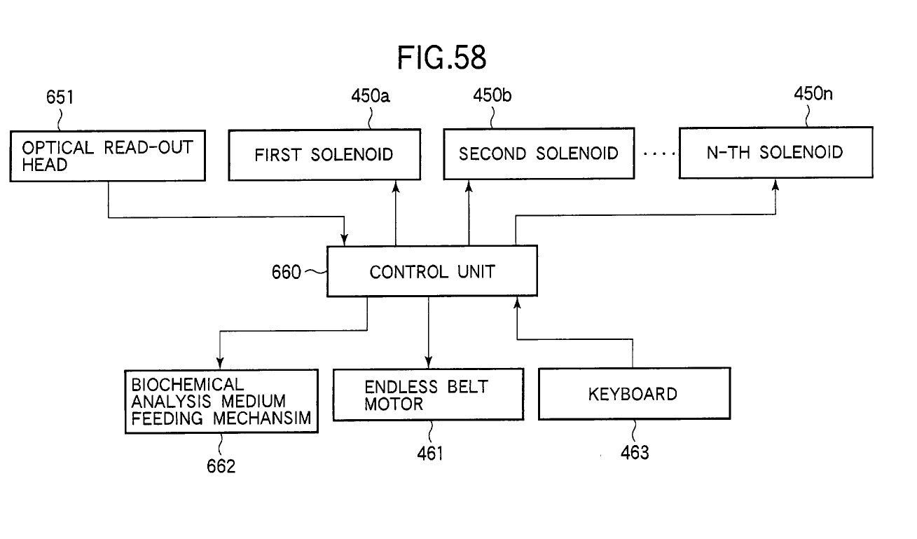

FIG. 57 is a schematic plan view showing a biochemical analysis medium sorting apparatus, which constitutes the second embodiment of the system for managing a biochemical analysis medium in accordance with the present invention. FIG. 58 is a block diagram showing a control system, a detecting system, a driving system, and an input system of the biochemical analysis medium sorting apparatus, which constitutes the second embodiment of the system for managing a biochemical analysis medium in accordance with the present invention.

As illustrated in FIGS. 57 and 58, the biochemical analysis medium sorting apparatus, which constitutes the second embodiment of the system for managing a biochemical analysis medium in accordance with the present invention, has basically the same constitution as that shown in FIGS. 46 and 47, except that an optical read-out head 651 is employed in lieu of the read-out head 681 for reading out the magnetic data. The optical read-out head 651 is capable of optically reading out the data having been recorded on the adsorptive plate 612 of the biochemical analysis unit 611 and the data having been recorded on the printable region 634 formed on the support 631 of the stimulable phosphor sheet 630.

The biochemical analysis medium sorting apparatus operates basically in the same manner as that in the biochemical analysis medium sorting apparatus shown in FIGS. 46 and 47, except that, in lieu of the magnetic data being read out from the magnetic recording layer 605 of the base plate 2 of the biochemical analysis unit 601 by the read-out head 681, the data concerning the date and hour of execution of the hybridization, the radioactive label data, and the data concerning the nuclide of the radioactive labeling substance are read out from the adsorptive plate 612 of the biochemical analysis unit 611 by the optical read-out head 651. In this manner, in accordance with the scrapping period of the biochemical analysis unit 611, at which the level of the radioactivity of the radioactive labeling substance contained in the spot-shaped region 614 of the biochemical analysis unit 611 attenuates to the level equal to at most the level that allows the biochemical analysis unit 611 to be scrapped, the biochemical analysis unit 611 is sorted and is collected into the biochemical analysis unit collecting box 445a, 445b, . . . , or 445n.

Also, in this embodiment, the biochemical analysis medium sorting apparatus operates basically in the same manner as that in the biochemical analysis medium sorting apparatus shown in FIGS. 46 and 47, except that, in lieu of the magnetic data being read out from the magnetic recording layer 94 of the support 91 of the stimulable phosphor sheet 90 by the read-out head 681, the data concerning the date and hour of execution of the exposure operation and the data concerning the nuclide of the radioactive labeling substance are read out from the printable region 634 of the support 631 of the stimulable phosphor sheet 630 by the optical read-out head 651. In this manner, in accordance with the scrapping period of the stimulable phosphor sheet 630, at which the level of the radioactivity of the radioactive labeling substance contained in the stimulable phosphor layer 632 of the stimulable phosphor sheet 630 attenuates to the level equal to at most the level that allows the stimulable phosphor sheet 630 to be scrapped, the stimulable phosphor sheet 630 is sorted and is collected into the stimulable phosphor sheet collecting box 446a, 446b, . . . , or 446n.

In this embodiment, when the hybridization is executed, with the printing head 628 of the hybridization apparatus 620, the pieces of data are printed on the adsorptive plate 612 of the biochemical analysis unit 611. The pieces of data include the data concerning the date and hour of execution of the hybridization, the radioactive label data, and the data concerning the nuclide of the radioactive labeling substance. The biochemical analysis unit 611 having been subjected N number of times to the hybridization is fed into the biochemical analysis medium sorting apparatus. The biochemical analysis medium sorting apparatus calculates the scrapping period of the biochemical analysis unit 611, at which the level of the radioactivity of the radioactive labeling substance contained in the spot-shaped region 614 of the adsorptive plate 612 of the biochemical analysis unit 611 attenuates to the level equal to at most the level that allows the biochemical analysis unit 611 to be scrapped. The calculation is made in accordance with the data concerning the date and hour of execution of the hybridization and the data concerning the nuclide of the radioactive labeling substance, which data have been printed on the adsorptive plate 612 of the biochemical analysis unit 611. The biochemical analysis unit 611 is sorted in accordance with the calculated scrapping period and collected into one of the biochemical analysis unit collecting box 445a, 445b, . . . , or 445n. Therefore, the biochemical analysis unit 611 is capable of being managed reliably until the scrapping period of the biochemical analysis unit 611, at which the level of the radioactivity of the radioactive labeling substance contained in the spot-shaped region 614 of the adsorptive plate 612 of the biochemical analysis unit 611 attenuates to the level equal to at most the level that allows the biochemical analysis unit 611 to be scrapped. Also, the biochemical analysis unit 611 is capable of being scrapped after the level of the radioactivity of the radioactive labeling substance contained in the spot-shaped region 614 of the adsorptive plate 612 of the biochemical analysis unit 611 has attenuated to the level equal to at most the level that allows the biochemical analysis unit 611 to be scrapped.

Further, with this embodiment, when the stimulable phosphor sheet 630 is subjected to the exposure operation, with the printing head 642 of the exposure apparatus, the data concerning the date and hour of execution of the exposure operation and the data concerning the nuclide of the radioactive labeling substance are printed on the printable region 634 of the support 631 of the stimulable phosphor sheet 630. The stimulable phosphor sheet 630, which has been subjected M number of times to the exposure operation, is fed into the biochemical analysis medium sorting apparatus. The biochemical analysis medium sorting apparatus calculates the scrapping period of the stimulable phosphor sheet 630, at which the level of the radioactivity of the radioactive labeling substance contained in the stimulable phosphor layer 632 of the stimulable phosphor sheet 630 attenuates to the level equal to at most the level that allows the stimulable phosphor sheet 630 to be scrapped. The calculation is made in accordance with the data concerning the date and hour of execution of the exposure operation and the data concerning the nuclide of the radioactive labeling substance, which data have been printed on the printable region 634 of the support 631 of the stimulable phosphor sheet 630. The stimulable phosphor sheet 630 is sorted in accordance with the calculated scrapping period and collected into one of the stimulable phosphor sheet collecting box 446a, 446b, . . . , or 446n. Therefore, the stimulable phosphor sheet 630 is capable of being managed reliably until the scrapping period of the stimulable phosphor sheet 630, at which the level of the radioactivity of the radioactive labeling substance contained in the stimulable phosphor layer 632 of the stimulable phosphor sheet 630 attenuates to the level equal to at most the level that allows the stimulable phosphor sheet 630 to be scrapped. Also, the stimulable phosphor sheet 630 is capable of being scrapped after the level of the radioactivity of the radioactive labeling substance contained in dot-shaped stimulable phosphor layer region 92 of the stimulable phosphor sheet 630 has attenuated to the level equal to at most the level that allows the stimulable phosphor sheet 630 to be scrapped.

The system for managing a biochemical analysis medium in accordance with the present invention may be embodied in various other ways.

For example, in the embodiment shown in FIGS. 50 to 58, with the printing head 628 of the hybridization apparatus 620, the pieces of data are printed on the adsorptive plate 612 of the biochemical analysis unit 611 provided with the spot-shaped regions 614, 614, . . . containing the specific binding substances. The pieces of data include the data concerning the date and hour of execution of the hybridization of the biochemical analysis unit 611, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. The data concerning the date and hour of execution of the hybridization, the radioactive label data, and the data concerning the nuclide of the radioactive labeling substance, which data have been printed on the adsorptive plate 612 of the biochemical analysis unit 611, are read out by the optical read-out head 651 of the biochemical analysis medium sorting apparatus. In accordance with the read-out data, the scrapping period of the biochemical analysis unit 611, at which the level of the radioactivity of the radioactive labeling substance contained in the spot-shaped region 614 of the biochemical analysis unit 611 attenuates to the level equal to at most the level that allows the biochemical analysis unit 611 to be scrapped, is calculated. The biochemical analysis unit 611 is sorted in accordance with the calculated scrapping period, stored, and managed. Alternatively, the data concerning the date and hour of execution of the hybridization, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance may be printed on the base plate 2 of the biochemical analysis unit 601 shown in FIG. 43, which is provided with the adsorptive regions 4, 4, . . . formed at a high density in the through-holes 3, 3, . . . having the approximately circular shape. Also, the data concerning the date and hour of execution of the hybridization, the radioactive label data, and the data concerning the nuclide of the radioactive labeling substance, which data have been printed on the base plate 2 of the biochemical analysis unit 601, may be read out with the optical read-out head 651 of the biochemical analysis medium sorting apparatus, and the biochemical analysis unit 601 may be sorted, stored, and managed in accordance with the read-out data.

Also, in the embodiment shown in FIGS. 50 to 58, with the printing head 628 of the hybridization apparatus 620, the pieces of data are printed on the printable region 634 of the support 631 of the stimulable phosphor sheet 630. The pieces of data include the data concerning the date and hour of execution of the exposure operation, the data concerning the number of times of use for the exposure operation, and the data concerning the nuclide of the radioactive labeling substance. The data concerning the date and hour of execution of the exposure operation and the data concerning the nuclide of the radioactive labeling substance, which data have been printed on the printable region 634 of the support 631 of the stimulable phosphor sheet 630, are read out by the optical read-out head 651 of the biochemical analysis medium sorting apparatus. In accordance with the read-out data, the scrapping period of the stimulable phosphor sheet 630, at which the level of the radioactivity of the radioactive labeling substance contained in the stimulable phosphor layer 632 of the stimulable phosphor sheet 630 attenuates to the level equal to at most the level that allows the stimulable phosphor sheet 630 to be scrapped, is calculated. The stimulable phosphor sheet 630 is sorted in accordance with the calculated scrapping period, stored, and managed. Alternatively, the data concerning the date and hour of execution of the exposure operation, the data concerning the number of times of use for the exposure operation, and the data concerning the nuclide of the radioactive labeling substance may be printed on the support 91 of the stimulable phosphor sheet 90 shown in FIG. 11, which is provided with the dot-shaped stimulable phosphor layer regions 92, 92, . . . formed at a high density in the through-holes 93, 93, . . . having the approximately circular shape. Also, the data concerning the date and hour of execution of the hybridization and the data concerning the nuclide of the radioactive labeling substance, which data have been printed on the support 91 of the stimulable phosphor sheet 90, may be read out with the optical read-out head 651 of the biochemical analysis medium sorting apparatus, and the stimulable phosphor sheet 90 may be sorted, stored, and managed in accordance with the read-out data.

Further, in the embodiment shown in FIGS. 50 to 58, with the printing head 628 of the hybridization apparatus 620, the pieces of data are printed on the adsorptive plate 612 of the biochemical analysis unit 611 provided with the spot-shaped regions 614, 614, . . . containing the specific binding substances. The pieces of data include the data concerning the date and hour of execution of the hybridization of the biochemical analysis unit 611, the data concerning the number of times of use for the hybridization, the radioactive label data representing the use of the radioactive labeling substance in cases where the radioactive labeling substance has been used as the labeling substance, and the data concerning the nuclide of the radioactive labeling substance in cases where the radioactive labeling substance has been used as the labeling substance. Alternatively, the pieces of data described above may be recorded on the adsorptive plate 612 of the biochemical analysis unit 611 with a technique other than the printing, such as marking.

In the embodiment shown in FIGS. 43 to 49, the base plate 2 of the biochemical analysis unit 601 is provided with the magnetic recording layer 605, and the ID data, the data concerning the date and hour of execution of the hybridization, the data concerning the number of times of use for the hybridization, the data representing whether the radioactive labeling substance has or has not been used, and the data concerning the nuclide of the radioactive labeling substance are recorded on the magnetic recording layer 605. Alternatively, in lieu of the magnetic recording layer 605, an optical recording layer may be provided, and the pieces of data described above, and the like, may be recorded on the optical recording layer. As another alternative, a data recording layer, on which the data is capable of being recorded with a recording technique other than the magnetic recording and the optical recording, may be employed.

Also, in the embodiment shown in FIGS. 43 to 49, the support 91 of the stimulable phosphor sheet 90 is provided with the magnetic recording layer 94, and the ID data, the data concerning the date and hour of execution of the exposure operation, the data concerning the number of times of use for the exposure operation, and the data concerning the nuclide of the radioactive labeling substance are recorded on the magnetic recording layer 94. Alternatively, in lieu of the magnetic recording layer 94, an optical recording layer may be provided, and the pieces of data described above, and the like, may be recorded on the optical recording layer. As another alternative, a data recording layer, on which the data is capable of being recorded with a recording technique other than the magnetic recording and the optical recording, may be employed.

In the embodiment shown in FIGS. 43 to 49, the data concerning the number of times of use for the hybridization is magnetically recorded on the magnetic recording layer 605 of the base plate 2 of the biochemical analysis unit 601. Also, in the embodiment shown in FIGS. 50 to 58, the data concerning the number of times of use for the hybridization is printed on the adsorptive plate 612 of the biochemical analysis unit 611. However, the data concerning the number of times of use for the hybridization need not necessarily be magnetically recorded on the magnetic recording layer 605 of the base plate 2 of the biochemical analysis unit 601 or printed on the adsorptive plate 612 of the biochemical analysis unit 611, and the number of times of use for the hybridization may be detected with other means.

Also, in the embodiment shown in FIGS. 43 to 49, the data concerning the number of times of use for the exposure operation is magnetically recorded on the magnetic recording layer 94 of the support 91 of the stimulable phosphor sheet 90. Also, in the embodiment shown in FIGS. 50 to 58, the data concerning the number of times of use for the exposure operation is printed on the support 631 of the stimulable phosphor sheet 630. However, the data concerning the number of times of use for the exposure operation need not necessarily be magnetically recorded on the magnetic recording layer 94 of the support 91 of the stimulable phosphor sheet 90 or printed on the support 631 of the stimulable phosphor sheet 630, and the number of times of use for the exposure operation may be detected with other means.

Further, in the embodiment shown in FIGS. 43 to 49, the data concerning the number of times of use for the hybridization is magnetically recorded on the magnetic recording layer 605 of the base plate 2 of the biochemical analysis unit 601. Also, in the embodiment shown in FIGS. 50 to 58, the data concerning the number of times of use for the hybridization is printed on the adsorptive plate 612 of the biochemical analysis unit 611. In cases where it has been judged, in accordance with the data concerning the number of times of use for the hybridization, that the biochemical analysis unit 601 or 611 has been used N number of times for the hybridization, the hybridization with the biochemical analysis unit 601 or 611 cannot be executed. However, the number of times of use of the biochemical analysis unit 601 or 611 for the hybridization need not necessarily be limited.

Furthermore, in the embodiment shown in FIGS. 43 to 49, the data concerning the number of times of use for the exposure operation is magnetically recorded on the magnetic recording layer 94 of the support 91 of the stimulable phosphor sheet 90. Also, in the embodiment shown in FIGS. 50 to 58, the data concerning the number of times of use for the exposure operation is printed on the support 631 of the stimulable phosphor sheet 630. In cases where it has been judged, in accordance with the data concerning the number of times of use for the exposure operation, that the stimulable phosphor sheet 90 or 630 has been used M number of times for the exposure operation, the exposure operation with the stimulable phosphor sheet 90 or 630 cannot be executed. However, the number of times of use of the stimulable phosphor sheet 90 or 630 for the exposure operation need not necessarily be limited.

In the embodiments of the system for managing a biochemical analysis medium in accordance with the present invention, the biochemical analysis unit 601 is accommodated in the cartridge 31 shown in FIG. 9. Also, by use of the hybridization apparatus 30 shown in FIGS. 8 and 10 or the hybridization apparatus 620 shown in FIGS. 52 and 53, the organism-originating substance having been labeled with the radioactive labeling substance and the organism-originating substance having been labeled with the fluorescent labeling substance are subjected to the selective hybridization with the specific binding substances contained in the adsorptive regions 4, 4, . . . of the biochemical analysis unit 601 or the spot-shaped regions 614, 614, . . . of the biochemical analysis unit 611. Alternatively, the selective hybridization may be executed with one of other hybridization apparatuses, which are capable of recording the data concerning the date and hour of execution of the hybridization, the radioactive label data, and the data concerning the nuclide of the radioactive labeling substance on the biochemical analysis unit 601 or 611.

Also, in the embodiments described above, the exposure operation using the stimulable phosphor sheet 90 and the biochemical analysis unit 601 is executed with the exposure apparatus shown in FIGS. 12, 13, and 42, and the exposure operation using the stimulable phosphor sheet 630 and the biochemical analysis unit 611 is executed with the exposure apparatus shown in FIGS. 55 and 56. Alternatively, the exposure operation may be executed with one of other exposure apparatuses, which are capable of recording the data concerning the date and hour of execution of the exposure operation and the data concerning the nuclide of the radioactive labeling substance on the stimulable phosphor sheet 90 or 630.

Further, in the embodiments of the system for managing a biochemical analysis medium in accordance with the present invention, the organism-originating substance having been labeled with the radioactive labeling substance and the organism-originating substance having been labeled with the fluorescent labeling substance are subjected to the selective hybridization with the specific binding substances contained in the adsorptive regions 4, 4, . . . of the biochemical analysis unit 601 or the spot-shaped regions 614, 614, . . . of the biochemical analysis unit 611. However, the selective hybridization need not necessarily be executed in this manner, and it is sufficient for the organism-originating substance having been labeled with at least the radioactive labeling substance to be subjected to the selective hybridization. Besides the organism-originating substance having been labeled with the fluorescent labeling substance, or in lieu of the organism-originating substance having been labeled with the fluorescent labeling substance, an organism-originating substance having been labeled with the labeling substance capable of producing the chemiluminescence when being brought into contact with a chemiluminescence substrate may be subjected to the selective hybridization. Alternatively, only the organism-originating substance having been labeled with the radioactive labeling substance may be subjected to the selective hybridization.

In the embodiment shown in FIGS. 43 to 49, when the hybridization is executed, the data concerning the date and hour of execution of the hybridization is recoded on the magnetic recording layer 605 of the base plate 2 of the biochemical analysis unit 601. Alternatively, when the hybridization is executed, the data concerning the date and hour of execution of the hybridization may be overwritten on the magnetic recording layer 605 of the base plate 2 of the biochemical analysis unit 601.

Also, in the embodiment shown in FIGS. 43 to 49, when the exposure operation is executed, the data concerning the date and hour of execution of the exposure operation is recoded on the magnetic recording layer 94 of the support 91 of the stimulable phosphor sheet 90. Alternatively, when the exposure operation is executed, the data concerning the date and hour of execution of the exposure operation may be overwritten on the magnetic recording layer 94 of the support 91 of the stimulable phosphor sheet 90.

In the embodiment shown in FIGS. 43 to 49, when the hybridization is executed, the data concerning the date and hour of execution of the hybridization is recoded on the magnetic recording layer 605 of the base plate 2 of the biochemical analysis unit 601. Also, in the embodiment shown in FIGS. 50 to 58, when the hybridization is executed, the data concerning the date and hour of execution of the hybridization is printed on the adsorptive plate 612 of the biochemical analysis unit 611. Alternatively, when the hybridization is executed, the data concerning the day of execution of the hybridization may be recorded on the magnetic recording layer 605 of the base plate 2 of the biochemical analysis unit 601 or may be printed on the adsorptive plate 612 of the biochemical analysis unit 611. Also, the biochemical analysis unit 601 or 611 may be managed in accordance with the data concerning the day of execution of the hybridization.

Also, in the embodiment shown in FIGS. 43 to 49, when the exposure operation is executed, the data concerning the date and hour of execution of the exposure operation is recoded on the magnetic recording layer 94 of the support 91 of the stimulable phosphor sheet 90. Also, in the embodiment shown in FIGS. 50 to 58, when the exposure operation is executed, the data concerning the date and hour of execution of the exposure operation is printed on the support 631 of the stimulable phosphor sheet 630. Alternatively, when the exposure operation is executed, the data concerning the day of execution of the exposure operation may be recorded on the magnetic recording layer 94 of the support 91 of the stimulable phosphor sheet 90 or may be printed on the support 631 of the stimulable phosphor sheet 630. Also, the stimulable phosphor sheet 90 or 630 may be managed in accordance with the data concerning the day of execution of the exposure operation.

In the embodiment shown in FIGS. 43 to 49, before the hybridization is executed, the data concerning the date and hour of execution of the hybridization, the radioactive label data, and the data concerning the nuclide of the radioactive labeling substance are recoded on the magnetic recording layer 605 of the base plate 2 of the biochemical analysis unit 601 by use of the hybridization apparatus 30. Also, in the embodiment shown in FIGS. 50 to 58, before the hybridization is executed, the data concerning the date and hour of execution of the hybridization, the radioactive label data, and the data concerning the nuclide of the radioactive labeling substance are printed on the adsorptive plate 612 of the biochemical analysis unit 611 by use of the hybridization apparatus 620. Alternatively, the recording or the printing of the data described above may be performed after the hybridization is executed.

Also, in the embodiment shown in FIGS. 43 to 49, before the exposure operation is executed, the data concerning the date and hour of execution of the exposure operation and the data concerning the nuclide of the radioactive labeling substance are recoded on the magnetic recording layer 94 of the support 91 of the stimulable phosphor sheet 90 by use of the exposure apparatus. Also, in the embodiment shown in FIGS. 50 to 58, before the exposure operation is executed, the data concerning the date and hour of execution of the exposure operation and the data concerning the nuclide of the radioactive labeling substance are printed on the printable region 634 of the support 631 of the stimulable phosphor sheet 630 by use of the exposure apparatus. Alternatively, the recording or the printing of the data described above may be performed after the exposure operation is executed.

In the embodiment shown in FIGS. 43 to 49, the data concerning the date and hour of execution of the hybridization, the radioactive label data, and the data concerning the nuclide of the radioactive labeling substance are recoded on the magnetic recording layer 605 of the base plate 2 of the biochemical analysis unit 601 by use of the hybridization apparatus 30. The biochemical analysis unit 601 is sorted and collected by the biochemical analysis medium sorting apparatus shown in FIGS. 46 and 47 and in accordance with the data having been recorded on the magnetic recording layer 605. Also, in the embodiment shown in FIGS. 50 to 58, the data concerning the date and hour of execution of the hybridization, the radioactive label data, and the data concerning the nuclide of the radioactive labeling substance are printed on the adsorptive plate 612 of the biochemical analysis unit 611 by use of the hybridization apparatus 620. The biochemical analysis unit 611 is sorted and collected by the biochemical analysis medium sorting apparatus shown in FIGS. 57 and 58 and in accordance with the data having been printed on the adsorptive plate 612 of the biochemical analysis unit 611. Alternatively, in lieu of the data concerning the date and hour of execution of the hybridization, the data concerning the date and hour, or the day, of formation of the radioactive labeling substance may be inputted from the keyboard 80 of the hybridization apparatus 30 and may be recorded on the magnetic recording layer 605 of the base plate 2 of the biochemical analysis unit 601. The biochemical analysis unit 601 may then be sorted and collected by the biochemical analysis medium sorting apparatus shown in FIGS. 46 and 47 and in accordance with the data concerning the date and hour, or the day, of formation of the radioactive labeling substance, the radioactive label data, and the data concerning the nuclide of the radioactive labeling substance, which data have been recorded on the magnetic recording layer 605. Also, in lieu of the data concerning the date and hour of execution of the hybridization, the data concerning the date and hour, or the day, of formation of the radioactive labeling substance may be inputted from the keyboard 80 of the hybridization apparatus 620 and may be printed on the adsorptive plate 612 of the biochemical analysis unit 611. The biochemical analysis unit 611 may then be sorted and collected by the biochemical analysis medium sorting apparatus shown in FIGS. 57 and 58 and in accordance with the data concerning the date and hour, or the day, of formation of the radioactive labeling substance, the radioactive label data, and the data concerning the nuclide of the radioactive labeling substance, which data have been printed on the adsorptive plate 612 of the biochemical analysis unit 611.

Also, in the embodiment shown in FIGS. 43 to 49, the data concerning the date and hour of execution of the exposure operation and the data concerning the nuclide of the radioactive labeling substance are recoded on the magnetic recording layer 94 of the support 91 of the stimulable phosphor sheet 90 by use of the exposure apparatus. The stimulable phosphor sheet 90 is sorted and collected by the biochemical analysis medium sorting apparatus shown in FIGS. 46 and 47 and in accordance with the data having been recorded on the magnetic recording layer 94. Also, in the embodiment shown in FIGS. 50 to 58, the data concerning the date and hour of execution of the exposure operation and the data concerning the nuclide of the radioactive labeling substance are printed on the printable region 634 of the support 631 of the stimulable phosphor sheet 630 by use of the exposure apparatus. The stimulable phosphor sheet 630 is sorted and collected by the biochemical analysis medium sorting apparatus shown in FIGS. 57 and 58 and in accordance with the data having been printed on the printable region 634 of the support 631 of the stimulable phosphor sheet 630. Alternatively, in lieu of the data concerning the date and hour of execution of the exposure operation, the data concerning the date and hour, or the day, of formation of the radioactive labeling substance may be inputted from the keyboard 115 of the exposure apparatus. Also, the data concerning the date and hour, or the day, of formation of the radioactive labeling substance and the data concerning the nuclide of the radioactive labeling substance may be recorded on the magnetic recording layer 94 of the support 91 of the stimulable phosphor sheet 90. The stimulable phosphor sheet 90 may then be sorted and collected by the biochemical analysis medium sorting apparatus shown in FIGS. 46 and 47 and in accordance with the data concerning the date and hour, or the day, of formation of the radioactive labeling substance and the data concerning the nuclide of the radioactive labeling substance, which data have been recorded on the magnetic recording layer 94. Also, in lieu of the data concerning the date and hour of execution of the exposure operation, the data concerning the date and hour, or the day, of formation of the radioactive labeling substance may be inputted from the keyboard 115 of the exposure apparatus. Also, the data concerning the date and hour, or the day, of formation of the radioactive labeling substance and the data concerning the nuclide of the radioactive labeling substance may be printed on the printable region 634 of the support 631 of the stimulable phosphor sheet 630. The stimulable phosphor sheet 630 may then be sorted and collected by the biochemical analysis medium sorting apparatus shown in FIGS. 57 and 58 and in accordance with the data concerning the date and hour, or the day, of formation of the radioactive labeling substance and the data concerning the nuclide of the radioactive labeling substance, which data have been printed on the printable region 634.

The embodiment shown in FIGS. 43 and 49 may be modified such that, instead of the data concerning the date and hour of execution of the hybridization, the radioactive label data, and the data concerning the nuclide of the radioactive labeling substance being recoded on the magnetic recording layer 605 of the base plate 2 of the biochemical analysis unit 601 by use of the hybridization apparatus 30, the cover member 101 may be closed after the biochemical analysis unit 601 has been set on the base 102 of the exposure apparatus, and the data concerning the date and hour, or the day, of execution of the exposure operation and the data concerning the nuclide of the radioactive labeling substance may be recorded on the magnetic recording layer 605 of the base plate 2 of the biochemical analysis unit 601 by use of the magnetic recording head 112 of the exposure apparatus. The biochemical analysis unit 601 may then be sorted and collected by the biochemical analysis medium sorting apparatus shown in FIGS. 46 and 47 and in accordance with the data concerning the date and hour, or the day, of execution of the exposure operation and the data concerning the nuclide of the radioactive labeling substance, which data have been recorded on the magnetic recording layer 605 of the base plate 2 of the biochemical analysis unit 601.

Also, the embodiment shown in FIGS. 50 to 58 may be modified such that, instead of the data concerning the date and hour of execution of the hybridization, the radioactive label data, and the data concerning the nuclide of the radioactive labeling substance being printed on the adsorptive plate 612 of the biochemical analysis unit 611 by use of the hybridization apparatus 620, the cover member 101 may be closed after the biochemical analysis unit 611 has been set on the base 102 of the exposure apparatus, and the data concerning the date and hour, or the day, of execution of the exposure operation and the data concerning the nuclide of the radioactive labeling substance may be printed on the adsorptive plate 612 of the biochemical analysis unit 611 by use of the printing head 642 of the exposure apparatus. The biochemical analysis unit 611 may then be sorted and collected by the biochemical analysis medium sorting apparatus shown in FIGS. 57 and 58 and in accordance with the data concerning the date and hour, or the day, of execution of the exposure operation and the data concerning the nuclide of the radioactive labeling substance, which data have been printed on the adsorptive plate 612 of the biochemical analysis unit 611.

Further, the embodiment shown in FIGS. 43 and 49 may be modified such that, instead of the data concerning the date and hour of execution of the hybridization, the radioactive label data, and the data concerning the nuclide of the radioactive labeling substance being recoded on the magnetic recording layer 605 of the base plate 2 of the biochemical analysis unit 601 by use of the hybridization apparatus 30, the data concerning the date and hour, or the day, of formation of the radioactive labeling substance, the radioactive label data, and the data concerning the nuclide of the radioactive labeling substance may be recorded on the magnetic recording layer 605 of the base plate 2 of the biochemical analysis unit 601 by use of the magnetic recording head 112 of the exposure apparatus. The biochemical analysis unit 601 may then be sorted and collected by the biochemical analysis medium sorting apparatus shown in FIGS. 46 and 47 and in accordance with the data concerning the date and hour, or the day, of formation of the radioactive labeling substance, the radioactive label data, and the data concerning the nuclide of the radioactive labeling substance, which data have been recorded on the magnetic recording layer 605 of the base plate 2 of the biochemical analysis unit 601.

Furthermore, the embodiment shown in FIGS. 50 to 58 may be modified such that, instead of the data concerning the date and hour of execution of the hybridization, the radioactive label data, and the data concerning the nuclide of the radioactive labeling substance being printed on the adsorptive plate 612 of the biochemical analysis unit 611 by use of the hybridization apparatus 620, the data concerning the date and hour, or the day, of formation of the radioactive labeling substance, the radioactive label data, and the data concerning the nuclide of the radioactive labeling substance may be printed on the adsorptive plate 612 of the biochemical analysis unit 611 by use of the printing head 642 of the exposure apparatus. The biochemical analysis unit 611 may then be sorted and collected by the biochemical analysis medium sorting apparatus shown in FIGS. 57 and 58 and in accordance with the data concerning the date and hour, or the day, of formation of the radioactive labeling substance, the radioactive label data, and the data concerning the nuclide of the radioactive labeling substance, which data have been printed on the adsorptive plate 612 of the biochemical analysis unit 611.

In the system for managing a biochemical analysis medium in accordance with the present invention, the biochemical analysis medium sorting apparatus is not limited to the biochemical analysis medium sorting apparatus shown in FIGS. 46 and 47 or in FIGS. 57 and 58 and may be one of various other biochemical analysis medium sorting apparatuses, which are capable of reading out the data concerning the date and hour of execution of the hybridization and the data concerning the nuclide of the radioactive labeling substance from the magnetic recording layer 605 of the biochemical analysis unit 601 or from the adsorptive plate 612 of the biochemical analysis unit 611, reading out the data concerning the date and hour of execution of the exposure operation and the data concerning the nuclide of the radioactive labeling substance from the magnetic recording layer 94 of the stimulable phosphor sheet 90 or from the support 631 of the stimulable phosphor sheet 630, calculating the scrapping period of the biochemical analysis unit 601 or 611 and the scrapping period of the stimulable phosphor sheet 90 or 630, automatically sorting the biochemical analysis unit 601 and the stimulable phosphor sheet 90, or the biochemical analysis unit 611 and the stimulable phosphor sheet 630, and collecting the thus sorted biochemical analysis media into different boxes in accordance with the scrapping periods.

In the embodiment shown in FIGS. 46 and 47, the biochemical analysis medium sorting apparatus is constituted to sort both the biochemical analysis unit 601 and the stimulable phosphor sheet 90. Also, in the embodiment shown in FIGS. 57 and 58, the biochemical analysis medium sorting apparatus is constituted to sort both the biochemical analysis unit 611 and the stimulable phosphor sheet 630. Alternatively, the biochemical analysis unit 601 and the stimulable phosphor sheet 90 may be sorted by two independent biochemical analysis medium sorting apparatuses. Also, the biochemical analysis unit 611 and the stimulable phosphor sheet 630 may be sorted by two independent biochemical analysis medium sorting apparatuses.

Further, in the embodiments described above, each of the biochemical analysis unit 601 or 611 and the stimulable phosphor sheet 90 or 630 is sorted in accordance with the month containing the date and hour, at which the biochemical analysis unit or the stimulable phosphor sheet is capable of being scrapped. Alternatively, each of the biochemical analysis unit 601 or 611 and the stimulable phosphor sheet 90 or 630 may be sorted in accordance with the day and the month, at which the biochemical analysis unit or the stimulable phosphor sheet is capable of being scrapped. In such cases, the year and the month, at the end of which month the biochemical analysis unit 601 or 611 to be accommodated is capable of being scrapped, may be indicated on each of the biochemical analysis unit collecting boxes 445a, 445b, . . . , and 445n. Also, the year and the month, at the end of which month the stimulable phosphor sheet 90 or 630 to be accommodated is capable of being scrapped, may be indicated on each of the stimulable phosphor sheet collecting boxes 446a, 446b, . . . , and 446n.

In the embodiment shown in FIGS. 43 to 49, it is sufficient for the dot-shaped stimulable phosphor layer regions 92, 92, . . . to be formed in the same location pattern as that of the adsorptive regions 4, 4, . . . of the base plate 2 of the biochemical analysis unit 601, and the dot-shaped stimulable phosphor layer regions 92, 92, . . . need not necessarily be located in the regular pattern.

Also, in the embodiment shown in FIGS. 43 to 49, each of the dot-shaped stimulable phosphor layer regions 92, 92, . . . need not necessarily have the approximately circular shape and may have one of other shapes, such as a rectangular shape.

Further, in the embodiment shown in FIGS. 43 to 49, the number and the size of the dot-shaped stimulable phosphor layer regions 92, 92, . . . may be set arbitrarily in accordance with the purposes of use. However, at least 10 dot-shaped stimulable phosphor layer regions 92, 92, . . . , each of which has a size smaller than 5 mm², should preferably be formed at a density of at least 10 regions/cm² in the support 91.

What is claimed is:

1. A biochemical analysis system, comprising:
i) specific binding means for:
obtaining a biochemical analysis unit, which comprises a base plate and a plurality of spot-shaped regions located at different positions on or in the base plate, and which is provided with inherent identification information, each of multiple kinds of specific binding substances, which are capable of specifically binding to organism-originating substances and whose structures or characteristics are known, having been fixed to one of the spot-shaped regions, and
subjecting an organism-originating substance, which has been labeled with at least one labeling substance selected from the group consisting of a radioactive labeling substance, a fluorescent labeling substance, and a labeling substance capable of producing chemiluminescence when being brought into contact with a chemiluminescence substrate, and which has inherent identification information, to selective, specific binding to the multiple kinds of the specific binding substances, each of which has been fixed to one of the spot-shaped regions of the biochemical analysis unit, at least one spot-shaped region among the plurality of the spot-shaped regions of the biochemical analysis unit being thereby selectively labeled with the labeling substance,
ii) label signal forming means for receiving the biochemical analysis unit, which has been processed by the specific binding means, and forming a label signal, which corresponds to a position of the spot-shaped region having been selectively labeled with the labeling substance, by use of the biochemical analysis unit,
iii) read-out means for reading out the label signal, which has been formed by the label signal forming means, in order to form data for a biochemical analysis, and
iv) analysis means for executing a biochemical analysis in accordance with the data for a biochemical analysis,
wherein the biochemical analysis system further comprises first correspondence relationship specifying means for acquiring the identification information of the biochemical analysis unit and the identification information of the organism-originating substance, and feeding the two pieces of the identification information into the analysis means such that the correspondence relationship between the two pieces of the identification information is clear.

2. A biochemical analysis system as defined in claim 1 wherein the base plate of the biochemical analysis unit is made from a material having radiation attenuating properties and/or light attenuating properties and has a plurality of holes,
an adsorptive region is formed in each of the plurality of the holes of the base plate, and
each of the multiple kinds of the specific binding substances is fixed to one of the adsorptive regions in order to form one of the spot-shaped regions.

3. A biochemical analysis system as defined in claim 1 wherein the organism-originating substance is labeled with the radioactive labeling substance,
the label signal forming means comprises:
a) exposure means for superposing a stimulable phosphor sheet, which has inherent identification information, and the biochemical analysis unit, which has been processed by the specific binding means, upon each other in order to expose the stimulable phosphor sheet to radiation radiated out from the radioactive labeling substance, and
b) stimulable phosphor sheet stimulating means for irradiating stimulating rays to the stimulable phosphor sheet, which has been exposed to the radiation radiated out from the radioactive labeling substance, the stimulating rays causing the stimulable phosphor sheet to emit light in proportion to the amount of energy stored on the stimulable phosphor sheet during the exposure of the stimulable phosphor sheet to the radiation, the light emitted by the stimulable phosphor sheet acting as the label signal, and
the read-out means photoelectrically detects the light, which is emitted by the stimulable phosphor sheet-, in order to form the data for a biochemical analysis.

4. A biochemical analysis system as defined in claim 3 wherein the stimulable phosphor sheet comprises a support and a plurality of dot-shaped stimulable phosphor layer regions, which are located at a spacing from one another on the support, and
positions and sizes of the plurality of the dot-shaped stimulable phosphor layer regions correspond respectively to the positions and the sizes of the spot-shaped regions of the biochemical analysis unit.

5. A biochemical analysis system as defined in claim 3 wherein the biochemical analysis system further comprises second correspondence relationship specifying means for acquiring the identification information of the biochemical analysis unit and the identification information of the stimulable phosphor sheet, and feeding the two pieces of the identification information into the analysis means such that the correspondence relationship between the two pieces of the identification information is clear.

6. A biochemical analysis system as defined in claim 1 wherein the organism-originating substance is labeled with the fluorescent labeling substance,
the label signal forming means is fluorescent substance exciting means for irradiating excitation light to the biochemical analysis unit, which has been processed by the specific binding means, the excitation light causing the fluorescent labeling substance to produce fluorescence, the fluorescence produced by the fluorescent labeling substance acting as the label signal, and
the read-out means photoelectrically detects the fluorescence, which is produced by the fluorescent labeling substance, in order to form the data for a biochemical analysis.

7. A biochemical analysis system as defined in claim 1 wherein the organism-originating substance is labeled with the labeling substance capable of producing the chemiluminescence when being brought into contact with a chemiluminescence substrate,
the label signal forming means is chemiluminescence producing means for bringing the chemiluminescence substrate into contact with the biochemical analysis unit, which has been processed by the specific binding means, the chemiluminescence substrate causing the labeling substance to produce chemiluminescence, the chemiluminescence produced by the labeling substance acting as the label signal, and
the read-out means photoelectrically detects the chemiluminescence, which is produced by the labeling substance, in order to form the data for a biochemical analysis.

8. A biochemical analysis system as defined in claim 1 wherein the biochemical analysis system further comprises spotting means for spotting each of the multiple kinds of the specific binding substances, which are capable of specifically binding to organism-originating substances and whose structures or characteristics are known, to one of different positions on or in the base plate in order to form the plurality of independent spot-shaped regions, and thereby preparing the biochemical analysis unit, and
third correspondence relationship specifying means for feeding spot information, which contains information representing the position of each of the spot-shaped regions, information representing the kind of the specific binding substance which forms each of the spot-shaped regions, and the like, and the identification information of the biochemical analysis unit into the analysis means such that the correspondence relationship between the spot information and the identification information of the biochemical analysis unit is clear.

9. A biochemical analysis system as defined in claim 1 wherein the identification information of the biochemical analysis unit contains identification information of the base plate.

10. A biochemical analysis unit, comprising a base plate and a data recording layer containing a data-rewritable recording medium, which data recording layer is formed on or in the base plate.

11. A biochemical analysis unit as defined in claim 10 wherein the base plate is provided with a plurality of spot-shaped regions, the spot-shaped regions having been formed by spotting specific binding substances, which are capable of specifically binding to organism-originating substances and whose base sequences, base lengths, compositions, and the like, are known, onto the base plate.

12. A biochemical analysis unit as defined in claim 10 wherein the data recording layer comprises a first data recording region, which is protected from data rewriting conducted by a user, and a second data recording region, on which the data is capable of being rewritten by the user.

13. A biochemical analysis unit as defined in claim 12 wherein data concerning kinds of specific binding substances and positions of spot-shaped regions of the base plate, each of which spot-shaped regions contains one of the specific binding substances, is recorded on the first data recording region of the data recording layer.

14. A biochemical analysis unit as defined in claim 12 wherein data concerning a number of times of use of the biochemical analysis unit is capable of being recorded on the first data recording region of the data recording layer.

15. A biochemical analysis unit as defined in claim 12 wherein data concerning a day of use of the biochemical analysis unit is capable of being recorded on the first data recording region and/or the second data recording region of the data recording layer.

16. A biochemical analysis unit as defined in claim 12 wherein data concerning a number of times of recycling of the biochemical analysis unit is capable of being recorded on the first data recording region of the data recording layer.

17. A biochemical analysis unit as defined in claim 10 wherein the base plate is provided with a plurality of adsorptive regions, which are located at a spacing from one another and in two-dimensional directions.

18. A biochemical analysis unit as defined in claim 17 wherein the base plate is provided with a plurality of holes, which are located at a spacing from one another and in two-dimensional directions, and
each of the plurality of the adsorptive regions is formed by filling an adsorptive material in one of the holes.

19. A biochemical analysis unit as defined in claim 18 wherein the base plate is provided with a plurality of through-holes, which are located at a spacing from one another and in two-dimensional directions, and
each of the plurality of the adsorptive regions is formed by press-fitting an adsorptive film, which contains an adsorptive material, into one of the plurality of the through-holes of the base plate.

20. A biochemical analysis unit as defined in claim 17 wherein the base plate is provided with a plurality of through-holes, which are located at a spacing from one another and in two-dimensional directions,
the biochemical analysis unit further comprises an adsorptive plate containing an adsorptive material,
the base plate is located on at least either one of two surfaces of the adsorptive plate such that the base plate is in close contact with the one surface of the adsorptive plate, and
each of the plurality of the adsorptive regions is constituted of a region of the adsorptive plate, which region is exposed within one of the plurality of the through-holes of the base plate.

21. A biochemical analysis unit as defined in claim 17 wherein the base plate is provided with a plurality of recesses, which are located at a spacing from one another and in two-dimensional directions, and
each of the plurality of the adsorptive regions is constituted of an adsorptive material layer, which is formed on an inner wall surface of one of the plurality of the recesses of the base plate.

22. A biochemical analysis unit as defined in claim 17 wherein the plurality of the adsorptive regions is formed on a surface of the base plate.

23. A biochemical analysis unit as defined in claim 17 wherein the base plate is provided with a plurality of protrusions, which are located at a spacing from one another and in two-dimensional directions, and
each of the plurality of the adsorptive regions is formed in the vicinity of a top end of one of the plurality of the protrusions of the base plate.

24. A biochemical analysis unit as defined in claim 17 wherein the base plate has radiation attenuating properties.

25. A biochemical analysis unit as defined in claim 17 wherein the base plate has light attenuating properties.

26. A biochemical analysis unit as defined in claim 12 wherein data concerning a plurality of adsorptive regions of the base plate is capable of being recorded on the first data recording region of the data recording layer.

27. A method of managing a biochemical analysis unit comprising a plurality of adsorptive regions located at a spacing from one another, each of the plurality of the adsorptive regions containing one of specific binding substances, whose structures or characteristics are known, the method comprising the step of:
recording management data that contains data concerning time acting as reference time for a calculation of an attenuation period, at which radiation from a radioactive labeling substance attenuates, on the biochemical analysis unit when a biochemical analysis is executed by use of the biochemical analysis unit.

28. A method of managing a biochemical analysis unit as defined in claim 27 wherein, when an organism-originating substance, which has been labeled with a radioactive labeling substance, is subjected by a hybridization apparatus to selective hybridization with the specific binding substances, each of which is contained in one of the plurality of the adsorptive regions of the biochemical analysis unit,
the management data that contains the data concerning the time acting as the reference time for the calculation of the attenuation period, at which the radiation from the radioactive labeling substance attenuates, is recorded on the biochemical analysis unit by the hybridization apparatus.

29. A method of managing a biochemical analysis unit as defined in claim 27 wherein, in cases where an organism-originating substance, which has been labeled with a radioactive labeling substance, is subjected by a hybridization apparatus to selective hybridization with the specific binding substances, each of which is contained in one of the plurality of the adsorptive regions of the biochemical analysis unit, and an exposure operation is thereafter executed with an exposure apparatus by superposing the biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective hybridization,
the management data that contains the data concerning the time acting as the reference time for the calculation of the attenuation period, at which the radiation from the radioactive labeling substance attenuates, is recorded on the biochemical analysis unit by the exposure apparatus when the exposure operation is executed.

30. A method of managing a biochemical analysis unit as defined in claim 27 wherein the data concerning the time acting as the reference time for the calculation of the attenuation period, at which the radiation from the radioactive labeling substance attenuates, contains data concerning a date and hour of formation of the radioactive labeling substance.

31. A method of managing a biochemical analysis unit as defined in claim 27 wherein the data concerning the time acting as the reference time for the calculation of the attenuation period, at which the radiation from the radioactive labeling substance attenuates, contains data concerning a day of formation of the radioactive labeling substance.

32. A method of managing a biochemical analysis unit as defined in claim 27 wherein the biochemical analysis unit further comprises a base plate provided with a plurality of holes, which are located at a spacing from one another,
each of the plurality of the adsorptive regions is formed by filling an adsorptive material in one of the holes of the base plate, and applying each of the specific binding substances to the adsorptive material, which has been filled in one of the holes of the base plate, and
the management data is recorded on the base plate of the biochemical analysis unit.

33. A method of managing a biochemical analysis unit as defined in claim 27 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is constituted of a region of the adsorptive plate, which region is exposed within one of the plurality of the through-holes of the base plate, and
the management data is recorded on the base plate of the biochemical analysis unit.

34. A method of managing a biochemical analysis unit as defined in claim 32 wherein the management data is recorded as visible data on the base plate of the biochemical analysis unit.

35. A method of managing a biochemical analysis unit as defined in claim 32 wherein the base plate of the biochemical analysis unit is provided with a magnetic recording layer, and the management data is magnetically recorded on the magnetic recording layer.

36. A method of managing a biochemical analysis unit as defined in claim 32 wherein the base plate of the biochemical analysis unit has radiation attenuating properties.

37. A method of managing a biochemical analysis unit as defined in claim 27 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate, and
the management data is recorded on the adsorptive plate of the biochemical analysis unit.

38. A method of managing a biochemical analysis unit as defined in claim 37 wherein the management data is recorded as visible data on the adsorptive plate of the biochemical analysis unit.

39. A method of managing a biochemical analysis unit as defined in claim 27 wherein, besides the data concerning the time acting as the reference time for the calculation of the attenuation period, at which the radiation from the radioactive labeling substance attenuates, the management data further contains data concerning a nuclide of the radioactive labeling substance.

40. A method of managing a biochemical analysis unit as defined in claim 39 wherein a scrapping period, at which the biochemical analysis unit is capable of being scrapped, is determined in accordance with the data concerning the time acting as the reference time for the calculation of the attenuation period, at which the radiation from the radioactive labeling substance attenuates, and in accordance with the data concerning the nuclide of the radioactive labeling substance, and
the biochemical analysis unit is sorted and managed in accordance with the scrapping period, at which the biochemical analysis unit is capable of being scrapped.

41. A system for managing a biochemical analysis unit comprising a plurality of adsorptive regions located at a spacing from one another, each of the plurality of the adsorptive regions containing one of specific binding substances, whose structures or characteristics are known, the system comprising:
a management data recording apparatus for recording management data that contains data concerning time acting as reference time for a calculation of an attenuation period, at which radiation from a radioactive labeling substance attenuates, on the biochemical analysis unit.

42. A system for managing a biochemical analysis unit as defined in claim 41 wherein the system further comprises a hybridization apparatus for subjecting an organism-originating substance, which has been labeled with a radioactive labeling substance, to selective hybridization with the specific binding substances, each of which is contained in one of the plurality of the adsorptive regions of the biochemical analysis unit, and
the hybridization apparatus is provided with the management data recording apparatus.

43. A system for managing a biochemical analysis unit as defined in claim 41 wherein the system further comprises an exposure apparatus for:
receiving the biochemical analysis unit comprising the plurality of the adsorptive regions, each of the plurality of the adsorptive regions containing one of the specific binding substances, whose structures or characteristics are known, an organism-originating substance, which has been labeled with at least the radioactive labeling substance, having been subjected to selective hybridization with the specific binding substances, each of which is contained in one of the plurality of the adsorptive regions of the biochemical analysis unit, and
executing an exposure operation by superposing the received biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective hybridization, and
the exposure apparatus is provided with the management data recording apparatus.

44. A system for managing a biochemical analysis unit as defined in claim 41 wherein the data concerning the time acting as the reference time for the calculation of the attenuation period, at which the radiation from the radioactive labeling substance attenuates, contains data concerning a date and hour of formation of the radioactive labeling substance.

45. A system for managing a biochemical analysis unit as defined in claim 41 wherein the data concerning the time acting as the reference time for the calculation of the attenuation period, at which the radiation from the radioactive labeling substance attenuates, contains data concerning a day of formation of the radioactive labeling substance.

46. A system for managing a biochemical analysis unit as defined in claim 41 wherein the biochemical analysis unit further comprises a base plate provided with a plurality of holes, which are located at a spacing from one another,
each of the plurality of the adsorptive regions is formed by filling an adsorptive material in one of the holes of the base plate, and applying each of the specific binding substances to the adsorptive material, which has been filled in one of the holes of the base plate, and
the management data recording apparatus records the management data on the base plate of the biochemical analysis unit.

47. A system for managing a biochemical analysis unit as defined in claim 41 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is constituted of a region of the adsorptive plate, which region is exposed within one of the plurality of the through-holes of the base plate, and
the management data recording apparatus records the management data on the base plate of the biochemical analysis unit.

48. A system for managing a biochemical analysis unit as defined in claim 46 wherein the management data recording apparatus records the management data as visible data on the base plate of the biochemical analysis unit.

49. A system for managing a biochemical analysis unit as defined in claim 46 wherein the base plate of the biochemical analysis unit is provided with a magnetic recording layer, and the management data recording apparatus magnetically records the management data on the magnetic recording layer.

50. A system for managing a biochemical analysis unit as defined in claim 41 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate, and
the management data recording apparatus records the management data on the adsorptive plate of the biochemical analysis unit.

51. A system for managing a biochemical analysis unit as defined in claim 50 wherein the management data recording apparatus records the management data as visible data on the adsorptive plate of the biochemical analysis unit.

52. A system for managing a biochemical analysis unit as defined in claim 41 wherein, besides the data concerning the time acting as the reference time for the calculation of the attenuation period, at which the radiation from the radioactive labeling substance attenuates, the management data further contains data concerning a nuclide of the radioactive labeling substance.

53. A system for managing a biochemical analysis unit as defined in claim 52 wherein the system further comprises a biochemical analysis unit sorting apparatus for:
determining a scrapping period, at which the biochemical analysis unit is capable of being scrapped, in accordance with the data concerning the time acting as the reference time for the calculation of the attenuation period, at which the radiation from the radioactive labeling substance attenuates, and in accordance with the data concerning the nuclide of the radioactive labeling substance, and
sorting and managing the biochemical analysis unit in accordance with the scrapping period, at which the biochemical analysis unit is capable of being scrapped.

54. A method of managing a biochemical analysis medium, comprising the steps of:
i) recording management data on a biochemical analysis medium, the management data containing data concerning time acting as reference time for a calculation of an attenuation period, at which radiation from a radioactive labeling substance attenuates, and data concerning a nuclide of the radioactive labeling substance,
ii) executing a biochemical analysis by use of the biochemical analysis medium, on which the management data has been recorded,
iii) reading out the management data, which has been recorded on the biochemical analysis medium, from the biochemical analysis medium after the biochemical analysis has been executed,
iv) calculating a scrapping period, at which a level of radioactivity of the radioactive labeling substance contained in the biochemical analysis medium decreases to a level equal to at most a level that allows the biochemical analysis medium to be scrapped, in accordance with the management data having been read out,
v) sorting the biochemical analysis medium in accordance with the scrapping period,
vi) accommodating the biochemical analysis medium in a storage box in accordance with the scrapping period, and
vii) storing and managing the biochemical analysis medium, which has been accommodated in the storage box.

55. A method of managing a biochemical analysis medium as defined in claim 54 wherein the scrapping period of the biochemical analysis medium, which has been accommodated in the storage box, is indicated on the storage box.

56. A method of managing a biochemical analysis medium as defined in claim 54 wherein the biochemical analysis medium is constituted of a biochemical analysis unit comprising a plurality of adsorptive regions located at a spacing from one another, each of the plurality of the adsorptive regions containing one of specific binding substances, whose structures or characteristics are known,
an organism-originating substance, which has been labeled with at least the radioactive labeling substance, is subjected by a hybridization apparatus to hybridization with the specific binding substances, each of which is contained in one of the plurality of the adsorptive regions of the biochemical analysis unit, and
the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the hybridization apparatus when the hybridization is executed.

57. A method of managing a biochemical analysis medium as defined in claim 56 wherein the biochemical analysis unit further comprises a base plate provided with a plurality of holes,
each of the plurality of the adsorptive regions is formed by filling an adsorptive material in one of the holes of the base plate, and applying each of the specific binding substances to the adsorptive material, which has been filled in one of the holes of the base plate,
the organism-originating substance, which has been labeled with at least the radioactive labeling substance, is subjected by the hybridization apparatus to the hybridization with the specific binding substances, each of which is contained in one of the plurality of the adsorptive regions of the biochemical analysis unit, and
the management data that contains the data concerning the time acting as the reference time for the calculation of the attenuation period, at which the radiation from the radioactive labeling substance attenuates, and the data concerning the nuclide of the radioactive labeling substance is recorded on the base plate of the biochemical analysis unit by the hybridization apparatus when the hybridization is executed.

58. A method of managing a biochemical analysis medium as defined in claim 56 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is formed by applying one of the specific binding substances to a region of the adsorptive plate, which region is exposed within one of the plurality of the through-holes of the base plate,
the organism-originating substance, which has been labeled with at least the radioactive labeling substance, is subjected by the hybridization apparatus to the hybridization with the specific binding substances, each of which is contained in one of the plurality of the adsorptive regions of the biochemical analysis unit, and
the management data that contains the data concerning the time acting as the reference time for the calculation of the attenuation period, at which the radiation from the radioactive labeling substance attenuates, and the data concerning the nuclide of the radioactive labeling substance is recorded on the base plate of the biochemical analysis unit by the hybridization apparatus when the hybridization is executed.

59. A method of managing a biochemical analysis medium as defined in claim 56 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate,
the organism-originating substance, which has been labeled with at least the radioactive labeling substance, is subjected by the hybridization apparatus to the hybridization with the specific binding substances, each of which is contained in one of the plurality of the adsorptive regions of the biochemical analysis unit, and
the management data that contains the data concerning the time acting as the reference time for the calculation of the attenuation period, at which the radiation from the radioactive labeling substance attenuates, and the data concerning the nuclide of the radioactive labeling substance is recorded on the adsorptive plate of the biochemical analysis unit by the hybridization apparatus when the hybridization is executed.

60. A method of managing a biochemical analysis medium as defined in claim 54 wherein the biochemical analysis medium is constituted of a biochemical analysis unit comprising a plurality of adsorptive regions located at a spacing from one another, each of the plurality of the adsorptive regions containing one of specific binding substances, whose structures or characteristics are known,
an organism-originating substance, which has been labeled with at least the radioactive labeling substance, is subjected to selective, specific binding to the specific binding substances, each of which is contained in one of the plurality of the adsorptive regions of the biochemical analysis unit,
an exposure operation is thereafter executed with an exposure apparatus by superposing the biochemical analysis unit and a stimulable phosphor sheet, which comprises a stimulable phosphor layer containing a stimulable phosphor, one upon the other, the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and
the management data that contains the data concerning the time acting as the reference time for the calculation of the attenuation period, at which the radiation from the radioactive labeling substance attenuates, and the data concerning the nuclide of the radioactive labeling substance is recorded on the biochemical analysis unit by the exposure apparatus when the exposure operation is executed.

61. A method of managing a biochemical analysis medium as defined in claim 60 wherein the biochemical analysis unit further comprises a base plate provided with a plurality of holes,
each of the plurality of the adsorptive regions is formed by filling an adsorptive material in one of the holes of the base plate, and applying each of the specific binding substances to the adsorptive material, which has been filled in one of the holes of the base plate,
the organism-originating substance, which has been labeled with at least the radioactive labeling substance, is subjected to the selective, specific binding to the specific binding substances, each of which is contained in one of the plurality of the adsorptive regions of the biochemical analysis unit,
the exposure operation is thereafter executed with the exposure apparatus by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the management data that contains the data concerning the time acting as the reference time for the calculation of the attenuation period, at which the radiation from the radioactive labeling substance attenuates, and the data concerning the nuclide of the radioactive labeling substance is recorded on the base plate of the biochemical analysis unit by the exposure apparatus when the exposure operation is executed.

62. A method of managing a biochemical analysis medium as defined in claim 60 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is formed by applying one of the specific binding substances to a region of the adsorptive plate, which region is exposed within one of the plurality of the through-holes of the base plate,
the organism-originating substance, which has been labeled with at least the radioactive labeling substance, is subjected to the selective, specific binding to the specific binding substances, each of which is contained in one of the plurality of the adsorptive regions of the biochemical analysis unit,
the exposure operation is thereafter executed with the exposure apparatus by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the management data that contains the data concerning the time acting as the reference time for the calculation of the attenuation period, at which the radiation from the radioactive labeling substance attenuates, and the data concerning the nuclide of the radioactive labeling substance is recorded on the base plate of the biochemical analysis unit by the exposure apparatus when the exposure operation is executed.

63. A method of managing a biochemical analysis medium as defined in claim 60 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate,
the organism-originating substance, which has been labeled with at least the radioactive labeling substance, is subjected to the selective, specific binding to the specific binding substances, each of which is contained in one of the plurality of the adsorptive regions of the biochemical analysis unit,
the exposure operation is thereafter executed with the exposure apparatus by superposing the biochemical analysis unit and the stimulable phosphor sheet, which comprises the stimulable phosphor layer containing the stimulable phosphor, one upon the other, the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the management data that contains the data concerning the time acting as the reference time for the calculation of the attenuation period, at which the radiation from the radioactive labeling substance attenuates, and the data concerning the nuclide of the radioactive labeling substance is recorded on the adsorptive plate of the biochemical analysis unit by the exposure apparatus when the exposure operation is executed.

64. A method of managing a biochemical analysis medium as defined in claim 57 wherein the base plate of the biochemical analysis unit is provided with a data recording layer, and
the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the base plate of the biochemical analysis unit.

65. A method of managing a biochemical analysis medium as defined in claim 57 wherein the base plate of the biochemical analysis unit has radiation attenuating properties.

66. A method of managing a biochemical analysis medium as defined in claim 54 wherein the biochemical analysis medium is constituted of a stimulable phosphor sheet comprising a support and a stimulable phosphor layer containing a stimulable phosphor, which stimulable phosphor layer is formed on or in the support,
a biochemical analysis unit comprising a plurality of adsorptive regions, each of the plurality of the adsorptive regions containing one of specific binding substances, whose structures or characteristics are known, is obtained, an organism-originating substance, which has been labeled with at least the radioactive labeling substance, having been subjected to selective, specific binding to the specific binding substances, each of which is contained in one of the plurality of the adsorptive regions of the biochemical analysis unit,
an exposure operation is executed with an exposure apparatus by superposing the biochemical analysis unit and the stimulable phosphor sheet one upon the other, the stimulable phosphor contained in the stimulable phosphor layer formed on or in the support of the stimulable phosphor sheet being thereby exposed to radiation radiated out from the radioactive labeling substance, which is contained selectively in at least one adsorptive region among the plurality of the adsorptive regions of the biochemical analysis unit as a result of the selective, specific binding, and
the management data that contains the data concerning the time acting as the reference time for the calculation of the attenuation period, at which the radiation from the radioactive labeling substance attenuates, and the data concerning the nuclide of the radioactive labeling substance is recorded on the support of the stimulable phosphor sheet by the exposure apparatus when the exposure operation is executed.

67. A method of managing a biochemical analysis medium as defined in claim 66 wherein the stimulable phosphor layer comprises a plurality of stimulable phosphor layer regions, which are located at a spacing from one another.

68. A method of managing a biochemical analysis medium as defined in claim 66 wherein the support of the stimulable phosphor sheet is provided with a data recording layer, on which data is capable of being recorded, and
the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the support of the stimulable phosphor sheet by the exposure apparatus.

69. A method of managing a biochemical analysis medium as defined in claim 66 wherein the support of the stimulable phosphor sheet has radiation attenuating properties.

70. A system for managing a biochemical analysis medium, comprising:
i) a radioactive label imparting apparatus for imparting a radioactive label to a biochemical analysis medium by use of a radioactive labeling substance, and
ii) a biochemical analysis medium sorting apparatus for sorting the biochemical analysis medium, which has been labeled with the radioactive labeling substance and has been used for a biochemical analysis,
wherein the radioactive label imparting apparatus is constituted to record management data on the biochemical analysis medium, the management data containing data concerning time acting as reference time for a calculation of an attenuation period, at which radiation from the radioactive labeling substance attenuates, and data concerning a nuclide of the radioactive labeling substance, and
the biochemical analysis medium sorting apparatus is constituted to perform the operations for:
reading out the management data, which has been recorded on the biochemical analysis medium, from the biochemical analysis medium,
calculating a scrapping period, at which a level of radioactivity of the radioactive labeling substance contained in the biochemical analysis medium decreases to a level equal to at most a level that allows the biochemical analysis medium to be scrapped, in accordance with the management data having been read out,
sorting the biochemical analysis medium in accordance with the scrapping period, and
accommodating the biochemical analysis medium in a storage box in accordance with the scrapping period.

71. A system for managing a biochemical analysis medium as defined in claim 70 wherein the scrapping period of the biochemical analysis medium, which has been accommodated in the storage box, is indicated on the storage box.

72. A system for managing a biochemical analysis medium as defined in claim 70 wherein the biochemical analysis medium is constituted of a biochemical analysis unit comprising a plurality of adsorptive regions located at a spacing from one another, each of the plurality of the adsorptive regions containing one of specific binding substances, whose structures or characteristics are known,
the radioactive label imparting apparatus is constituted of a hybridization apparatus for subjecting an organism-originating substance, which has been labeled with at least the radioactive labeling substance, to hybridization with the specific binding substances, each of which is contained in one of the plurality of the adsorptive regions of the biochemical analysis unit, and
the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit when the hybridization of the organism-originating substance, which has been labeled with at least the radioactive labeling substance, with the specific binding substances, each of which is contained in one of the plurality of the adsorptive regions of the biochemical analysis unit, is executed.

73. A system for managing a biochemical analysis medium as defined in claim 72 wherein the biochemical analysis unit further comprises a base plate provided with a plurality of holes,
each of the plurality of the adsorptive regions is formed by filling an adsorptive material in one of the holes of the base plate, and applying each of the specific binding substances to the adsorptive material, which has been filled in one of the holes of the base plate, and
the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the base plate of the biochemical analysis unit.

74. A system for managing a biochemical analysis medium as defined in claim 72 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is formed by applying one of the specific binding substances to a region of the adsorptive plate, which region is exposed within one of the plurality of the through-holes of the base plate, and
the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the base plate of the biochemical analysis unit.

75. A system for managing a biochemical analysis medium as defined in claim 72 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate, and
the hybridization apparatus records the management data, the management data containing the data concerning the time acting as the reference time for the calculation of the attenuation period, at which the radiation from the radioactive labeling substance attenuates, and the data concerning the nuclide of the radioactive labeling substance, on the adsorptive plate of the biochemical analysis unit.

76. A system for managing a biochemical analysis medium as defined in claim 70 wherein the biochemical analysis medium is constituted of a biochemical analysis unit comprising a plurality of adsorptive regions located at a spacing from one another, each of the plurality of the adsorptive regions containing one of specific binding substances, whose structures or characteristics are known,
the radioactive label imparting apparatus is constituted of an exposure apparatus for:
receiving the biochemical analysis unit comprising the plurality of the adsorptive regions, each of the plurality of the adsorptive regions containing one of the specific binding substances, whose structures or characteristics are known, an organism-originating substance, which has been labeled with at least the radioactive labeling substance, having been subjected to selective, specific binding to the specific binding substances, each of which is contained in one of the plurality of the adsorptive regions of the biochemical analysis unit, and
executing an exposure operation by superposing the received biochemical analysis unit and a stimulable phosphor sheet, which comprises a stimulable phosphor layer containing a stimulable phosphor, one upon the other, the stimulable phosphor contained in the stimulable phosphor layer of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the biochemical analysis unit when the exposure operation for exposing the stimulable phosphor contained in the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

77. A system for managing a biochemical analysis medium as defined in claim 76 wherein the biochemical analysis unit further comprises a base plate provided with a plurality of holes,
each of the plurality of the adsorptive regions is formed by filling an adsorptive material in one of the holes of the base plate, and applying each of the specific binding substances to the adsorptive material, which has been filled in one of the holes of the base plate,
the radioactive label imparting apparatus is constituted of the exposure apparatus for:
receiving the biochemical analysis unit comprising the plurality of the adsorptive regions, each of the plurality of the adsorptive regions containing one of the specific binding substances, whose structures or characteristics are known, the organism-originating substance, which has been labeled with at least the radioactive labeling substance, having been subjected to the selective, specific binding to the specific binding substances, each of which is contained in one of the plurality of the adsorptive regions of the biochemical analysis unit, and
executing the exposure operation by superposing the received biochemical analysis unit and the stimulable phosphor sheet, which comprises the stimulable phosphor layer containing the stimulable phosphor, one upon the other, the stimulable phosphor contained in the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the base plate of the biochemical analysis unit when the exposure operation for exposing the stimulable phosphor contained in the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

78. A system for managing a biochemical analysis medium as defined in claim 76 wherein the biochemical analysis unit further comprises a base plate, which has radiation attenuating properties and is provided with a plurality of through-holes, and an adsorptive plate made from an adsorptive material, the base plate being located on at least either one of two surfaces of the adsorptive plate such that the base plate is in close contact with the one surface of the adsorptive plate,
each of the plurality of the adsorptive regions is formed by applying one of the specific binding substances to a region of the adsorptive plate, which region is exposed within one of the plurality of the through-holes of the base plate,
the radioactive label imparting apparatus is constituted of the exposure apparatus for:
receiving the biochemical analysis unit comprising the plurality of the adsorptive regions, each of the plurality of the adsorptive regions containing one of the specific binding substances, whose structures or characteristics are known, the organism-originating substance, which has been labeled with at least the radioactive labeling substance, having been subjected to the selective, specific binding to the specific binding substances, each of which is contained in one of the plurality of the adsorptive regions of the biochemical analysis unit, and
executing the exposure operation by superposing the received biochemical analysis unit and the stimulable phosphor sheet, which comprises the stimulable phosphor layer containing the stimulable phosphor, one upon the other, the stimulable phosphor contained in the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the base plate of the biochemical analysis unit when the exposure operation for exposing the stimulable phosphor contained in the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

79. A system for managing a biochemical analysis medium as defined in claim 76 wherein the biochemical analysis unit further comprises an adsorptive plate made from an adsorptive material,
each of the plurality of the adsorptive regions is formed with a process for spotting a liquid containing one of the specific binding substances, whose structures or characteristics are known, onto the adsorptive plate,
the radioactive label imparting apparatus is constituted of the exposure apparatus for:
receiving the biochemical analysis unit comprising the plurality of the adsorptive regions, each of the plurality of the adsorptive regions containing one of the specific binding substances, whose structures or characteristics are known, the organism-originating substance, which has been labeled with at least the radioactive labeling substance, having been subjected to the selective, specific binding to the specific binding substances, each of which is contained in one of the plurality of the adsorptive regions of the biochemical analysis unit, and
executing the exposure operation by superposing the received biochemical analysis unit and the stimulable phosphor sheet, which comprises the stimulable phosphor layer containing the stimulable phosphor, one upon the other, the stimulable phosphor contained in the stimulable phosphor layer of the stimulable phosphor sheet being thereby exposed to the radiation radiated out from the radioactive labeling substance, which is now contained selectively in at least one adsorptive region among the plurality of the adsorptive regions of the biochemical analysis unit as a result of the selective, specific binding, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the adsorptive plate of the biochemical analysis unit when the exposure operation for exposing the stimulable phosphor contained in the stimulable phosphor layer of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

80. A system for managing a biochemical analysis medium as defined in claim 73 wherein the base plate of the biochemical analysis unit is provided with a data recording layer, and
the management data that contains the data concerning the time acting as the reference time for the calculation of the attenuation period, at which the radiation from the radioactive labeling substance attenuates, and the data concerning the nuclide of the radioactive labeling substance is recorded on the data recording layer of the base plate of the biochemical analysis unit.

81. A system for managing a biochemical analysis medium as defined in claim 70 wherein the biochemical analysis medium is constituted of a stimulable phosphor sheet comprising a support and a stimulable phosphor layer containing a stimulable phosphor, which stimulable phosphor layer is formed on or in the support,
the radioactive label imparting apparatus is constituted of an exposure apparatus for:
receiving a biochemical analysis unit comprising a plurality of adsorptive regions, each of the plurality of the adsorptive regions containing one of specific binding substances, whose structures or characteristics are known, an organism-originating substance, which has been labeled with at least the radioactive labeling substance, having been subjected to selective, specific binding to the specific binding substances, each of which is contained in one of the plurality of the adsorptive regions of the biochemical analysis unit, and
executing an exposure operation by superposing the received biochemical analysis unit and the stimulable phosphor sheet one upon the other, the stimulable phosphor contained in the stimulable phosphor layer formed on or in the support of the stimulable phosphor sheet being thereby exposed to radiation radiated out from the radioactive labeling substance, which is now contained selectively in at least one adsorptive region among the plurality of the adsorptive regions of the biochemical analysis unit as a result of the selective, specific binding, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the support of the stimulable phosphor sheet when the exposure operation for exposing the stimulable phosphor contained in the stimulable phosphor layer formed on or in the support of the stimulable phosphor sheet to the radiation radiated out from the radioactive labeling substance, which is now contained selectively in at least one adsorptive region among the plurality of the adsorptive regions of the biochemical analysis unit as a result of the selective, specific binding, is executed.

82. A system for managing a biochemical analysis medium as defined in claim 81 wherein the stimulable phosphor layer comprises a plurality of stimulable phosphor layer regions, which are located at a spacing from one another.

83. A system for managing a biochemical analysis medium as defined in claim 82 wherein the support of the stimulable phosphor sheet is provided with a data recording layer, on which data is capable of being recorded, and
the exposure apparatus records the management data, the management data containing the data concerning the time acting as the reference time for the calculation of the attenuation period, at which the radiation from the radioactive labeling substance attenuates, and the data concerning the nuclide of the radioactive labeling substance, on the data recording layer of the support of the stimulable phosphor sheet.
